(19) United States
(12) Patent Application Publication
(10) Pub. No.: US 2026/0218238 A1
(43) Pub. Date: Jul. 30, 2026

(54) VIRAL OPEN READING FRAMES, USES THEREOF, AND METHODS OF DETECTING THE SAME

(71) Applicants: The Broad Institute, Inc., Cambridge, MA (US); President and Fellows of Harvard College, Cambridge, MA (US)

(72) Inventors: Pardis Sabeti, Cambridge, MA (US); Shira Weingarten-Gabbay, Cambridge, MA (US); Matthew Bauer, Cambridge, MA (US); Alexandra Stanton, Cambridge, MA (US)

(21) Appl. No.: 19/573,370

(22) Filed: Mar. 20, 2026

Related U.S. Application Data
(63) Continuation of application No. PCT/US2024/048478, filed on Sep. 25, 2024.
(60) Provisional application No. 63/540,279, filed on Sep. 25, 2023.

(51) Int. Cl. C12N 15/86; A61K 39/29; C12N 15/10

(52) U.S. Cl. CPC C12N 15/86; A61K 39/292; C12N 15/1058; C12N 15/1089; C12N 2730/10143

(57) ABSTRACT

Described in certain example embodiments herein are compositions, such as immunogenic compositions that can contain or more viral polynucleotides and/or polypeptides. In some embodiments, the one or more viral polynucleotide is a non-canonical viral open reading frame (ORF). Also described herein are methods to identify one or more viral polynucleotides and/or polypeptides such as a non-canonical viral ORF, such as massively parallel ribosome profiling.

### CROSS-REFERENCE TO RELATED APPLICATIONS

This application is a continuation application of International Patent Application No. PCT/US2024/048478, filed Sep. 25, 2024, which claims the benefit of and priority to U.S. Provisional Patent Application No. 63/540,279, filed on Sep. 25, 2023, the contents of which is incorporated by reference herein in its entirety.

### STATEMENT REGARDING FEDERALLY SPONSORED RESEARCH

This invention was made with government support under Grant No. U19AI110818 awarded by the National Institutes of Health and 58-3022-2-031 awarded by the United States Department of Agriculture. The government has certain rights in the invention.

### SEQUENCE LISTING

This application contains a sequence listing filed in electronic form as an xml file entitled “BROD_5825US_ST26.xml”, created on Feb. 20, 2026, and having a size of 25,189,988 bytes. The content of the sequence listing is incorporated herein in its entirety.

### TECHNICAL FIELD

The subject matter disclosed herein is generally directed to massively parallel methods and techniques for evaluating open reading frames in genomes, particularly viral genomes.

### BACKGROUND

Recent pandemics and epidemics, e.g., SARS-CoV2, Ebola, and Zika are stark reminders of the enormous impact of viruses on public health, emphasizing the great need for transformative vaccines and therapeutics. Despite many recent technological advances, systematic methods to investigate virology have lagged behind, so that many of the global principles underlying viral infection remain unknown. As such there exists a need for improved compositions and techniques for combating viral and other pathogens.

Citation or identification of any document in this application is not an admission that such a document is available as prior art to the present invention.

### SUMMARY

Described in certain embodiments herein are polynucleotides comprising one or more viral polynucleotides from SEQ ID NOS: 1-9890 or SEQ ID NOS: 9891-16277.

Described in certain example embodiments herein are polynucleotides and/or polypeptides identified using the method described herein.

Described in certain example embodiments herein are polynucleotides, such as engineered polynucleotides, comprising one or more viral polynucleotides selected from those polynucleotides identified by a method described herein and/or SEQ ID NOS: 1-9890 or SEQ ID NOS: 9891-16277.

Described in certain example embodiments herein are vectors comprising a polynucleotide of any one of the preceding paragraphs or as described elsewhere herein. In certain example embodiments, the vector is an expression vector. In certain example embodiments, the vector is a eukaryotic vector, prokaryotic vector, or viral vector.

Described in certain example embodiments herein are polypeptides encoded by a polynucleotide or vector as in any one of the preceding paragraphs or as described elsewhere herein.

Described in certain example embodiments herein are delivery vehicles comprising a polynucleotide, a vector, and/or polypeptide as in any one of the preceding paragraphs or as described elsewhere herein. In certain example embodiments, the delivery vehicle comprises a particle, lipid particle, liposome, exosome, virus particle, virus-like particle, capsid, cell penetrating peptide, DNA nanoclew, supercharged protein, self-assembling nanoparticle, spherical nucleic acid, streptolysin, lipoplex, polyplex, sugar-based particle, stable nucleic-acid particles, mRNA vaccine, and any combination thereof.

Described in certain example embodiments herein are cells comprising a polynucleotide, a vector, polypeptide, and/or delivery vehicle as in any one of the preceding paragraphs or as described elsewhere herein.

Described in certain example embodiments herein are immunogenic compositions comprising a polynucleotide, a vector, polypeptide, delivery vehicle, and/or cell as in any one of the preceding paragraphs or as described elsewhere herein. In certain example embodiments, the polynucleotide and/or the polypeptide of the immunogenic composition is capable of stimulating a B-cell and/or T-cell response in a subject to which it is delivered. In certain example embodiments, the B-cell response comprises antibody production.

Described in certain example embodiments herein are therapeutic compositions comprising an immunogenic composition as in any one of the preceding paragraphs or as described elsewhere herein; and an anti-viral therapeutic. In certain example embodiments, the one or more polynucleotides is a synthetic mRNA vaccine.

Described in certain example embodiments herein are formulations comprising a polynucleotide, a vector, polypeptide, delivery vehicle, cell, and/or immunogenic composition as in any one of the preceding paragraphs or as described elsewhere herein, and a pharmaceutically acceptable carrier.

Described in certain example embodiments herein are methods of inducing a B-cell response and/or T-cell response to a virus in a subject in need thereof, comprising administering, to the subject, the immunogenic composition or the therapeutic composition, or a pharmaceutical formulation thereof as in any one of the preceding paragraphs or as described elsewhere herein. In certain example embodiments, the B cell response comprises antibody production.

Described in certain example embodiments herein are methods of treating a viral infection in a subject in need thereof comprising administering, to the subject in need thereof, the immunogenic composition or the therapeutic composition, or a pharmaceutical formulation thereof as in any one of the preceding paragraphs or as described elsewhere herein in combination with an antiviral therapeutic.

Described in certain example embodiments herein are methods an infection status of a subject comprising contacting immune cells derived from a subject with the immunogenic composition or a pharmaceutical formulation thereof as in any one of the preceding paragraphs or as described elsewhere herein; and detecting cross-reactivity of the immune cells to the immunogenic composition.

Described in certain example embodiments herein are methods of massively parallel antigen profiling comprising delivering to and expressing in a plurality of cells a pan-genomic library or a polynucleotide as in any one of the preceding paragraphs or as described elsewhere herein, wherein the pan-genomic library comprises a plurality of synthetic library expression constructs, each comprising a short synthetic polynucleotide and a pair of constant primers flanking the short synthetic polynucleotide, wherein the short synthetic polynucleotide has a sequence corresponding to a region of a genome; and determining the antigens presented by the cells. In certain example embodiments, delivery comprises infecting the plurality of cells with viral particles comprising the pan-genomic library or a polynucleotide as in any one of the preceding paragraphs or as described elsewhere herein. In certain example embodiments, determining the antigens comprises protein sequencing, mass spectrometry, Raman spectroscopy, immunodetection, chromatography, centrifugation, isoelectric focusing, or any combination thereof. In certain example embodiments, determining the antigens comprises isolating MHC complexes from the cells and detecting peptides loaded in the MHC, wherein the MHC is optionally an HLA. In certain example embodiments, the method further comprises evaluating an immune response to the antigens presented.

Described in certain example embodiments herein are vectors comprising a synthetic library expression construct comprising: a short synthetic polynucleotide; a pair of constant primers flanking the short synthetic polynucleotide, the pair of constant primers comprising a forward constant primer and a reverse constant primer, wherein the forward constant primer and the reverse constant primer are each independently coupled to the 5′ end or the 3′ end of the short synthetic polynucleotide; a stop codon polynucleotide comprising one or more stop codons, wherein the stop codon polynucleotide is coupled to the constant primer coupled to the 3′ end of the short synthetic polynucleotide such that the constant primer coupled to the 3′ end of the short synthetic polynucleotide is between the multiple stop codon polynucleotide and the short synthetic polynucleotide; a poly A signal, wherein the poly A signal is operably coupled to the short synthetic polynucleotide, the pair of constant primers, and the stop codon polynucleotide; and a promoter, wherein the promoter is operably coupled to the short synthetic polynucleotide, the pair of constant primers, and the stop codon polynucleotide, and the poly A signal. In certain example embodiments, the short synthetic polynucleotide is about 200 nucleotides. In certain example embodiments, the short synthetic polynucleotide has a sequence corresponding to a region of a genome. In certain example embodiments, the short synthetic polynucleotide has a sequence corresponding to a region of a human genome, a non-human animal genome, a plant genome, a microbial genome, or a viral genome. In certain example embodiments, the short synthetic polynucleotide has a sequence corresponding to a (a) an annotated region of a genome; (b) an unannotated region of a genome; (c) a mutation; (d) a 5′UTR; (e) a 3′UTR; (f) an open reading frame; or (g) any combination thereof. In certain example embodiments, the short synthetic polynucleotide (a) is a sequence according to a sequence in SEQ ID NOS: 1-9890 or SEQ ID NOS: 9891-16277; (b) is a synthetic in silico designed sequence; (d) does not have 100 percent identity to a genome sequence, optionally a human genome, a non-human animal genome, a plant genome, a microbial genome, or a viral genome; (e) is not native to any genome, optionally a human genome, a non-human animal genome, a plant genome, a microbial genome, or a viral genome; or (f) any combination thereof. In certain example embodiments, the vector comprises two or more synthetic library expression constructs. In certain example embodiments, at least two of the two or more synthetic library expression constructs comprises a different short synthetic polynucleotide. In certain example embodiments, each of the two or more synthetic library expression constructs comprises a different short synthetic polynucleotide. In certain example embodiments, at least two of the two or more synthetic library constructs comprises the same short synthetic polynucleotide. In certain example embodiments, the vector further comprises an additional regulatory element, a reporter gene, an origin of replication, a cloning sites, an internal ribosome entry sites, a transcription termination sequence, an inverted terminal repeat, a long terminal repeats, a trans-activating response elements, a central polypurine tract, a Psi element, a Rev response element, a packaging protein gene, a polymerase gene, an envelope protein gene, a capsid protein gene, a Rep protein gene, a U3 element, a repeat element (R), a unique 5′ element (U5), an untranslated region stabilization element, or any combination thereof. In certain example embodiments, the vector further comprises a Woodchuck hepatitis virus post-transcriptional regulatory element (WPRE), wherein the WPRE is operably coupled to the short synthetic polynucleotide and the pair of constant primers. In certain example embodiments, the vector is a eukaryotic expression vector. In certain example embodiments, the vector is a viral expression vector. In certain example embodiments, the promoter is a constitutive promoter. In certain example embodiments, the promoter is an inducible promoter or a conditional promoter.

Described in certain example embodiments herein are vector libraries each comprising a plurality of vectors as in any one of the preceding paragraphs or as described elsewhere herein. In certain example embodiments, at least two or more of the vectors comprise different short synthetic polynucleotides. In certain example embodiments, each vector of the plurality of vectors comprise different short synthetic polynucleotides. In certain example embodiments, at least two of the vectors comprise the same short synthetic polynucleotides.

Described in certain example embodiments herein are cells comprising a vector or vector library as in any one of the preceding paragraphs or as described elsewhere herein. In certain example embodiments, the cell is a eukaryotic cell or a prokaryotic cell.

Described in certain example embodiments herein are high throughput methods of determining translated sequences, the method comprising expressing a vector as in any one of the preceding paragraphs or as described elsewhere herein or a vector library as in any one of the preceding paragraphs or as described elsewhere herein in one or more cells under conditions sufficient to produce translation products; obtaining a sample of ribosomes comprising ribosome-protected mRNA fragments (RPFs) from the cells; recovering ribosome footprints consisting essentially of the RPFs from the sample of ribosomes; and determining the sequence of the RPFs thereby identifying translated sequences. In certain example embodiments, determining the sequence of the RPFs comprises nucleotide sequencing. In certain example embodiments, sequencing comprises RNA sequencing. In certain example embodiments, sequencing comprises generating cDNA from the RPFs for form RPF cDNA and DNA sequencing the RPF cDNA. In certain example embodiments the method further comprises digesting unprotected mRNA prior to recovering ribosome footprints. In certain example embodiments, the method further comprises removing rRNA from the sample containing ribosomes.

Described in certain example embodiments herein are methods of determining translational regulation comprising determining a first set of translated sequences by a method comprising expressing a pan-genomic library in a first plurality of cells, wherein the pan-genomic library comprises a plurality of synthetic library expression constructs, each comprising a short synthetic polynucleotide and a pair of constant primers flanking the short synthetic polynucleotide, wherein the short synthetic polynucleotide has a sequence corresponding to a region of a genome; obtaining a sample of ribosomes comprising ribosome-protected mRNA fragments (RPFs) from the first plurality of cells; recovering ribosome footprints consisting essentially of the RPFs from the sample of ribosomes; and determining the sequence of the RPFs thereby identifying translated sequences to form the first set of translated sequences; determining a second set of translated sequences by a method comprising expressing the pan-genomic library in a second plurality of cells; applying a stress to the second plurality of cells; obtaining a sample of ribosomes comprising ribosome-protected mRNA fragments (RPFs) from the second plurality of cells; recovering ribosome footprints consisting essentially of the RPFs from the sample of ribosomes; and determining the sequence of the RPFs thereby identifying translated sequences to form the second set of translated sequences, whereby similarities and differences in the sequences of the first and the second set of translated sequences indicates sequences that are translationally regulated or regulate translation. In certain example embodiments, the short synthetic polynucleotide is about 200 nucleotides. In certain example embodiments, the short synthetic polynucleotide has a sequence corresponding to a region of a genome. In certain example embodiments, the short synthetic polynucleotide has a sequence corresponding to a region of a human genome, a non-human animal genome, a plant genome, a microbial genome, or a viral genome. In certain example embodiments, the short synthetic polynucleotide has a sequence corresponding to (a) an annotated region of a genome; (b) an unannotated region of a genome; (c) a mutation; (d) a 5′UTR; (e) a 3′UTR; (f) an open reading frame; or (g) any combination thereof. In certain example embodiments herein, the short synthetic polynucleotide (a) is a sequence according to a sequence in SEQ ID NOS: 1-9890 or SEQ ID NOS: 9891-16277; (b) is a synthetic in silico designed sequence; (d) does not have 100 percent identity to a genome sequence, optionally a human genome, a non-human animal genome, a plant genome, a microbial genome, or a viral genome; (e) is not native to any genome, optionally a human genome, a non-human animal genome, a plant genome, a microbial genome, or a viral genome; or (f) any combination thereof. In certain example embodiments herein, expressing a pan-genomic library in a first plurality of cells and expressing a pan-genomic library in a second plurality of cells comprises expressing one or more vectors as in any one of the preceding paragraphs or elsewhere herein or a vector library as in any one of the preceding paragraphs or elsewhere herein in the first plurality of cells and the second plurality of cells. In certain example embodiments, the stress is a small molecule agent, a biologic agent, a physical stress, a chemical stress, or any combination thereof. In certain example embodiments, the stress is arsenite.

Described in certain example embodiments herein are vectors comprising an optically active reporter construct comprising a test polynucleotide; a first optically active protein encoding polynucleotide, wherein the first optically active protein encoding polynucleotide is operatively coupled in-frame with the test polynucleotide; a second optically active protein encoding polynucleotide, wherein the second optically active protein is a different optically active protein that the first optically active protein; a promoter, wherein the test polynucleotide, the first optically active protein encoding polynucleotide, and the second optically active protein encoding polynucleotide are operatively coupled to the promoter; and an internal ribosomal entry site (IRES), wherein the IRES is between the first optically active protein encoding polynucleotide and the second optically active protein encoding polynucleotide. In certain example embodiments, the method further comprises one or more additional regulatory elements, one or more additional reporters, viral replication elements and/or encoding polynucleotides, viral packaging elements and/or encoding polynucleotides, viral envelope protein encoding polynucleotides, long-terminal repeats, viral poly or any combination thereof. In certain example embodiments, the promoter is a constitutive promoter or an inducible promoter. In certain example embodiments, the method further comprises one or more untranslated regions (UTRs), wherein the one or more untranslated regions are operatively coupled at the 5′ and/or 3′ end of the test polynucleotide and/or the first optically active protein encoding polynucleotide. In certain example embodiments, the one or more untranslated regions comprise or consist of 5′ UTRs, 3′ UTRs, or both. In certain example embodiments, the test polynucleotide comprises or consists of a short synthetic polynucleotide. In certain example embodiments, the short synthetic polynucleotide is about 200 nucleotides. In certain example embodiments, the short synthetic polynucleotide has a sequence corresponding to a region of a genome. In certain example embodiments, the short synthetic polynucleotide has a sequence corresponding to a region of a human genome, a non-human animal genome, a plant genome, a microbial genome, or a viral genome. In certain example embodiments, the short synthetic polynucleotide has a sequence corresponding to (a) an annotated region of a genome; (b) an unannotated region of a genome; (c) a mutation; (d) a 5′UTR; (e) a 3′UTR; (f) an open reading frame; or (g) any combination thereof. In certain example embodiments, the short synthetic polynucleotide (a) is a sequence according to a sequence in SEQ ID NOS: 1-9890 or SEQ ID NOS: 9891-16277; (b) is a synthetic in silico designed sequence; (d) does not have 100 percent identity to a genome sequence, optionally a human genome, a non-human animal genome, a plant genome, a microbial genome, or a viral genome; (e) is not native to a genome, optionally a human genome, a non-human animal genome, a plant genome, a microbial genome, or a viral genome; or (f) any combination thereof. In certain example embodiments, the vector is a viral vector, optionally a retroviral vector, lentiviral vector, adenoviral vector, or adeno-associated viral vector.

Described in certain example embodiments, are vector systems comprising a vector comprising an optically active reporter construct of any one of the preceding paragraphs and as described elsewhere herein, and optionally one or more a viral envelope protein vectors, one or more viral packaging vectors, or any combination thereof. In certain example embodiments, the vectors or vector systems are capable of producing viral particles comprising the optically active reporter construct.

Described in certain example embodiments, are an optically active reporter construct library, optionally a pan-genome or pan-viral genomic optically active reporter construct library comprising a plurality of vectors each comprising an optically active reporter construct as in any one of the preceding paragraphs, wherein at least two test polynucleotides are different or the same, or wherein each test polynucleotide is different, and the test polynucleotides in each of the vectors of the plurality of vectors comprises or consists of a short synthetic polynucleotide.

Described in certain example embodiments herein are engineered viral particles each comprising a cargo comprising an optically active reporter construct, wherein the optically active reporter construct cargo comprises a test polynucleotide; a first optically active protein encoding polynucleotide, wherein the first optically active protein encoding polynucleotide is operatively coupled in-frame with the test polynucleotide; a second optically active protein encoding polynucleotide, wherein the second optically active protein is a different optically active protein that the first optically active protein; a promoter, wherein the test polynucleotide, the first optically active protein encoding polynucleotide, and the second optically active protein encoding polynucleotide are operatively coupled to the promoter; and an internal ribosomal entry site (IRES), wherein the IRES is between the first optically active protein encoding polynucleotide and the second optically active protein encoding polynucleotide. In certain example embodiments, the promoter is a constitutive promoter or an inducible promoter. In certain example embodiments, the optically active reporter construct further comprises one or more untranslated regions (UTRs), wherein the one or more untranslated regions are operatively coupled at the 5′ and/or 3′ end of the test polynucleotide and/or the first optically active protein encoding polynucleotide. In certain example embodiments, the one or more untranslated regions comprise or consist of 5′ UTRs, 3′ UTRs, or both. In certain example embodiments, the test polynucleotide comprises or consists of a short synthetic polynucleotide. In certain example embodiments, the short synthetic polynucleotide is about 200 nucleotides. In certain example embodiments, the short synthetic polynucleotide has a sequence corresponding to a region of a genome. In certain example embodiments, the short synthetic polynucleotide has a sequence corresponding to a region of a human genome, a non-human animal genome, a plant genome, a microbial genome, or a viral genome. In certain example embodiments, the short synthetic polynucleotide has a sequence corresponding to a) an annotated region of a genome; (b) an unannotated region of a genome; (c) a mutation; (d) a 5′UTR; (e) a 3′UTR; (f) an open reading frame; or (g) any combination thereof.

The engineered viral particle of any one of the preceding paragraphs or as described elsewhere herein, wherein the short synthetic polynucleotide has a sequence corresponding to (a) an annotated region of a genome; (b) an unannotated region of a genome; (c) a mutation; (d) a 5′UTR; (e) a 3′UTR; (f) an open reading frame; or (g) any combination thereof. In certain example embodiments, the short synthetic polynucleotide (a) is a sequence according to a sequence in SEQ ID NOS: 1-9890 or SEQ ID NOS: 9891-16277; (b) is a synthetic in silico designed sequence; (d) does not have 100 percent identity to a genome sequence, optionally a human genome, a non-human animal genome, a plant genome, a microbial genome, or a viral genome; (e) is not native to a genome, optionally a human genome, a non-human animal genome, a plant genome, a microbial genome, or a viral genome; or (f) any combination thereof. In certain example embodiments, the engineered viral particle is a viral particle, optionally a retroviral viral particle, lentiviral viral particle, adenoviral viral particle, or adeno-associated viral particle.

Described in certain example embodiments herein are methods comprising: (a) transducing one or more cells with one or more viral particles or a pan-viral engineered viral particle library comprising an optically active reporter construct as described herein and allowing for genomic integration and expression of the optically active reporter construct in the one or more cells; (b) selecting cells for genomic integration of the optically active reporter construct via sorting by detecting an optical signal produced from the first optically active protein and selecting cells that produce the optical signal from the second optically active protein; (c) sorting selected cells from (b) into expression bins, wherein sorting selected cells from (b) comprises detecting an optical signal produced by the first optically active protein; (d) sequencing one or more nucleic acids of the sorted selected cells by expression bin; and (e) computing an expression score for each test polynucleotide by expression bin. In certain example embodiments, sequencing comprises DNA sequencing, RNA sequencing, or both. In certain example embodiments, sequencing comprises genomic DNA sequencing. In certain example embodiments, sequencing comprises next generation sequencing or third generation sequencing. In certain example embodiments, sequencing comprises deep sequencing. In certain example embodiments, sequencing comprises single cell sequencing.

Described in certain example embodiments herein are methods of screening test agents and/or conditions comprising (a) transducing a first set of one or more cells with one or more viral particles or a pan-viral engineered viral particle library having an optically active reporter construct as described in any one of the preceding claims and elsewhere herein and allowing for genomic integration and expression of the optically active reporter construct in the one or more cells; (b) transducing a second set of the one or more cells with the one or more viral particles or a pan-viral engineered viral particle library having an optically active reporter construct as in any one of the preceding paragraphs or elsewhere herein and allowing for genomic integration and expression of the optically active reporter construct in the one or more cells, wherein the second set of one or more cells comprises the same one or more cells as the first set of one or more cells, and wherein the one or more viral particles or the pan-viral engineered viral particle library used to transduce the second set of one or more cells is the same as the one or more viral particles or the pan-viral engineered viral particle library used to transduce the first set of one or more cells; (c) exposing the second set of one or more cells to one or more test agents and/or conditions; (d) selecting cells in each of the first set of one or more cells and second set of one or more cells for genomic integration of the optically active reporter construct via sorting by detecting an optical signal produced from the first optically active protein and selecting cells that produce the optical signal from the second optically active protein; (e) sorting selected cells from (d) into expression bins, wherein sorting selected cells from (d) comprises detecting an optical signal produced by the first optically active protein; (f) sequencing one or more nucleic acids of the sorted selected cells by expression bin; (g) computing an expression score for each test polynucleotide by expression bin; and (h) comparing expression scores for each test polynucleotide between the first and second set of cells to determine an effect of the test agent and/or condition. In certain example embodiments, the test agent or condition is a small molecule agent, a biologic agent, a physical stress, a chemical stress, or any combination thereof. In certain example embodiments, sequencing comprises DNA sequencing, RNA sequencing, or both. In certain example embodiments, sequencing comprises genomic DNA sequencing. In certain example embodiments, sequencing comprises next generation sequencing or third generation sequencing. In certain example embodiments, sequencing comprises deep sequencing. In certain example embodiments, sequencing comprises single cell sequencing.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Sources for MHC-I presented antigens. See also Cardinaud et al. J Exp Med (2004) 199 (8): 1053-1063; Ingolia et al., Cell Reports 8, 1365-1379; Antón and Yewdell. J Leukoc Biol. 2014 April; 95(4):551-62; Starck et al., Science. 2016 Jan. 29; 351(6272); Starck and Shastri. Immunol Rev. 2016 July; 272(1):8-16.

FIG. 2—Systematic approach for characterization of viral antigens in hundreds of viruses.

FIG. 3—Schematic for synthetic libraries for systemic investigation of viral genomes.

FIG. 4A-4D—Exemplary high throughput measurements of antigen translation and presentation using synthetic libraries of designed oligos.

FIG. 5—Schematic for massively parallel ribosome profiling. A synthetic designed oligo construct is used to synthesize a synthetic designed oligo library. The library is expressed in cells. Ribosome protected fragments are sequenced and sequencing reads are mapped to identify open reading frames (ORFs) present in the expressed synthetic oligo library.

FIG. 6—Construct design for synthetic library expression plasmids. The synthetic designed oligo (variable region) is about 200 nucleotides. Two constant primers are coupled directly to 5′ and 3′ ends of the variable region. The 3′ end constant primer is followed by multiple frame shifted stop codons. This can be followed by a polyadenylation signal and/or WPRE element. A promoter is used to drive expression of the construct.

FIG. 7A-7B—Design and measurement of a ~5,000 oligo library towards previously reported viral ORFs to demonstrate accuracy of detection of ORFS using the massively parallel ribosome profiling approach of FIG. 5.

FIG. 8—A reduction in ribosome occupancy is obtained when the reported start codon is mutated in the synthetic oligo.

FIGS. 9A-9B—Detection of trinucleotides periodicity in ORFs encoded from synthetic oligos (SEQ ID NO: 16277) and (SEQ ID NO: 16278).

FIG. 10—Design of a 15,000 oligo pan-viral library and its use for identifying uORFs (upstream open reading frames) and short ORFs (SEQ ID NO: 16279).

FIG. 11—Reproducible and specific measurements of ribosome footprints.

FIG. 12—Predicting novel ORFs using ribosome profiling measurements of synthetic oligos.

FIGS. 13—Accurate detection of annotated coding sequences using massively parallel ribosome profiling—Ribosome footprint analysis.

FIG. 14—Accurate detection of annotated coding sequences using massively parallel ribosome profiling—Metagene analysis.

FIG. 15A-15B—Accurate detection of annotated coding sequences using massively parallel ribosome profiling—Inhibition of ribosome elongation with cycloheximide (FIG. 15A) or lactimidoycin (FIG. 15B).

FIG. 16—Pan-viral library measurements uncover ~7,000 viral ORFs.

FIG. 17—Ribosome density measurements across thousands of annotated viral coding sequences and their 5′ untranslated region (UTR).

FIG. 18A-18C—Discovery of hundreds of viral uORFs that negatively regulated the translation of the main coding sequence.

FIGS. 19-20—Ribosomes are transitioned from the detected uORFs to the main coding sequences in response to eIF2alpha phosphorylation.

FIG. 21—Exemplary optically active constructs for quantitative measurement of translation of viral ORFs.

FIG. 22—Quantitative measurements of viral ORFs translation using an optically active reporter construct. The reporter construct can have at least two different optically active polypeptides.

FIG. 23A-23B—Inferring RFP expression of individual oligos from their representation in sorted expression bins.

FIG. 24—Developing a Massively Parallel Reporter Assay for quantitative translation measurements of 15,000 viral sequences.

FIG. 25—Constant expression of one of the at least two different optically active proteins of the construct of FIG. 22 can be used to control for multiplicity of infection (MOI).

FIG. 26—Uniform coverage of sequencing reads across library oligos was achieved.

FIG. 27—Computing weighted average for each oligo from reads distribution across expression bins.

FIG. 28—Assessing the accuracy of pooled expression measurements.

FIG. 29—Detection of annotated viral ORFs using a Massively Parallel Reporter Assay (MPRA).

FIG. 30—Translation of viral ORFs is lower in genes with upstream ORFs in their 5′ UTRs.

FIG. 31—Development of a high-throughput method for systematic investigation of gene expression regulation.

FIG. 32—ORF discovery in hundreds of viral genomes using Massively Parallel Ribosome Profiling (MPRP).

FIGS. 33-35—Assessment of synthetic oligo library performance using a predefined set of viral ORFs from ribosome profiling experiments.

FIG. 36—Experimental measurements of pan-viral library composing 4,274 genes from 679 human viruses.

FIG. 37—Massively Parallel Ribosome Profiling of pan-viral library successfully detects annotated ORFs of human Coronaviruses.

FIG. 38—Massively Parallel Ribosome Profiling detects the translation of ORF9b in the SARS-CoV-1 genome.

FIG. 39—MPRP on pan-viral library detects internal out-of-frame ORFs in the nucleocapsid region of SARS-CoV-1 and HCoV-HKU1.

FIG. 40—A synthetic approach for the discovery of ORFs in hundreds of viral genomes.

FIG. 41—Summary of evaluation of viral ORFs. Massively Parallel Ribosome Profiling can be used to evaluate viral ORFs. This approach can be coupled with additional methodologies to evaluate the viral ORFs on additional cellular processes such as gene expression regulation and antigen presentation on infected cells.

FIG. 42—Other approaches that rely on tailing whole transcripts with 200 nt oligos can result in false positive discovery of internal ORFs.

FIGS. 43-44—ORFs discovery in hundreds of viral genomes using Massively Parallel Ribosome Profiling.

FIG. 45—Although ORF9b is conserved in SARS-CoV-1 it is missing from SARS-CoV-2 genomic annotations (SEQ ID NOS: 16280-16283).

FIG. 46—Understanding of the repertoire of proteins translated from the viral genome extends beyond the canonical ORFs, which includes identifying non-canonical ORFs and understanding their effects, including on other cellular processes such as gene expression regulation and antigen presentation (See e.g., FIG. 41).

FIG. 47—Researching HLA-I peptidome of HCMV uncovers peptides from non-canonical ORFs detected by the pan-viral library (SEQ ID NOS: 16284-16285).

FIG. 48A-48E—Design of oligonucleotide synthetic library and MPRP measurements. (FIG. 48A) (FIG. 48A) Illustration of the Massively Parallel Ribosome Profiling experiment (MPRP). (1) Synthetic library amplification using constant primers. (2) Cloning library into overexpression vector. (3) Transient transfection of plasmid pool into HEK293T or A549 cells for 24 h. (4) Treating cells with either LTM or CHX and performing ribosome profiling protocol. (5) Mapping deep sequencing reads (representing ribosome footprints) to the synthetic library. (6) Inferring translated ORFs using PRICE (Erhard et al. 2018). (FIG. 48B) Design of the tested synthetic oligonucleotides: (i) ORFs that were identified by ribosome profiling in infected cells with either intact start codon or a GCC mutation. (ii) Tailing oligos encompassing complete viral genomes/transcripts. (iii) Oligos spanning the 5′UTR and the first 140 nt of annotated viral CDSs. For the region containing the CDS, two oligos were designed: the wild type sequence and a start codon mutated oligo. (FIG. 48C) Comparing the number of ribosome footprints mapped to 15,000 oligos in two biological replicates of MPRP experiment in HEK293T cells. R=0.92, Pearson correlation. (FIG. 48D) Comparing the number of ribosome footprints mapped to 15,000 oligos in the MPRP experiment in HEK293T and A549 cells. R=0.89, Pearson correlation. (FIG. 48E) Comparing the number of ribosome footprints mapped to 1,163 identical oligos in two synthetic libraries that were independently cloned, transfected and measured using MPRP in HEK293T cells. R=0.79, Pearson correlation.

FIG. 49A-49F—Annotated CDSs measurements and ORF discovery. (FIG. 49A) (Left) Example of four individual oligos representing genes from the Herpesviridae family and the number of ribosome footprints observed in the MPRP assay. (Right) Metagene analysis showing the average of ribosome footprints in each position along 1,103 genes from the Herpesviridae family. Different colors, as represented in greyscale, represent the three reading frames (blue, 0; orange, +1; gray, −1). (FIG. 49B) Average number of ribosome footprints in each position for six additional viral families. A virus representing each family is illustrated in each plot. (FIG. 49C) Comparing the average number of ribosome footprints between oligos containing the wt start codon (upper graph) to those in which the annotated start codon was mutated to GCC (lower graph). Shown are the average ribosome footprints in each position across 3,777 oligos. The region with maximum information from footprints containing the wt or mutated start codon (position −3 to +15) is highlighted in red. (FIG. 49D) Pairwise analysis of the number of ribosome footprints on 3,777 viral CDSs with either the wt start codon or a GCC mutant. Showing the number of footprints in position −3 to +15 relative to the annotated start codon in each oligo. p<10⁻²⁸⁸, Wilcoxon signed-rank test. (FIG. 49E) ORF discovery using PRICE. Showing the number of ORFs that were detected in each position (referring to the ORF start position). (FIG. 49F) Comparing the number of reads estimated by PRICE for the detected CDSs in two biological replicates. R=0.93, Pearson correlation.

FIG. 50A-50E—MPRP measurements of canonical and non-canonical ORFs that were identified by traditional ribosome profiling. (FIG. 50A-50C) Metagene analysis of oligos containing the sequence of ORFs that were identified by ribosome profiling of HCMV-infected cells done by Stern-Ginossar et al. (Stern-Ginossar et al. 2012). Showing the average of ribosome footprints in each position across 143 annotated canonical ORFs (FIG. 50A), 573 non-canonical (FIG. 50B), and 245 non-canonical ORFs in the length of 20 aa or shorter (FIG. 50C). Different colors, as represented in greyscale, represent the three reading frames (blue, 0; orange, +1; gray, −1). (FIG. 50D) Comparing the average number of ribosome footprints between oligos containing the wt start codon (upper graph) to those in which the reported start codon was mutated to GCC (lower graph). Shown are the average ribosome footprints in each position across 284 oligos, containing Ribo-seq ORFs in the length of 7-45 aa. The region with maximum information from footprints containing the wt or mutated start codon (position −3 to +15) is highlighted in red, as represented in greyscale. (FIG. 50E) ORF discovery using PRICE. Showing the number of ORFs that were detected in each position (referring to the ORF start position).

FIG. 51A-51C—HLA-I peptides derived from non-canonical ORFs in HCMV and VACV. (FIG. 51A) (Left) Illustration of the HLA-I immunopeptidome profiling performed by Erhard et al. and Lorente et al. for HCMV- and VACV-infected cells, respectively. (Right) The dataset that we built to research the mass spectrometry raw data including non-canonical ORFs that were identified in the MPRP assay. (FIG. 51B) HLA-I peptides that were detected in four non-canonical ORFs of HCMV identified by MPRP: two uORFs in the 5′UTRs of UL4 and UL148, an uoORF in the 5′UTR and coding region of UL135, and an N-extended isoform of UL36 (SEQ ID NOS: 16286-16297). (FIG. 51C) HLA-I peptide that was detected in an uORF in the non-coding region upstream of the I7L coding sequence. Ribosome densities on uORFs and CDSs in non-stressed cells and in response to eIF2alpha phosphorylation (SEQ ID NOS: 16298-16302).

FIG. 52A-52F—(FIG. 52A) Heatmap showing ribosome footprint densities across 2,418 viral oligos. Each line represents a single viral gene and each column represents the position relative to the annotated start codon. Genes in the upper cluster (purple, as represented in greyscale) had more footprints in the 5′UTR region than the CDS region, and genes in the lower cluster (blue, as represented in greyscale) had more footprints in the CDS than the 5′UTR. (FIG. 52B) Example of two individual genes from each cluster and the distribution of ribosome footprints observed in each position. (FIG. 52C) Metagene analysis showing the average ribosome footprints in each position along 2,473 uORFs detected by PRICE, relative to the uORF start position. Shown for CHX (Left) and LTM (right) inhibitors. Different colors, as represented in greyscale, represent the three reading frames (blue, 0; orange, +1; gray, −1). (FIG. 52D) Illustration of uORF-mediated attenuation of translation initiation from canonical CDSs. Ribosomes that initiate at the uORF start codon are less likely to efficiently reinitiate at the downstream start codon of the main CDS. Upon stress, eIF2alpha is phosphorylated as part of integrated stress response, which includes cellular response to viral infection. Ribosomes are more likely to “miss” the uORF start codon and initiate successfully at the start codon of the main CDS. (FIG. 52E) Western blot analysis lysates from HEK293T cells treated with 40 uM Sodium-Arsenite for 30 and 60 min. Phosphorylated eIF2alpha was detected with a monoclonal phospho S51 antibody (Upper panel). ATF4 protein was detected using a polyclonal antibody (Lower panel). In both membranes, Vinculin was used as a loading control. (FIG. 52F) Repeating the MPRP experiment in HEK293T cells that were treated with 40 uM Sodium-Arsenite and in non-treated cells. Shown are heatmaps of ribosome densities across viral oligos and clusters similarly to the analysis in (FIG. 52A).

FIG. 53A-53C—Ribosome profiling of exogenous ORF from overexpression plasmid. (FIG. 53A) Illustration of EmGFP plasmid transfection followed by ribosome profiling. (FIG. 53B) Mapping ribosome footprints to EmGFP plasmids using Integrative Genomics Viewer (IGV). (FIG. 53C) (Top) The design of a truncated EmGFP construct mimicking the synthetic library oligos including the constant primers and the plasmid cloning site. (Bottom) Mapping ribosome footprints to the truncated EmGFP oligo using IGV.

FIG. 54A-54B—MPRP measurements of tailing oligos across 30 transcripts of HCMV and HSV-1. (FIG. 54A) The design of tailing oligo across 30 mRNAs. (FIG. 54B) Showing the total number of ribosome footprints in each position relative to the CDS start codon or the stop codon in 30 annotated mRNAs (14 of HSV-1 and 16 of HCMV).

FIG. 55—Biological replicate of MPRP measurements of the pilot library. Comparing the number of ribosome footprints mapped to 5,170 oligos of the pilot library in two biological replicates of MPRP experiment in HEK293T cells. R=0.81, Pearson correlation.

FIG. 56—Metagene analysis of elongating ribosome footprints across 21 viral families. Average number of ribosome footprints in each position for 21 viral families observed in MPRP measurements after treatment with CHX to inhibit elongation ribosome. Different colors, as represented in greyscale, represent the three reading frames (blue, 0; orange, +1; gray, −1).

FIG. 57—Metagene analysis of initiating ribosome footprints across 21 viral families. Average number of ribosome footprints in each position for 21 viral families observed in MPRP measurements after treatment with LTM to inhibit initiating ribosome. Different colors, as represented in greyscale, represent the three reading frames (blue, 0; orange, +1; gray, −1).

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further embodiment. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range. General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g., a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader embodiments discussed herein. One embodiment described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

Recent epidemics of Ebola and Zika are stark reminders of the enormous impact of viruses on public health, emphasizing the great need for transformative vaccines and therapeutics. Despite many recent technological advances, systematic methods to investigate virology have lagged behind, so that many of the global principles underlying viral infection remain unknown. One critical area of study is how viruses evade the host immune response. During infection, viral peptides are uploaded on the major histocompatibility class I molecule (MHC-I) and presented to cytotoxic T-cells. Some viruses respond by inhibiting the MHC-I pathway globally, but many infected host cells maintain an active presentation pathway, suggesting the existence of additional response mechanisms. Although it was thought that the MHC-I repertoire reflects the intracellular protein pool, recent studies demonstrate that many of the presented antigens result from newly synthesized peptides from non-functional sequences. Among these are alternative reading frames (ARFs), upstream open reading frames (uORFs), short open reading frames (sORFs) and defective ribosomal products (DRiPs). These sources vastly increase the availability of viral peptides for presentation and immune evasion and imply an important role for translational regulation in this process. Since traditional studies investigate only a few peptides at a time, a comprehensive view of the collection of functional ORFs and immunogenic peptides in each virus is not fully characterized and, in turn, the causal effect of viral mutations on immunogenicity.

Applicant has developed high-throughput techniques to evaluate the translation of hundreds of viral genomes. Exemplary embodiments disclosed herein provide massively parallel ribosome profiling, massively parallel antigen presentation assays, and massively parallel reporter assays for high throughput analysis of viral translatomes and/or immunopeptidome. Additionally, Applicant provides non-canonical viral ORFs and compositions, such as vaccines and other immunogenic compositions, including or capable of producing the non-canonical viral ORFs. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Viral Polynucleotides, Polypeptides, and Compositions

Described in certain embodiments herein are polynucleotides comprising one or more viral polynucleotides from SEQ ID NOS: 1-9890 or SEQ ID NOS: 9891-16277. Described in certain example embodiments herein are polynucleotides, such as engineered polynucleotides, comprising one or more viral polynucleotides selected from those polynucleotides identified by a method described herein and/or SEQ ID NOS: 1-9890 or SEQ ID NOS: 9891-16277. Described in certain example embodiments herein are polypeptides encoded by the polynucleotides of the present invention such as one or more of those set forth in SEQ ID NOS: 1-9890 or SEQ ID NOS: 9891-16277 and/or as identified by a method of the present invention described in greater detail elsewhere herein.

The polynucleotide and/or polypeptides of the present invention can be included in a cell, vector, and/or delivery vehicle. Described in certain example embodiments herein are immunogenic compositions comprising a polynucleotide, a vector, polypeptide, delivery vehicle, and/or cell as in any one of the preceding paragraphs or as described elsewhere herein. In certain example embodiments, the polynucleotide and/or the polypeptide of the immunogenic composition is capable of stimulating a B-cell and/or T-cell response in a subject to which it is delivered. In certain example embodiments, the B-cell response comprises antibody production. The immunogenic compositions may elicit an immunological response in the host to which the immunogenic compositions are administered. Such immunological response may be a T cell-mediated (e.g., cytotoxic T cell-mediated) immune response to the immunogenic compositions. In certain embodiments, the immunogenic compositions may be combined with one or more antigenic components and/or anti-viral therapeutics. In some examples, such combination may elicit cellular and/or antibody-mediated immune response, e.g., production or activation of antibodies, B cells, helper T cells, suppressor T cells, and/or cytotoxic T cells and/or gamma-delta T cells.

In some embodiments, the composition may be used to treat viral infection of a subject. For example, the composition may be used to remove infected cells in the subject. In some embodiments, the composition may be used to prevent viral infection or reduce the impact of viral infection on the subject (e.g., clinical signs normally displayed by an infected host, a quicker recovery time and/or a lowered duration of infectivity or lowered pathogen titer in the tissues or body fluids or excretions of the infected subject). In some cases, the subject displays a protective immunological response such that resistance to new infection may be enhanced and/or the clinical severity of the disease may be reduced.

Described in certain embodiments elsewhere herein are methods, such as massively parallel ribosome profiling, antigen presentation profiling, and/or massively parallel reporter assays. Such assays can be used to identify viral polypeptides and encoding polynucleotides, including but not limited to, non-canonical open reading frames. In certain example embodiments, such non-canonical open reading frames are alternative reading frames (ARFs), upstream open reading frames (uORFs), short open reading frames (sORFs) and defective ribosomal products (DRiPs). Described in certain example embodiments herein are polynucleotides and/or polypeptides identified using the method described herein, such as the massively parallel ribosome profiling, immunopeptidome profiling, and/or massively parallel reporter assays described elsewhere herein.

### Polynucleotides and Polypeptides

Described in certain example embodiments herein are polynucleotides, such as engineered polynucleotides, comprising one or more viral polynucleotides selected from those polynucleotides identified by a method described herein and/or SEQ ID NOS: 1-9890 or SEQ ID NOS: 9891-16277. In some embodiments, the polynucleotides are codon optimized for expression in humans or non-human animals. In some embodiments, the one or more viral polynucleotides has a sequence corresponding to a (a) an annotated region of a genome; (b) an unannotated region of a genome; (c) a mutation; (d) a 5′UTR; (e) a 3′UTR; (f) an open reading frame; or (g) any combination thereof. In certain example embodiments, the short synthetic polynucleotide (a) is a sequence according to a sequence in SEQ ID NOS: 1-9890 or SEQ ID NOS: 9891-16277; (b) is a synthetic in silico designed sequence; (d) does not have 100 percent identity to a genome sequence, optionally a human genome, a non-human animal genome, a plant genome, a microbial genome, or a viral genome; (e) is not native to any genome, optionally a human genome, a non-human animal genome, a plant genome, a microbial genome, or a viral genome; or (f) any combination thereof.

In some embodiments, a polynucleotide of the present invention has 50-100% identity to a polynucleotide of SEQ ID NOS: 1-9890 or SEQ ID NOS: 9891-16277 or a polynucleotide identified using a method of the present invention described elsewhere herein. In some embodiments, a polynucleotide of the present invention has 50%, to/or 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identity to a polynucleotide of SEQ ID NOS: 1-9890 or SEQ ID NOS: 9891-16277 or a polynucleotide identified using a method of the present invention described elsewhere herein. The terms “percent (%) sequence identity”, and the like, generally refer to the degree of identity or correspondence between different nucleotide sequences of nucleic acid molecules or amino acid sequences of polypeptides that may or may not share a common evolutionary origin. Sequence identity can be determined using any of a number of publicly available sequence comparison algorithms, such as BLAST, FASTA, DNA Strider, GCG (Genetics Computer Group, Program Manual for the GCG Package, Version 7, Madison, Wis.), etc. In some embodiments, a polypeptide of the present invention has 50%, to 100% identity to a polypeptide encoded by a polynucleotide of SEQ ID NOS: 1-9890 or SEQ ID NOS: 9891-16277 or a polynucleotide identified using a method of the present invention described elsewhere herein. In some embodiments, a polypeptide of the present invention has 50%, to/or 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identity to a polypeptide encoded by a polynucleotide of SEQ ID NOS: 1-9890 or SEQ ID NOS: 9891-16277 or a polynucleotide identified using a method of the present invention described elsewhere herein.

In some embodiments, the one or more viral polynucleotides corresponds to an open reading frame (ORF) in a viral genome. The one or more viral polypeptides may be expressed from one or more open reading frames (ORFs) in a viral genome. An ORF refers to a polynucleotide that encodes a protein, or a portion of a protein. An open reading frame usually begins with a start codon and is read in codon-triplets until the frame ends with a STOP codon. In some embodiments, the ORFs are canonical ORFs. A canonical ORF is an ORF that is most prevalent, most similar to orthologous sequences found in other species, by virtue of its length or amino acid composition, allows for the clearest description of domains, isoforms, polymorphisms, post-translational modifications, or in the absence of other information is the longest sequence. In some embodiments, the ORFs are non-canonical ORFs. In some embodiments, the non-canonical ORFs are alternative reading frames (ARFs), upstream open reading frames (uORFs), short open reading frames (sORFs), defective ribosomal products (DRiPs), out-of-frame ORFs, or other non-canonical ORFs.

### Exemplary Viruses

The one or more polynucleotides and/or polypeptides may be derived from one or more polynucleotides (e.g., viral genome) or proteins of a virus. A polypeptide derived from a protein has an amino acid sequence that is a portion or the full-length of the protein's amino acid sequence. In some examples, the one or more polypeptides may be peptides resulting from digestion or degradation of a viral protein in cells infected by the virus. The virus may be a DNA virus, an RNA virus, or a retrovirus.

The one or more proteins of a virus may be protein(s) of a coronavirus. The coronavirus may be a positive-sense single stranded RNA family of viruses, infecting a variety of animals and humans. In one example, the one or more proteins are protein(s) of SARS-CoV-2. SARS-CoV is one type of coronavirus infection, as well as MERS-CoV. Example sequences of the SARS-CoV-2 are available at GISAID accession no. EPI_ISL_402124 and EPI_ISL_402127-402130, and described in DOI: 10.1101/2020.01.22.914952. Further deposits of the example SARS-CoV2 are deposited in the GISAID platform include EP_ISL_402119-402121 and EP_ISL_402123-402124; see also GenBank Accession No. MN908947.3.

The one or more polypeptides may be derived from proteins of other viruses. Examples of such viruses include Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In certain example embodiments, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bornavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwera virus, Caliciviridae virus. California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-1, Human herpesvirus 1-8, Human immunodeficiency virus 1/2, Human mastadenovirus A-G, Human papillomavirus, Human parainfluenza virus 1-4, Human paraechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2\.225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O'nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Boma disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

### SARS-CoV-2 Variants

The present disclosure relates to and/or involves SARS-CoV-2. More particularly the disclosure describes, inter alia, SARS-CoV-2 variant immunogenic polypeptides and encoding polynucleotides. As described herein are vaccines that include the SARS-CoV-2 variant immunogenic polypeptides and/or encoding polynucleotides. Such vaccines can be effective against one or more SARS-CoV-2 variants.

As used herein, the term “variant” refers to any virus having one or more mutations as compared to a known virus. A strain is a genetic variant or subtype of a virus. The terms ‘strain’, ‘variant’, and ‘isolate’ may be used interchangeably. In certain embodiments, a variant has developed a “specific group of mutations” that causes the variant to behave differently than that of the strain it originated from. While there are many thousands of variants of SARS-CoV-2, (Koyama, Takahiko Koyama; Platt, Daniela; Parida, Laxmi (June 2020). “Variant analysis of SARS-CoV-2 genomes”. Bulletin of the World Health Organization. 98: 495-504) there are also much larger groupings called clades. Several different clade nomenclatures for SARS-CoV-2 have been proposed. As of December 2020, GISAID, referring to SARS-CoV-2 as hCoV-19 identified seven clades (0, S, L, V, G, GH, and GR) (Alm E, Broberg E K, Connor T, et al. Geographical and temporal distribution of SARS-CoV-2 clades in the WHO European Region, January to June 2020 [published correction appears in Euro Surveill. 2020 August; 25(33):]. Euro Surveill. 2020; 25(32):2001410). Also as of December 2020, Nextstrain identified five (19A, 19B, 20A, 20B, and 20C) (Cited in Alm et al. 2020). Guan et al. identified five global clades (G614, 584, V251, 1378 and D392) (Guan Q, Sadykov M, Mfarrej S, et al. A genetic barcode of SARS-CoV-2 for monitoring global distribution of different clades during the COVID-19 pandemic. Int J Infect Dis. 2020; 100:216-223). Rambaut et al. proposed the term “lineage” in a 2020 article in Nature Microbiology; as of December 2020, there have been five major lineages (A, B, B.1, B.1.1, and B.1.777) identified (Rambaut, A.; Holmes, E. C.; O'Toole, A.; et al. “A dynamic nomenclature proposal for SARS-CoV-2 lineages to assist genomic epidemiology”. 5: 1403-1407).

Genetic variants of SARS-CoV-2 have been emerging and circulating around the world throughout the COVID-19 pandemic (see, e.g., The US Centers for Disease Control and Prevention; www.cdc.gov/coronavirus/2019-ncov/variants/variant-info.html). Exemplary, non-limiting variants applicable to the present disclosure include variants of SARS-CoV-2, particularly those having substitutions of therapeutic concern. Table 1 shows exemplary, non-limiting genetic substitutions in SARS-CoV-2 variants.

TABLE 1

| | Common Pango Lineages with |
|---|---|
| Spike Protein Substitution | Spike Protein Substitutions |
| L452R | A.2.5, B.1, B.1.429, B.1.427, B.1.617.1, |
| | B.1.526.1, B.1.617.2, C.36.3 |
| E484K | B.1.1.318, B.1.1.7, B.1.351, B.1.525, |
| | B.1.526, B.1.621, B.1.623, P.1, P.1.1, |
| | P.1.2, R.1 |
| K417N, E484K, N501Y | B.1.351, B.1.351.3 |
| K417T, E484K, N501Y | P.1, P.1.1, P.1.2 |

Phylogenetic Assignment of Named Global Outbreak (PANGO) Lineages is software tool developed by members of the Rambaut Lab. The associated web application was developed by the Centre for Genomic Pathogen Surveillance in South Cambridgeshire and is intended to implement the dynamic nomenclature of SARS-CoV-2 lineages, known as the PANGO nomenclature. It is available at cov-lineages.org.

In some embodiments, the SARS-CoV-2 variant is and/or includes: B.1.1.7, also known as Alpha (WHO) or UK variant, having the following spike protein substitutions: 69del, 70del, 144del, (E484K*), (S494P*), N501Y, A570D, D614G, P681H, T716I, S982A, and D1118H (K1191N*); B.1.351, also known as Beta (WHO) or South Africa variant, having the following spike protein substitutions: D80A, D215G, 241del, 242del, 243del, K417N, E484K, N501Y, D614G, and A701V; B.1.427, also known as Epsilon (WHO) or US California variant, having the following spike protein substitutions: L452R, and D614G; B.1.429, also known as Epsilon (WHO) or US California variant, having the following spike protein substitutions: S13I, W152C, L452R, and D614G; B.1.617.2, also known as Delta (WHO) or India variant, having the following spike protein substitutions: T19R, (G142D), 156del, 157del, R158G, L452R, T478K, D614G, P681R, and D950N; and P.1, also known as Gamma (WHO) or Japan/Brazil variant, having the following spike protein substitutions: L18F, T20N, P26S, D138Y, R190S, K417T, E484K, N501Y, D614G, H655Y, and T1027I, or any combination thereof.

In some embodiments, the SARS-CoV-2 variant is classified and/or otherwise identified as a Variant of Concern (VOC) by the World Health Organization and/or the U.S. Centers for Disease Control. A VOC is a variant for which there is evidence of an increase in transmissibility, more severe disease (e.g., increased hospitalizations or deaths), significant reduction in neutralization by antibodies generated during previous infection or vaccination, reduced effectiveness of treatments or vaccines, or diagnostic detection failures.

In some embodiments, the SARS-Cov-2 variant is classified and/or otherwise identified as a Variant of High Consequence (VHC) by the World Health Organization and/or the U.S. Centers for Disease Control. A variant of high consequence has clear evidence that prevention measures or medical countermeasures (MCMs) have significantly reduced effectiveness relative to previously circulating variants.

In some embodiments, the SARS-Cov-2 variant is classified and/or otherwise identified as a Variant of Interest (VOI) by the World Health Organization and/or the U.S. Centers for Disease Control. A VOI is a variant with specific genetic markers that have been associated with changes to receptor binding, reduced neutralization by antibodies generated against previous infection or vaccination, reduced efficacy of treatments, potential diagnostic impact, or predicted increase in transmissibility or disease severity.

In some embodiments, the SARS-Cov-2 variant is classified and/or is otherwise identified as a Variant of Note (VON). As used herein, VON refers to both “variants of concern” and “variants of note” as the two phrases are used and defined by Pangolin (cov-lineages.org) and provided in their available “VOC reports” available at cov-lineages.org.

In some embodiments the SARS-Cov-2 variant is a VOC. In some embodiments, the SARS-CoV-2 variant is or includes an Alpha variant (e.g., Pango lineage B.1.1.7), a Beta variant (e.g., Pango lineage B.1.351, B.1.351.1, B.1.351.2, and/or B.1.351.3), a Delta variant (e.g., Pango lineage B.1.617.2, AY.1, AY.2, AY.3 and/or AY.3.1); a Gamma variant (e.g., Pango lineage P.1, P.1.1, P.1.2, P.1.4, P.1.6, and/or P.1.7), or any combination thereof.

In some embodiments, the SARS-Cov-2 variant is a VOI. In some embodiments, the SARS-CoV-2 variant is or includes an Eta variant (e.g., Pango lineage B.1.525 (Spike protein substitutions A67V, 69del, 70del, 144del, E484K, D614G, Q677H, F888L)); an Iota variant (e.g., Pango lineage B.1.526 (Spike protein substitutions L5F, (D80G*), T95I, (Y144-*), (F157S*), D253G, (L452R*), (S477N*), E484K, D614G, A701V, (T859N*), (D950H*), (Q957R*))); a Kappa variant (e.g., Pango lineage B.1.617.1 (Spike protein substitutions (T95I), G142D, E154K, L452R, E484Q, D614G, P681R, Q1071H)); Pango lineage variant B.1.617.2 (Spike protein substitutions T19R, G142D, L452R, E484Q, D614G, P681R, D950N)), Lambda (e.g., Pango lineage C.37); or any combination thereof.

In some embodiments, SARS-Cov-2 variant is a VON. In some embodiments, the SARS-Cov-2 variant is or includes Pango lineage variant P.1 (alias, B.1.1.28.1.) as described in Rambaut et al. 2020. Nat. Microbiol. 5:1403-1407) (spike protein substitutions: T20N, P26S, D138Y, R190S, K417T, E484K, N501Y, H655Y, TI027I)); an Alpha variant (e.g., Pango lineage B.1.1.7); a Beta variant (e.g., Pango lineage B.1.351, B.1.351.1, B.1.351.2, and/or B.1.351.3); Pango lineage variant B.1.617.2 (Spike protein substitutions T19R, G142D, L452R, E484Q, D614G, P681R, D950N)); an Eta variant (e.g., Pango lineage B.1.525); Pango lineage variant A.23.1 (as described in Bugembe et al. medRxiv. 2021. doi: https://doi.org/10.1101/2021.02.08.21251393) (spike protein substitutions: F157L, V367F, Q613H, P681R); or any combination thereof.

### Major Histocompatibility Complex (MHC) Binding Polypeptides

### MHC I

The polypeptide(s) described herein or as identified by a method of the present invention, such as those encoded by a polynucleotide described herein, can bind, or is capable of binding, to Major Histocompatibility Complex (MHC) class I, e.g., Human Leukocyte Antigen class I. In some cases, after binding, the MHC class I may present the one or more polypeptides to activate cytotoxic T cells. As used herein, MHC refers to protein complexes capable of binding peptides resulting from the proteolytic cleavage of protein antigens and representing potential T-cell epitopes, transporting them to the cell surface and presenting them there to specific cells, in particular cytotoxic T-lymphocytes or T-helper cells.

MHC class I, or MHC-I, function mainly in antigen presentation to CD8+ T lymphocytes or cytotoxic T cells and may be heterodimers comprising two polypeptide chains, an alpha chain and β2-microglobulin.

In some embodiments, the MHC class I may be Human Leukocyte Antigen (HLA) class I, which is the MHC class I in human. HLA class I may comprises an alpha chain and β2-microglobulin. The alpha chain may be HLA-A, HLA-B, or HLA-C. In one example, the one or more peptides binds, or is capable of binding, to HLA-A. In another example, the one or more peptides binds, or is capable of binding, to HLA-B. In another example, the one or more peptides binds, or is capable of binding, to HLA-C. In another example, the one or more peptides binds, or is capable of binding, to β2-microglobulin of HLA-1.

### MHC II

The polypeptide(s) described herein or as identified by a method of the present invention, such as those encoded by a polynucleotide described herein, can bind, or is capable of binding, to Major Histocompatibility Complex (MHC) class II, e.g., Human Leukocyte Antigen class II. Like MHC class I molecules, class II molecules are also heterodimers, but in this case consist of two homogenous peptides, an α and β chain, both of which are encoded in the MHC. The subdesignation α1, α2, etc. refers to separate domains within the HLA gene; each domain is usually encoded by a different exon within the gene, and some genes have further domains that encode leader sequences, transmembrane sequences, etc. These molecules have both extracellular regions as well as a transmembrane sequence and a cytoplasmic tail. The α1 and β1 regions of the chains come together to make a membrane-distal peptide-binding domain, while the α2 and β2 regions, the remaining extracellular parts of the chains, form a membrane-proximal immunoglobulin-like domain. The antigen binding groove, where the antigen or peptide binds, is made up of two α-helixes walls and β-sheet. See also e.g., Jones et al., Nature Reviews. Immunology. 6 (4): 271-82. doi:10.1038/nri1805. PMID 16557259. S2CID 131777.

In some embodiments, the MHC class II may be Human Leukocyte Antigen (HLA) class II, which is the MHC class II in human. In some embodiments, the HLA II that binds a polypeptide of the present invention is HLA-DM, HLA-DO, HLA-DP, HLA-DQ, or HLA-DR. In some embodiments, the HLA II that binds a polypeptide of the present invention comprises an alpha chain selected from HLA-DMA, HLA-DOA, HLA-DPA1, HLA-DQA1, HLA-DQA2, or HLA-DRA. In some embodiments, the HLA II that binds a polypeptide of the present invention comprises a beta chain selected from HLA-DMB, HLA-DOB, HLA-DPB1, HLA-DQB1, HLA-DQB2, HLA-DRB1, HLA-DRB3, HLA-DRB4, or HLA-DRB5.

### HLA Alleles

The one or more peptides may bind, or may be capable of binding, to proteins encoded by certain HLA alleles. HLA genes may be polymorphic and have many different alleles, allowing them to fine-tune the immune system. The nomenclature of HLA genes are well known in the art, e.g., as described in Marsh S G E et al., Nomenclature for factors of the HLA system, 2010, Tissue Antigens. 2010 April; 75(4): 291-455, which is incorporated by reference in its entirety.

The HLA alleles may encode HLA protein capable of epitope binding. In some cases, the HLA alleles may have a ranking cut-off as determined by a machine learning predictor of HLA (e.g., HLA-I or HLA-II) epitope binding. For example, the HLA alleles may have a ranking cut-off of at least 0.1%, 0 at least 0.5%, or at least 1.0% as determined by HLAthena. Examples of methods for determining the ranking include those described in Sarkizova S. et al., A large peptidome dataset improves HLA class I epitope prediction across most of the human population, Nat Biotechnol. 2020 February; 38(2):199-209; and Abelin J G et al., Mass Spectrometry Profiling of HLA-Associated Peptidomes in Mono-allelic Cells Enables More Accurate Epitope Prediction, Immunity. 2017 Feb. 21; 46(2):315-326, which are incorporated by reference herein in their entireties.

In some embodiments, the proteins encoded by HLA alleles include HLA proteins encoded by HLA-A*02:01, HLA-A*25:01, HLA-A*30:01, HLA-B*18:01, HLA-B*44:03, HLA-C*12:03, HLA-B*16:01, HLA-A*02:01, HLA-B*07:02, HLA-C*07:02; HLA-A*01:01; HLA-A*02:06; HLA-A*26:01; HLA-A*02:07; HLA-A*29:02; HLA-A*02:03; HLA-A*30:02; HLA-A*32:01; HLA-A*68:02; HLA-A*02:05; HLA-A*02:02; HLA-A*36:01; HLA-A*02:11; HLA-A*02:04; HLA-B*35:01; HLA-B*51:01; HLA-B*40:01; HLA-B*40:02; HLA-B*07:02; HLA-B*07:04; HLA-B*08:01; HLA-B*13:01; HLA-B*46:01; HLA-B*52:01; HLA-B*44:02; HLA-B*40:06; HLA-B*13:02; HLA-B*56:01; HLA-B*54:01; HLA-B*15:02; HLA-B*35:07; HLA-B*27:05; HLA-B*15:03; HLA-B*42:01; HLA-B*55:02; HLA-B*45:01; HLA-B*50:01; HLA-B*35:03; HLA-B*49:01; HLA-B*58:02; HLA-B*15:17; HLA-C*57:02; HLA-C*04:01; HLA-C*03:04; HLA-C*01:02; HLA-C*07:01; HLA-C*06:02; HLA-C*03:03; HLA-C*08:01; HLA-C*15:02; HLA-C*12:02; HLA-C*02:02; HLA-C*05:01; HLA-C*03:02; HLA-C*16:01; HLA-C*08:02; HLA-C*04:03; HLA-C*17:01; or HLA-C*17:04. In some embodiments, the HLA-1 is encoded by HLA-A*02:01, HLA-A*25:01, HLA-A*30:01, HLA-B*18:01, HLA-B*44:03, HLA-C*12:03, HLA-B*16:01, HLA-A*02:01, HLA-B*07:02, or HLA-C*07:02. In some embodiments, the proteins encoded by HLA alleles include HLA proteins encoded by HLA-A*02:01, HLA-A*25:01, HLA-A*30:01, HLA-B*18:01, HLA-B*44:03, HLA-C*12:03, HLA-B*16:01, HLA-A*02:01, HLA-B*07:02, HLA-C*07:02, or a combination thereof.

In one example, the one or more polypeptides binds, or is capable of binding, to an HLA protein encoded by HLA-A*02:01. In another example, the one or more polypeptides binds, or is capable of binding, to an HLA protein encoded by HLA-A*25:01. In another example, the one or more polypeptides binds, or is capable of binding, to an HLA protein encoded by HLA-A*30:01. In another example, the one or more polypeptides binds, or is capable of binding, to an HLA protein encoded by HLA-B*18:01. In another example, the one or more polypeptides binds, or is capable of binding, to an HLA protein encoded by HLA-B*44:03. In another example, the one or more polypeptides binds, or is capable of binding, to an HLA protein encoded by HLA-C*12:03. In another example, the one or more polypeptides binds, or is capable of binding, to an HLA protein encoded by HLA-B*16:01. In another example, the one or more polypeptides binds, or is capable of binding, to an HLA protein encoded by HLA-A*02:01. In another example, the one or more polypeptides binds, or is capable of binding, to an HLA protein encoded by HLA-B*07:02. In another example, the one or more polypeptides binds, or is capable of binding, to an HLA protein encoded by HLA-C*07:02. In another example, the one or more polypeptides binds, or is capable of binding, to an HLA protein encoded by HLA-A*01:01. In another example, the one or more polypeptides binds, or is capable of binding, to an HLA protein encoded by HLA-A*02:06. In another example, the one or more polypeptides binds, or is capable of binding, to an HLA protein encoded by HLA-A*26:01. In another example, the one or more polypeptides binds, or is capable of binding, to an HLA protein encoded by HLA-A*02:07. In another example, the one or more polypeptides binds, or is capable of binding, to an HLA protein encoded by HLA-A*29:02. In another example, the one or more polypeptides binds, or is capable of binding, to an HLA protein encoded by HLA-A*02:03. In another example, the one or more polypeptides binds, or is capable of binding, to an HLA protein encoded by HLA-A*30:02. In another example, the one or more polypeptides binds, or is capable of binding, to an HLA protein encoded by HLA-A*32:01. In another example, the one or more polypeptides binds, or is capable of binding, to an HLA protein encoded by HLA-A*68:02. In another example, the one or more polypeptides binds, or is capable of binding, to an HLA protein encoded by HLA-A*02:05. In another example, the one or more polypeptides binds, or is capable of binding, to an HLA protein encoded by HLA-A*02:02. In another example, the one or more polypeptides binds, or is capable of binding, to an HLA protein encoded by HLA-A*36:01. In another example, the one or more polypeptides binds, or is capable of binding, to an HLA protein encoded by HLA-A*02:11. In another example, the one or more polypeptides binds, or is capable of binding, to an HLA protein encoded by HLA-A*02:04. In another example, the one or more polypeptides binds, or is capable of binding, to an HLA protein encoded by HLA-B*35:01. In another example, the one or more polypeptides binds, or is capable of binding, to an HLA protein encoded by HLA-B*51:01. In another example, the one or more polypeptides binds, or is capable of binding, to an HLA protein encoded by HLA-B*40:01. In another example, the one or more polypeptides binds, or is capable of binding, to an HLA protein encoded by HLA-B*40:02. In another example, the one or more polypeptides binds, or is capable of binding, to an HLA protein encoded by HLA-B*07:02. In another example, the one or more polypeptides binds, or is capable of binding, to an HLA protein encoded by HLA-B*07:04. In another example, the one or more polypeptides binds, or is capable of binding, to an HLA protein encoded by HLA-B*08:01. In another example, the one or more polypeptides binds, or is capable of binding, to an HLA protein encoded by HLA-B*13:01. In another example, the one or more polypeptides binds, or is capable of binding, to an HLA protein encoded by HLA-B*46:01. In another example, the one or more polypeptides binds, or is capable of binding, to an HLA protein encoded by HLA-B*52:01. In another example, the one or more polypeptides binds, or is capable of binding, to an HLA protein encoded by HLA-B*44:02. In another example, the one or more polypeptides binds, or is capable of binding, to an HLA protein encoded by HLA-B*40:06. In another example, the one or more polypeptides binds, or is capable of binding, to an HLA protein encoded by HLA-B*13:02. In another example, the one or more polypeptides binds, or is capable of binding, to an HLA protein encoded by HLA-B*56:01. In another example, the one or more polypeptides binds, or is capable of binding, to an HLA protein encoded by HLA-B*54:01. In another example, the one or more polypeptides binds, or is capable of binding, to an HLA protein encoded by HLA-B*15:02. In another example, the one or more polypeptides binds, or is capable of binding, to an HLA protein encoded by HLA-B*35:07. In another example, the one or more polypeptides binds, or is capable of binding, to an HLA protein encoded by HLA-B*27:05. In another example, the one or more polypeptides binds, or is capable of binding, to an HLA protein encoded by HLA-B*15:03. In another example, the one or more polypeptides binds, or is capable of binding, to an HLA protein encoded by HLA-B*42:01. In another example, the one or more polypeptides binds, or is capable of binding, to an HLA protein encoded by HLA-B*55:02. In another example, the one or more polypeptides binds, or is capable of binding, to an HLA protein encoded by HLA-B*45:01. In another example, the one or more polypeptides binds, or is capable of binding, to an HLA protein encoded by HLA-B*50:01. In another example, the one or more polypeptides binds, or is capable of binding, to an HLA protein encoded by HLA-B*35:03. In another example, the one or more polypeptides binds, or is capable of binding, to an HLA protein encoded by HLA-B*49:01. In another example, the one or more polypeptides binds, or is capable of binding, to an HLA protein encoded by HLA-B*58:02. In another example, the one or more polypeptides binds, or is capable of binding, to an HLA protein encoded by HLA-B*15:17. In another example, the one or more polypeptides binds, or is capable of binding, to an HLA protein encoded by HLA-C*57:02. In another example, the one or more polypeptides binds, or is capable of binding, to an HLA protein encoded by HLA-C*04:01. In another example, the one or more polypeptides binds, or is capable of binding, to an HLA protein encoded by HLA-C*03:04. In another example, the one or more polypeptides binds, or is capable of binding, to an HLA protein encoded by HLA-C*01:02. In another example, the one or more polypeptides binds, or is capable of binding, to an HLA protein encoded by HLA-C*07:01. In another example, the one or more polypeptides binds, or is capable of binding, to an HLA protein encoded by HLA-C*06:02. In another example, the one or more polypeptides binds, or is capable of binding, to an HLA protein encoded by HLA-C*03:03. In another example, the one or more polypeptides binds, or is capable of binding, to an HLA protein encoded by HLA-C*08:01. In another example, the one or more polypeptides binds, or is capable of binding, to an HLA protein encoded by HLA-C*15:02. In another example, the one or more polypeptides binds, or is capable of binding, to an HLA protein encoded by HLA-C*12:02. In another example, the one or more polypeptides binds, or is capable of binding, to an HLA protein encoded by HLA-C*02:02. In another example, the one or more polypeptides binds, or is capable of binding, to an HLA protein encoded by HLA-C*05:01. In another example, the one or more polypeptides binds, or is capable of binding, to an HLA protein encoded by HLA-C*03:02. In another example, the one or more polypeptides binds, or is capable of binding, to an HLA protein encoded by HLA-C*16:01. In another example, the one or more polypeptides binds, or is capable of binding, to an HLA protein encoded by HLA-C*08:02. In another example, the one or more polypeptides binds, or is capable of binding, to an HLA protein encoded by HLA-C*04:03. In another example, the one or more polypeptides binds, or is capable of binding, to an HLA protein encoded by HLA-C*17:01. In another example, the one or more polypeptides binds, or is capable of binding, to an HLA protein encoded by HLA-C*17:04.

In some embodiments, the proteins encoded by HLA alleles include HLA proteins encoded by HLA-DR, HLA-DQ, HLA-DM, or HLA-DP. In another example, the one or more polypeptides of the present invention binds, or is capable of binding, to an HLA protein encoded by HLA-DR, HLA-DQ, HLA-DM, or HLA-DP.

### Sizes of Polynucleotides and Polypeptides

In some embodiments, the polynucleotides may be any length reasonable to encode an epitope. In some embodiments, the polynucleotides range in length from about 10 to about 200 or more polynucleotides. In some embodiments, the polynucleotides in length from 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144,145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, to/or 200 nucleotides in length.

In some embodiments, the polypeptides may be any length that is reasonable for an epitope. For example, the polypeptides may have a size of from 5 to 30 or more, e.g., from 5 to 25, from 5 to 20, from 5 to 15, from 5 to 10, from 6 to 10, from 7 to 9, or from 8 to 9 amino acids. For example, the polypeptides may have 5, 6, 7, 8, 9, 10, 11, 12, 13, 14, or 15 amino acids. In some embodiments, the optimal length of a polypeptide may be determined based the immunogenicity of the polypeptides of different lengths when introduced to a cell or subject.

### Modifications on Polypeptides

In some embodiments, polypeptides of the present invention herein may comprise one or more modifications (e.g., post-translational modifications). In some cases, the polypeptides may comprise cysteinylated Cysteine. Other examples of modifications include ubiquitination, phosphorylation, sulfonation, glycosylation, acetylation, methylation, ADP-ribosylation, methionine oxidation, cysteine oxidation, cysteine lipidation, farnesylation, geranylation, pyroglutamation, and deamidation. In some embodiments, the polypeptide comprises one or more (e.g., 1, 2, 3, 4, 5, 6, 7, 8, 9, 10, or more) amino acids that are each independently modified with an ubiquitination, phosphorylation, sulfonation, glycosylation, acetylation, methylation, ADP-ribosylation, methionine oxidation, cysteine oxidation, cysteine lipidation, farnesylation, geranylation, pyroglutamation, or deamidation.

Synthetic mRNA

In some embodiments, the polynucleotide of the present invention is mRNA, e.g., synthetic mRNA. In some embodiments, the synthetic mRNA may comprise coding sequence(s) for one or more polypeptides herein. In some embodiments, the synthetic mRNA is encoded by a polynucleotide of SEQ ID NOS: 1-9890 or SEQ ID NOS: 9891-16277 and/or identified using a method of the present invention described elsewhere herein. A synthetic mRNA may be an mRNA produced through an in vitro transcription reaction or through artificial (non-natural) chemical synthesis or through a combination thereof. In some embodiments, the synthetic mRNA further comprises a poly A tail, a Kozak sequence, a 3′ untranslated region, a 5′ untranslated region, or any combination thereof. Poly A tails in particular can be added to a synthetic RNA using a variety of art-recognized techniques, e.g., using poly A polymerase, using transcription directly from PCR products, or by ligating to the 3′ end of a synthetic RNA with RNA ligase.

The synthetic mRNA may comprise one or more stabilizing elements that maintain or enhance the stabilities of mRNA, e.g., reducing or preventing degradation of the mRNA. Examples of stabilizing elements include untranslated regions (UTR) at their 5′-end (5′UTR) and/or at their 3′-end (3′UTR), in addition to other structural features, such as a 5′-cap structure or a 3′-poly(A) tail. The stabilizing elements may be a histone stem-loop, e.g., a histone stem loop added by a stem-loop binding protein (SLBP).

### Delivery Vehicles

Described in certain example embodiments herein delivery vehicles, including but not limited to, vectors and virus particles that can deliver a polynucleotide and/or polypeptide of the present invention, which is also generally referred to as “cargo” in this context. It will be appreciated that other molecules can also be included as cargo. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses (e.g., virus particles), non-viral vehicles, and other delivery reagents described herein.

The delivery vehicles described herein can have a greatest dimension or greatest average dimension (e.g., diameter or greatest average diameter) of less than 100 microns (μm). In some embodiments, the delivery vehicles have a greatest dimension or greatest average dimension of less than 10 μm. In some embodiments, the delivery vehicles may have a greatest dimension or greatest average dimension of less than 2000 nanometers (nm). In some embodiments, the delivery vehicles may have a greatest dimension or greatest average dimension of less than 1000 nanometers (nm). In some embodiments, the delivery vehicles may have a greatest dimension or greatest average dimension (e.g., diameter or average diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension or greatest average dimension ranging between 25 nm and 200 nm.

In some embodiments, the delivery vehicles may be or comprise particles. For example, the delivery vehicle may be or comprise nanoparticles (e.g., particles with a greatest dimension or greatest average dimension (e.g., diameter or greatest average diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

In some embodiments, the delivery vehicles are nanoparticles. Exemplary nanoparticles are described in WO 2008042156, US 20130185823, and WO2015089419. In general, a “nanoparticle” refers to any particle having a diameter of less than 1000 nm. In certain embodiments, nanoparticles of the invention have a greatest dimension or greatest average dimension (e.g., diameter or average diameter) of 500 nm or less. In other embodiments, nanoparticles of the invention have a greatest dimension or greatest average dimension ranging between 25 nm and 200 nm. In other embodiments, nanoparticles of the invention have a greatest dimension or greatest average dimension of 100 nm or less. In other embodiments, nanoparticles of the invention have a greatest dimension or greatest average dimensions ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured, and are within the scope of the present invention. Nanoparticles with one half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry(MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (herein cargo refers to e.g., one or more components of CRISPR-Cas system e.g., CRISPR enzyme or mRNA or guide RNA, or any combination thereof, and may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS). Mention is made of U.S. Pat. Nos. 8,709,843; 6,007,845; 5,855,913; 5,985,309; 5,543,158; and the publication by James E. Dahlman and Carmen Barnes et al. Nature Nanotechnology (2014) published online 11 May 2014, doi:10.1038/nnano.2014.84, describing particles, methods of making and using them and measurements thereof.

### Vectors and Vector Systems

Also provided herein are vectors that can contain one or more of the polynucleotides of the present invention described elsewhere herein. In certain embodiments, the vector can contain one or more polynucleotides encoding one or more polypeptides, such as a viral polypeptide, of the present invention described elsewhere herein. The vectors can be useful in producing bacterial, fungal, yeast, plant cells, animal cells, and transgenic animals that can express one or more polynucleotides and/or polypeptides of the present invention described elsewhere herein. Within the scope of this disclosure are vectors containing one or more of the polynucleotide sequences described herein. The vectors and/or vector systems can be used, for example, to express one or more of the polynucleotides in a cell, such as a producer cell, to produce virus particles containing one or more polynucleotide(s) of the present invention described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g. a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In an embodiment, the vector can be a viral vector. In certain embodiments, the viral vector is an is an adeno-associated virus (AAV), adenovirus vector, a retroviral vector, or lentiviral vector.

These and others are further detailed and described elsewhere herein.

### Cell-Based Vector Amplification and Expression

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In some embodiments, a prokaryote is used to amplify copies of a vector and express one or more nucleic acids, such as to provide a source of one or more proteins for delivery to a host cell or host organism.

Vectors can be designed for expression of the polynucleotides and/or polypeptides of the present invention described herein (e.g. nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, and mammalian cells. In some embodiments, the suitable host cell is a eukaryotic cell.

In some embodiments, the suitable host cell is a suitable bacterial cell. Suitable bacterial cells include but are not limited to bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif (1990).

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2 plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. In some embodiments, the suitable host cell is an insect cell. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more polynucleotides of the present invention so as to drive expression of the one or more polynucleotides of the present invention described herein.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET lid (Studier et al., GENE EXPRESSION TECHNOLOGY: METHODS IN ENZYMOLOGY 185, Academic Press, San Diego, Calif. (1990) 60-89).

In some embodiments, one or more vectors driving expression of one or more polynucleotides of the present invention described herein are introduced into a cell, such as a host cell for viral particle production and/or a target cell to which a polypeptide of the present invention is to be expressed.

### Cell-Free Vector and Polynucleotide Expression

In some embodiments, the polynucleotide encoding one or more polynucleotides of the present invention can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In certain embodiments, the polynucleotides and/or vectors thereof described herein (such as the polynucleotides of the present invention, such as a viral polynucleotides of the present invention) can include one or more regulatory elements that can be operatively linked to the polynucleotide. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization signals). Such regulatory elements are described, for example, in Goeddel, GENE EXPRESSION TECHNOLOGY: METHODS IN ENZYMOLOGY 185, Academic Press, San Diego, Calif (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and International Patent Publication No. WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdx1, Alx3, Ppy), cardiac specific promoters (e.g. Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Fer114), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g. Desmin). Other tissue and/or cell specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g. a promoter that is repressed (e.g. bound by a repressor) until the repressor condition of the promotor is removed (e.g. inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the components of the CRISPR-Cas system described herein are typically placed under control of a plant promoter, i.e. a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the polynucleotides of the present invention are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for expression of one or more polynucleotides of the present invention in plants can be found in e.g., Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome)., such as a Light Inducible Transcriptional Effector (LITE) that direct changes in transcriptional activity in a sequence-specific manner. The components of a light inducible system may include one or more polynucleotides of the present invention described herein, a light-responsive cytochrome heterodimer (e.g., from Arabidopsis thaliana), and a transcriptional activation/repression domain. In some embodiments, the vector can include one or more of the inducible DNA binding proteins provided in International Patent Publication No. WO 2014/018423 and US Patent Publication Nos., 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

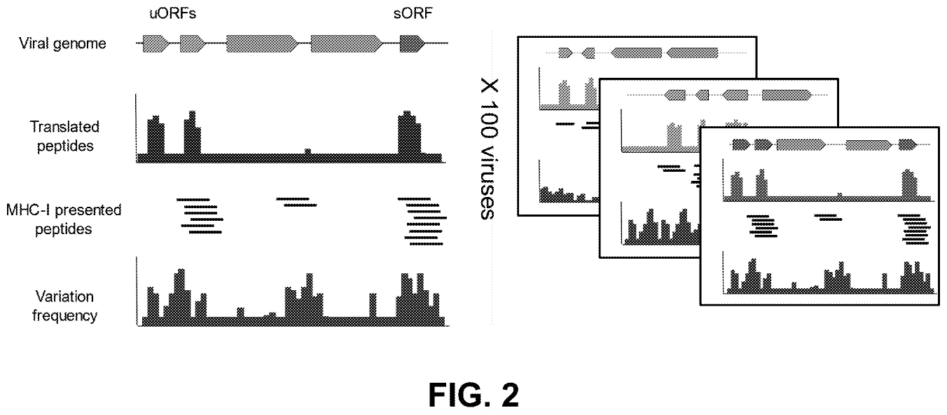

In some embodiments, the polynucleotide, vector or system thereof can include one or more elements capable of translocating and/or expressing one or more polynucleotides of the present invention to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., http://genome.unmc.edu/LocSigDB/ and Negi et al., 2015. Database. 2015: bav003; doi: 10.1093/database/bav003), nuclear export signals (e.g., LXXXLXXLXL and others described elsewhere herein), endoplasmic reticulum localization/retention signals (e.g., KDEL, KDXX, KKXX, KXX, and others described elsewhere herein; and see e.g. Liu et al. 2007 Mol. Biol. Cell. 18(3):1073-1082 and Gorleku et al., 2011. J. Biol. Chem. 286:39573-39584), mitochondria (see e.g., Cell Reports. 22:2818-2826, particularly at FIG. 2; Doyle et al. 2013. PLoS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430, peroxisome (e.g. (S/A/C)-(K/R/H)-(L/A), SLK, (R/K)-(L/V/I)-XXXXX-(H/Q)-(L/A/F). Suitable protein targeting motifs can also be designed or identified using any suitable database or prediction tool, including but not limited to Minimotif Miner (http:minimotifminer.org, http://mitominer.mrc-mbu.cam.ac.uk/release-4.0/embodiment.do?name=Protein %20MTS), LocDB (see above), PTSs predictor ( ), TargetP-2.0 (http://www.cbs.dtu.dk/services/TargetP/), ChloroP (http://www.cbs.dtu.dk/services/ChloroP/); NetNES (http://www.cbs.dtu.dk/services/NetNES/), Predotar (https://urgi.versailles.inra.fr/predotar/), and SignalP (http://www.cbs.dtu.dk/services/SignalP/).

### Selectable Markers and Tags

One or more of the polynucleotides of the present invention can be operably linked, fused to, or otherwise modified to include a polynucleotide that encodes or is a selectable marker or tag, which can be a polynucleotide or polypeptide. In some embodiments, the polynucleotide encoding a polypeptide selectable marker can be incorporated with the polynucleotide of the present invention, such as a viral polynucleotide, such that the selectable marker polypeptide, when translated, is inserted between two amino acids between the N- and C-terminus of the polypeptide of the present invention or is present at the N- and/or C-terminus of the polypeptide of the present invention. In some embodiments, the selectable marker or tag is a polynucleotide barcode or unique molecular identifier (UMI).

It will be appreciated that the polynucleotide encoding such selectable markers or tags can be incorporated into a polynucleotide encoding one or more polypeptides of the present invention, such as a viral polypeptide, described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more polypeptides of the present invention herein via suitable linker, such as a glycine or glycine serine linkers as short as GS or GG up to (GGGGG)₃ (SEQ ID NO: 16303) or (GGGGS)₃ (SEQ ID NO: 16304). Other suitable linkers are described elsewhere herein.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the polynucleotide(s) and/or products expressed therefrom (e.g., polypeptides) include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated polynucleotide(s) and/or polypeptides of the present invention to specific cells, tissues, organs, etc.

### Codon Optimization of Vector Polynucleotides

As described elsewhere herein, the polynucleotide encoding one or more polypeptides of the present invention described herein can be codon optimized. In some embodiments, one or more polynucleotides contained in a vector (“vector polynucleotides”) described herein that are in addition to an optionally codon optimized polynucleotide encoding one or more polypeptides of the present invention, such as viral polypeptides, described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g. a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g. astrocytes, glial cells, Schwann cells etc.), muscle cells (e.g. cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. nAAV vectors are discussed elsewhere herein.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. When multiple different guide polynucleotides are used, a single expression construct may be used to target nucleic acid-targeting activity to multiple different, corresponding target sequences within a cell. For example, a single vector may comprise about or more than about 1, 2, 3, 4, 5, 6, 7, 8, 9, 10, 15, 20, or more guide s polynucleotides. In some embodiments, about or more than about 1, 2, 3, 4, 5, 6, 7, 8, 9, 10, or more such guide-polynucleotide-containing vectors may be provided, and optionally delivered to a cell.

Delivery vehicles, vectors, particles, nanoparticles, formulations and components thereof for expression of one or more polynucleotides and/or polypeptides of the present invention, such as one or more viral polynucleotides and/or polypeptides, described herein are as used in the foregoing documents, such as International Patent Publication No. WO 2014/093622 (PCT/US2013/074667) and are discussed in greater detail herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as a viral polynucleotide of the present invention, into a virus particle and producing said virus particle when used alone or with one or more other viral vectors (such as in a viral vector system). Viral vectors and systems thereof can be used for producing viral particles for delivery of and/or expression of one or more polynucleotides and/or polypeptides of the present invention described herein. The viral vector can be part of a viral vector system involving multiple vectors. In some embodiments, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produce therefrom are described elsewhere herein. In some embodiments, the viral vectors are configured to produce replication incompetent viral particles for improved safety of these systems.

In certain embodiments, the virus structural component, which can be encoded by one or more polynucleotides in a viral vector or vector system, comprises one or more capsid proteins including an entire capsid. In certain embodiments, such as wherein a viral capsid comprises multiple copies of different proteins, the delivery system can provide one or more of the same protein or a mixture of such proteins. For example, AAV comprises three (3) capsid proteins, VP1, VP2, and VP3, thus delivery systems of the invention can comprise one or more of VP1, and/or one or more of VP2, and/or one or more of VP3. Accordingly, the present invention is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Thus, a virus within the family Adenoviridae is contemplated as within the invention with discussion herein as to adenovirus applicable to other family members. Target-specific AAV capsid variants can be used or selected. Non-limiting examples include capsid variants selected to bind to chronic myelogenous leukemia cells, human CD34 PBPC cells, breast cancer cells, cells of lung, heart, dermal fibroblasts, melanoma cells, stem cell, glioblastoma cells, coronary artery endothelial cells and keratinocytes. See, e.g., Buning et al, 2015, Current Opinion in Pharmacology 24, 94-104. From teachings herein and knowledge in the art as to modifications of adenovirus (see, e.g., U.S. Pat. Nos. 9,410,129, 7,344,872, 7,256,036, 6,911,199, 6,740,525; Matthews, “Capsid-Incorporation of Antigens into Adenovirus Capsid Proteins for a Vaccine Approach,” Mol Pharm, 8(1): 3-11 (2011)), as well as regarding modifications of AAV, the skilled person can readily obtain a modified adenovirus that has a large payload protein, despite that heretofore it was not expected that such a large protein could be provided on an adenovirus. And as to the viruses related to adenovirus mentioned herein, as well as to the viruses related to AAV mentioned elsewhere herein, the teachings herein as to modifying adenovirus and AAV, respectively, can be applied to those viruses without undue experimentation from this disclosure and the knowledge in the art.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the CRISPR-Cas systems can include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). Selection of a retroviral gene transfer system may therefore depend on the target tissue.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-HLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In some embodiments, an HIV-based lentiviral vector system can be used. In some embodiments, a FIV-based lentiviral vector system can be used.

In some embodiments, the lentiviral vector is an EIAV-based lentiviral vector or vector system. EIAV vectors have been used to mediate expression, packaging, and/or delivery in other contexts, such as for ocular gene therapy (see, e.g., Balagaan, J Gene Med 2006; 8: 275-285). In another embodiment, RetinoStat®, (see, e.g., Binley et al., HUMAN GENE THERAPY 23:980-991 (September 2012)), which describes RetinoStat®, an equine infectious anemia virus-based lentiviral gene therapy vector that expresses angiostatic proteins endostatin and angiostatin that is delivered via a subretinal injection for the treatment of the wet form of age-related macular degeneration. Any of these vectors described in these publications can be modified for polynucleotides and/or polypeptides of the present invention described herein.

In some embodiments, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In some embodiments, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In some embodiments, the second-generation vector lacks one or more accessory virulence factors (e.g., vif, vprm, vpu, nef, and combinations thereof). Unlike the first-generation lentiviral vectors, no single second generation lentiviral vector includes all features necessary to express and package a polynucleotide into a virus particle. In some embodiments, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In some embodiments, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included up-stream of the LTRs), and they can include one or more deletions in the 3′LTR to create self-inactivating (SIN) vectors having disrupted promoter/enhancer activity of the LTR. In some embodiments, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g. gag, pol, and rev) and upstream regulatory sequences (e.g. promoter(s)) to drive expression of the features present on the packaging vector, and (iii) an “envelope vector” that contains one or more envelope protein genes and upstream promoters. In certain embodiments, the third-generation lentiviral vector system can include at least two packaging vectors, with the gag-pol being present on a different vector than the rev gene.

In some embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used/and or adapted to the polypeptides and/or polynucleotides of the present invention described elsewhere herein.

In some embodiments, the pseudotype and infectivity or tropisim of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In some embodiments, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD 114) (see e.g., Hanawa et al. Molec. Ther. 2002 5(3) 242-251), modified Sindbis virus envelope proteins (see e.g., Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124: 1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In some embodiments, the tropism of the resulting lentiviral particle can be tuned by incorporating cell targeting peptides into a lentiviral vector such that the cell targeting peptides are expressed on the surface of the resulting lentiviral particle. In some embodiments, a lentiviral vector can contain an envelope protein that is fused to a cell targeting protein (see e.g., Buchholz et al. 2015. Trends Biotechnol. 33:777-790; Bender et al. 2016. PLoS Pathog. 12(e1005461); and Friedrich et al. 2013. Mol. Ther. 2013. 21: 849-859.

In some embodiments, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233. In these embodiments, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In some embodiments, a covalent-bond-forming protein-peptide pair can be incorporated into one or more of the lentiviral vectors described herein to conjugate a cell targeting peptide to the virus particle (see e.g., Kasaraneni et al. 2018. Sci. Reports (8) No. 10990). In some embodiments, a lentiviral vector can include an N-terminal PDZ domain of InaD protein (PDZ1) and its pentapeptide ligand (TEFCA) from NorpA, which can conjugate the cell targeting peptide to the virus particle via a covalent bond (e.g., a disulfide bond). In some embodiments, the PDZ1 protein can be fused to an envelope protein, which can optionally be binding deficient and/or fusion competent virus envelope protein and included in a lentiviral vector. In some embodiments, the TEFCA can be fused to a cell targeting peptide and the TEFCA-CPT fusion construct can be incorporated into the same or a different lentiviral vector as the PDZ1-envenlope protein construct. During virus production, specific interaction between the PDZ1 and TEFCA facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015. Any of these systems or a variant thereof can be used to deliver a polynucleotide of the present invention described herein to a cell.

In some embodiments, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (Ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

In another embodiment, Cocal vesiculovirus envelope pseudotyped retroviral or lentiviral vector particles are contemplated (see, e.g., US Patent Publication No. 20120164118 assigned to the Fred Hutchinson Cancer Research Center). Cocal virus is in the Vesiculovirus genus, and is a causative agent of vesicular stomatitis in mammals. Cocal virus was originally isolated from mites in Trinidad (Jonkers et al., Am. J. Vet. Res. 25:236-242 (1964)), and infections have been identified in Trinidad, Brazil, and Argentina from insects, cattle, and horses. Many of the vesiculoviruses that infect mammals have been isolated from naturally infected arthropods, suggesting that they are vector-borne. Antibodies to vesiculoviruses are common among people living in rural areas where the viruses are endemic and laboratory-acquired; infections in humans usually result in influenza-like symptoms. The Cocal virus envelope glycoprotein shares 71.5% identity at the amino acid level with VSV-G Indiana, and phylogenetic comparison of the envelope gene of vesiculoviruses shows that Cocal virus is serologically distinct from, but most closely related to, VSV-G Indiana strains among the vesiculoviruses. Jonkers et al., Am. J. Vet. Res. 25:236-242 (1964) and Travassos da Rosa et al., Am. J. Tropical Med. & Hygiene 33:999-1006 (1984). The Cocal vesiculovirus envelope pseudotyped retroviral vector particles may include, for example, lentiviral, alpharetroviral, betaretroviral, gammaretroviral, deltaretroviral, and epsilonretroviral vector particles that may comprise retroviral Gag, Pol, and/or one or more accessory protein(s) and a Cocal vesiculovirus envelope protein. In certain embodiments of these embodiments, the Gag, Pol, and accessory proteins are lentiviral and/or gammaretroviral. In some embodiments, a retroviral vector can contain encoding polypeptides for one or more Cocal vesiculovirus envelope proteins such that the resulting viral or pseudoviral particles are Cocal vesiculovirus envelope pseudotyped. Adenoviral vectors, Helper-dependent Adenoviral vectors, and Hybrid Adenoviral Vectors

In some embodiments, the vector can be an adenoviral vector. In some embodiments, the adenoviral vector can include elements such that the virus particle produced using the vector or system thereof can be serotype 2 or serotype 5. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In some embodiments, the vector can be a helper-dependent adenoviral vector or system thereof. These are also referred to in the art as “gutless” or “gutted” vectors and are a modified generation of adenoviral vectors (see e.g., Thrasher et al. 2006. Nature. 443:E5-7). In certain embodiments of the helper-dependent adenoviral vector system one vector (the helper) can contain all the viral genes required for replication but contains a conditional gene defect in the packaging domain. The second vector of the system can contain only the ends of the viral genome, one or more polynucleotides of the present invention described elsewhere herein, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the polynucleotides of the present invention described herein. In some embodiments, the polynucleotide to be delivered via the viral particle produced from a helper-dependent adenoviral vector or system thereof can be up to about 37 kb. Thus, in some embodiments, an adenoviral vector can include a DNA polynucleotide to be delivered that can range in size from about 0.001 kb to about 37 kb (see e.g. Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use for delivering the polynucleotide of the present invention described elsewhere herein. In some embodiments, a hybrid-adenoviral vector can include one or more features of a retrovirus and/or an adeno-associated virus. In some embodiments the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use for delivering one or more polynucleotides of the present invention described herein. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the CRISPR-Cas system of the present invention.

### Adeno Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments the AAV can integrate into a specific site on chromosome 19 of a human cell with no observable side effects. In some embodiments, the capacity of the AAV vector, system thereof, and/or AAV particles can be up to about 4.7 kb. The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In some embodiments, the AAV capsid can contain 60 capsid proteins. In some embodiments, the ratio of VP1:VP2:VP3 in a capsid can be about 1:1:10.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E40RF6, and VA RNAs. In some embodiments, a producing host cell line expresses one or more of the adenovirus helper factors.

The AAV vector or system thereof can be configured to produce AAV particles having a specific serotype. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5 or any combination thereof. One can select the AAV of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the second plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5.

A tabulation of certain AAV serotypes as to these cells can be found in Grimm, D. et al, J. Virol. 82: 5887-5911 (2008). The AAV can be any one of the serotypes.

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the CRISPR-Cas system polynucleotide(s)).

In some embodiments, the AAV vectors are produced in in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, an AAV vector or vector system can contain or consists essentially of one or more polynucleotides encoding one or more polynucleotides of the present invention, such as one or more viral polynucleotides.

In another embodiment, the invention provides a polypeptide of the present invention operatively coupled with Adeno Associated Virus (AAV), e.g., an AAV comprising a polypeptide of the present invention as a fusion, with or without a linker, to or with an AAV capsid protein such as VP1, VP2, and/or VP3. More in particular, modifying the knowledge in the art, e.g., Rybniker et al., “Incorporation of Antigens into Viral Capsids Augments Immunogenicity of Adeno-Associated Virus Vector-Based Vaccines,” J Virol. Dec 2012; 86(24): 13800-13804, Lux K, et al. 2005. Green fluorescent protein-tagged adeno-associated virus particles allow the study of cytosolic and nuclear trafficking. J. Virol. 79:11776-11787, Munch R C, et al. 2012. “Displaying high-affinity ligands on adeno-associated viral vectors enables tumor cell-specific and safe gene transfer.” Mol. Ther. [Epub ahead of print.]doi:10.1038/mt.2012.186 and Warrington K H, Jr, et al. 2004. Adeno-associated virus type 2 VP2 capsid protein is nonessential and can tolerate large peptide insertions at its N terminus. J. Virol. 78:6595-6609, each incorporated herein by reference, one can obtain a modified AAV capsid of the invention. It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3). One can modify the cap gene to have expressed at a desired location a non-capsid protein advantageously a large payload protein, such as a polypeptide of the present invention. Likewise, these can be fusions, with the protein, e.g., large payload protein such as a polypeptide of the present invention fused in a manner analogous to prior art fusions. See, e.g., US Patent Publication 20090215879; Nance et al., “Perspective on Adeno-Associated Virus Capsid Modification for Duchenne Muscular Dystrophy Gene Therapy,” Hum Gene Ther. 26(12):786-800 (2015) and documents cited therein, incorporated herein by reference. The skilled person, from this disclosure and the knowledge in the art can make and use modified AAV or AAV capsid as in the herein invention, and through this disclosure one knows now that payload proteins, such as large payload proteins, can be fused to the AAV capsid. Accordingly, this approach is also applicable to a virus in the genus Dependoparvovirus or in the family Parvoviridae, for instance, AAV, or a virus of Amdoparvovirus, e.g., Carnivore amdoparvovirus 1, a virus of Aveparvovirus, e.g., Galliform aveparvovirus 1, a virus of Bocaparvovirus, e.g., Ungulate bocaparvovirus 1, a virus of Copiparvovirus, e.g., Ungulate copiparvovirus 1, a virus of Dependoparvovirus, e.g., Adeno-associated dependoparvovirus A, a virus of Erythroparvovirus, e.g., Primate erythroparvovirus 1, a virus of Protoparvovirus, e.g., Rodent protoparvovirus 1, a virus of Tetraparvovirus, e.g., Primate tetraparvovirus 1. Thus, a virus of within the family Parvoviridae or the genus Dependoparvovirus or any of the other foregoing genera within Parvoviridae is contemplated as within the invention with discussion herein as to AAV applicable to such other viruses.

In one embodiment, the invention provides a non-naturally occurring or engineered composition comprising a polypeptide of the present invention which is part of or tethered to an AAV capsid domain, i.e., VP1, VP2, or VP3 domain of Adeno-Associated Virus (AAV) capsid. In some embodiments, part of or tethered to an AAV capsid domain includes associated with associated with a AAV capsid domain. In some embodiments, the polypeptide of the present invention may be fused to the AAV capsid domain. In some embodiments, the fusion may be to the N-terminal end of the AAV capsid domain. As such, in some embodiments, the C-terminal end of the polypeptide of the present invention is fused to the N-terminal end of the AAV capsid domain. In some embodiments, an NLS and/or a linker (such as a GlySer linker) may be positioned between the C-terminal end of the polypeptide of the present invention and the N-terminal end of the AAV capsid domain. In some embodiments, the fusion may be to the C-terminal end of the AAV capsid domain. In some embodiments, this is not preferred due to the fact that the VP1, VP2 and VP3 domains of AAV are alternative splices of the same RNA and so a C-terminal fusion may affect all three domains. In some embodiments, the AAV capsid domain is truncated. In some embodiments, some or all of the AAV capsid domain is removed. In some embodiments, some of the AAV capsid domain is removed and replaced with a linker (such as a GlySer linker), typically leaving the N-terminal and C-terminal ends of the AAV capsid domain intact, such as the first 2, 5 or 10 amino acids. In this way, the internal (non-terminal) portion of the VP3 domain may be replaced with a linker. It is particularly preferred that the linker is fused to the polypeptide of the present invention. A branched linker may be used, with the polypeptide of the present invention fused to the end of one of the branches. This allows for some degree of spatial separation between the capsid and the polypeptide of the present invention. In this way, the polypeptide of the present invention is part of (or fused to) the AAV capsid domain.

In other embodiments, the polypeptide of the present invention may be fused in frame within, i.e., internal to, the AAV capsid domain. Thus, in some embodiments, the AAV capsid domain again preferably retains its N-terminal and C-terminal ends. In this case, a linker is preferred, in some embodiments, either at one or both ends of the polypeptide of the present invention. In this way, the polypeptide of the present invention is again part of (or fused to) the AAV capsid domain. In certain embodiments, the positioning of the polypeptide of the present invention is such that the polypeptide of the present invention is at the external surface of the viral capsid once formed. In one embodiment, the invention provides a non-naturally occurring or engineered composition comprising a polypeptide of the present invention associated with a AAV capsid domain of Adeno-Associated Virus (AAV) capsid. Here, associated may mean in some embodiments fused, or in some embodiments bound to, or in some embodiments tethered to. The polypeptide of the present invention may, in some embodiments, be tethered to the VP1, VP2, or VP3 domain. This may be via a connector protein or tethering system such as the biotin-streptavidin system. In one example, a biotinylation sequence (15 amino acids) could therefore be fused to the polypeptide of the present invention. When a fusion of the AAV capsid domain, especially the N-terminus of the AAV capsid domain, with streptavidin is also provided, the two will therefore associate with very high affinity. Thus, in some embodiments, provided is a composition or system comprising a polypeptide of the present invention-biotin fusion and a streptavidin-AAV capsid domain arrangement, such as a fusion. The polypeptide of the present invention-biotin and streptavidin-AAV capsid domain forms a single complex when the two parts are brought together. NLSs may also be incorporated between the polypeptide of the present invention and the biotin; and/or between the streptavidin and the AAV capsid domain.

As such, provided is a fusion of a polypeptide of the present invention with a connector protein specific for a high affinity ligand for that connector, whereas the AAV VP2 domain is bound to said high affinity ligand. For example, streptavidin may be the connector fused to the polypeptide of the present invention, while biotin may be bound to the AAV VP2 domain. Upon co-localization, the streptavidin will bind to the biotin, thus connecting the polypeptide of the present invention to the AAV VP2 domain. The reverse arrangement is also possible. In some embodiments, a biotinylation sequence (15 amino acids) could therefore be fused to the AAV VP2 domain, especially the N-terminus of the AAV VP2 domain. A fusion of the polypeptide of the present invention with streptavidin is also preferred, in some embodiments. In some embodiments, the biotinylated AAV capsids with streptavidin-polypeptide of the present invention are assembled in vitro. This way the AAV capsids should assemble in a straightforward manner and the polypeptide of the present invention-streptavidin fusion can be added after assembly of the capsid. In other embodiments a biotinylation sequence (15 amino acids) could therefore be fused to the polypeptide of the present invention, together with a fusion of the AAV VP2 domain, especially the N-terminus of the AAV VP2 domain, with streptavidin. For simplicity, a fusion of the polypeptide of the present invention and the AAV VP2 domain is preferred in some embodiments. In some embodiments, the fusion may be to the N-terminal end of the polypeptide of the present invention. In other words, in some embodiments, the AAV and polypeptide of the present invention are associated via fusion. In some embodiments, the AAV and polypeptide of the present invention are associated via fusion including a linker. Suitable linkers are discussed herein but include Gly Ser linkers. Fusion to the N-term of AAV VP2 domain is preferred, in some embodiments. In some embodiments, the polypeptide of the present invention comprises at least one Nuclear Localization Signal (NLS). In a further embodiment, the present invention provides compositions comprising the polypeptide of the present invention and associated AAV VP2 domain or the polynucleotides or vectors described herein. Such compositions and formulations are discussed elsewhere herein.

An alternative tether may be to fuse or otherwise associate the AAV capsid domain to an adaptor protein which binds to or recognizes to a corresponding RNA sequence or motif. In some embodiments, the adaptor is or comprises a binding protein which recognizes and binds (or is bound by) an RNA sequence specific for said binding protein. In some embodiments, a preferred example is the MS2 (see Konermann et al. Dec 2014, cited infra, incorporated herein by reference) binding protein which recognizes and binds (or is bound by) an RNA sequence specific for the MS2 protein.

With the AAV capsid domain associated with the adaptor protein, the polypeptide of the present invention may, in some embodiments, be tethered to the adaptor protein of the AAV capsid domain. The polypeptide of the present invention may, in some embodiments, be tethered to the adaptor protein of the AAV capsid domain via the polypeptide of the present invention being in a complex with a modified guide, see Konermann et al. The modified guide is, in some embodiments, a sgRNA. In some embodiments, the modified guide comprises a distinct RNA sequence; see, e.g., International Patent Application No. PCT/US14/70175, incorporated herein by reference. In some embodiments, distinct RNA sequence is an aptamer.

In certain embodiments, the positioning of the polypeptide of the present invention is such that the polypeptide of the present invention is at the internal surface of the viral capsid once formed. In one embodiment, the invention provides a non-naturally occurring or engineered composition comprising a polypeptide of the present invention associated with an internal surface of an AAV capsid domain. Here again, associated may mean in some embodiments fused, or in some embodiments bound to, or in some embodiments tethered to. The polypeptide of the present invention may, in some embodiments, be tethered to the VP1, VP2, or VP3 domain such that it locates to the internal surface of the viral capsid once formed. This may be via a connector protein or tethering system such as the biotin-streptavidin system as described above and/or elsewhere herein.

### Herpes Simplex Viral Vectors

In some embodiments, the vector can be a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g., 2009. Trobridge. Exp. Opin. Biol. Ther. 9:1427-1436, whose techniques and vectors described therein can be modified and adapted for use in the CRISPR-Cas system of the present invention. In some embodiments where an HSV vector or system thereof is utilized, the host cell can be a complementing cell. In some embodiments, HSV vector or system thereof can be capable of producing virus particles capable of delivering a polynucleotide cargo of up to 150 kb. Thus, in some embodiment the polynucleotide(s) of the present invention included in the HSV-based viral vector or system thereof can sum from about 0.001 to about 150 kb. HSV-based vectors and systems thereof have been successfully used in several contexts including various models of neurologic disorders. See e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use to delivery one or more polynucleotides and/or polypeptides of the present invention of the present invention.

### Poxvirus Vectors

In some embodiments, the vector can be a poxvirus vector or system thereof. In some embodiments, the poxvirus vector can result in cytoplasmic expression of one or more polynucleotides and/or polypeptides of the present invention. In some embodiments, the capacity of a poxvirus vector or system thereof can be about 25 kb or more. In some embodiments, a poxvirus vector or system thereof can include one or more polynucleotides of the present invention described herein.

### Viral Vectors for Delivery to Plants

The systems and compositions may be delivered to plant cells using viral vehicles. In particular embodiments, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

Virus Particle Production from Viral Vectors

### Retroviral Production

In some embodiments, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g., pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., a viral polynucleotide of the present invention), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g. NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ particles/mL.

Lentiviruses may be prepared from any lentiviral vector or vector system described herein. In one example embodiment, after cloning pCasES10 (which contains a lentiviral transfer plasmid backbone), HEK293FT at low passage (p=5) can be seeded in a T-75 flask to 50% confluence the day before transfection in DMEM with 10% fetal bovine serum and without antibiotics. After 20 hours, the media can be changed to OptiMEM (serum-free) media and transfection of the lentiviral vectors can done 4 hours later. Cells can be transfected with 10 μg of lentiviral transfer plasmid (pCasES10) and the appropriate packaging plasmids (e.g., 5 μg of pMD2.G (VSV-g pseudotype), and 7.5 ug of psPAX2 (gag/pol/rev/tat)). Transfection can be carried out in 4 mL OptiMEM with a cationic lipid delivery agent (50 uL Lipofectamine 2000 and 100 ul Plus reagent). After 6 hours, the media can be changed to antibiotic-free DMEM with 10% fetal bovine serum. These methods can use serum during cell culture, but serum-free methods are preferred.

Following transfection and allowing the producing cells (also referred to as packaging cells) to package and produce virus particles with packaged cargo, the lentiviral particles can be purified. In an exemplary embodiment, virus-containing supernatants can be harvested after 48 hours. Collected virus-containing supernatants can first be cleared of debris and filtered through a 0.45 um low protein binding (PVDF) filter. They can then be spun in an ultracentrifuge for 2 hours at 24,000 rpm. The resulting virus-containing pellets can be resuspended in 50 ul of DMEM overnight at 4 degrees C. They can be then aliquoted and used immediately or immediately frozen at −80 degrees C. for storage.

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., one or more viral polynucleotide(s) of the present invention). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., one or more viral polynucleotide(s) of the present invention) between 2 ITRs; (2) a vector that carries the AAV Rep-Cap encoding polynucleotides; and (helper polynucleotides. One of skill in the art will appreciate various methods and variations thereof that are both helper and -helper free and as well as the different advantages of each system.

### Non-Viral Vectors

In some embodiments, the vector is a non-viral vector or vector system. The term of art “Non-viral vector” and as used herein in this context refers to molecules and/or compositions that are vectors but that are not based on one or more component of a virus or virus genome (excluding any nucleotide to be delivered and/or expressed by the non-viral vector) that can be capable of incorporating polynucleotide(s) of the present invention and delivering said polynucleotide(s) to a cell and/or expressing the polynucleotide in the cell. It will be appreciated that this does not exclude vectors containing a polynucleotide designed to target a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors can include, without limitation, naked polynucleotides and polynucleotide (non-viral) based vector and vector systems.

### Naked Polynucleotides

In some embodiments, one or more polynucleotides of the present invention, e.g., one or more viral polynucleotides, described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the polynucleotides of the present invention described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g. ribozymes), and the like. In some embodiments, the naked polynucleotide contains only the polynucleotide(s) of the present invention. In some embodiments, the naked polynucleotide can contain other nucleic acids and/or polynucleotides in addition to the polynucleotide(s) of the present invention. The naked polynucleotides can include one or more elements of a transposon system. Transposons and system thereof are described in greater detail elsewhere herein.

### Non-Viral Polynucleotide Vectors

In some embodiments, one or more of the polynucleotides of the present invention, such as a viral polynucleotide of the present invention described elsewhere herein, can be included in a non-viral polynucleotide vector. Suitable non-viral polynucleotide vectors include, but are not limited to, transposon vectors and vector systems, plasmids, bacterial artificial chromosomes, yeast artificial chromosomes, AR(antibiotic resistance)-free plasmids and miniplasmids, circular covalently closed vectors (e.g. minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In certain embodiments, the S/MAR sequence is located downstream of an actively transcribed polynucleotide (e.g., one or more polynucleotides of the present invention) included in the non-viral polynucleotide vector. In some embodiments, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments, a non-viral polynucleotide transposon vector system can include a first polynucleotide vector that contains the polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell, the transposase can be expressed from the second vector and can transpose the material between the TIRs on the first vector (e.g., the polynucleotide(s) of the present invention) and integrate it into one or more positions in the host cell's genome. In some embodiments the transposon vector or system thereof can be configured as a gene trap. In some embodiments, the TIRs can be configured to flank a strong splice acceptor site followed by a reporter and/or other gene (e.g., one or more of the polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include Sleeping Beauty transposon system (Tc1/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tc1/mariner superfamily) (see e.g., Miskey et al. 2003 Nucleic Acid Res. 31(23):6873-6881) and variants thereof.

### Non-Vector Delivery Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples, LNPs may be used for delivering DNA molecules (e.g., those comprising polynucleotides of the present invention and/or polypeptides they encode).

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al., Molecular Therapy, vol. 19, no. 12, pages 1286-2200, Dec. 2011).

In some embodiments, an LNP delivery vehicle can be used to deliver a virus particle containing a polynucleotides and/or polypeptides of the present invention. In some embodiments, the virus particle(s) can be adsorbed to the lipid particle, such as through electrostatic interactions, and/or can be attached to the liposomes via a linker.

In some embodiments, the LNP contains a nucleic acid, wherein the charge ratio of nucleic acid backbone phosphates to cationic lipid nitrogen atoms is about 1:1.5-7 or about 1:4.

In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxyethylglucose (HEG) based polymers, polyhydroxyethyl starch (polyHES) and polypropylene. In some embodiments, the PEG, HEG, polyHES, and a polypropylene weight between about 500 to 10,000 Da or between about 2000 to 5000 Da. In some embodiments, the shielding compound is PEG2000 or PEG5000.

In some embodiments, the LNP can include one or more helper lipids. In some embodiments, the helper lipid can be a phosphor lipid or a steroid. In some embodiments, the helper lipid is between about 20 mol % to 80 mol % of the total lipid content of the composition. In some embodiments, the helper lipid component is between about 35 mol % to 65 mol % of the total lipid content of the LNP. In some embodiments, the LNP includes lipids at 50 mol % and the helper lipid at 50 mol % of the total lipid content of the LNP.

Other non-limiting, exemplary LNP delivery vehicles are described in U.S. Patent Publication Nos. US 20160174546, US 20140301951, US 20150105538, US 20150250725, Wang et al., J. Control Release, 2017 Jan. 31. pii: S0168-3659(17)30038-X. doi: 10.1016/j.jconrel.2017.01.037. [Epub ahead of print]; Altmoglu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthc Mater., 3(9):1398-403, Sep. 2014; and Wang et al., Agnew Chem Int Ed Engl., 53(11):2893-8, Mar. 10, 2014; James E. Dahlman and Carmen Barnes et al. Nature Nanotechnology (2014) published online 11 May 2014, doi:10.1038/nnano.2014.84; Coelho et al., N Engl J Med 2013; 369:819-29; Aleku et al., Cancer Res., 68(23): 9788-98 (Dec. 1, 2008), Strumberg et al., Int. J. C/in. Pharmacol. Ther., 50(1): 76-8 (Jan. 2012), Schultheis et al., J. C/in. Oncol., 32(36): 4141-48 (Dec. 20, 2014), and Fehring et al., Mol. Ther., 22(4): 811-20 (Apr. 22, 2014); Novobrantseva, Molecular Therapy—Nucleic Acids (2012) 1, e4; doi:10.1038/mtna.2011.3; WO2012135025; US 20140348900; US 20140328759; US 20140308304; WO 2005/105152; WO 2006/069782; WO 2007/121947; US 2015/082080; US 20120251618; 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316;

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In some embodiments, a liposome delivery vehicle can be used to deliver a virus particle containing polynucleotides and/or polypeptides of the present invention described elsewhere herein. In some embodiments, the virus particle(s) can be adsorbed to the liposome, such as through electrostatic interactions, and/or can be attached to the liposomes via a linker.

In some embodiments, the liposome can be a Trojan Horse liposome (also known in the art as Molecular Trojan Horses), see e.g. http://cshprotocols.cshlp.org/content/2010/4/pdb.prot5407.long, the teachings of which can be applied and/or adapted to generated and/or deliver the polynucleotides and/or polypeptides of the present invention described elsewhere herein.

Other non-limiting, exemplary liposomes can be those as set forth in Wang et al., ACS Synthetic Biology, 1, 403-07 (2012); Wang et al., PNAS, 113(11) 2868-2873 (2016); Spuch and Navarro, Journal of Drug Delivery, vol. 2011, Article ID 469679, 12 pages, 2011. doi:10.1155/2011/469679; WO 2008/042973; U.S. Pat. No. 8,071,082; WO 2014/186366; 20160257951; US20160129120; US 20160244761; 20120251618; WO2013/093648; Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMAo).

Other non-limiting, exemplary SNALPs that can be used to deliver the polynucleotides and/or polypeptides of the present invention described elsewhere herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375: 1896-905; Judge, J. Clin. Invest. 119:661-673 (2009); and Semple et al., Nature Niotechnology, Volume 28 Number 2 Feb. 2010, pp. 172-177.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

In some embodiments, the delivery vehicle can be or include a lipidoid, such as any of those set forth in, for example, US 20110293703.

In some embodiments, the delivery vehicle can be or include an amino lipid, such as any of those set forth in, for example, Jayaraman, Angew. Chem. Int. Ed. 2012, 51, 8529-8533.

In some embodiments, the delivery vehicle can be or include a lipid envelope, such as any of those set forth in, for example, Korman et al., 2011. Nat. Biotech. 29:154-157.

### Lipoplexes Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2 [symbol] (e.g., forming DNA/Ca²⁺ microcomplexes), polyethenimine (PEI) (e.g., branched PEI), and poly(L-lysine) (PLL).

### Sugar-Based Particles

In some embodiments, the delivery vehicle can be a sugar-based particle. In some embodiments, the sugar-based particles can be or include GalNAc, such as any of those described in WO2014118272; US 20020150626; Nair, J K et al., 2014, Journal of the American Chemical Society 136 (49), 16958-16961; Ostergaard et al., Bioconjugate Chem., 2015, 26 (8), pp 1451-1455.

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have palindromic sequences to be partially complementary to one or more of the polynucleotides of the present invention described elsewhere herein. A DNA nanoclew may be coated, e.g., coated with PEI to induce endosomal escape.

### Metal Nanoparticles

In some embodiments, the delivery vehicles comprise gold nanoparticles (also referred to as AuNPs or colloidal gold). Gold nanoparticles may form complex with cargos, e.g., polynucleotides and/or polypeptides of the present invention described elsewhere herein. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal particles include tungsten, palladium, rhodium, platinum, and iridium particles. Other non-limiting, exemplary metal nanoparticles are described in US 20100129793.

iTOP

In some embodiments, the delivery vehicles comprise iTOP. iTOP refers to a combination of small molecules, drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids (siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the polynucleotides and/or polypeptides of the present invention described elsewhere herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi:10.13140/RG.2.2.23912.16642. Other exemplary and non-limiting polymeric particles are described in US 20170079916, US 20160367686, US 20110212179, US 20130302401, 6,007,845, 5,855,913, 5,985,309, 5,543,158, WO2012135025, US 20130252281, US 20130245107, US 20130244279; US 20050019923, and/or 20080267903.

### Streptolvsin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevices (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and deliver various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome may form a complex (e.g., by binding directly or indirectly) to one or more components of the cargo. In certain examples, a molecule of an exosome may be fused with a first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr 28. doi: 10.1039/d0bm00427h.

Other non-limiting, exemplary exosomes include any of those set forth in Alvarez-Erviti et al. 2011, Nat Biotechnol 29: 341; [1401]; El-Andaloussi et al. (Nature Protocols 7:2112-2126(2012); and Wahlgren et al. (Nucleic Acids Research, 2012, Vol. 40, No. 17 e130).

### Spherical Nucleic Acids (SNAs)

In some embodiments, the delivery vehicle can be a SNA. SNAs are three dimensional nanostructures that can be composed of densely functionalized and highly oriented nucleic acids that can be covalently attached to the surface of spherical nanoparticle cores. The core of the spherical nucleic acid can impart the conjugate with specific chemical and physical properties, and it can act as a scaffold for assembling and orienting the oligonucleotides into a dense spherical arrangement that gives rise to many of their functional properties, distinguishing them from all other forms of matter. In some embodiments, the core is a crosslinked polymer. Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., and Small, 10:186-192.

### Self-Assembling Nanoparticles

In some embodiments, the delivery vehicle is a self-assembling nanoparticle. The self-assembling nanoparticles can contain one or more polymers. The self-assembling nanoparticles can be PEGylated. Self-assembling nanoparticles are known in the art. Non-limiting, exemplary self-assembling nanoparticles can be any as set forth in Schiffelers et al., Nucleic Acids Research, 2004, Vol. 32, No. 19, Bartlett et al. (PNAS, Sep. 25, 2007, vol. 104, no. 39; and Davis et al., Nature, Vol 464, 15 Apr. 2010.

### Supercharged Proteins

In some embodiments, the delivery vehicle can be a supercharged protein. As used herein “supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112.

### Targeted Delivery

In some embodiments, the delivery vehicle can allow for targeted delivery to a specific cell, tissue, organ, or system. In such embodiments, the delivery vehicle can include one or more targeting moieties that can direct targeted delivery of the cargo(s). In an embodiment, the delivery vehicle comprises a targeting moiety, such as active targeting of a lipid entity of the invention, e.g., lipid particle or nanoparticle or liposome or lipid bilayer of the invention comprising a targeting moiety for active targeting.

With regard to targeting moieties, mention is made of Deshpande et al., “Current trends in the use of liposomes for tumor targeting,” Nanomedicine (Lond). 8(9), doi:10.2217/nnm.13.118 (2013), and the documents it cites, all of which are incorporated herein by reference and the teachings of which can be applied and/or adapted for targeted delivery of one or more polynucleotides and/or polypeptides of the present invention described elsewhere herein. Mention is also made of International Patent Publication No. WO 2016/027264, and the documents it cites, all of which are incorporated herein by reference, the teachings of which can be applied and/or adapted for targeted delivery of one or more polynucleotides and/or polypeptides of the present invention described elsewhere herein. And mention is made of Lorenzer et al., “Going beyond the liver: Progress and challenges of targeted delivery of siRNA therapeutics,” Journal of Controlled Release, 203: 1-15 (2015), and the documents it cites, all of which are incorporated herein by reference, the teachings of which can be applied and/or adapted for targeted delivery of one or more polynucleotides and/or polypeptides of the present invention described elsewhere herein.

An actively targeting lipid particle or nanoparticle or liposome or lipid bilayer delivery system (generally as to embodiments of the invention, “lipid entity of the invention” delivery systems) are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these embodiments are within the ambit of the skilled artisan. In the field of active targeting, there are a number of cell-, e.g., tumor-, specific targeting ligands.

Also, as to active targeting, with regard to targeting cell surface receptors such as cancer cell surface receptors, targeting ligands on liposomes can provide attachment of liposomes to cells, e.g., vascular cells, via a noninternalizing epitope; and this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid bilayers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which can enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirus or AAV. Tf is a monomeric serum glycoprotein of approximately 80 KDa involved in the transport of iron throughout the body. Tf binds to the TfR and translocates into cells via receptor-mediated endocytosis. The expression of TfR can be higher in certain cells, such as tumor cells (as compared with normal cells) and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, and cells of the mouth such as oral tumor cells.

Also, as to active targeting, a lipid entity of the invention can be multifunctional, i.e., employ more than one targeting moiety such as CPP, along with Tf; a bifunctional system, e.g., a combination of Tf and poly-L-arginine which can provide transport across the endothelium of the blood-brain barrier. EGFR is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2 is encoded by the ERBB2 gene. The invention comprehends a HER-2-targeting lipid entity of the invention, e.g., an anti-HER-2-antibody, or binding fragment thereof, a lipid entity of the invention, a HER-2-targeting-PEGylated lipid entity of the invention (e.g., having an anti-HER-2-antibody or binding fragment thereof), a HER-2-targeting-maleimide-PEG polymer lipid entity of the invention (e.g., having an anti-HER-2-antibody or binding fragment thereof). Upon cellular association, the receptor-antibody complex can be internalized by formation of an endosome for delivery to the cytoplasm.

With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bilayer). The microenvironment of a cell mass, such as a tumor microenvironment, can be targeted; for instance, it may be advantageous to target cell mass vasculature, such as the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG, such as APRPG-PEG-modified. VCAM, the vascular endothelium, plays a key role in the pathogenesis of inflammation, thrombosis and atherosclerosis. CAMs are involved in inflammatory disorders, including cancer, and are a logical target. E- and P-selectins, VCAM-1 and ICAMs, can be used to target a lipid entity of the invention., e.g., with PEGylation.

Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention, such as for an antihuman MT1-MMP monoclonal antibody linked to a lipid entity of the invention, e.g., via a spacer such as a PEG spacer. ap-integrins or integrins are a group of transmembrane glycoprotein receptors that mediate attachment between a cell and its surrounding tissues or extracellular matrix.

Integrins contain two distinct chains (heterodimers) called α- and β-subunits. The tumor tissue-specific expression of integrin receptors can be utilized for targeted delivery in the invention, e.g., whereby the targeting moiety can be an RGD peptide such as a cyclic RGD.

Aptamers are ssDNA or RNA oligonucleotides that impart high affinity and specific recognition of the target molecules by electrostatic interactions, hydrogen bonding and hydrophobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer).

Also, as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by a buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA, cholesteryl-GALA and PEG-GALA may show a highly efficient endosomal release; a pore-forming protein listeriolysin O may provide an endosomal escape mechanism; and histidine-rich peptides have the ability to fuse with the endosomal membrane, resulting in pore formation, and can buffer the proton pump causing membrane lysis.

The invention comprehends a lipid entity of the invention modified with CPP(s), for intracellular delivery that may proceed via energy dependent macropinocytosis followed by endosomal escape. The invention further comprehends organelle-specific targeting. A lipid entity of the invention surface-functionalized with the triphenylphosphonium (TPP) moiety or a lipid entity of the invention with a lipophilic cation, rhodamine 123 can be effective in delivery of cargo to mitochondria. DOPE/sphingomyelin/stearyl-octa-arginine can delivers cargos to the mitochondrial interior via membrane fusion. A lipid entity of the invention surface modified with a lysosomotropic ligand, octadecyl rhodamine B can deliver cargo to lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

It should be understood that as to each possible targeting or active targeting moiety herein-discussed, there is an embodiment of the invention wherein the delivery system comprises such a targeting or active targeting moiety. Likewise, Table 2 provides exemplary targeting moieties that can be used in the practice of the invention an as to each an embodiment of the invention provides a delivery system that comprises such a targeting moiety.

TABLE 2

| Targeting Moiety | Target Molecule | Target Cell or Tissue |
|---|---|---|
| folate | folate receptor | cancer cells |
| transferrin | transferrin receptor | cancer cells |
| Antibody CC52 | rat CC531 | rat colon adenocarcinoma CC531 |
| anti- HER2 antibody | HER2 | HER2 -overexpressing tumors |
| anti-GD2 | GD2 | neuroblastoma, melanoma |
| anti-EGFR | EGFR | tumor cells overexpressing EGFR |
| pH-dependent fusogenic | | ovarian carcinoma |
| peptide diINF-7 | | |
| anti-VEGFR | VEGF Receptor | tumor vasculature |
| anti-CD19 | CD19 (B cell marker) | leukemia, lymphoma |
| cell-penetrating peptide | | blood-brain barrier |
| cyclic arginine-glycine- | avβ3 | glioblastoma cells, human |
| aspartic acid-tyrosine- | | umbilical vein endothelial cells, |
| cysteine peptide | | tumor angiogenesis |
| (c(RGDyC)-LP) (SEQ | | |
| ID NO: 16305) | | |
| ASSHN peptide | | endothelial progenitor cells; anti- |
| | | cancer |
| PR_b peptide | α₅β₁ integrin | cancer cells |
| AG86 peptide | α₆β₄ integrin | cancer cells |
| KCCYSL (P6.1 | HER-2 receptor | cancer cells |
| peptide) (SEQ ID NO: | | |
| 16306) | | |
| affinity peptide LN | Aminopeptidase N | APN-positive tumor |
| (YEVGHRC) (SEQ ID | (APN/CD13) | |
| NO: 16307 | | |
| synthetic somatostatin | Somatostatin receptor 2 | breast cancer |
| analogue | (SSTR2) | |
| anti-CD20 monoclonal | B-lymphocytes | B cell lymphoma |
| antibody | | |

Thus, in an embodiment of the delivery system, the targeting moiety comprises a receptor ligand, such as, for example, hyaluronic acid for CD44 receptor, galactose for hepatocytes, or antibody or fragment thereof such as a binding antibody fragment against a desired surface receptor, and as to each of a targeting moiety comprising a receptor ligand, or an antibody or fragment thereof such as a binding fragment thereof, such as against a desired surface receptor, there is an embodiment of the invention wherein the delivery system comprises a targeting moiety comprising a receptor ligand, or an antibody or fragment thereof such as a binding fragment thereof, such as against a desired surface receptor, or hyaluronic acid for CD44 receptor, galactose for hepatocytes (see, e.g., Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8):1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao J et al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), each of which and the documents cited therein are hereby incorporated herein by reference), the teachings of which can be applied and/or adapted for targeted delivery of one or more CRISPR-Cas molecules described herein.

Other exemplary targeting moieties are described elsewhere herein, such as epitope tags and the like.

### Responsive Delivery

In some embodiments, the delivery vehicle can allow for responsive delivery of the cargo(s), e.g., one or more polynucleotides and/or polypeptides of the present invention described elsewhere herein. Responsive delivery, as used in this context herein, refers to delivery of cargo(s) by the delivery vehicle in response to an external stimuli. Examples of suitable stimuli include, without limitation, an energy (light, heat, cold, and the like), a chemical stimuli (e.g. chemical composition, etc.), and a biologic or physiologic stimuli (e.g. environmental pH, osmolarity, salinity, biologic molecule, etc.). In some embodiments, the targeting moiety can be responsive to an external stimuli and facilitate responsive delivery. In other embodiments, responsiveness is determined by a non-targeting moiety component of the delivery vehicle.

The delivery vehicle can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)).

Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes.

The invention also comprehends redox-triggered delivery. The difference in redox potential between normal and inflamed or tumor tissues, and between the intra- and extracellular environments has been exploited for delivery, e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment.

Enzymes can also be used as a trigger to release payload. Enzymes, including MMPs (e.g., MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln) (SEQ ID NO: 16308) can be incorporated into a linker, and can have antibody targeting, e.g., antibody 2C5.

The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or γ-Fe2O3, e.g., those that are less than 10 nm in size. Targeted delivery can be then by exposure to a magnetic field.

### Cells

In some embodiments, the present disclosure provides cells and organisms comprising the compositions, such as the polynucleotides, polypeptides, vectors, delivery vehicles, etc. described herein. In some embodiments, the cells are producer cells and are capable of generating virus particles or other delivery vehicles (e.g., exosomes) containing the one or more polynucleotides and/or polypeptides of the present invention. The cells may be in tissue, organ, or isolated cells. Such cells may be of a unique type of cells or a group of different types of cells, such as cultured cell lines, primary cells and proliferative cells. The cells may be prokaryotic cells, lower eukaryotic cells such as yeast, and other eukaryotic cells such as insect cells, plant and mammalian (e.g., human or non-human) cells, as well as cells capable of producing the vector of the invention (e.g., 293, HER96, PERC.6 cells, Vero, HeLa, CEF, duck cell lines, etc.). The cells may include cells which can be or has been the recipient of the vector described herein as well as progeny of such cells. Host cells can be cultured in conventional fermentation bioreactors, flasks, and petri plates. Culturing can be carried out at a temperature, pH and oxygen content appropriate for a given cell. No attempts will be made here to describe in detail the various prokaryote and eukaryotic host cells and methods known for the production of the polypeptides and vectors herein.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, a polynucleotide, polypeptide, vector, delivery vehicle, and/or cell of the present invention described in greater detail elsewhere herein.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a viral infection or a symptom thereof. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof. In some embodiments, the pharmaceutical formulation comprises an effective amount of one or more anti-viral agents. Exemplary antiviral-agents include without limitation, nucleoside analogues with reverse transcriptase activity (e.g., tenofovir, emtricitabine, lamivudine, abacavir, stavudine, didanosine, and zidovudine), nonnucleoside reverse transcriptase inhibitors (e.g., delavirdine, efavirenz, etravirine, nevirapine, and rilpivirine), protease inhibitors (atazanavir, darunavir, indinavir, ritonavir, tipranavir and many others), and miscellaneous agents such as maraviroc, enfuvirtide (fusion inhibitor), and integrase inhibitors (raltegravir, elvitegravir and dolutegravir), emdesivir, molnupiravir, Paxlovid (nirmatrelvir and ritonavir), Tecovirimat, seltamivir (oral), zanamivir (by inhalation) and peramivir (intravenous), baloxavir (oral), valacyclovir, cidofovir, famciclovir, ganciclovir, valganciclovir, foscarnet, simeprevir, paritaprevir, grazoprevir, glecaprevir, sofosbuvir, dasabuvir, daclatasvir, elbasvir, lepidasvir, velpatsvir, ombitasvir, pibrentasvor, Alpha interferon, peginterferon, monoclonal antibodies targeting specific viruses, and any combination thereof.

### Effective Amounts

In some embodiments, the amount of the primary active agent and/or optional secondary agent can be an effective amount, least effective amount, and/or therapeutically effective amount. As used herein, “effective amount”, “effective concentration”, and/or the like refers to the amount, concentration, etc. of the primary and/or optional secondary agent included in the pharmaceutical formulation that achieve one or more therapeutic effects or desired effect. As used herein, “least effective”, “least effective concentration”, and/or the like amount refers to the lowest amount, concentration, etc. of the primary and/or optional secondary agent that achieves the one or more therapeutic or other desired effects. As used herein, “therapeutically effective amount”, “therapeutically effective concentration” and/or the like refers to the amount, concentration, etc. of the primary and/or optional secondary agent included in the pharmaceutical formulation that achieves one or more therapeutic effects. In some embodiments, the one or more therapeutic effects are inducing an immune response in a subject to which they are delivered, inducing a B- and/or T-cell response in a subject to which it is delivered, treating or preventing a viral infection in a subject to which it is delivered.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments where a cell or cell population is present in the pharmaceutical formulation (e.g., as a primary and/or or secondary active agent), the effective amount of cells can be any amount ranging from about 1 or 2 cells to 1×10¹ cells/mL, 1×10²⁰ cells/mL or more, such as about 1×10¹ cells/mL, 1×10² cells/mL, 1×10³ cells/mL, 1×10⁴ cells/mL, 1×10⁵ cells/mL, 1×10⁶ cells/mL, 1×10⁷ cells/mL, 1×10⁸ cells/mL, 1×10⁹ cells/mL, 1×10¹⁰ cells/mL, 1×10¹¹ cells/mL, 1×10¹² cells/mL, 1×10¹³ cells/mL, 1×10¹⁴ cells/mL, 1×10¹⁵ cells/mL, 1×10¹⁶ cells/mL, 1×10¹⁷ cells/mL, 1×10¹⁸ cells/mL, 1×10¹⁹ cells/mL, to/or about 1×10²⁰/cells/mL or any numerical value or subrange within any of these ranges.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be about 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In some embodiments, the amount or effective amount of the one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include, but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof. In some embodiments, the co-therapy and/or combination therapy comprises an effective amount of one or more anti-viral agents. Exemplary antiviral-agents include without limitation, nucleoside analogues with reverse transcriptase activity (e.g., tenofovir, emtricitabine, lamivudine, abacavir, stavudine, didanosine, and zidovudine), nonnucleoside reverse transcriptase inhibitors (e.g., delavirdine, efavirenz, etravirine, nevirapine, and rilpivirine), protease inhibitors (atazanavir, darunavir, indinavir, ritonavir, tipranavir and many others), and miscellaneous agents such as maraviroc, enfuvirtide (fusion inhibitor), and integrase inhibitors (raltegravir, elvitegravir and dolutegravir), emdesivir, molnupiravir, Paxlovid (nirmatrelvir and ritonavir), Tecovirimat, seltamivir (oral), zanamivir (by inhalation) and peramivir (intravenous), baloxavir (oral), valacyclovir, cidofovir, famciclovir, ganciclovir, valganciclovir, foscarnet, simeprevir, paritaprevir, grazoprevir, glecaprevir, sofosbuvir, dasabuvir, daclatasvir, elbasvir, lepidasvir, velpatsvir, ombitasvir, pibrentasvor, Alpha interferon, peginterferon, monoclonal antibodies targeting specific viruses, and any combination thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Immunogenic Compositions

Described in certain example embodiments herein are immunogenic compositions comprising a polynucleotide, a vector, polypeptide, delivery vehicle, and/or cell of the present invention as described elsewhere herein. In some embodiments, the pharmaceutical formulation described herein is an immunogenic composition. In certain example embodiments, the polynucleotide and/or the polypeptide of the immunogenic composition is capable of stimulating a B-cell and/or T-cell response in a subject to which it is delivered. In certain example embodiments, the B-cell response comprises antibody production.

mRNA Vaccines

In some embodiments, the pharmaceutical formulations and/or immunogenic composition described herein are mRNA vaccines. In some embodiments, one or more polynucleotides encoding the one or more polypeptides of the present invention described herein are included in an mRNA vaccine composition. In some embodiments, the polypeptides are immunogenic polypeptides. The mRNA vaccine composition can be administered to a subject in need thereof. In some embodiments, the vaccine is administered to a subject in an effective amount to induce an immune response in the subject.

Described herein are pharmaceutical compositions that include one or more isolated messenger ribonucleic (mRNA) polynucleotides encoding at least one viral antigenic polypeptide or an immunogenic fragment thereof (e.g., an immunogenic fragment capable of inducing an immune response to the antigenic polypeptide), such as any of those polynucleotides described in greater detail elsewhere herein, where the isolated mRNA is formulated in a lipid nanoparticle. As used herein “antigenic polypeptide” encompasses immunogenic fragments of the antigenic polypeptide (an immunogenic fragment that is induces (or is capable of inducing) an immune response to a virus or virus variant. The mRNA encoding at least one viral antigenic polypeptide or immunogenic fragment thereof can include an open reading frame that encodes the at least one viral antigenic polypeptide or immunogenic fragment thereof. In some embodiments, the open reading frame encodes at least two, at least five, or at least ten viral antigenic polypeptides and/or immunogenic fragments thereof. In some embodiments, the open reading frame encodes at least 100 antigenic polypeptides. In some embodiments, the open reading frame encodes 2-100 viral antigenic polypeptides and/or immunogenic fragments thereof.

In some embodiments, the pharmaceutical composition comprises a plurality of lipid nanoparticles comprising a cationic lipid, a neutral lipid, a cholesterol, and a PEG lipid, wherein the plurality of lipid nanoparticles optionally has a mean particle size of between 80 nm and 160 nm; and wherein the lipid nanoparticles comprise one or more polynucleotides encoding at least one viral antigenic polypeptide or an immunogenic fragment thereof.

In some embodiments, the mRNA vaccine is multivalent. In some embodiments, the mRNA of the mRNA vaccine is codon-optimized. In some embodiments, an RNA (e.g., mRNA) vaccine further includes an adjuvant.

In some embodiments, the isolated mRNA is not self-replicating.

In some embodiment, the isolated mRNA comprises and/or encodes one or more 5′terminal cap (or cap structure), 3′terminal cap, 5′untranslated region, 3′untranslated region, a tailing region, or any combination thereof.

In some embodiments, the capping region of the isolated mRNA region may be from 1 to 10, e.g., 2-9, 3-8, 4-7, 1-5, 5-10, or at least 2, or 10 or fewer nucleotides in length. In some embodiments, the cap is absent.

In some embodiments, a 5′-cap structure is cap0, cap1, ARCA, inosine, N1-methyl-guanosine, 2′-fluoro-guanosine, 7-deaza-guanosine, 8-oxo-guanosine, 2-amino-guanosine, LNA-guanosine, or 2-azido-guanosine.

In some embodiments, the 5′terminal cap is 7mG(5′)ppp(5′)NlmpNp, m7GpppG cap, N7-methylguanine. In some embodiments, the 3′terminal cap is a 3′-O-methyl-m7GpppG.

In some embodiments, the 3′-UTR is an alpha-globin 3′-UTR. in some embodiments, the 5′-UTR comprises a Kozak sequence.

In some embodiments, the tailing sequence may range from absent to 500 nucleotides in length (e.g., at least 60, 70, 80, 90, 120, 140, 160, 180, 200, 250, 300, 350, 400, 450, or 500 nucleotides). In some embodiments, the tailing region is or includes a polyA tail. Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional. In some embodiments, the poly-A tail is at least 160 nucleotides in length.

In some embodiments, the at least one viral antigenic polypeptide linked to or fused to a signal peptide. In some embodiments, the isolated mRNA encoding a viral antigenic polypeptide or immunogenic fragment thereof further includes a polynucleotide sequence encoding a signal peptide. In some embodiments, the signal peptide is selected from: a HuIgGk signal peptide (METPAQLLFLLLLWLPDTTG) (SEQ ID NO: 16309); IgE heavy chain epsilon-1 signal peptide (MDWTWILFLVAAATRVHS) (SEQ ID NO: 16310); Japanese encephalitis PRM signal sequence (MLGSNSGQRVVFTILLLLVAPAYS) (SEQ ID NO: 16311), VSVg protein signal sequence (MKCLLYLAFLFIGVNCA) (SEQ ID NO: 16312) and Japanese encephalitis JEV signal sequence (MWLVSLAIVTACAGA) (SEQ ID NO: 16313). In some embodiments, the signal peptide is fused to the N-terminus of at least one viral antigenic polypeptide. In some embodiments, a signal peptide is fused to the C-terminus of at least one viral antigenic polypeptide.

In some embodiments, the polynucleotides of the mRNA vaccine composition are structurally modified and/or chemically modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In some embodiments, the polynucleotide, e.g., an mRNA of an mRNA vaccine composition described herein comprises at least one chemical modification. In some embodiments, the polynucleotide, e.g., an mRNA of an mRNA vaccine composition does not comprise a chemical or structural modification.

In some embodiments, the at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, the chemical modification is in the 5-position of the uracil. In some embodiments, the chemical modification is a N1-methylpseudouridine. In some embodiments, the chemical modification is a N1-ethylpseudouridine.

In some embodiments, about 10%, 15%, 20%, 24%, 30%, 35%, 40%, 45%, 50%, 55%, 60%, 65%, 70%, 75%, 80%, 85%, 90%, 95%, to/or about 100% of the uracil in of the SARS-CoV-2 antigenic polypeptide or immunogenic fragment thereof encoding polynucleotide, such in the open reading frame, have a chemical modification, In some embodiments, 100% of the uracil in the open reading frame of the viral antigenic polypeptide or immunogenic fragment thereof encoding polynucleotide have a N1-methyl pseudouridine in the 5-position of the uracil.

In some embodiments, the mRNA polynucleotide includes a stabilization element. In some embodiments, the stabilization element is a histone stem-loop. In some embodiments, the stabilization element is a nucleic acid sequence having increased GC content relative to wild type sequence.

In one embodiment, the mRNA polynucleotide may include a sequence encoding a self-cleaving peptide. The self-cleaving peptide may be, but is not limited to, a 2A peptide. As a non-limiting example, the 2A peptide may have the protein sequence: GSGATNFSLLKQAGDVEENPGP (SEQ ID NO: 16314), fragments or variants thereof. In one embodiment, the 2A peptide cleaves between the last glycine and last proline. As another non-limiting example, the polynucleotides of the present invention may include a polynucleotide sequence encoding the 2A peptide having the protein sequence GSGATNFSLLKQAGDVEENPGP (SEQ ID NO: 16314) fragments or variants thereof.

One such polynucleotide sequence encoding the 2A peptide is GGAAGCGGAGCTACTAACTTCAGCCTGCTGAAGCAGGCTGGAGACGTGGAG GAGAACCCTGGACCT (SEQ ID NO: 16315). The polynucleotide sequence of the 2A peptide may be modified or codon optimized by the methods described herein and/or are known in the art.

In one embodiment, this sequence may be used to separate the coding region of two or more polypeptides of interest. As a non-limiting example, the sequence encoding the 2A peptide may be between a first coding region A and a second coding region B (A-2Apep-B). The presence of the 2 A peptide would result in the cleavage of one long protein into protein A, protein B and the 2A peptide. Protein A and protein B may be the same or different peptides or polypeptides of interest. In another embodiment, the 2A peptide may be used in the polynucleotides of the present invention to produce two, three, four, five, six, seven, eight, nine, ten or more proteins.

In some embodiments, the length of an mRNA included in the mRNA vaccine is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000, or up to and including 100,000 nucleotides).

In some embodiments, the length of an mRNA included in the mRNA vaccine includes from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

In some embodiments, the polynucleotides are linear. In yet another embodiment, the polynucleotides of the present invention that are circular are known as “circular polynucleotides” or “circP.” As used herein, “circular polynucleotides” or “circP” means a single stranded circular polynucleotide which acts substantially like, and has the properties of, an R A. The term “circular” is also meant to encompass any secondary or tertiary configuration of the circP.

Other RNA modifications for mRNA vaccines and production of mRNA can be as described e.g., U.S. Pat. Nos. 8,278,036, 8,691,966, 8,748,089, 9,750,824, 10,232,055, 10,703,789, 10,702,600, 10,577,403, 10,442,756, 10,266,485, 10,064,959, 9,868,692, 10,064,959, 10,272,150; U.S. Publication Nos. US20130197068, US20170043037, US20130261172, US20200030460, US20150038558, US20190274968, US20180303925, US20200276300; International Patent Application Publication Nos. WO/2018/081638A1, WO/2016/176330A1, which are incorporated herein by reference.

In some embodiments, the mRNA vaccine includes one or more additional mRNAs that encode a polypeptide adjuvant. In some embodiments, the mRNA vaccine includes one or more additional mRNAs that encode a non-viral antigen, such as an antigen to another disease causing agent.

In some embodiments, the one or more additional mRNAs that encode a polypeptide adjuvant encode a flagellin polypeptide. In some embodiments, at least one flagellin polypeptide (e.g., encoded flagellin polypeptide) is an immunogenic flagellin fragment. In some embodiments at least one flagellin polypeptide has at least 80%, at least 85%, at least 90%, or at least 95% identity to a flagellin polypeptide having a sequence identified by any one of SEQ ID NO: 54-56 of U.S. Pat. No. 10,272,150.

In some embodiments, at least one flagellin polypeptide and at least one viral and/or additional antigenic polypeptide are encoded by a single RNA (e.g., mRNA) polynucleotide. In other embodiments, at least one flagellin polypeptide and at least one viral and/or additional antigenic polypeptide are each encoded by a different RNA polynucleotide.

The isolated mRNA(s) can be made in part or using only in vitro transcription. Methods of making polynucleotides by in vitro transcription are known in the art and are described in U.S. Provisional Patent Application Nos. 61/618,862, 61/681,645, 61/737,130, 61/618,866, 61/681,647, 61/737,134, 61/618,868, 61/681,648, 61/737,135, 61/618,873, 61/681,650, 61/737,147, 61/618,878, 61/681,654, 61/737,152, 61/618,885, 61/681,658, 61/737,155, 61/618,896, 61/668,157, 61/681,661, 61/737,160, 61/618,911, 61/681,667, 61/737,168, 61/618,922, 61/681,675, 61/737,174, 61/618,935, 61/681,687, 61/737,184, 61/618,945, 61/681,696, 61/737,191, 61/618,953, 61/681,704, 61/737,203; International Publication Nos. WO 2013/151666, WO 2013/151668, WO 2013/151663, WO 2013/151669, WO 2013/151670, WO 2013/151664, WO 2013/151665, WO 2013/151736, WO 2013/151672, WO 2013/151671 WO 2013/151667, and WO 2020/205793A1; the contents of each of which are herein incorporated by reference in their entireties. Lipid Nanoparticles

The isolated mRNAs and other polynucleotides of the mRNa vaccine can be formulated in a lipid nanoparticle. In some embodiments, the lipid nanoparticle is a cationic lipid nanoparticle.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, the cationic lipid is a biodegradable cationic lipid. In some embodiments, the biodegradable cationic lipid comprises an ester linkage. In some embodiments, the biodegradable cationic lipid comprises DLin-DMA with an internal ester, DLin-DMA with a terminal ester, DLin-MC3-DMA with an internal ester, or DLin-MC3-DMA with a terminal ester.

In some embodiments, a lipid nanoparticle comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)-butanoyl)oxy)heptadecanedioate (L319), (12Z,15Z)-N,N-dimethyl-2-nonylhenicosa-12,15-dien-1-amine (L608), and N,N-dimethyl-1-[(1S,2R)-2-octylcyclopropyl]heptadecan-8-amine (L530). In some embodiments, the neutral lipid is 1,2-distearoyl-sn-glycero-3-phosphocholine (DSPC), the sterol is cholesterol, and the PEG-modified lipid is 1,2-dimyristoyl-racalycero-3-methoxypolyethylene glycol-2000 (PEG-DMG) or PEG-cDMA.

In some embodiments, the lipid nanoparticle is any nanoparticle described in U.S. Pat. No. 10,442,756, and/or comprises any compound described in U.S. Pat. No. 10,442,756, including, but not limited to, a nanoparticle according to any one of Formulas (IA) or (II) described therein.

In some embodiments, the lipid nanoparticle is any nanoparticle described in e.g., U.S. Pat. No. 10,266,485, and/or comprises any compound described in U.S. Pat. No. 10,266,485, including, but not limited to, a nanoparticle according to Formula (II) described therein.

In some embodiments, the lipid nanoparticle is a nanoparticle described in U.S. Pat. No. 9,868,692, and/or comprises a compound described in e.g., U.S. Pat. No. 9,868,692, including, but not limited to, a nanoparticle according to Formula (I), (1A), (II), (IIa), (IIb), (IIc), (IId), (IIe),

In some embodiments, a lipid nanoparticle comprises compounds of Formula (I) and/or Formula (II) as described in U.S. patent Ser. No. 10/272,150.

In some embodiments, the mRNA vaccine is formulated in a lipid nanoparticle that comprises a compound selected from Compounds 3, 18, 20, 25, 26, 29, 30, 60, 108-112 and 122 of U.S. Pat. No. 10,272,150.

In some embodiments, at least 80% (e.g., 85%, 90%, 95%, 98%, 99%) of the uracil in the open reading frame have a chemical modification, optionally wherein the vaccine is formulated in a lipid nanoparticle (e.g., a lipid nanoparticle comprises a cationic lipid, a PEG-modified lipid, a sterol and a non-cationic lipid).

In some embodiments, the lipid nanoparticle has a mean diameter of 50-200 nm.

In some embodiments, a lipid nanoparticle comprises compounds of Formula (I) and/or Formula (II), as discussed below.

In some embodiments, a lipid nanoparticle comprises Compounds 3, 18, 20, 25, 26, 29, 30, 60, 108-112, or 122 as set forth in U.S. Pat. No. 10,272,150.

In some embodiments, the lipid nanoparticle has a polydispersity value of less than 0.4 (e.g., less than 0.3, 0.2 or 0.1).

In some embodiments, a plurality of lipid nanoparticles, such as when contained in a formulation, has a mean PDI of between 0.02 and 0.2.

In some embodiments, a plurality of lipid nanoparticles, such as when contained in a formulation comprising one or more polynucleotide(s), has a mean lipid to polynucleotide ratio (wt/wt) of between 10 and 20.

In some embodiments, the lipid nanoparticle has a net neutral charge at a neutral pH value.

Methods of mRNA Vaccination

The compositions described herein can be used to induce an antigen specific immune response to a virus or a viral variant. Exemplary viruses are described elsewhere herein.

In some embodiments, the methods of inducing an antigen specific immune response in a subject include administering to the subject any of the RNA (e.g., mRNA) vaccine as provided herein in an amount effective to produce an antigen-specific immune response.

In some embodiments, an antigen-specific immune response comprises a T cell response and/or a B cell response.

In some embodiments, a method of producing an antigen-specific immune response comprises administering to a subject a single dose (no booster dose) of a RNA (e.g., mRNA) vaccine of the present disclosure.

In some embodiments, the RNA (e.g., mRNA) vaccine is a combination vaccine comprising a combination of an mRNA vaccine described herein and at least one other mRNA vaccine. The at least one other mRNA vaccine can be against the same or a different virus or disease-causing agent.

In some embodiments, a method further comprises administering to the subject a second (booster) dose of an RNA (e.g., mRNA) vaccine. Additional doses of a RNA (e.g., mRNA) vaccine may be administered.

In some embodiments, the subject exhibits a seroconversion rate of at least 80% (e.g., at least 85%, at least 90%, or at least 95%) following the first dose or the second (booster) dose of the vaccine. Seroconversion is the time period during which a specific antibody develops and becomes detectable in the blood. After seroconversion has occurred, a virus can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection.

In some embodiments, an RNA (e.g., mRNA) vaccine described herein is administered to a subject by intradermal, subcutaneous, or intramuscular injection. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

In some embodiments, the anti-antigenic polypeptide antibody titer produced in the subject is increased by at least 1 log relative to a control. In some embodiments, the anti-antigenic polypeptide antibody titer produced in the subject is increased by 1-3 log relative to a control.

In some embodiments, the anti-antigenic polypeptide antibody titer produced in a subject is increased at least 2 times relative to a control. In some embodiments, the anti-antigenic polypeptide antibody titer produced in the subject is increased at least 5 times relative to a control. In some embodiments, the anti-antigenic polypeptide antibody titer produced in the subject is increased at least 10 times relative to a control. In some embodiments, the anti-antigenic polypeptide antibody titer produced in the subject is increased 2-10 times relative to a control.

In some embodiments, the control is an anti-antigenic polypeptide antibody titer produced in a subject who has not been administered an RNA (e.g., mRNA) vaccine of the present disclosure. In some embodiments, the control is an anti-antigenic polypeptide antibody titer produced in a subject who has been administered a live attenuated or inactivated vaccine against a virus or wherein the control is an anti-antigenic polypeptide antibody titer produced in a subject who has been administered a recombinant or purified viral protein vaccine.

In some embodiments, the control is an anti-antigenic polypeptide antibody titer produced in a subject who has been administered a virus-like particle (VLP) vaccine comprising structural proteins of the virus.

The RNA (e.g., mRNA) vaccine of the present disclosure can be administered to a subject in an effective amount (e.g., an amount effective to induce an immune response in the subject).

In some embodiments, the RNA (e.g., mRNA) vaccine is formulated in an effective amount to produce an antigen specific immune response in a subject.

In some embodiments, the effective amount is a total dose of 25 g to 1000 g, or 50 g to 1000 g. In some embodiments, the effective amount is a total dose of 100 g. In some embodiments, the effective amount is a dose of 25 g administered to the subject a total of two times. In some embodiments, the effective amount is a dose of 100 g administered to the subject a total of two times. In some embodiments, the effective amount is a dose of 400 g administered to the subject a total of two times. In some embodiments, the effective amount is a dose of 500 g administered to the subject a total of two times.

In some embodiments, the efficacy (or effectiveness) of an RNA (e.g., mRNA) vaccine is greater than 60%.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas: Efficacy=(ARU−ARV)/ARU×100; and Efficacy=(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination: Effectiveness=(1−OR)×100.

In some embodiments, the efficacy (or effectiveness) of an RNA (e.g., mRNA) vaccine is at least 65%, at least 70%, at least 75%, at least 80%, at least 85%, or at least 90%.

In some embodiments, the vaccine immunizes the subject against one or more viral variants. Exemplary viruses and variants are described elsewhere herein.

In some embodiments, the subject to which the mRNA vaccine of the present disclosure is administered is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject to which the mRNA vaccine of the present disclosure is administered was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, a RNA (e.g., mRNA) vaccine may be administered later in life, for example, at the age of about 6 months to about 5 years, or older.

In some embodiments, the subject to which the mRNA vaccine of the present disclosure is administered the subject to which the mRNA vaccine of the present disclosure is administered is pregnant (e.g., in the first, second or third trimester) when administered an RNA (e.g., mRNA) vaccine.

In some embodiments, the subject to which the mRNA vaccine of the present disclosure is administered is a young adult between the ages of about 20 years and about 50 years (e.g., about 20, 25, 30, 35, 40, 45 or 50 years old).

In some embodiments, the subject to which the mRNA vaccine of the present disclosure is administered is an elderly subject about 60 years old, about 70 years old, or older (e.g., about 60, 65, 70, 75, 80, 85, 90, or about 100 or more years old).

In some embodiments, the subject to which the mRNA vaccine of the present disclosure is administered ihas a chronic pulmonary disease (e.g., chronic obstructive pulmonary disease (COPD) or asthma). Two forms of COPD include chronic bronchitis, which involves a long-term cough with mucus, and emphysema, which involves damage to the lungs over time. Thus, a subject administered a RNA (e.g., mRNA) vaccine may have chronic bronchitis or emphysema.

In some embodiments, the subject to which the mRNA vaccine of the present disclosure is administered is immunocompromised (has an impaired immune system, e.g., has an immune disorder or autoimmune disorder).

In some embodiments, the mRNA vaccine of the present disclosure is delivered to a subject at a dosage of between 10 μg/kg and 400 μg/kg of the nucleic acid vaccine is administered to the subject. In some embodiments the dosage of the RNA polynucleotide is 1-g, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 ag, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the subject can receive 1, 2, 3, 4, 5, 6, 7, or more doses. After the initial dose (given at day zero) the subject can receive one or more (e.g., 1, 2, 3, 4, 5, 6, 7, 8, 9, 10, or more additional doses, referred to in the art as “booster” doses. The booster doses can follow the initial dose at any suitable time interval such as within days, weeks, months, or even years. In some embodiments, multiple booster doses are needed close in time after the inital dose(such as within 1, 2, 3, or 4 weeks after the initial dose) followed by a larger gap in time (e.g., months or years before subsequent booster doses are needed). In some embodiments, a first dose of the mRNA vaccine is administered to the subject on day zero. In some embodiments, a second dose of the mRNA vaccine ais administered to the subject on day 14, 21, 28, 35, 42, 49, 56, 63, 70, 77, 84 or more days after the first dose. In some embodiments, a third dose of the mRNA vaccine ais administered to the subject on day 14, 21, 28, 35, 42, 49, 56, 63, 70, 77, 84 or more days after the first and/or second dose.

In some embodiments, the mRNA vaccine confers an antibody titer superior to the criterion for seroprotection for a SARS-CoV-2 variant for an acceptable percentage of human subjects. In some embodiments, the antibody titer produced by the mRNA vaccines of the invention is a neutralizing antibody titer. In some embodiments the neutralizing antibody titer is greater than a protein vaccine. In other embodiments the neutralizing antibody titer produced by the mRNA vaccines of the invention is greater than an adjuvanted protein vaccine. In yet other embodiments the neutralizing antibody titer produced by the mRNA vaccines of the invention is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques.

In some embodiments, a unit of use vaccine comprises between 10 μg and 400 μg of one or more RNA polynucleotides encoding the SARS-Cov-2 antigenic polypeptide(s) and/or immunogenic fragment(s) thereof and a pharmaceutically acceptable carrier or excipient, formulated for delivery to a human subject. In some embodiments, the vaccine further comprises a cationic lipid nanoparticle.

Aspects of the invention provide methods of creating, maintaining or restoring antigenic memory to a SARS-CoV-2 variant in an individual or population of individuals comprising administering to said individual or population an mRNA vaccine described herein.

In some embodiments, the methods of vaccinating a subject comprising administering to the subject a single dosage of between 25 μg/kg and 400 μg/kg of an mRNA vaccine comprising one or more RNA polynucleotides encoding a SARS-CoV-2 antigenic polypeptide and/or an immunogenic fragment thereof in an effective amount to vaccinate the subject.

In some embodiments, the mRNA vaccines comprising one or more RNA polynucleotides encoding a SARS-CoV-2 antigenic polypeptide and/or an immunogenic fragment thereof, wherein the RNA comprises at least one chemical modification, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

In some embodiments, the mRNA vaccine comprises an LNP formulated RNA polynucleotide having an open reading frame comprising no nucleotide modifications (unmodified), the open reading frame one or more RNA polynucleotides encoding a SARS-CoV-2 antigenic polypeptide and/or an immunogenic fragment thereof, wherein the vaccine has at least 10 fold less RNA polynucleotide than is required for an unmodified mRNA vaccine not formulated in a LNP to produce an equivalent antibody titer. In some embodiments, the RNA polynucleotide is present in a dosage of 25-100 micrograms.

In some embodiments, the mRNA vaccine comprises an LNP formulated RNA polynucleotide having an open reading frame comprising one or more modifications, the open reading frame one or more RNA polynucleotides encoding a SARS-CoV-2 antigenic polypeptide and/or an immunogenic fragment thereof, wherein the vaccine has at least 10 fold less RNA polynucleotide than is required for an unmodified mRNA vaccine not formulated in a LNP to produce an equivalent antibody titer. In some embodiments, the RNA polynucleotide is present in a dosage of 25-100 micrograms.

In some embodiments, the method includes vaccinating a subject with a combination vaccine including at least two nucleic acid sequences encoding respiratory antigens, wherein at least one encodes a SARS-CoV-2 antigen wherein the dosage for the vaccine is a combined therapeutic dosage wherein the dosage of each individual nucleic acid encoding an antigen is a sub therapeutic dosage. In some embodiments, the combined dosage is 25 micrograms of the RNA polynucleotide in the nucleic acid vaccine administered to the subject. In some embodiments, the combined dosage is 100 micrograms of the RNA polynucleotide in the nucleic acid vaccine administered to the subject. In some embodiments the combined dosage is 50 micrograms of the RNA polynucleotide in the nucleic acid vaccine administered to the subject. In some embodiments, the combined dosage is 75 micrograms of the RNA polynucleotide in the nucleic acid vaccine administered to the subject. In some embodiments, the combined dosage is 150 micrograms of the RNA polynucleotide in the nucleic acid vaccine administered to the subject. In some embodiments, the combined dosage is 400 micrograms of the RNA polynucleotide in the nucleic acid vaccine administered to the subject. In some embodiments, the sub therapeutic dosage of each individual nucleic acid encoding an antigen is 1, 2, 3, 4, 5, 6, 7, 8, 9, 10, 11, 12, 13, 14, 15, 16, 17, 18, 19, or 20 micrograms.

In some embodiments, vaccines of the invention (e.g., LNP-encapsulated mRNA vaccines) produce prophylactically- and/or therapeutically-efficacious levels, concentrations and/or titers of antigen-specific antibodies in the blood or serum of a vaccinated subject. As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, etc.

In some embodiments, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.)

In some embodiments, antigen-specific antibodies are measured in units of g/ml or are measured in units of IU/L (International Units per liter) or mIU/ml (milli International Units per ml). In exemplary embodiments of the invention, an efficacious vaccine produces >0.5 μg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary embodiments of the invention, an efficacious vaccine produces >10 mIU/ml, >20 mIU/ml, >50 mIU/ml, >100 mIU/ml, >200 mIU/ml, >500 mIU/ml or >1000 mIU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay.

### Methods of Treating and Prophylaxis

In another aspect, the present disclosure provides methods of treating and or preventing (e.g., immunizing) an infection (e.g., viral infection) in a subject, and/or disease and conditions related to the infection. Generally, the methods may comprise administering a pharmaceutically effective (e.g., therapeutically effective amount or prophylactically effective amount)) amount of the composition herein to a subject, e.g., a subject in need thereof. In some cases, the method comprise administering the composition(s), the polynucleotide(s), and/or the vector(s) herein to a subject. A pharmaceutically effective amount refers to an amount which can elicit a biological, medicinal, or immunological response in a tissue, system, or subject (e.g., animal or human) that can prevent or alleviate one or more of the local or systemic symptoms or features of a disease or condition being treated.

Described in certain example embodiments herein are methods of inducing a B-cell response and/or T-cell response to a virus in a subject in need thereof, comprising administering, to the subject, the immunogenic composition or the therapeutic composition, or a pharmaceutical formulation thereof of the present invention described elsewhere herein. In certain example embodiments, the B cell response comprises antibody production. Described in certain example embodiments herein are methods of treating a viral infection in a subject in need thereof comprising administering, to the subject in need thereof, the immunogenic composition or the therapeutic composition, or a pharmaceutical formulation thereof of the present invention as described elsewhere herein in combination with an antiviral therapeutic. Described in certain example embodiments herein are methods an infection status of a subject comprising contacting immune cells derived from a subject with the immunogenic composition or a pharmaceutical formulation thereof of the present invention as described elsewhere herein; and detecting cross-reactivity of the immune cells to the immunogenic composition.

In certain examples, the methods may be used to induce a T cell response and/or antibody response to a virus in a subject. Exemplary viruses are described in greater detail elsewhere herein. In some cases, the methods may comprise administering the polypeptides and antigenic agents, and/or anti-viral therapeutics, or polynucleotides encoding thereof to a subject (e.g., a subject in need thereof).

Methods of administrating to a subject include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment and prophylaxis; this may be achieved by, for example, local infusion during surgery, topical application, by injection, by means of a catheter, by means of a suppository, or by means of an implant.

The term “subject” or “patient” is intended to include mammalian organisms. Examples of subjects/patients include humans and non-human mammals, e.g., non-human primates, dogs, cows, horses, pigs, sheep, goats, cats, mice, rabbits, rats, and transgenic non-human animals. In specific embodiments of the invention, the subject is a human.

In some cases, the methods comprise administering to a subject the pharmaceutical compositions alone or in concert with other therapeutic agents at appropriate dosages defined by routine testing in order to obtain optimal efficacy while minimizing any potential toxicity. The dosage regimen utilizing a pharmaceutical composition may be selected in accordance with a variety of factors including type, species, age, weight, sex, medical condition of the patient; the severity of the condition to be treated; the route of administration; the renal and hepatic function of the patient; and the particular pharmaceutical composition employed.

Optimal precision in achieving concentrations of the therapeutic regimen within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical composition and various therapeutic agents may be independently optimized and combined to achieve a synergistic result wherein the pathology is reduced more than it would be if either was used alone.

In particular, toxicity and therapeutic efficacy of the pharmaceutical composition may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ratio LD₅₀/ED₅₀. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED₅₀ with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC50 (the concentration of the test composition that achieves a half-maximal inhibition of symptoms) as determined in cell culture. Such information may be used to accurately determine useful doses in humans. Levels in plasma may be measured, for example, by high performance liquid chromatography.

The methods may comprise administering a booster agent in addition to the administration of the composition therein. A booster agent may be an extra administration of the composition herein or a different agent. A booster (or booster vaccine) may be given after an earlier administration of the composition. The time of administration between the initial administration of the composition and the booster may be at least 1 minute, at least 5 minutes, at least 10 minutes, at least 20 minutes, at least 30 minutes, at least 40 minutes, at least 50 minutes, at least 1 hour, at least 2 hours, at least 4 hours, at least 8 hours, at least 12 hours, at least 1 day, at least 1 week, at least 2 week, at least 3 week, at least 1 month, at least 2 months, at least 3 months, at least 4 months, at least 5 months, at least 6 months, at least 1 year, at least 5 years, at least 10 years, and any time period in-between.

### Delivery to Cells and Organisms

The present disclosure also provides delivery systems for introducing the compositions herein to cells, tissues, organs, or organisms. A delivery system may comprise one or more delivery vehicles and/or cargos. Exemplary delivery systems and methods include those described in paragraphs [00117] to [00278] of Feng Zhang et al., (WO2016106236A1), and pages 1241-1251 and Table 1 of Lino C A et al., Delivering CRISPR: a review of the challenges and approaches, DRUG DELIVERY, 2018, VOL. 25, NO. 1, 1234-1257, which are incorporated by reference herein in their entireties.

### Cargos

The delivery systems may comprise one or more cargos (such as the polynucleotides, polypeptides, vectors, cells etc. of the present invention described elsewhere herein). A cargo may comprise one or more of the following: i) one or more polypeptides herein, ii) one or more polynucleotides encoding the polypeptide(s) or vectors comprising the polynucleotides; (iii) mRNA molecules encoding the one or more polypeptides; iv) cells comprising i), ii) and/or iii). In some examples, a cargo may comprise a plasmid encoding one or more engineered proteins herein.

### Physical Delivery

In some embodiments, the cargos may be introduced to cells by physical delivery methods. Examples of physical methods include microinjection, electroporation, and hydrodynamic delivery. Both nucleic acid and proteins may be delivered using such methods. For example, the polypeptides and polynucleotides may be prepared in vitro, isolated, (refolded, purified if needed), and introduced to cells.

### Microinjection

Microinjection of the cargo directly to cells can achieve high efficiency, e.g., above 90% or about 100%. In some embodiments, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Polynucleotides and vectors comprising coding sequences for the polypeptides may be microinjected. In some cases, microinjection may be used i) to deliver DNA directly to a cell nucleus, and/or ii) to deliver mRNA (e.g., in vitro transcribed) to a cell nucleus or cytoplasm.

Microinjection may be used to generate genetically modified animals. For example, gene editing cargos may be injected into zygotes to allow for efficient germline modification. Such approach can yield normal embryos and full-term mouse pups harboring the desired modification(s).

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

Delivery also include delivery vehicles, which are described in greater detail elsewhere herein.

### Methods of Identifying Viral Polypeptides

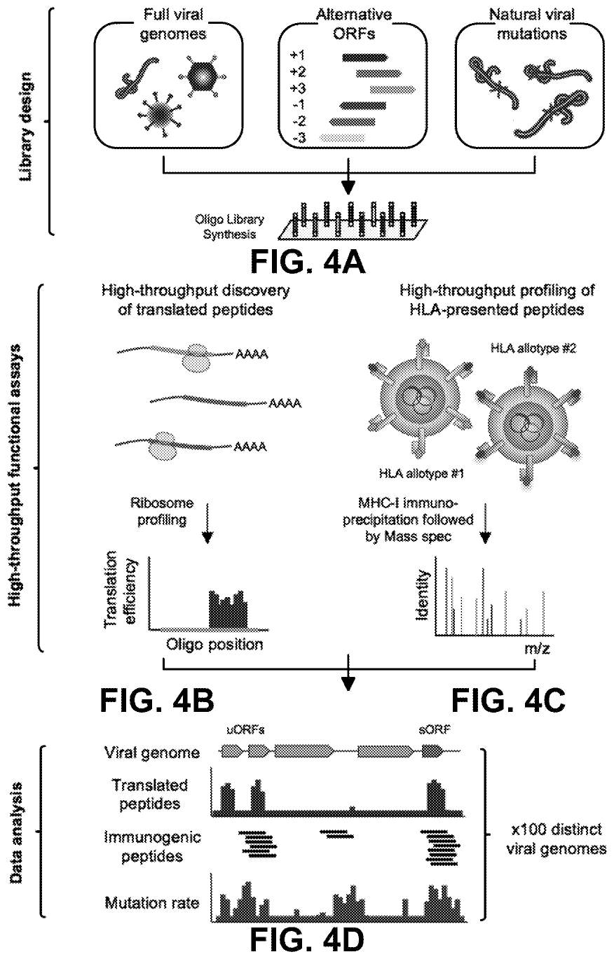

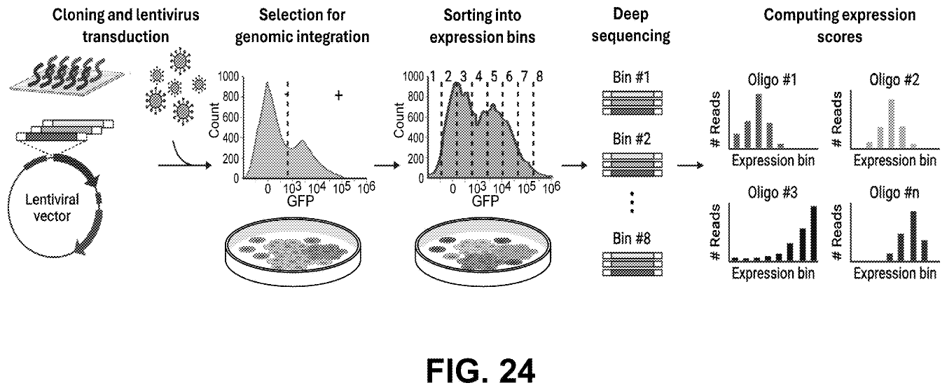

Described herein are high-throughput methods of identifying determining translated genomic sequences, such as translated viral genomic sequences, and/or one or more effects or functions (such as gene expression regulation of other viral sequences and/or mediating B- and/or T-cell responses in a host) of said translated genomic sequences and/or products produced therefrom (e.g., peptides, polypeptides). Such methods include massively parallel ribosome profiling, high-through put antigen presentation profiling, and/or massively parallel reporter assays. See e.g., FIG. 4A-4D and FIG. 24. The identified sequences and/or peptides they encode can be used in compositions, such as pharmaceutical formulations and/or immunogenic compositions described elsewhere herein.

### Massively Parallel Ribosome Profiling (MPRP)

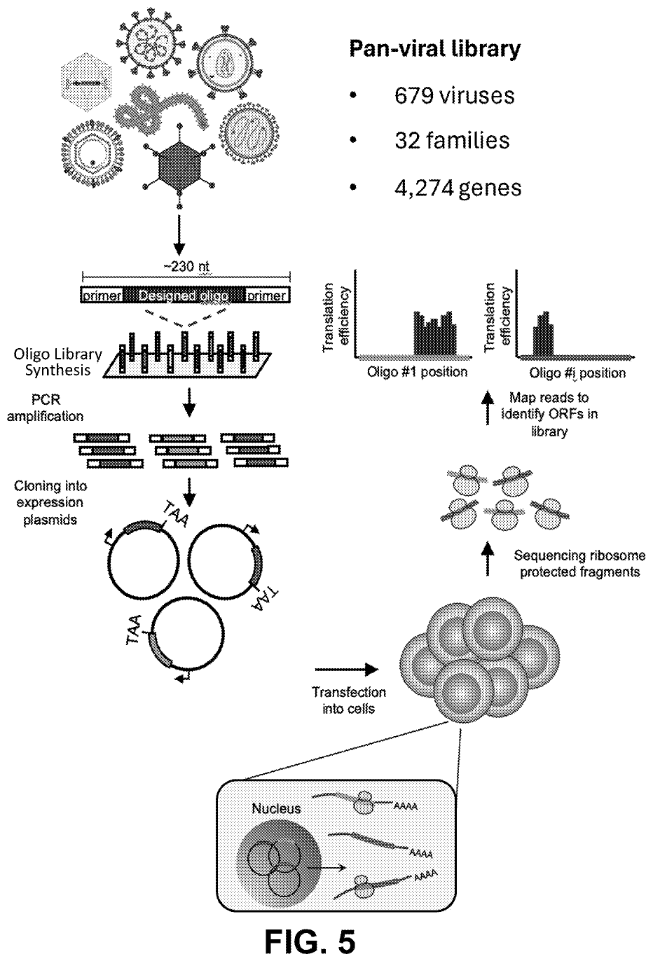

In some embodiments, one or more open reading frame, such as one or more non-canonical open reading frame, can be identified using a high throughput method of determining translated sequences. As described in the Working Examples herein, Applicant has developed a high throughput assay also referred to herein as “massively parallel ribosome profiling” that can identify from hundreds of genomic sequences, such as viral genomes, translated sequences. See e.g., FIGS. 4-5.

Described in certain example embodiments herein are high throughput methods of determining translated sequences, comprising expressing a vector as described elsewhere herein or a vector library (such as one that has a short synthetic sequence) as described elsewhere herein in one or more cells under conditions sufficient to produce translation products; obtaining a sample of ribosomes comprising ribosome-protected mRNA fragments (RPFs) from the cells; recovering ribosome footprints consisting essentially of the RPFs from the sample of ribosomes; and determining the sequence of the RPFs thereby identifying translated sequences. Methods of culturing cells under conditions sufficient to produce translation products are generally known in the art. In some embodiments, the cell is a eukaryotic cell. In some embodiments, the cell is a human cell. In some embodiments, the cell is a non-human animal cell. In some embodiments, the cell is a prokaryotic cell.

In certain example embodiments, determining the sequence of the RPFs comprises nucleotide sequencing. In certain example embodiments, sequencing comprises RNA sequencing. In certain example embodiments, sequencing comprises generating cDNA from the RPFs to form RPF cDNA and DNA sequencing the RPF cDNA. Specific suitable sequencing techniques are generally known in the art and include without limitation any next generation or third generation sequencing technique. See e.g., Hu et al., Hum Immunol. 2021 November; 82(11):801-811. doi: 10.1016/j.humimm.2021.02.012; E. R. Mardis. Annu Rev Genomics Hum Genet. 2008; 9:387-402. doi: 10.1146/annurev.genom.9.081307.164359; McCombie et al., Cold Spring Harb Perspect Med. 2019 Nov. 1; 9(11):a036798. doi: 10.1101/cshperspect.a036798; Gu et al., Annu Rev Pathol. 2019 Jan. 24; 14:319-338. doi: 10.1146/annurev-pathmechdis-012418-012751; Kumar et al., Semin Thromb Hemost. 2019 October; 45(7):661-673. doi: 10.1055/s-0039-1688446; van Dijk et al., Trends Genet. 2018 September; 34(9):666-681. doi: 10.1016/j.tig.2018.05.008; Rothberg and Rothberg. Clin Chem. 2015 July; 61(7):997-8. doi: 10.1373/clinchem.2014.237461; Athanasopoulou et al., Life (Basel). 2021 Dec. 26; 12(1):30. doi: 10.3390/life12010030; Pervez et al., Biomed Res Int. 2022 Sep. 29; 2022:3457806. doi: 10.1155/2022/3457806.

In certain example embodiments, the method further comprises digesting unprotected mRNA prior to recovering ribosome footprints. In some embodiments, digesting comprises RNase digestion. In some embodiments, the RNase is RNase A, RNase H, RNase III, RNase P, RNase E, RNase L, RNase T, RNase Z, RNase D, RNase P/RP, RNase I, RNase S7, RNase T1, or any combination thereof. In certain example embodiments, the method further comprises removing rRNA from the sample containing ribosomes. In some embodiments, RNAse digestion comprises exposing ribosome bound RNA to an RNase for an amount of time under conditions that allow for activity of the RNase. The ribosomes protect the RNA bound to the ribosome from RNase digestion. In some embodiments, the amount of time ranges from 15 minutes, 30 minutes, 45 minutes, 1, 2, 3, 4, 5, 6, 7, 8, 9, 10, 11, 12, 13, 14, 15, 16, 17, 18, 19, 20, 21, 22, 23, 24, or more hours.

In some embodiments, the method comprises immobilizing ribosomes complexes on RNA. In some embodiments immobilizing comprises chemical immobilization. Suitable chemicals and methods for immobilizing ribosome complexes are generally known and include without limitation cycloheximide.

In some embodiments, the method comprises introducing a translation inhibitor to prevent translation of the ribosome bound RNA. In some embodiments, the same effect can be achieved by utilizing translation-incompetent lysis conditions.

In some embodiments, the method comprises one or more filtration or separation techniques, such as centrifugation/ultracentrifugation, sucrose gradient separation, immunoprecipitation, size separation, liquid chromatography (including high-performance liquid chromatography), or any combination thereof. In some embodiments, ribosome bound RNA is separated from cells, cell components, and/or digested RNA using one or more filtration or separation techniques.

In some embodiments, the method comprises cell lysis. In some embodiments cell lysis after expressing a vector as described elsewhere herein or a vector library as described elsewhere herein in one or more cells. Methods of cell lysis are generally known in the art. Exemplary methods of cell lysis include, but are not limited to, freeze-thaw methods, sonication, centrifugation, chemical lysis, and/or enzymatic lysis.

In some embodiments, the method comprises removing the ribosomes from the protected RNA fragments. Any suitable purification method can be used, including but not limited to phenol/chloroform extraction.

In some embodiments, the method comprises ligating or otherwise adding one or more nucleic acid and/or sequencing adaptors, barcodes, and other nucleic acids to the one or more previously protected RNA fragments. Suitable barcoding techniques and/or sequencing adaptors are generally known. See e.g., Antil et al., Mol Biol Rep. 2023 January; 50(1):761-775. doi: 10.1007/s11033-022-08015-7; Kress and Erickson. Proceedings of the National Academy of Sciences. 105 (8) 2761-2762.

Recovering ribosome footprints consisting essentially of the RPFs from the sample of ribosomes and determining the sequence of the RPFs thereby identifying translated sequences can include one or more computational analyses. In some embodiments, ORFs are identified in the ribosome footprints using the PRICE algorithm as described in Erhard et al., Nature Methods volume 15, pages 363-366 (2018). Other methods of analysis will be appreciated by those of ordinary skill in the art in view of the description herein. In some embodiments, the ORFs identified can be, without limitation, annotated ORFs, upstream ORFs, upstream overlapping ORFS (uoORFs), internal overlapping ORFs, N-extended ORFs.

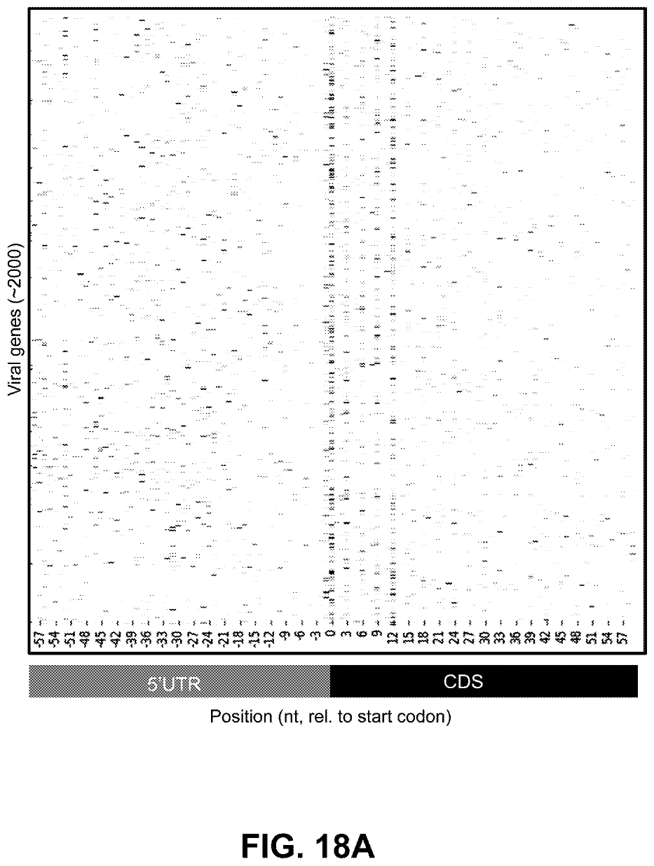

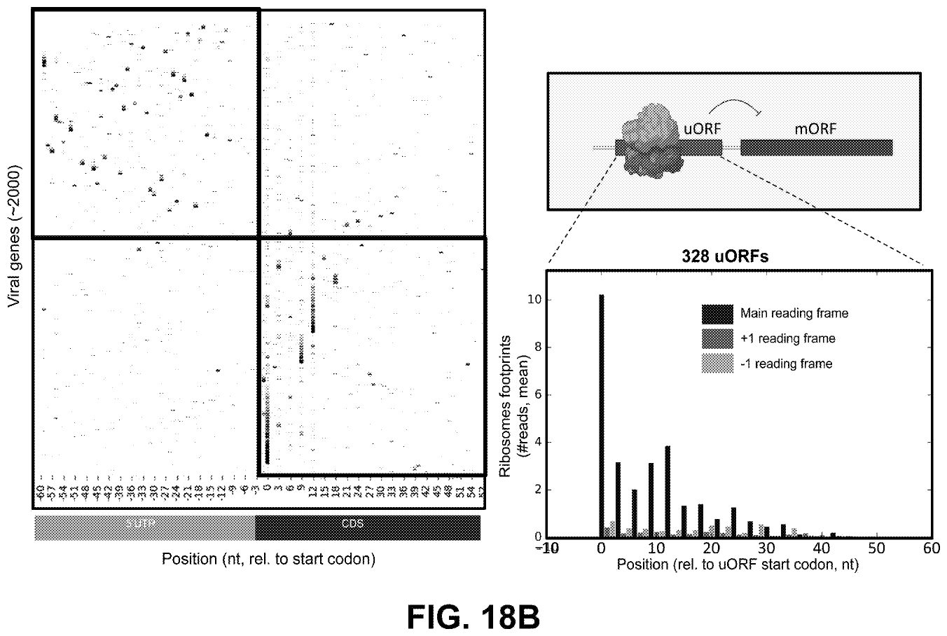

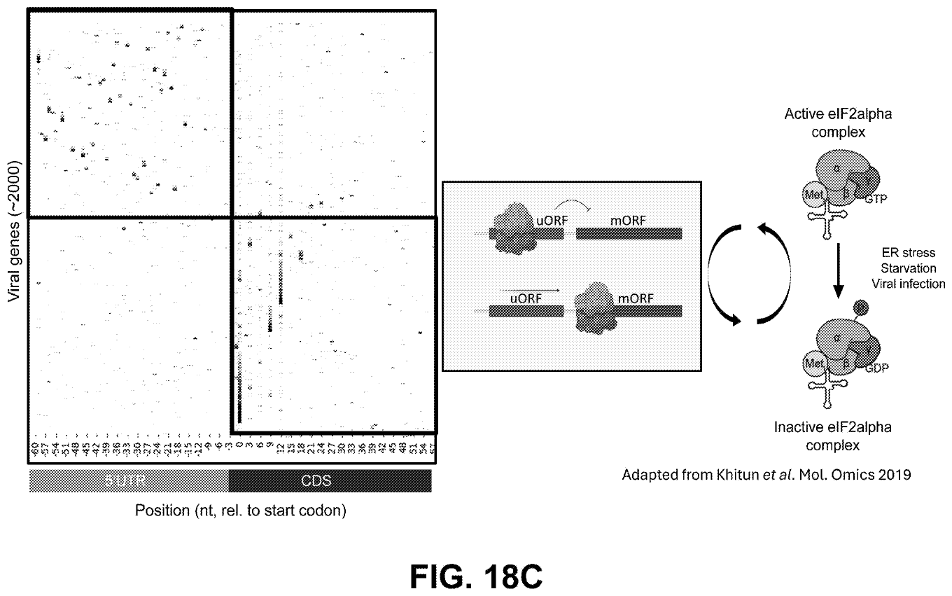

In some embodiments, the massively parallel ribosome profiling method can be coupled with one or more other techniques so as to determine one or more functionalities or effects of the one or more identified sequences. In some embodiments, the cells can be treated with one or more agents or conditions and effect on the translated sequences can be observed using the MPRP assay performed with and without exposure to the agent and/or condition. See e.g., FIGS. 18A-18C, 19, 20. Described in certain example embodiments herein are methods of determining translational regulation comprising determining a first set of translated sequences by a method comprising expressing a pan-genomic library in a first plurality of cells, wherein the pan-genomic library comprises a plurality of synthetic library expression constructs, each comprising a short synthetic polynucleotide and a pair of constant primers flanking the short synthetic polynucleotide, wherein the short synthetic polynucleotide has a sequence corresponding to a region of a genome; obtaining a sample of ribosomes comprising ribosome-protected mRNA fragments (RPFs) from the first plurality of cells; recovering ribosome footprints consisting essentially of the RPFs from the sample of ribosomes; and determining the sequence of the RPFs thereby identifying translated sequences to form the first set of translated sequences; determining a second set of translated sequences by a method comprising expressing the pan-genomic library in a second plurality of cells; applying a stress to the second plurality of cells; obtaining a sample of ribosomes comprising ribosome-protected mRNA fragments (RPFs) from the second plurality of cells; recovering ribosome footprints consisting essentially of the RPFs from the sample of ribosomes; and determining the sequence of the RPFs thereby identifying translated sequences to form the second set of translated sequences, whereby similarities and differences in the sequences of the first and the second set of translated sequences indicates sequences that are translationally regulated or regulate translation. In certain example embodiments herein, expressing a pan-genomic library in a first plurality of cells and expressing a pan-genomic library in a second plurality of cells comprises expressing one or more vectors as described elsewhere herein or a vector library as in any one of the preceding paragraphs or elsewhere herein in the first plurality of cells and the second plurality of cells.

### Exemplary Stresses

In certain example embodiments, the stress applied to the cells can be a test agent or condition that results in a stress on the cell. In some embodiments, the stress is a small molecule agent, a biologic agent, a chemical agent, a physical stress, a chemical stress, or any combination thereof. In certain example embodiments, the stress is a small molecule agent, a biologic agent, a physical stress, a chemical stress, or any combination thereof. In certain example embodiments, the stress is arsenite. “Small molecule” is a term of art that is generally referred to small molecules ranging from about 0.1 to 1 kDA in size. In some embodiments, the small molecules are biologic molecules such as proteins or nucleic acids. In some embodiments, the small molecules are chemical compounds. Biologic compounds include, but are not limited to, DNA (single and double stranded), RNA (single and double stranded), hybrid DNA:RNA molecules, peptides, polypeptides, amino acids, lipids, and/or any combination thereof. Other exemplary agents include, but are not limited to Drug compounds include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, radiation sensitizers, chemotherapeutics.

Suitable hormones include, but are not limited to, amino-acid derived hormones (e.g. melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eicosanoids (e.g. arachidonic acid, lipoxins, and prostaglandins), and steroid hormones (e.g. estradiol, testosterone, tetrahydro testosterone, cortisol).

Suitable immunomodulators include, but are not limited to, prednisone, azathioprine, 6-MP, cyclosporine, tacrolimus, methotrexate, interleukins (e.g., IL-2, IL-7, and IL-12), cytokines (e.g. interferons (e.g. IFN-α, IFN-β, IFN-ε, IFN-K, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylate, and sodium salicylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepressants, and monoamine oxidase inhibitors), mebicar, afobazole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene. Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H1-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g. cimetidine, famotidine, lafutidine, nizatidine, rafitidine, and roxatidine), tritoqualine, catechin, cromoglicate, nedocromil, and p2-adrenergic agonists.

Suitable anti-infectives include, but are not limited to, amebicides (e.g. nitazoxanide, paromomycin, metronidazole, tinidazole, chloroquine, miltefosine, amphotericin b, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxaxillin, dicloxacillin, and nafcillin), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

Suitable radiation sensitizers include, but are not limited to, 5-fluorouracil, platinum analogs (e.g. cisplatin, carboplatin, and oxaliplatin), gemcitabine, DNA topoisomerase I-targeting drugs (e.g. camptothecin derivatives (e.g. topotecan and irinotecan)), epidermal growth factor receptor blockade family agents (e.g. cetuximab, gefitinib), farnesyltransferase inhibitors (e.g., L-778-123), COX-2 inhibitors (e.g. rofecoxib, celecoxib, and etoricoxib), bFGF and VEGF targeting agents (e.g. bevazucimab and thalidomide), NBTXR3, Nimoral, trans sodium crocetinate, NVX-108, and combinations thereof. See also e.g., Kvols, L. K., J Nucl Med 2005; 46:187S-190S.

In some embodiments, the stress is one or more anti-viral agents. Exemplary antiviral-agents include without limitation, nucleoside analogues with reverse transcriptase activity (e.g., tenofovir, emtricitabine, lamivudine, abacavir, stavudine, didanosine, and zidovudine), nonnucleoside reverse transcriptase inhibitors (e.g., delavirdine, efavirenz, etravirine, nevirapine, and rilpivirine), protease inhibitors (atazanavir, darunavir, indinavir, ritonavir, tipranavir and many others), and miscellaneous agents such as maraviroc, enfuvirtide (fusion inhibitor), and integrase inhibitors (raltegravir, elvitegravir and dolutegravir), emdesivir, molnupiravir, Paxlovid (nirmatrelvir and ritonavir), Tecovirimat, seltamivir (oral), zanamivir (by inhalation) and peramivir (intravenous), baloxavir (oral), valacyclovir, cidofovir, famciclovir, ganciclovir, valganciclovir, foscarnet, simeprevir, paritaprevir, grazoprevir, glecaprevir, sofosbuvir, dasabuvir, daclatasvir, elbasvir, lepidasvir, velpatsvir, ombitasvir, pibrentasvor, Alpha interferon, peginterferon, monoclonal antibodies targeting specific viruses, and any combination thereof.

In some embodiments, the stress condition is applied to the cell during and/or after transduction of a viral particle. The stress condition can be any stress applied to the cell, including but not limited to a thermal stress (e.g., applying hot or cold environmental conditions to the cell), oxygen or other gas depletion or enrichment; a strain, a tension, a pressure, a change in salinity, osmolarity, pH, or other cell culture media condition, and/or the like. Such a stress can be applied continuously or be applied for one or more periods of time. Other stresses will be appreciated by one of ordinary skill in the art in view of the description herein.

### Short Synthetic Sequences

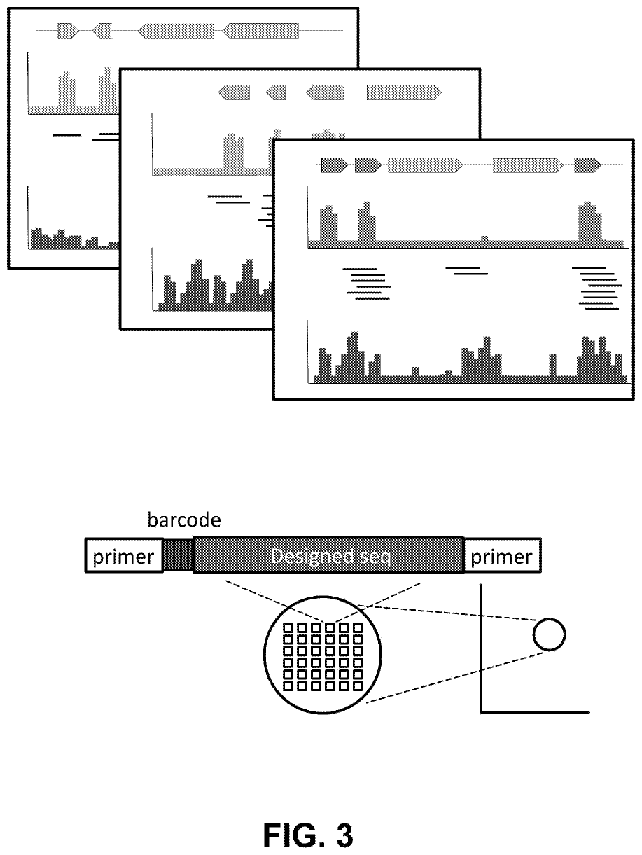

In certain example embodiments of the MPRP, the short synthetic polynucleotide is about 200 nucleotides. The short synthetic sequence can be that designed or variable sequence included in the pan-genomic library, which is discussed in greater detail elsewhere herein. See e.g., FIGS. 3-6. In certain example embodiments, the short synthetic polynucleotide has a sequence corresponding to a region of a genome. In certain example embodiments, the short synthetic polynucleotide has a sequence corresponding to a region of a human genome, a non-human animal genome, a plant genome, a microbial genome, or a viral genome. In some embodiments, the viral genome is from a virus set for the elsewhere herein, see e.g., section “Exemplary viruses” elsewhere herein. In certain example embodiments, the short synthetic polynucleotide has a sequence corresponding to (a) an annotated region of a genome; (b) an unannotated region of a genome; (c) a mutation; (d) a 5′UTR; (e) a 3′UTR; (f) an open reading frame; or (g) any combination thereof. In certain example embodiments herein, the short synthetic polynucleotide (a) is a sequence according to a sequence in SEQ ID NOS: 1-9890 or SEQ ID NOS: 9891-16277; (b) is a synthetic in silico designed sequence; (d) does not have 100 percent identity to a genome sequence, optionally a human genome, a non-human animal genome, a plant genome, a microbial genome, or a viral genome; (e) is not native to any genome, optionally a human genome, a non-human animal genome, a plant genome, a microbial genome, or a viral genome; or (f) any combination thereof. These are further discussed with respect to the vectors and vector libraries herein.

The short synthetic sequences can be about 200 nucleotides. In some embodiments, the designed oligos range from about 10 to about 200 oligonucleotides, such as 10, to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134,135, 136, 137, 138, 139,140, 141, 142, 143, 144,145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 nucleotides. In some embodiments, the short synthetic sequence is 80-100 percent identical (e.g., 80%, to/or 81%, 82%, 83%, 84%, 85%, 86%, 87%, 88%, 89%, 90%, 91%, 92%, 93%, 94%, 95%, 96%, 97%, 98%, 99%, 100% identical) to a polynucleotide set forth in SEQ ID NOS: 1-9890 or SEQ ID NOS: 9891-16277.

### Vectors and Vector Libraries for MPRP

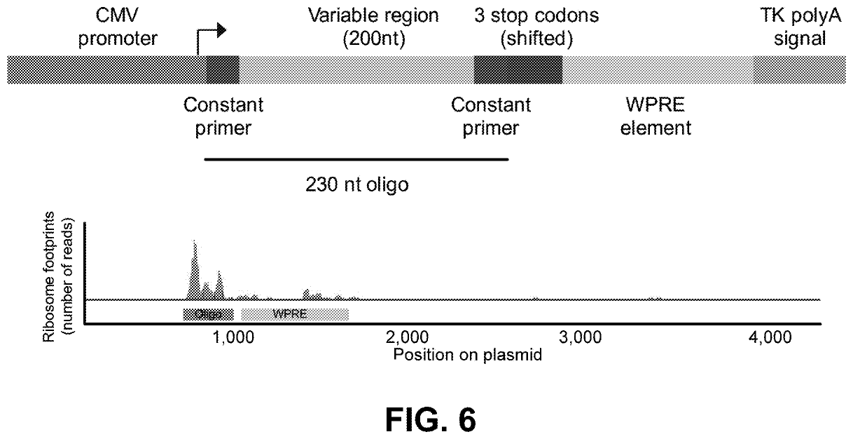

As shown in FIGS. 3-5, a synthetic oligo library can be prepared and synthesized. Unlike conventional approaches, the short synthetic oligo (also referred to as “designed oligos”) is flanked by two constant primers and is followed by a shifted stop codons and polyA signal (see e.g., FIG. 6). The short synthetic oligos can be about 200 nucleotides. In some embodiments, the short synthetic oligos range from about 10 to about 200 oligonucleotides, such as 10, to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125,126, 127, 128, 129, 130, 131, 132, 133, 134, 135, 136, 137,138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 nucleotides. The sources of the synthetic oligonucleotides can be annotated genomic sequences, such as (a) an annotated region of a genome; (b) an unannotated region of a genome; (c) a mutation; (d) a 5′UTR; (e) a 3′UTR; (f) an open reading frame; or (g) any combination thereof. In certain example embodiments herein, the short synthetic polynucleotide (a) is a sequence according to a sequence in SEQ ID NOS: 1-9890 or SEQ ID NOS: 9891-16277; (b) is a synthetic in silico designed sequence; (d) does not have 100 percent identity to a genome sequence, optionally a human genome, a non-human animal genome, a plant genome, a microbial genome, or a viral genome; (e) is not native to any genome, optionally a human genome, a non-human animal genome, a plant genome, a microbial genome, or a viral genome; or (f) any combination thereof.

Described in certain example embodiments herein are vectors comprising a synthetic library expression construct comprising a short synthetic polynucleotide; a pair of constant primers flanking the short synthetic polynucleotide, the pair of constant primers comprising a forward constant primer and a reverse constant primer, wherein the forward constant primer and the reverse constant primer are each independently coupled to the 5′ end or the 3′ end of the short synthetic polynucleotide; a stop codon polynucleotide comprising one or more stop codons, wherein the stop codon polynucleotide is coupled to the constant primer coupled to the 3′ end of the short synthetic polynucleotide such that the constant primer coupled to the 3′ end of the short synthetic polynucleotide is between the multiple stop codon polynucleotide and the short synthetic polynucleotide; a poly A signal, wherein the poly A signal is operably coupled to the short synthetic polynucleotide, the pair of constant primers, and the stop codon polynucleotide; and a promoter, wherein the promoter is operably coupled to the short synthetic polynucleotide, the pair of constant primers, and the stop codon polynucleotide, and the poly A signal. In certain example embodiments, the short synthetic polynucleotide is about 200 nucleotides. In certain example embodiments, the short synthetic polynucleotide has a sequence corresponding to a region of a genome. In certain example embodiments, the short synthetic polynucleotide has a sequence corresponding to a region of a human genome, a non-human animal genome, a plant genome, a microbial genome, or a viral genome. In certain example embodiments, the short synthetic polynucleotide has a sequence corresponding to a (a) an annotated region of a genome; (b) an unannotated region of a genome; (c) a mutation; (d) a 5′UTR; (e) a 3′UTR; (f) an open reading frame; or (g) any combination thereof. In certain example embodiments, the short synthetic polynucleotide (a) is a sequence according to a sequence in SEQ ID NOS: 1-9890 or SEQ ID NOS: 9891-16277; (b) is a synthetic in silico designed sequence; (d) does not have 100 percent identity to a genome sequence, optionally a human genome, a non-human animal genome, a plant genome, a microbial genome, or a viral genome; (e) is not native to any genome, optionally a human genome, a non-human animal genome, a plant genome, a microbial genome, or a viral genome; or (f) any combination thereof.

In certain example embodiments, the vector comprises two or more synthetic library expression constructs. In certain example embodiments, at least two of the two or more synthetic library expression constructs comprises a different short synthetic polynucleotide. In certain example embodiments, each of the two or more synthetic library expression constructs comprises a different short synthetic polynucleotide. In certain example embodiments, at least two of the two or more synthetic library constructs comprises the same short synthetic polynucleotide. In certain example embodiments, the vector further comprises an additional regulatory element, a reporter gene, an origin of replication, a cloning sites, an internal ribosome entry sites, a transcription termination sequence, an inverted terminal repeat, a long terminal repeats, a trans-activating response elements, a central polypurine tract, a Psi element, a Rev response element, a packaging protein gene, a polymerase gene, an envelope protein gene, a capsid protein gene, a Rep protein gene, a U3 element, a repeat element (R), a unique 5′ element (U5), an untranslated region stabilization element, or any combination thereof. In certain example embodiments, the vector further comprises a Woodchuck hepatitis virus post-transcriptional regulatory element (WPRE), wherein the WPRE is operably coupled to the short synthetic polynucleotide and the pair of constant primers. In certain example embodiments, the vector is a eukaryotic expression vector. In certain example embodiments, the vector is a viral expression vector. In certain example embodiments, the promoter is a constitutive promoter. In certain example embodiments, the promoter is an inducible promoter or a conditional promoter. Exemplary vectors and additional vector components are described in greater detail elsewhere herein.

Described in certain example embodiments herein are synthetic oligo libraries comprising a plurality of short synthetic oligos optionally flanked by a forward and reverse constant primers. Short synthetic oligos are described in greater detail elsewhere herein. Described in certain example embodiments herein are vector libraries each comprising a plurality of vectors as described elsewhere herein.

In some embodiments, the synthetic oligo library contains 100, to/or 150, 200, 250, 300, 350, 400, 450, 500, 550, 600, 650, 700, 750, 800, 850, 900, 950, 1000, 1000, 2000, 3000, 4000, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, 100000, 101000, 102000, 103000, 104000, 105000, 106000, 107000, 108000, 109000, 110000, 111000, 112000, 113000, 114000, 115000, 116000, 117000, 118000, 119000, 120000, 121000, 122000, 123000, 124000, 125000, 126000, 127000, 128000, 129000, 130000, 131000, 132000, 133000, 134000, 135000, 136000, 137000, 138000, 139000, 140000, 141000, 142000, 143000, 144000, 145000, 146000, 147000, 148000, 149000, 150000, 151000, 152000, 153000, 154000, 155000, 156000, 157000, 158000, 159000, 160000, 161000, 162000, 163000, 164000, 165000, 166000, 167000, 168000, 169000, 170000, 171000, 172000, 173000, 174000, 175000, 176000, 177000, 178000, 179000, 180000, 181000, 182000, 183000, 184000, 185000, 186000, 187000, 188000, 189000, 190000, 191000, 192000, 193000, 194000, 195000, 196000, 197000, 198000, 199000, 200000, 201000, 202000, 203000, 204000, 205000, 206000, 207000, 208000, 209000, 210000, 211000, 212000, 213000, 214000, 215000, 216000, 217000, 218000, 219000, 220000, 221000, 222000, 223000, 224000, 225000, 226000, 227000, 228000, 229000, 230000, 231000, 232000, 233000, 234000, 235000, 236000, 237000, 238000, 239000, 240000, 241000, 242000, 243000, 244000, 245000, 246000, 247000, 248000, 249000, 250000, 251000, 252000, 253000, 254000, 255000, 256000, 257000, 258000, 259000, 260000, 261000, 262000, 263000, 264000, 265000, 266000, 267000, 268000, 269000, 270000, 271000, 272000, 273000, 274000, 275000, 276000, 277000, 278000, 279000, 280000, 281000, 282000, 283000, 284000, 285000, 286000, 287000, 288000, 289000, 290000, 291000, 292000, 293000, 294000, 295000, 296000, 297000, 298000, 299000, 300000, 301000, 302000, 303000, 304000, 305000, 306000, 307000, 308000, 309000, 310000, 311000, 312000, 313000, 314000, 315000, 316000, 317000, 318000, 319000, 320000, 321000, 322000, 323000, 324000, 325000, 326000, 327000, 328000, 329000, 330000, 331000, 332000, 333000, 334000, 335000, 336000, 337000, 338000, 339000, 340000, 341000, 342000, 343000, 344000, 345000, 346000, 347000, 348000, 349000, 350000, 351000, 352000, 353000, 354000, 355000, 356000, 357000, 358000, 359000, 360000, 361000, 362000, 363000, 364000, 365000, 366000, 367000, 368000, 369000, 370000, 371000, 372000, 373000, 374000, 375000, 376000, 377000, 378000, 379000, 380000, 381000, 382000, 383000, 384000, 385000, 386000, 387000, 388000, 389000, 390000, 391000, 392000, 393000, 394000, 395000, 396000, 397000, 398000, 399000, 400000, 401000, 402000, 403000, 404000, 405000, 406000, 407000, 408000, 409000, 410000, 411000, 412000, 413000, 414000, 415000, 416000, 417000, 418000, 419000, 420000, 421000, 422000, 423000, 424000, 425000, 426000, 427000, 428000, 429000, 430000, 431000, 432000, 433000, 434000, 435000, 436000, 437000, 438000, 439000, 440000, 441000, 442000, 443000, 444000, 445000, 446000, 447000, 448000, 449000, 450000, 451000, 452000, 453000, 454000, 455000, 456000, 457000, 458000, 459000, 460000, 461000, 462000, 463000, 464000, 465000, 466000, 467000, 468000, 469000, 470000, 471000, 472000, 473000, 474000, 475000, 476000, 477000, 478000, 479000, 480000, 481000, 482000, 483000, 484000, 485000, 486000, 487000, 488000, 489000, 490000, 491000, 492000, 493000, 494000, 495000, 496000, 497000, 498000, 499000, 500000, 501000, 502000, 503000, 504000, 505000, 506000, 507000, 508000, 509000, 510000, 511000, 512000, 513000, 514000, 515000, 516000, 517000, 518000, 519000, 520000, 521000, 522000, 523000, 524000, 525000, 526000, 527000, 528000, 529000, 530000, 531000, 532000, 533000, 534000, 535000, 536000, 537000, 538000, 539000, 540000, 541000, 542000, 543000, 544000, 545000, 546000, 547000, 548000, 549000, 550000, 551000, 552000, 553000, 554000, 555000, 556000, 557000, 558000, 559000, 560000, 561000, 562000, 563000, 564000, 565000, 566000, 567000, 568000, 569000, 570000, 571000, 572000, 573000, 574000, 575000, 576000, 577000, 578000, 579000, 580000, 581000, 582000, 583000, 584000, 585000, 586000, 587000, 588000, 589000, 590000, 591000, 592000, 593000, 594000, 595000, 596000, 597000, 598000, 599000, 600000, 601000, 602000, 603000, 604000, 605000, 606000, 607000, 608000, 609000, 610000, 611000, 612000, 613000, 614000, 615000, 616000, 617000, 618000, 619000, 620000, 621000, 622000, 623000, 624000, 625000, 626000, 627000, 628000, 629000, 630000, 631000, 632000, 633000, 634000, 635000, 636000, 637000, 638000, 639000, 640000, 641000, 642000, 643000, 644000, 645000, 646000, 647000, 648000, 649000, 650000, 651000, 652000, 653000, 654000, 655000, 656000, 657000, 658000, 659000, 660000, 661000, 662000, 663000, 664000, 665000, 666000, 667000, 668000, 669000, 670000, 671000, 672000, 673000, 674000, 675000, 676000, 677000, 678000, 679000, 680000, 681000, 682000, 683000, 684000, 685000, 686000, 687000, 688000, 689000, 690000, 691000, 692000, 693000, 694000, 695000, 696000, 697000, 698000, 699000, 700000, 701000, 702000, 703000, 704000, 705000, 706000, 707000, 708000, 709000, 710000, 711000, 712000, 713000, 714000, 715000, 716000, 717000, 718000, 719000, 720000, 721000, 722000, 723000, 724000, 725000, 726000, 727000, 728000, 729000, 730000, 731000, 732000, 733000, 734000, 735000, 736000, 737000, 738000, 739000, 740000, 741000, 742000, 743000, 744000, 745000, 746000, 747000, 748000, 749000, 750000, 751000, 752000, 753000, 754000, 755000, 756000, 757000, 758000, 759000, 760000, 761000, 762000, 763000, 764000, 765000, 766000, 767000, 768000, 769000, 770000, 771000, 772000, 773000, 774000, 775000, 776000, 777000, 778000, 779000, 780000, 781000, 782000, 783000, 784000, 785000, 786000, 787000, 788000, 789000, 790000, 791000, 792000, 793000, 794000, 795000, 796000, 797000, 798000, 799000, 800000, 801000, 802000, 803000, 804000, 805000, 806000, 807000, 808000, 809000, 810000, 811000, 812000, 813000, 814000, 815000, 816000, 817000, 818000, 819000, 820000, 821000, 822000, 823000, 824000, 825000, 826000, 827000, 828000, 829000, 830000, 831000, 832000, 833000, 834000, 835000, 836000, 837000, 838000, 839000, 840000, 841000, 842000, 843000, 844000, 845000, 846000, 847000, 848000, 849000, 850000, 851000, 852000, 853000, 854000, 855000, 856000, 857000, 858000, 859000, 860000, 861000, 862000, 863000, 864000, 865000, 866000, 867000, 868000, 869000, 870000, 871000, 872000, 873000, 874000, 875000, 876000, 877000, 878000, 879000, 880000, 881000, 882000, 883000, 884000, 885000, 886000, 887000, 888000, 889000, 890000, 891000, 892000, 893000, 894000, 895000, 896000, 897000, 898000, 899000, 900000, 901000, 902000, 903000, 904000, 905000, 906000, 907000, 908000, 909000, 910000, 911000, 912000, 913000, 914000, 915000, 916000, 917000, 918000, 919000, 920000, 921000, 922000, 923000, 924000, 925000, 926000, 927000, 928000, 929000, 930000, 931000, 932000, 933000, 934000, 935000, 936000, 937000, 938000, 939000, 940000, 941000, 942000, 943000, 944000, 945000, 946000, 947000, 948000, 949000, 950000, 951000, 952000, 953000, 954000, 955000, 956000, 957000, 958000, 959000, 960000, 961000, 962000, 963000, 964000, 965000, 966000, 967000, 968000, 969000, 970000, 971000, 972000, 973000, 974000, 975000, 976000, 977000, 978000, 979000, 980000, 981000, 982000, 983000, 984000, 985000, 986000, 987000, 988000, 989000, 990000, 991000, 992000, 993000, 994000, 995000, 996000, 997000, 998000, 999000, 1000000, or more synthetic oligonucleotide sequences.

In some embodiments, the vector library comprises 100, to/or 150, 200, 250, 300, 350, 400, 450, 500, 550, 600, 650, 700, 750, 800, 850, 900, 950, 1000, 1000, 2000, 3000, 4000, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, 100000, 101000, 102000, 103000, 104000, 105000, 106000, 107000, 108000, 109000, 110000, 111000, 112000, 113000, 114000, 115000, 116000, 117000, 118000, 119000, 120000, 121000, 122000, 123000, 124000, 125000, 126000, 127000, 128000, 129000, 130000, 131000, 132000, 133000, 134000, 135000, 136000, 137000, 138000, 139000, 140000, 141000, 142000, 143000, 144000, 145000, 146000, 147000, 148000, 149000, 150000, 151000, 152000, 153000, 154000, 155000, 156000, 157000, 158000, 159000, 160000, 161000, 162000, 163000, 164000, 165000, 166000, 167000, 168000, 169000, 170000, 171000, 172000, 173000, 174000, 175000, 176000, 177000, 178000, 179000, 180000, 181000, 182000, 183000, 184000, 185000, 186000, 187000, 188000, 189000, 190000, 191000, 192000, 193000, 194000, 195000, 196000, 197000, 198000, 199000, 200000, 201000, 202000, 203000, 204000, 205000, 206000, 207000, 208000, 209000, 210000, 211000, 212000, 213000, 214000, 215000, 216000, 217000, 218000, 219000, 220000, 221000, 222000, 223000, 224000, 225000, 226000, 227000, 228000, 229000, 230000, 231000, 232000, 233000, 234000, 235000, 236000, 237000, 238000, 239000, 240000, 241000, 242000, 243000, 244000, 245000, 246000, 247000, 248000, 249000, 250000, 251000, 252000, 253000, 254000, 255000, 256000, 257000, 258000, 259000, 260000, 261000, 262000, 263000, 264000, 265000, 266000, 267000, 268000, 269000, 270000, 271000, 272000, 273000, 274000, 275000, 276000, 277000, 278000, 279000, 280000, 281000, 282000, 283000, 284000, 285000, 286000, 287000, 288000, 289000, 290000, 291000, 292000, 293000, 294000, 295000, 296000, 297000, 298000, 299000, 300000, 301000, 302000, 303000, 304000, 305000, 306000, 307000, 308000, 309000, 310000, 311000, 312000, 313000, 314000, 315000, 316000, 317000, 318000, 319000, 320000, 321000, 322000, 323000, 324000, 325000, 326000, 327000, 328000, 329000, 330000, 331000, 332000, 333000, 334000, 335000, 336000, 337000, 338000, 339000, 340000, 341000, 342000, 343000, 344000, 345000, 346000, 347000, 348000, 349000, 350000, 351000, 352000, 353000, 354000, 355000, 356000, 357000, 358000, 359000, 360000, 361000, 362000, 363000, 364000, 365000, 366000, 367000, 368000, 369000, 370000, 371000, 372000, 373000, 374000, 375000, 376000, 377000, 378000, 379000, 380000, 381000, 382000, 383000, 384000, 385000, 386000, 387000, 388000, 389000, 390000, 391000, 392000, 393000, 394000, 395000, 396000, 397000, 398000, 399000, 400000, 401000, 402000, 403000, 404000, 405000, 406000, 407000, 408000, 409000, 410000, 411000, 412000, 413000, 414000, 415000, 416000, 417000, 418000, 419000, 420000, 421000, 422000, 423000, 424000, 425000, 426000, 427000, 428000, 429000, 430000, 431000, 432000, 433000, 434000, 435000, 436000, 437000, 438000, 439000, 440000, 441000, 442000, 443000, 444000, 445000, 446000, 447000, 448000, 449000, 450000, 451000, 452000, 453000, 454000, 455000, 456000, 457000, 458000, 459000, 460000, 461000, 462000, 463000, 464000, 465000, 466000, 467000, 468000, 469000, 470000, 471000, 472000, 473000, 474000, 475000, 476000, 477000, 478000, 479000, 480000, 481000, 482000, 483000, 484000, 485000, 486000, 487000, 488000, 489000, 490000, 491000, 492000, 493000, 494000, 495000, 496000, 497000, 498000, 499000, 500000, 501000, 502000, 503000, 504000, 505000, 506000, 507000, 508000, 509000, 510000, 511000, 512000, 513000, 514000, 515000, 516000, 517000, 518000, 519000, 520000, 521000, 522000, 523000, 524000, 525000, 526000, 527000, 528000, 529000, 530000, 531000, 532000, 533000, 534000, 535000, 536000, 537000, 538000, 539000, 540000, 541000, 542000, 543000, 544000, 545000, 546000, 547000, 548000, 549000, 550000, 551000, 552000, 553000, 554000, 555000, 556000, 557000, 558000, 559000, 560000, 561000, 562000, 563000, 564000, 565000, 566000, 567000, 568000, 569000, 570000, 571000, 572000, 573000, 574000, 575000, 576000, 577000, 578000, 579000, 580000, 581000, 582000, 583000, 584000, 585000, 586000, 587000, 588000, 589000, 590000, 591000, 592000, 593000, 594000, 595000, 596000, 597000, 598000, 599000, 600000, 601000, 602000, 603000, 604000, 605000, 606000, 607000, 608000, 609000, 610000, 611000, 612000, 613000, 614000, 615000, 616000, 617000, 618000, 619000, 620000, 621000, 622000, 623000, 624000, 625000, 626000, 627000, 628000, 629000, 630000, 631000, 632000, 633000, 634000, 635000, 636000, 637000, 638000, 639000, 640000, 641000, 642000, 643000, 644000, 645000, 646000, 647000, 648000, 649000, 650000, 651000, 652000, 653000, 654000, 655000, 656000, 657000, 658000, 659000, 660000, 661000, 662000, 663000, 664000, 665000, 666000, 667000, 668000, 669000, 670000, 671000, 672000, 673000, 674000, 675000, 676000, 677000, 678000, 679000, 680000, 681000, 682000, 683000, 684000, 685000, 686000, 687000, 688000, 689000, 690000, 691000, 692000, 693000, 694000, 695000, 696000, 697000, 698000, 699000, 700000, 701000, 702000, 703000, 704000, 705000, 706000, 707000, 708000, 709000, 710000, 711000, 712000, 713000, 714000, 715000, 716000, 717000, 718000, 719000, 720000, 721000, 722000, 723000, 724000, 725000, 726000, 727000, 728000, 729000, 730000, 731000, 732000, 733000, 734000, 735000, 736000, 737000, 738000, 739000, 740000, 741000, 742000, 743000, 744000, 745000, 746000, 747000, 748000, 749000, 750000, 751000, 752000, 753000, 754000, 755000, 756000, 757000, 758000, 759000, 760000, 761000, 762000, 763000, 764000, 765000, 766000, 767000, 768000, 769000, 770000, 771000, 772000, 773000, 774000, 775000, 776000, 777000, 778000, 779000, 780000, 781000, 782000, 783000, 784000, 785000, 786000, 787000, 788000, 789000, 790000, 791000, 792000, 793000, 794000, 795000, 796000, 797000, 798000, 799000, 800000, 801000, 802000, 803000, 804000, 805000, 806000, 807000, 808000, 809000, 810000, 811000, 812000, 813000, 814000, 815000, 816000, 817000, 818000, 819000, 820000, 821000, 822000, 823000, 824000, 825000, 826000, 827000, 828000, 829000, 830000, 831000, 832000, 833000, 834000, 835000, 836000, 837000, 838000, 839000, 840000, 841000, 842000, 843000, 844000, 845000, 846000, 847000, 848000, 849000, 850000, 851000, 852000, 853000, 854000, 855000, 856000, 857000, 858000, 859000, 860000, 861000, 862000, 863000, 864000, 865000, 866000, 867000, 868000, 869000, 870000, 871000, 872000, 873000, 874000, 875000, 876000, 877000, 878000, 879000, 880000, 881000, 882000, 883000, 884000, 885000, 886000, 887000, 888000, 889000, 890000, 891000, 892000, 893000, 894000, 895000, 896000, 897000, 898000, 899000, 900000, 901000, 902000, 903000, 904000, 905000, 906000, 907000, 908000, 909000, 910000, 911000, 912000, 913000, 914000, 915000, 916000, 917000, 918000, 919000, 920000, 921000, 922000, 923000, 924000, 925000, 926000, 927000, 928000, 929000, 930000, 931000, 932000, 933000, 934000, 935000, 936000, 937000, 938000, 939000, 940000, 941000, 942000, 943000, 944000, 945000, 946000, 947000, 948000, 949000, 950000, 951000, 952000, 953000, 954000, 955000, 956000, 957000, 958000, 959000, 960000, 961000, 962000, 963000, 964000, 965000, 966000, 967000, 968000, 969000, 970000, 971000, 972000, 973000, 974000, 975000, 976000, 977000, 978000, 979000, 980000, 981000, 982000, 983000, 984000, 985000, 986000, 987000, 988000, 989000, 990000, 991000, 992000, 993000, 994000, 995000, 996000, 997000, 998000, 999000, 1000000, or more vectors.

In certain example embodiments of the vector library, at least two vectors of the plurality of vectors comprises different short synthetic oligos. In certain example embodiments of the vector library, at least two vectors of the plurality of vectors comprises the same synthetic oligos. In certain example embodiments of the vector library, all the vectors of the plurality of vectors comprise different short synthetic oligos.

In certain example embodiments of the synthetic oligo library, at least two synthetic oligos of the plurality of synthetic oligos are different. In certain example embodiments of the synthetic oligo library, are the same synthetic oligos. In certain example embodiments of the synthetic oligo library, all synthetic oligo of the plurality of vectors comprise different short synthetic oligos.

### Cells and Delivery Vehicles

Described in certain example embodiments herein are cells comprising a synthetic oligo, synthetic oligo library, vector, or vector library as described elsewhere herein. In certain example embodiments, the cell is a eukaryotic cell or a prokaryotic cell. In some embodiments, the cell is capable of producing viral or other particles containing a synthetic oligo, synthetic oligo library, vector, or vector library. Exemplary cells and delivery vehicles are described in greater detail elsewhere herein and can be used in context with a synthetic oligo, synthetic oligo library, vector, or vector library described herein.

### Massively Parallel Antigen Presentation

In some embodiments, the short synthetic oligos and/or any viral sequences, such as ORFs, identified using a method herein can be further analyzed for their ability to be presented in associate with a MHCI or MHCII molecule. See e.g., FIG. 4A-4D. Generally, a library is expressed in associate with a MHCI and/or MHC II molecule. This is followed by MHCI or MHCII immune precipitation. Peptides associated with the MHCI or MHCII are then analyzed using a suitable mass spectrometry or other appropriate protein analysis technique to identify immunogenic peptides. MHCs are described in greater detail elsewhere herein. Such an approach is further detailed in US 2022/0088180, which is incorporated by reference as if expressed in its entirety herein. Thus, described in certain example embodiments herein are methods of massively parallel antigen profiling comprising delivering to and expressing in a plurality of cells a pan-genomic library or a polynucleotide as in any one of the preceding paragraphs or as described elsewhere herein, wherein the pan-genomic library comprises a plurality of synthetic library expression constructs, each comprising a short synthetic polynucleotide and a pair of constant primers flanking the short synthetic polynucleotide, wherein the short synthetic polynucleotide has a sequence corresponding to a region of a genome; and determining the antigens presented by the cells. In certain example embodiments, delivery comprises infecting the plurality of cells with viral particles comprising the pan-genomic library or a polynucleotide as in any one of the preceding paragraphs or as described elsewhere herein. In certain example embodiments, determining the antigens comprises protein sequencing, mass spectrometry, Raman spectroscopy, immunodetection, chromatography, centrifugation, isoelectric focusing, or any combination thereof. In certain example embodiments, determining the antigens comprises isolating MHC complexes from the cells and detecting peptides loaded in the MHC, wherein the MHC is optionally an HLA, such as an HLAI or HLAII. In certain example embodiments, the method further comprises evaluating an immune response to the antigens presented. Such approaches are further detailed in e.g., US 2022/0088180.

Synthetic oligo and vector libraries are further described elsewhere herein, such as in connection with the MPRP assay previously described. Such libraries can be used in connection with the massively parallel antigen presentation assay described herein. In some embodiments, a subset of sequences can be used in the massively parallel antigen presentation assay described herein. In some embodiments, the subset are ORFs that are identified by the MPRP assay or massively parallel reporter assay described elsewhere herein.

### Massively Parallel Reporter Assay

Also described herein are massively parallel reporter assays that rely on dual optically active reporter proteins to allow for determining oligo expression post infection. A general exemplary assay methodology is set forth in e.g., FIG. 24. Thus, described in certain example embodiments herein are methods comprising: (a) transducing one or more cells with one or more viral particles or a pan-viral engineered viral particle library comprising an optically active reporter construct as described herein and allowing for genomic integration and expression of the optically active reporter construct in the one or more cells; (b) selecting cells for genomic integration of the optically active reporter construct via sorting by detecting an optical signal produced from the first optically active protein and selecting cells that produce the optical signal from the second optically active protein; (c) sorting selected cells from (b) into expression bins, wherein sorting selected cells from (b) comprises detecting an optical signal produced by the first optically active protein; (d) sequencing one or more nucleic acids of the sorted selected cells by expression bin; and (e) computing an expression score for each test polynucleotide by expression bin.

In some embodiments, the first and second optically active proteins are fluorescent proteins or luminescent proteins. Exemplary fluorescent and luminescent proteins include, without limitation, green fluorescent protein (GFP), cyan fluorescent protein (CFP), yellow fluorescent protein (YFP), red fluorescent protein (RFP), near infrared fluorescent proteins, luciferase, and variants thereof, such as those with modified optical properties. See also e.g., Chudakov et la., Physiol Rev. 2010 July; 90(3):1103-63. doi: 10.1152/physrev.00038.2009; Lukyanov, K. A., Biochem Biophys Res Commun. 2022 Dec. 10; 633:29-32. doi: 10.1016/j.bbrc.2022.08.089; Karasev et al., Biochemistry (Mosc). 2019 January; 84(Suppl 1):S32-S50. doi: 10.1134/S0006297919140037, any of which can be adapted for use with the present invention. In some embodiments, sorting and/or selecting cells is done via fluorescence activated cell sorting (FACS). Techniques and methods of FACS are generally known in the art (see e.g., Lanier et al., J Immunol. 2014 Sep. 1; 193(5):2043-4. doi: 10.4049/jimmunol.1401725; Schoendube et al., Int J Mol Sci. 2015 Jul. 24; 16(8):16897-919. doi: 10.3390/ijms160816897; Manohar et al., Bioanalysis. 2021 February; 13(3):181-198. doi: 10.4155/bio-2020-0267; McKinnon, M. K., Curr Protoc Immunol. 2018 Feb. 21; 120:5.1.1-5.1.11. doi: 10.1002/cpim.40; Robinson, J. P., Biotechniques. 2022 April; 72(4):159-169. doi: 10.2144/btn-2022-0005; Ibrahim et al., Adv Biochem Eng Biotechnol. 2007; 106:19-39. doi: 10.1007/10_2007_073; and Montante and Brinkman. Int J Lab Hematol. 2019 May; 41 Suppl 1:56-62. doi: 10.1111/ijlh.13016).

In certain example embodiments, sequencing comprises DNA sequencing, RNA sequencing, or both. In certain example embodiments, sequencing comprises genomic DNA sequencing. In certain example embodiments, sequencing comprises next generation sequencing or third generation sequencing. In certain example embodiments, sequencing comprises deep sequencing. In certain example embodiments, sequencing comprises single cell sequencing. Exemplary sequencing methods are generally known in the art. See, e.g., Hu et al., Hum Immunol. 2021 November; 82(11):801-811. doi: 10.1016/j.humimm.2021.02.012; E. R. Mardis. Annu Rev Genomics Hum Genet. 2008; 9:387-402. doi: 10.1 146/annurev.genom.9.081307.164359; McCombie et al., Cold Spring Harb Perspect Med. 2019 Nov. 1; 9(11):a036798. doi: 10.1101/cshperspect.a036798; Gu et al., Annu Rev Pathol. 2019 Jan. 24; 14:319-338. doi: 10.1146/annurev-pathmechdis-012418-012751; Kumar et al., Semin Thromb Hemost. 2019 October; 45(7):661-673. doi: 10.1055/s-0039-1688446; van Dijk et al., Trends Genet. 2018 September; 34(9):666-681. doi: 10.1016/j.tig.2018.05.008; Rothberg and Rothberg. Clin Chem. 2015 July; 61(7):997-8. doi: 10.1373/clinchem.2014.237461; Athanasopoulou et al., Life (Basel). 2021 Dec. 26; 12(1):30. doi: 10.3390/life12010030; Pervez et al., Biomed Res Int. 2022 Sep. 29; 2022:3457806. doi: 10.1155/2022/3457806; Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012; Picelli, S. et al., 2014, “Full-length RNA-seq from single cells using Smart-seq2” Nature protocols 9, 171-181, doi:10.1038/nprot.2014.006; Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273; Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International patent application number PCT/US2016/059239, published as WO2017164936 on Sep. 28, 2017; International patent application number PCT/US2018/060860, published as WO/2019/094984 on May 16, 2019; International patent application number PCT/US2019/055894, published as WO/2020/077236 on Apr. 16, 2020; and Drokhlyansky, et al., “The enteric nervous system of the human and mouse colon at a single-cell resolution,” bioRxiv 746743; doi: doi.org/10.1101/746743; Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1) and others set forth elsewhere herein.

### Screening Test Agents

The MPRP can be used to screen test agents and/or conditions for their effect on expression of the one or more polynucleotides, such as any of those in synthetic oligo, vector, viral particle, or pan viral library described herein. Generally, the method can include performing the MPRP in the presence and absence of the test agent or condition and comparing the outputs.

Described in certain example embodiments herein are methods of screening test agents and/or conditions comprising (a) transducing a first set of one or more cells with one or more viral particles or a pan-viral engineered viral particle library having an optically active reporter construct as described in any one of the preceding claims and elsewhere herein and allowing for genomic integration and expression of the optically active reporter construct in the one or more cells; (b) transducing a second set of the one or more cells with the one or more viral particles or a pan-viral engineered viral particle library having an optically active reporter construct as in any one of the preceding paragraphs or elsewhere herein and allowing for genomic integration and expression of the optically active reporter construct in the one or more cells, wherein the second set of one or more cells comprises the same one or more cells as the first set of one or more cells, and wherein the one or more viral particles or the pan-viral engineered viral particle library used to transduce the second set of one or more cells is the same as the one or more viral particles or the pan-viral engineered viral particle library used to transduce the first set of one or more cells; (c) exposing the second set of one or more cells to one or more test agents and/or conditions; (d) selecting cells in each of the first set of one or more cells and second set of one or more cells for genomic integration of the optically active reporter construct via sorting by detecting an optical signal produced from the first optically active protein and selecting cells that produce the optical signal from the second optically active protein; (e) sorting selected cells from (d) into expression bins, wherein sorting selected cells from (d) comprises detecting an optical signal produced by the first optically active protein; (f) sequencing one or more nucleic acids of the sorted selected cells by expression bin; (g) computing an expression score for each test polynucleotide by expression bin; and (h) comparing expression scores for each test polynucleotide between the first and second set of cells to determine an effect of the test agent and/or condition. In certain example embodiments, sequencing comprises DNA sequencing, RNA sequencing, or both. In certain example embodiments, sequencing comprises genomic DNA sequencing. In certain example embodiments, sequencing comprises next generation sequencing or third generation sequencing. In certain example embodiments, sequencing comprises deep sequencing. In certain example embodiments, sequencing comprises single cell sequencing. These and other techniques are described in greater detail elsewhere herein.

### Exemplary Test Agents and Conditions

In certain example embodiments, the test agent or condition is a small molecule agent, a biologic agent, a chemical agent, a physical stress, a chemical stress, or any combination thereof “Small molecule” is a term of art that is generally referred to small molecules ranging from about 0.1 to 1 kDA in size. In some embodiments, the small molecules are biologic molecules such as proteins or nucleic acids. In some embodiments, the small molecules are chemical compounds. Biologic compounds include, but are not limited to, DNA (single and double stranded), RNA (single and double stranded), hybrid DNA:RNA molecules, peptides, polypeptides, amino acids, lipids, and/or any combination thereof. Other exemplary agents include, but are not limited to Drug compounds include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, radiation sensitizers, chemotherapeutics.

Suitable hormones include, but are not limited to, amino-acid derived hormones (e.g. melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eicosanoids (e.g. arachidonic acid, lipoxins, and prostaglandins), and steroid hormones (e.g. estradiol, testosterone, tetrahydro testosterone, cortisol).

Suitable immunomodulators include, but are not limited to, prednisone, azathioprine, 6-MP, cyclosporine, tacrolimus, methotrexate, interleukins (e.g., IL-2, IL-7, and IL-12), cytokines (e.g. interferons (e.g. IFN-α, IFN-β, IFN-ε, IFN-K, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylate, and sodium salicylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepressants, and monoamine oxidase inhibitors), mebicar, afobazole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene. Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H1-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g. cimetidine, famotidine, lafutidine, nizatidine, rafitidine, and roxatidine), tritoqualine, catechin, cromoglicate, nedocromil, and p2-adrenergic agonists.

Suitable anti-infectives include, but are not limited to, amebicides (e.g. nitazoxanide, paromomycin, metronidazole, tinidazole, chloroquine, miltefosine, amphotericin b, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxaxillin, dicloxacillin, and nafcillin), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

Suitable radiation sensitizers include, but are not limited to, 5-fluorouracil, platinum analogs (e.g. cisplatin, carboplatin, and oxaliplatin), gemcitabine, DNA topoisomerase I-targeting drugs (e.g. camptothecin derivatives (e.g. topotecan and irinotecan)), epidermal growth factor receptor blockade family agents (e.g. cetuximab, gefitinib), farnesyltransferase inhibitors (e.g., L-778-123), COX-2 inhibitors (e.g. rofecoxib, celecoxib, and etoricoxib), bFGF and VEGF targeting agents (e.g. bevazucimab and thalidomide), NBTXR3, Nimoral, trans sodium crocetinate, NVX-108, and combinations thereof. See also e.g., Kvols, L. K., J Nucl Med 2005; 46:187S-190S.

In some embodiments, the test agent comprises one or more anti-viral agents. Exemplary antiviral-agents include without limitation, nucleoside analogues with reverse transcriptase activity (e.g., tenofovir, emtricitabine, lamivudine, abacavir, stavudine, didanosine, and zidovudine), nonnucleoside reverse transcriptase inhibitors (e.g., delavirdine, efavirenz, etravirine, nevirapine, and rilpivirine), protease inhibitors (atazanavir, darunavir, indinavir, ritonavir, tipranavir and many others), and miscellaneous agents such as maraviroc, enfuvirtide (fusion inhibitor), and integrase inhibitors (raltegravir, elvitegravir and dolutegravir), emdesivir, molnupiravir, Paxlovid (nirmatrelvir and ritonavir), Tecovirimat, seltamivir (oral), zanamivir (by inhalation) and peramivir (intravenous), baloxavir (oral), valacyclovir, cidofovir, famciclovir, ganciclovir, valganciclovir, foscarnet, simeprevir, paritaprevir, grazoprevir, glecaprevir, sofosbuvir, dasabuvir, daclatasvir, elbasvir, lepidasvir, velpatsvir, ombitasvir, pibrentasvor, Alpha interferon, peginterferon, monoclonal antibodies targeting specific viruses, and any combination thereof.

In some embodiments, the test condition is applied to the cell during and/or after transduction of a viral particle. The test condition can be any stress applied to the cell, including but not limited to a thermal stress (e.g., applying hot or cold environmental conditions to the cell), oxygen or other gas depletion or enrichment; a strain, a tension, a pressure, a change in salinity, osmolarity, pH, or other cell culture media condition, and/or the like. Such a stress can be applied continuously or be applied for one or more periods of time. Other stresses will be appreciated by one of ordinary skill in the art in view of the description herein.

### Vector and Viral Libraries for the MPRP

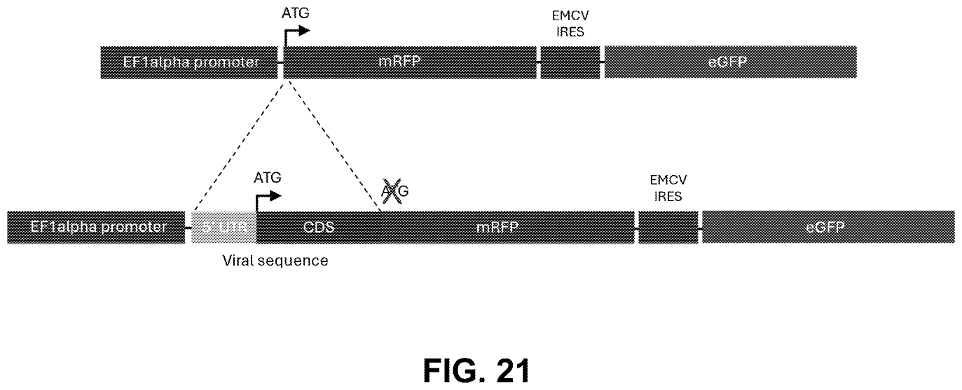

The MPRP utilizes vectors that include an optically active reporter constructs. See e.g., FIGS. 21, 22, and 29. Thus, described in certain example embodiments herein are vectors comprising an optically active reporter construct comprising a test polynucleotide; a first optically active protein encoding polynucleotide, wherein the first optically active protein encoding polynucleotide is operatively coupled in-frame with the test polynucleotide; a second optically active protein encoding polynucleotide, wherein the second optically active protein is a different optically active protein that the first optically active protein; a promoter, wherein the test polynucleotide, the first optically active protein encoding polynucleotide, and the second optically active protein encoding polynucleotide are operatively coupled to the promoter; and an internal ribosomal entry site (IRES), wherein the IRES is between the first optically active protein encoding polynucleotide and the second optically active protein encoding polynucleotide.

In certain example embodiments, the method further comprises one or more additional regulatory elements, one or more additional reporters, viral replication elements and/or encoding polynucleotides, viral packaging elements and/or encoding polynucleotides, viral envelope protein encoding polynucleotides, long-terminal repeats, viral poly or any combination thereof. In certain example embodiments, the promoter is a constitutive promoter or an inducible promoter. In certain example embodiments, the method further comprises one or more untranslated regions (UTRs), wherein the one or more untranslated regions are operatively coupled at the 5′ and/or 3′ end of the test polynucleotide and/or the first optically active protein encoding polynucleotide. In certain example embodiments, the one or more untranslated regions comprise or consist of 5′ UTRs, 3′ UTRs, or both.

In certain example embodiments, the test polynucleotide comprises or consists of a short synthetic polynucleotide. In certain example embodiments, the short synthetic polynucleotide is about 200 nucleotides. In certain example embodiments, the short synthetic polynucleotide is about 10 to about 200 nucleotides.

In certain example embodiments, the short synthetic polynucleotide has a sequence corresponding to a region of a genome. In certain example embodiments, the short synthetic polynucleotide has a sequence corresponding to a region of a human genome, a non-human animal genome, a plant genome, a microbial genome, or a viral genome. In certain example embodiments, the short synthetic polynucleotide has a sequence corresponding to (a) an annotated region of a genome; (b) an unannotated region of a genome; (c) a mutation; (d) a 5′UTR; (e) a 3′UTR; (f) an open reading frame; or (g) any combination thereof. In certain example embodiments, the short synthetic polynucleotide (a) is a sequence according to a sequence in SEQ ID NOS: 1-9890 or SEQ ID NOS: 9891-16277; (b) is a synthetic in silico designed sequence; (d) does not have 100 percent identity to a genome sequence, optionally a human genome, a non-human animal genome, a plant genome, a microbial genome, or a viral genome; (e) is not native to a genome, optionally a human genome, a non-human animal genome, a plant genome, a microbial genome, or a viral genome; or (f) any combination thereof. In certain example embodiments, the vector is a viral vector, optionally a retroviral vector, lentiviral vector, adenoviral vector, or adeno-associated viral vector.

Described in certain example embodiments, are vector systems comprising a vector comprising an optically active reporter construct as described herein, and optionally one or more a viral envelope protein vectors, one or more viral packaging vectors, or any combination thereof. In certain example embodiments, the vectors or vector systems are capable of producing viral particles comprising the optically active reporter construct. In some embodiments, the system is delivered to a plurality of cells.

As previously described the MPRP utilizes a viral or pan-viral library to transduce cells. The viral or pan-viral library comprises a plurality viral particles that contain optically active reporter constructs to be evaluated. Such reporter constructs are as previously described. Described in certain example embodiments, is an optically active reporter construct library, optionally a pan-genome or pan-viral genomic optically active reporter construct library comprising a plurality of vectors each comprising an optically active reporter construct as in any described herein, wherein at least two test polynucleotides are different or the same. In certain example embodiments, each test polynucleotide is different. In certain example embodiments, the test polynucleotides in each of the vectors of the plurality of vectors comprises or consists of a short synthetic polynucleotide.

In certain example embodiments, the test polynucleotide comprises or consists of a short synthetic polynucleotide. In certain example embodiments, the short synthetic polynucleotide is about 200 nucleotides. In certain example embodiments, the short synthetic polynucleotide is about 10 to about 200 nucleotides. In certain example embodiments, the short synthetic polynucleotide has a sequence corresponding to a region of a genome. In certain example embodiments, the short synthetic polynucleotide has a sequence corresponding to a region of a human genome, a non-human animal genome, a plant genome, a microbial genome, or a viral genome. In certain example embodiments, the short synthetic polynucleotide has a sequence corresponding to (a) an annotated region of a genome; (b) an unannotated region of a genome; (c) a mutation; (d) a 5′UTR; (e) a 3′UTR; (f) an open reading frame; or (g) any combination thereof. In certain example embodiments, the short synthetic polynucleotide (a) is a sequence according to a sequence in SEQ ID NOS: 1-9890 or SEQ ID NOS: 9891-16277; (b) is a synthetic in silico designed sequence; (d) does not have 100 percent identity to a genome sequence, optionally a human genome, a non-human animal genome, a plant genome, a microbial genome, or a viral genome; (e) is not native to a genome, optionally a human genome, a non-human animal genome, a plant genome, a microbial genome, or a viral genome; or (f) any combination thereof. In certain example embodiments, the vector is a viral vector, optionally a retroviral vector, lentiviral vector, adenoviral vector, or adeno-associated viral vector.

In some embodiments, the viral library or viral particle library (e.g. pan viral library or pan viral particle library) contains 100, to/or 150, 200, 250, 300, 350, 400, 450, 500, 550, 600, 650, 700, 750, 800, 850, 900, 950, 1000, 1000, 2000, 3000, 4000, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, 100000, 101000, 102000, 103000, 104000, 105000, 106000, 107000, 108000, 109000, 110000, 111000, 112000, 113000, 114000, 115000, 116000, 117000, 118000, 119000, 120000, 121000, 122000, 123000, 124000, 125000, 126000, 127000, 128000, 129000, 130000, 131000, 132000, 133000, 134000, 135000, 136000, 137000, 138000, 139000, 140000, 141000, 142000, 143000, 144000, 145000, 146000, 147000, 148000, 149000, 150000, 151000, 152000, 153000, 154000, 155000, 156000, 157000, 158000, 159000, 160000, 161000, 162000, 163000, 164000, 165000, 166000, 167000, 168000, 169000, 170000, 171000, 172000, 173000, 174000, 175000, 176000, 177000, 178000, 179000, 180000, 181000, 182000, 183000, 184000, 185000, 186000, 187000, 188000, 189000, 190000, 191000, 192000, 193000, 194000, 195000, 196000, 197000, 198000, 199000, 200000, 201000, 202000, 203000, 204000, 205000, 206000, 207000, 208000, 209000, 210000, 211000, 212000, 213000, 214000, 215000, 216000, 217000, 218000, 219000, 220000, 221000, 222000, 223000, 224000, 225000, 226000, 227000, 228000, 229000, 230000, 231000, 232000, 233000, 234000, 235000, 236000, 237000, 238000, 239000, 240000, 241000, 242000, 243000, 244000, 245000, 246000, 247000, 248000, 249000, 250000, 251000, 252000, 253000, 254000, 255000, 256000, 257000, 258000, 259000, 260000, 261000, 262000, 263000, 264000, 265000, 266000, 267000, 268000, 269000, 270000, 271000, 272000, 273000, 274000, 275000, 276000, 277000, 278000, 279000, 280000, 281000, 282000, 283000, 284000, 285000, 286000, 287000, 288000, 289000, 290000, 291000, 292000, 293000, 294000, 295000, 296000, 297000, 298000, 299000, 300000, 301000, 302000, 303000, 304000, 305000, 306000, 307000, 308000, 309000, 310000, 311000, 312000, 313000, 314000, 315000, 316000, 317000, 318000, 319000, 320000, 321000, 322000, 323000, 324000, 325000, 326000, 327000, 328000, 329000, 330000, 331000, 332000, 333000, 334000, 335000, 336000, 337000, 338000, 339000, 340000, 341000, 342000, 343000, 344000, 345000, 346000, 347000, 348000, 349000, 350000, 351000, 352000, 353000, 354000, 355000, 356000, 357000, 358000, 359000, 360000, 361000, 362000, 363000, 364000, 365000, 366000, 367000, 368000, 369000, 370000, 371000, 372000, 373000, 374000, 375000, 376000, 377000, 378000, 379000, 380000, 381000, 382000, 383000, 384000, 385000, 386000, 387000, 388000, 389000, 390000, 391000, 392000, 393000, 394000, 395000, 396000, 397000, 398000, 399000, 400000, 401000, 402000, 403000, 404000, 405000, 406000, 407000, 408000, 409000, 410000, 411000, 412000, 413000, 414000, 415000, 416000, 417000, 418000, 419000, 420000, 421000, 422000, 423000, 424000, 425000, 426000, 427000, 428000, 429000, 430000, 431000, 432000, 433000, 434000, 435000, 436000, 437000, 438000, 439000, 440000, 441000, 442000, 443000, 444000, 445000, 446000, 447000, 448000, 449000, 450000, 451000, 452000, 453000, 454000, 455000, 456000, 457000, 458000, 459000, 460000, 461000, 462000, 463000, 464000, 465000, 466000, 467000, 468000, 469000, 470000, 471000, 472000, 473000, 474000, 475000, 476000, 477000, 478000, 479000, 480000, 481000, 482000, 483000, 484000, 485000, 486000, 487000, 488000, 489000, 490000, 491000, 492000, 493000, 494000, 495000, 496000, 497000, 498000, 499000, 500000, 501000, 502000, 503000, 504000, 505000, 506000, 507000, 508000, 509000, 510000, 511000, 512000, 513000, 514000, 515000, 516000, 517000, 518000, 519000, 520000, 521000, 522000, 523000, 524000, 525000, 526000, 527000, 528000, 529000, 530000, 531000, 532000, 533000, 534000, 535000, 536000, 537000, 538000, 539000, 540000, 541000, 542000, 543000, 544000, 545000, 546000, 547000, 548000, 549000, 550000, 551000, 552000, 553000, 554000, 555000, 556000, 557000, 558000, 559000, 560000, 561000, 562000, 563000, 564000, 565000, 566000, 567000, 568000, 569000, 570000, 571000, 572000, 573000, 574000, 575000, 576000, 577000, 578000, 579000, 580000, 581000, 582000, 583000, 584000, 585000, 586000, 587000, 588000, 589000, 590000, 591000, 592000, 593000, 594000, 595000, 596000, 597000, 598000, 599000, 600000, 601000, 602000, 603000, 604000, 605000, 606000, 607000, 608000, 609000, 610000, 611000, 612000, 613000, 614000, 615000, 616000, 617000, 618000, 619000, 620000, 621000, 622000, 623000, 624000, 625000, 626000, 627000, 628000, 629000, 630000, 631000, 632000, 633000, 634000, 635000, 636000, 637000, 638000, 639000, 640000, 641000, 642000, 643000, 644000, 645000, 646000, 647000, 648000, 649000, 650000, 651000, 652000, 653000, 654000, 655000, 656000, 657000, 658000, 659000, 660000, 661000, 662000, 663000, 664000, 665000, 666000, 667000, 668000, 669000, 670000, 671000, 672000, 673000, 674000, 675000, 676000, 677000, 678000, 679000, 680000, 681000, 682000, 683000, 684000, 685000, 686000, 687000, 688000, 689000, 690000, 691000, 692000, 693000, 694000, 695000, 696000, 697000, 698000, 699000, 700000, 701000, 702000, 703000, 704000, 705000, 706000, 707000, 708000, 709000, 710000, 711000, 712000, 713000, 714000, 715000, 716000, 717000, 718000, 719000, 720000, 721000, 722000, 723000, 724000, 725000, 726000, 727000, 728000, 729000, 730000, 731000, 732000, 733000, 734000, 735000, 736000, 737000, 738000, 739000, 740000, 741000, 742000, 743000, 744000, 745000, 746000, 747000, 748000, 749000, 750000, 751000, 752000, 753000, 754000, 755000, 756000, 757000, 758000, 759000, 760000, 761000, 762000, 763000, 764000, 765000, 766000, 767000, 768000, 769000, 770000, 771000, 772000, 773000, 774000, 775000, 776000, 777000, 778000, 779000, 780000, 781000, 782000, 783000, 784000, 785000, 786000, 787000, 788000, 789000, 790000, 791000, 792000, 793000, 794000, 795000, 796000, 797000, 798000, 799000, 800000, 801000, 802000, 803000, 804000, 805000, 806000, 807000, 808000, 809000, 810000, 811000, 812000, 813000, 814000, 815000, 816000, 817000, 818000, 819000, 820000, 821000, 822000, 823000, 824000, 825000, 826000, 827000, 828000, 829000, 830000, 831000, 832000, 833000, 834000, 835000, 836000, 837000, 838000, 839000, 840000, 841000, 842000, 843000, 844000, 845000, 846000, 847000, 848000, 849000, 850000, 851000, 852000, 853000, 854000, 855000, 856000, 857000, 858000, 859000, 860000, 861000, 862000, 863000, 864000, 865000, 866000, 867000, 868000, 869000, 870000, 871000, 872000, 873000, 874000, 875000, 876000, 877000, 878000, 879000, 880000, 881000, 882000, 883000, 884000, 885000, 886000, 887000, 888000, 889000, 890000, 891000, 892000, 893000, 894000, 895000, 896000, 897000, 898000, 899000, 900000, 901000, 902000, 903000, 904000, 905000, 906000, 907000, 908000, 909000, 910000, 911000, 912000, 913000, 914000, 915000, 916000, 917000, 918000, 919000, 920000, 921000, 922000, 923000, 924000, 925000, 926000, 927000, 928000, 929000, 930000, 931000, 932000, 933000, 934000, 935000, 936000, 937000, 938000, 939000, 940000, 941000, 942000, 943000, 944000, 945000, 946000, 947000, 948000, 949000, 950000, 951000, 952000, 953000, 954000, 955000, 956000, 957000, 958000, 959000, 960000, 961000, 962000, 963000, 964000, 965000, 966000, 967000, 968000, 969000, 970000, 971000, 972000, 973000, 974000, 975000, 976000, 977000, 978000, 979000, 980000, 981000, 982000, 983000, 984000, 985000, 986000, 987000, 988000, 989000, 990000, 991000, 992000, 993000, 994000, 995000, 996000, 997000, 998000, 999000, 1000000, or more viral particles.

Additionally described herein are viral particles that contain the optically active reporter construct. Described in certain example embodiments herein are engineered viral particles each comprising a cargo comprising an optically active reporter construct, wherein the optically active reporter construct cargo comprises a test polynucleotide; a first optically active protein encoding polynucleotide, wherein the first optically active protein encoding polynucleotide is operatively coupled in-frame with the test polynucleotide; a second optically active protein encoding polynucleotide, wherein the second optically active protein is a different optically active protein that the first optically active protein; a promoter, wherein the test polynucleotide, the first optically active protein encoding polynucleotide, and the second optically active protein encoding polynucleotide are operatively coupled to the promoter; and an internal ribosomal entry site (IRES), wherein the IRES is between the first optically active protein encoding polynucleotide and the second optically active protein encoding polynucleotide. In certain example embodiments, the promoter is a constitutive promoter or an inducible promoter. In certain example embodiments, the optically active reporter construct further comprises one or more untranslated regions (UTRs), wherein the one or more untranslated regions are operatively coupled at the 5′ and/or 3′ end of the test polynucleotide and/or the first optically active protein encoding polynucleotide. In certain example embodiments, the one or more untranslated regions comprise or consist of 5′ UTRs, 3′ UTRs, or both. In certain example embodiments, the test polynucleotide comprises or consists of a short synthetic polynucleotide. In certain example embodiments, the short synthetic polynucleotide is about 200 nucleotides. In certain example embodiments, the short synthetic polynucleotide has a sequence corresponding to a region of a genome. In certain example embodiments, the short synthetic polynucleotide has a sequence corresponding to a region of a human genome, a non-human animal genome, a plant genome, a microbial genome, or a viral genome. In certain example embodiments, the short synthetic polynucleotide has a sequence corresponding to a) an annotated region of a genome; (b) an unannotated region of a genome; (c) a mutation; (d) a 5′UTR; (e) a 3′UTR; (f) an open reading frame; or (g) any combination thereof.

In some embodiments, the short synthetic polynucleotide has a sequence corresponding to (a) an annotated region of a genome; (b) an unannotated region of a genome; (c) a mutation; (d) a 5′UTR; (e) a 3′UTR; (f) an open reading frame; or (g) any combination thereof. In certain example embodiments, the short synthetic polynucleotide (a) is a sequence according to a sequence in SEQ ID NOS: 1-9890 or SEQ ID NOS: 9891-16277; (b) is a synthetic in silico designed sequence; (d) does not have 100 percent identity to a genome sequence, optionally a human genome, a non-human animal genome, a plant genome, a microbial genome, or a viral genome; (e) is not native to a genome, optionally a human genome, a non-human animal genome, a plant genome, a microbial genome, or a viral genome; or (f) any combination thereof. In certain example embodiments, the engineered viral particle is a viral particle, optionally a retroviral viral particle, lentiviral viral particle, adenoviral viral particle, or adeno-associated viral particle.

Additional vectors and vector elements are described in greater detail elsewhere herein. Methods of producing viral particles are generally known in the art and described in greater detail elsewhere herein.

Further embodiments are illustrated in the following Examples, which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—Massively Parallel Ribosome Profiling

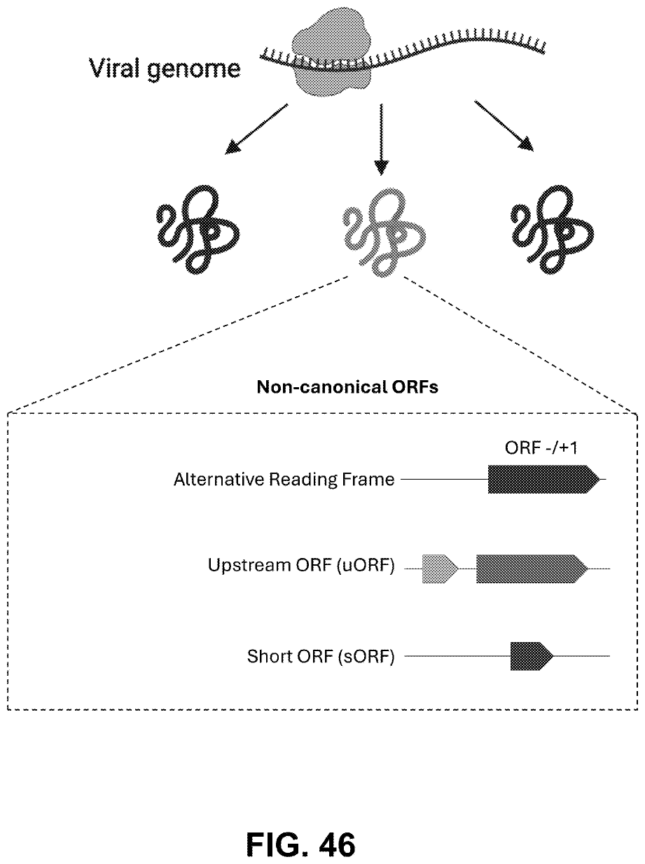

Viruses contained condensed genomes that has evolved to encode multiple functions that allow the virus to e.g., replicate in a host by producing infectious virions in a host and/or vector and/or evading the host immune response. The landscape of translated regions is unknown for most viruses. Even less characterized and understood is what, if any, additional functions these translated regions have, particularly non-canonical translated regions (e.g., non-canonical ORFs). Among these are alternative reading frames (ARFs), upstream open reading frames (uORFs), short open reading frames (sORFs) and defective ribosomal products (DRiPs). Decoding the complete set of viral proteins, including non-canonical ORFs, is essential to understand the biology of viruses. These sources vastly increase the availability of viral peptides for presentation and immune evasion and imply an important role for translational regulation in this process. Applicant and others have shown that viral non-canonical ORFs play a critical role in antigen presentation to cytotoxic T cells, modulating virus infection, and regulating virions production. However, the landscape of translated regions remains largely unknown for many viruses (see e.g., FIG. 46), as traditional studies investigate only a few peptides at a time, we have not achieved a comprehensive view of the collection of immunogenic peptides in each virus and, in turn, the causal effect of viral mutations on immunogenicity.

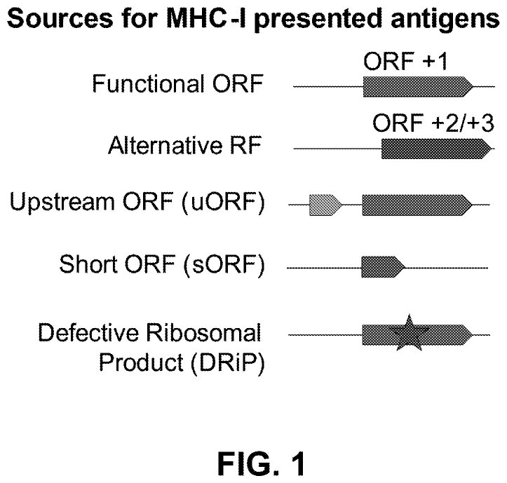

Mapping the translatome can expand the understanding of the viral life cycle and immune response, which can lead to improved strategies to prevent and treat viral infection as well as better engineer viruses for various commercial and clinical applications. Mapping can uncover new viral proteins, such as those translated from non-canonical ORFS, uncovering alternative gene expression regulation mechanisms, and new epitopes that are involved in T-cell and/or B-cell responses. This can in turn lead to improved understanding of T-cell and B-cell targets, which can be translated into new compositions, such as vaccines, for prevention and treatment of infection and disease. Indeed, such translated regions may be sources for MHC-I antigen presentation (see e.g., FIG. 1 and Fetten et al., J Immunol 1991; Yang et al., J Immunol 2016; Mayrand et al., J Immunol 1998; Cardinaud et al., J Exp Med (2004) 199 (8): 1053-1063; Ho et al., J Immunol 2006; Maness et al., J Exp Med. 2007; Bansal et al., J Exp Med. 2010; Neefjes et al., Nature Reviews Immunology volume 11, pages 823-836 (2011); Ingolia et al., Cell Reports 2014; Anton and Yewdell. J Leukoc Biol 2014 April; 95(4):551-62; Starck et al., Science. 2016 Jan. 29; 351(6272):aad3867. doi: 10.1126/science.aad3867; and Starck and Shastri. Immunol Rev. 2016 July; 272(1):8-16. doi: 10.1111/imr.12434). Other approaches to understanding non-canonical viral ORFs have already demonstrated the value in mapping non-canonical viral ORFs. For example, Weingarten-Gabbay et al., (Cell. 2021 Jul. 22; 184(15): 3962-3980.e17) used mass spectrometry (MS)-based HLA-I immunopeptidomics to map HLA-I immunopeptidome of SARS-CoV-2 and uncovered non-canonical ORFs that produced previously uncharacterized SARS-CoV-2 peptides involved in mediating the T-cell response during infection.

Ribosome profiling is a method of taking genome-wide measurements of translated regions of the genome. See e.g., Ingolia et al., Science. 2009 Apr. 10; 324(5924):218-23. doi: 10.1126/science.1168978 and Brar and Weissman. Nat Rev Mol Cell Biol. 2015 November; 16(11):651-64.doi: 10.1038/nrm4069. Traditional ribosome profiling has limitations. For example, only one virus can be profiled in a single experiment, some viruses cannot be cultured in a lab and some require stringent safety precautions (e.g., require BSL 3 or 4), and provide a limited capacity to infer causal effect of emerging mutations. Many other high-throughput methods for systematic evaluation of gene expression regulation have been generated, such as for discovering internal ribosome entry sites (see e.g., Weingarten-Gabbay et al., Science. 2016 Jan. 15; 351(6270):aad4939; Weingarten-Gabbay and Segal. RNA Biol. 2016 Oct. 2; 13(10):927-933. doi: 10.1080/15476286.2016.1212802; Gritsenko A A, Weingarten-Gabbay S, Elias-Kirma S, Nir R, de Ridder D, Segal E (2017) Sequence features of viral and human Internal Ribosome Entry Sites predictive of their activity. PLoS Comput Biol 13(9): e1005734. https://doi.org/10.1371/journal.pcbi.1005734; Weingarten-Gabbay et al., Oncogene. 2014 Jan. 30; 33(5):611-8. doi: 10.1038/onc.2012.626) and for systemic interrogation of human core promoters (see e.g., Weingarten-Gabbay et al., Genome Res. 2019 February; 29(2):171-183; Weingarten-Gabbay and Segal. Hum Genet. 2014 June; 133(6):701-11. doi: 10.1007/s00439-013-1413-1; and Slutskin et al., Nat Commun. 2018 Feb. 6; 9(1):529. doi: 10.1038/s41467-018-02980-z).

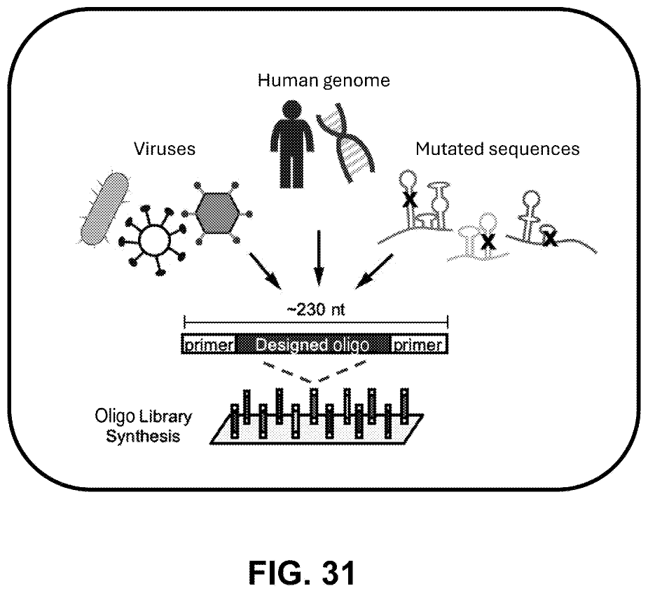

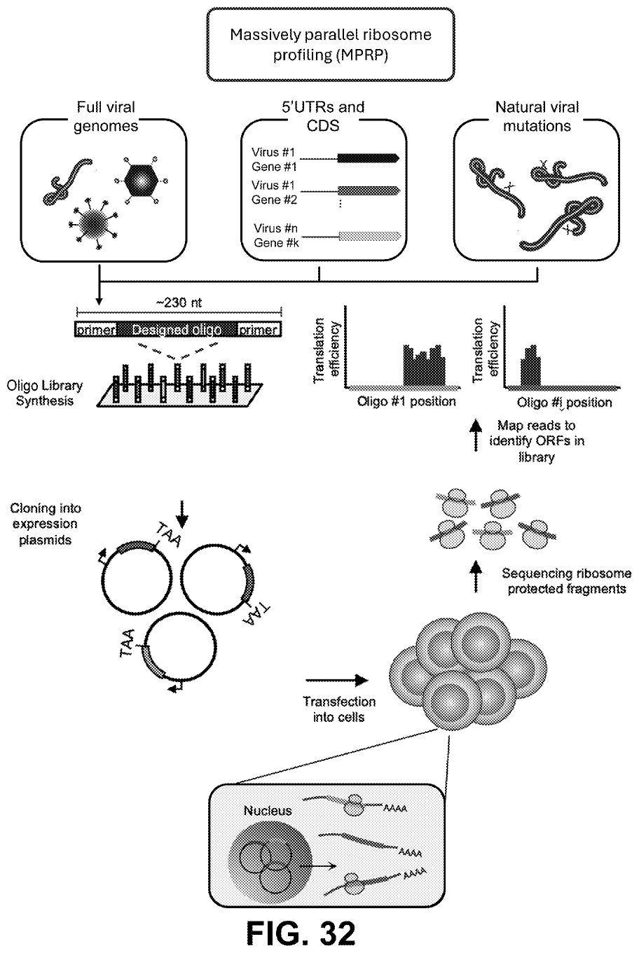

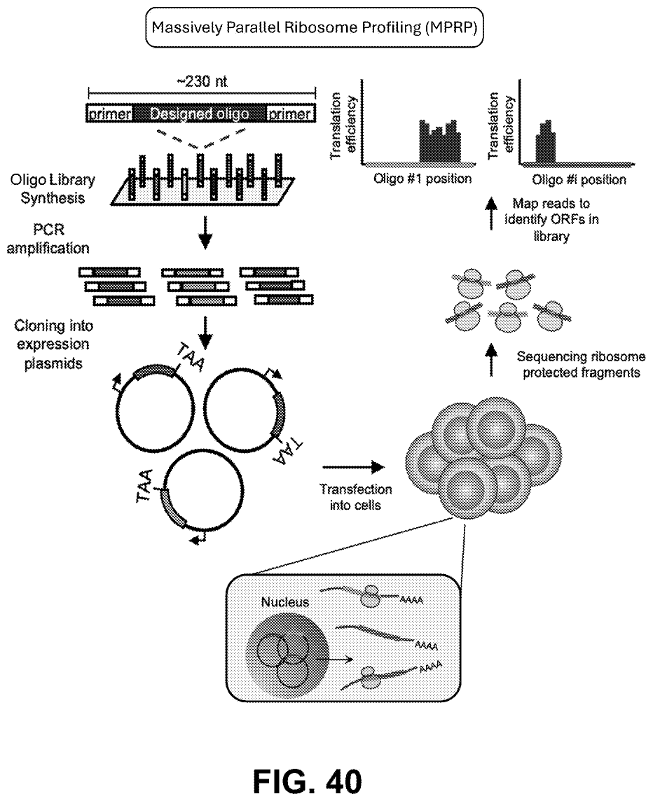

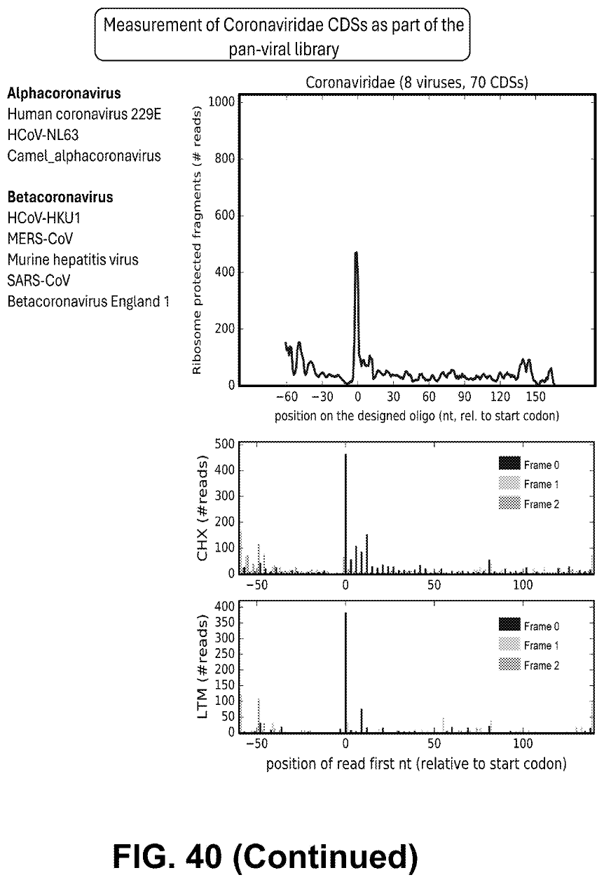

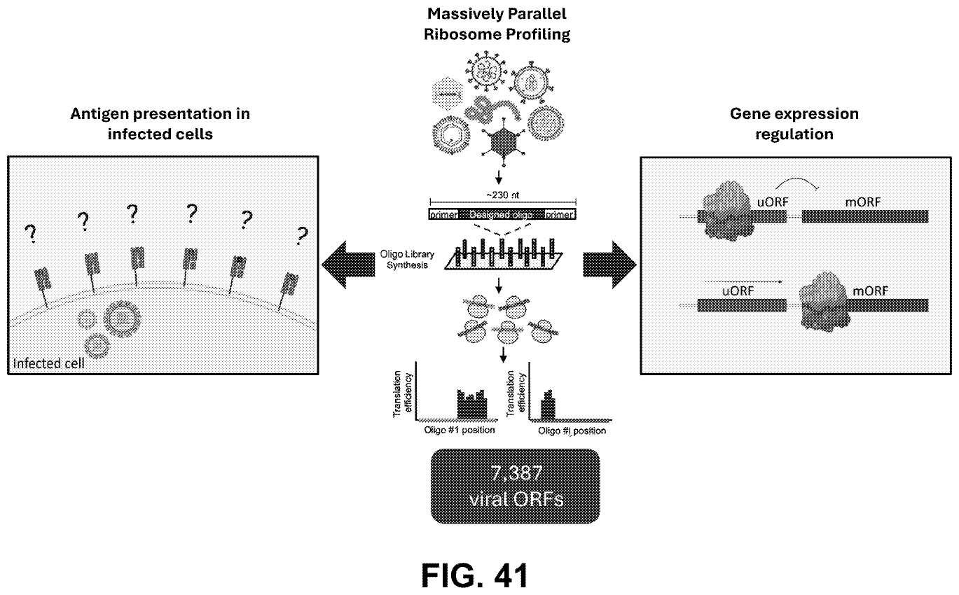

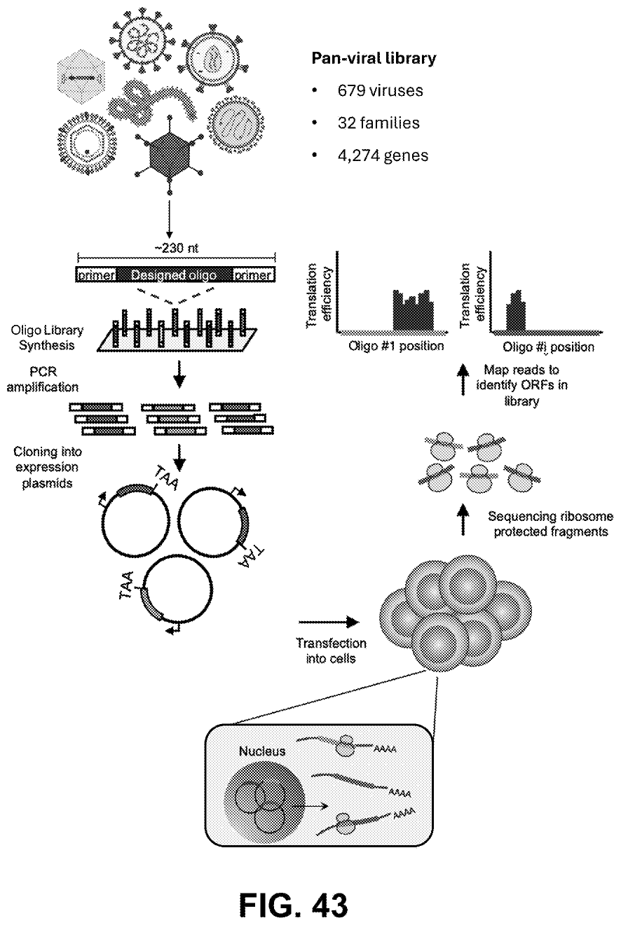

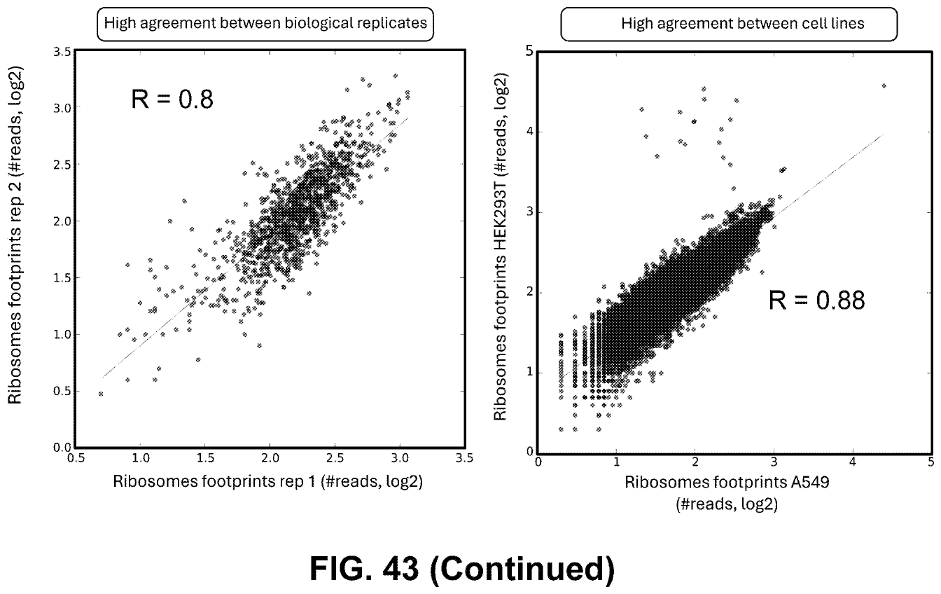

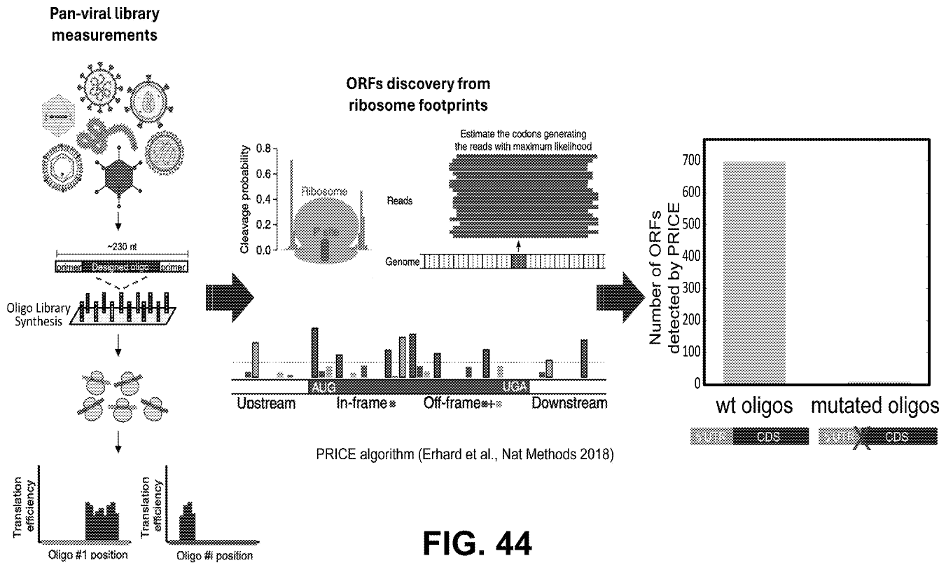

This Example describes and demonstrates massively parallel ribosome profiling (MPRP) for detecting and mapping translated regions of genomes, including, but not limited to, non-canonical ORFs, from e.g., viruses. See e.g., FIG. 4A-4D, which shows a general schematic for high-throughput methods of measuring antigen translation and presentation using a designed synthetic oligo library. Here, Applicant developed Massively Parallel Ribosome Profiling (MPRP) to quantitatively measure the translation of tens of thousands of viral sequences in one experiment. Using this method, Applicant exposed 7,387 ORFs in 679 viruses pathogenic to humans. Applicant uncovered 328 of upstream ORFs (uORFs) in the 5′UTR of viral genes that negatively regulate the translation of the main coding sequence (CDS) on the same transcript. This approach employs a synthetic designed oligo library, which includes oligonucleotides (“oligos”) that represent native genomes of hundreds of different viruses. See e.g., FIGS. 3 and 31. This can provide an unbiased screen of fragments from different regions of the viral genome. This can allow for functional assays of viruses that would normally require stringent safety protocols. Further this can allow for causal testing of effects of the translated regions and mutations thereof (such as CTL-escape mutations) and an understanding of viral heterogenicity by e.g., allowing for comparison of viral quasispecies. FIGS. 5, 32, 40, 43-44 shows a more detailed schematic for massively parallel ribosome profiling. A synthetic designed oligo construct is used to synthesize a synthetic designed oligo library. The library is expressed in cells. Ribosome protected fragments are sequenced and sequencing reads are mapped to identify open reading frames (ORFs) present in the expressed synthetic oligo library. As summarized in FIG. 41, this MPRP assay can be coupled with additional techniques to further evaluate functionality of the uncovered translated regions, such as in antigen presentation or gene expression regulation.

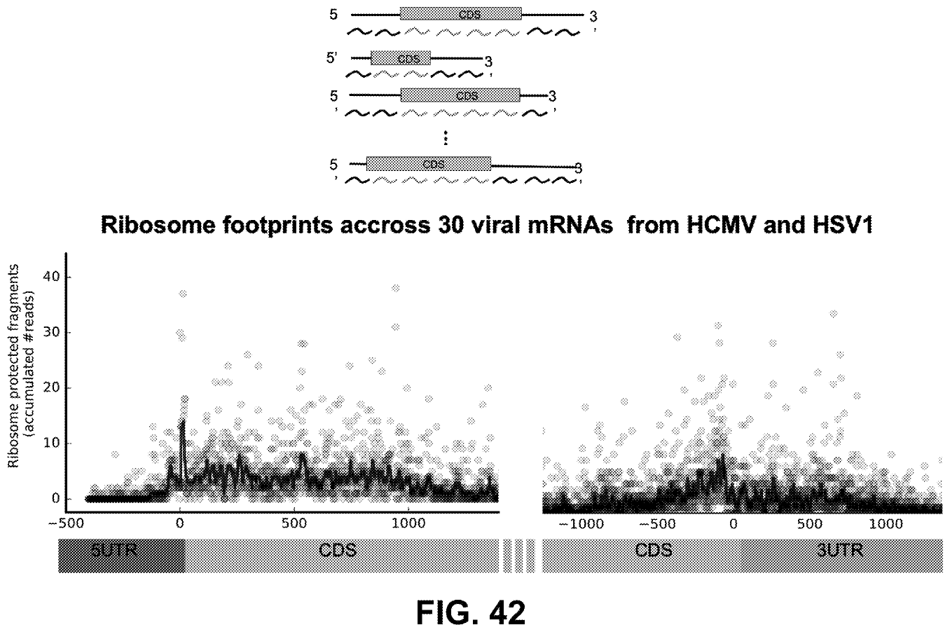

Other approaches that rely on tailing whole transcripts with 200 nt oligos can result in false positive discovery of internal ORFs (FIG. 42). In contrast, the constructs for generating the synthetic oligo library for the MPRP assay includes two constant primers flanking a variable region that is the synthetic oligonucleotide that corresponds to a region of a viral genome. This was followed by multiple shifted stop codons. For this Example, the following 3 stop codons were included: #1 TAG; #2 TGA; #3 TAA. The construct is driven by a promoter. See e.g., FIG. 6. The construct can also include other elements such as WPRE and poly A signal.

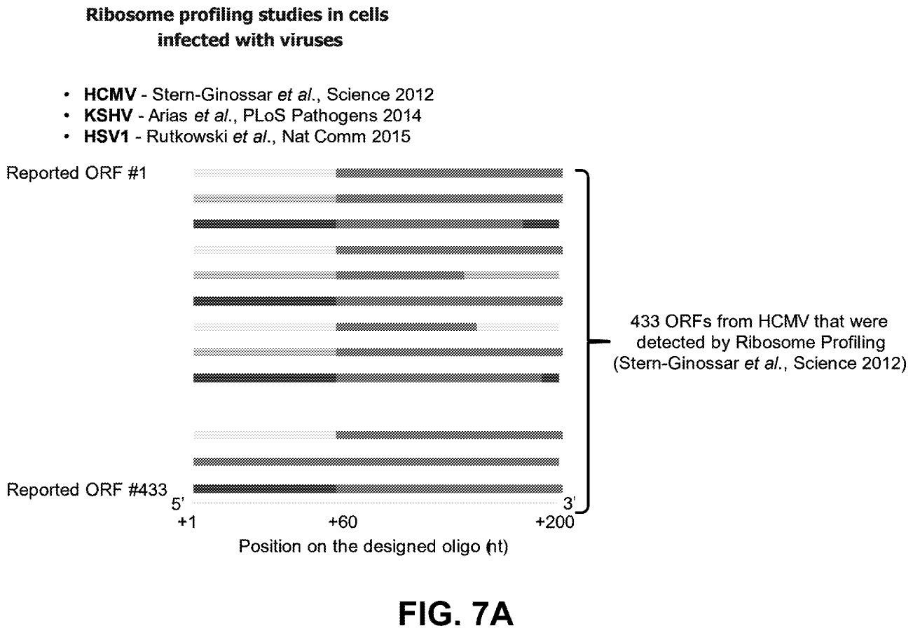

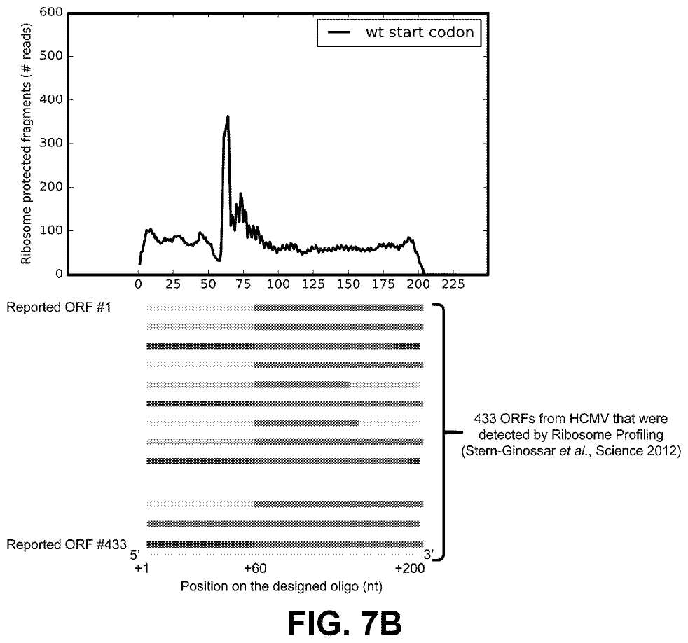

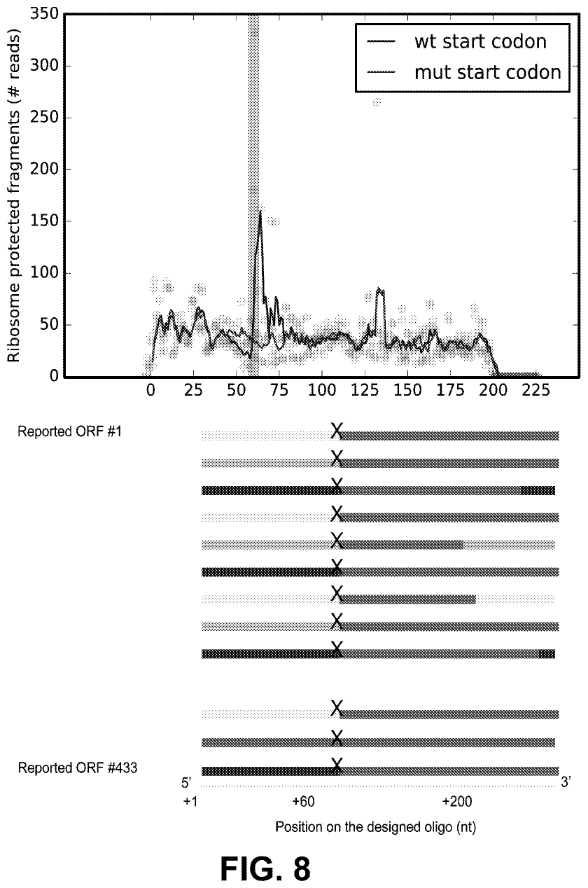

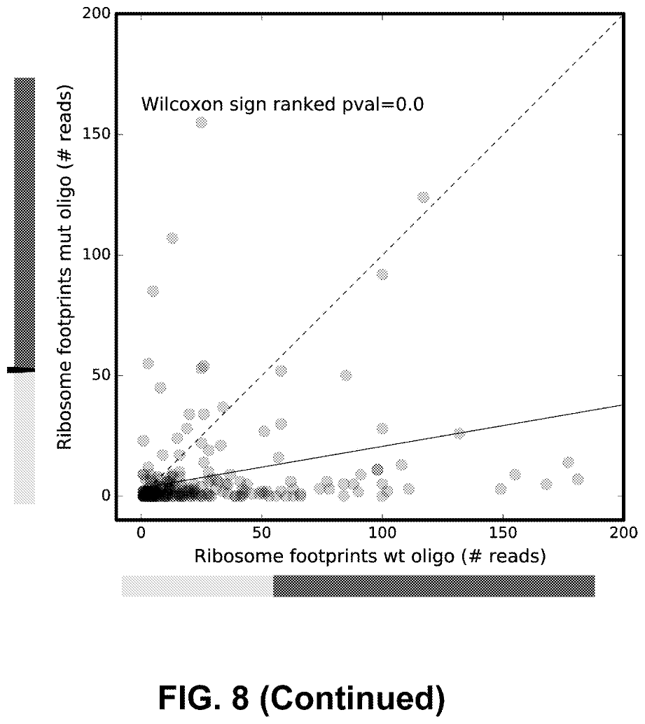

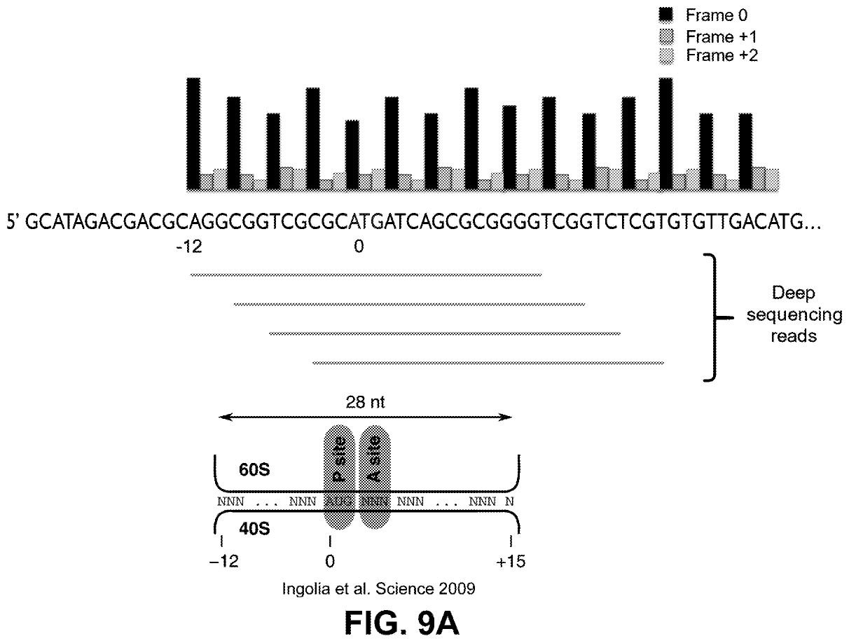

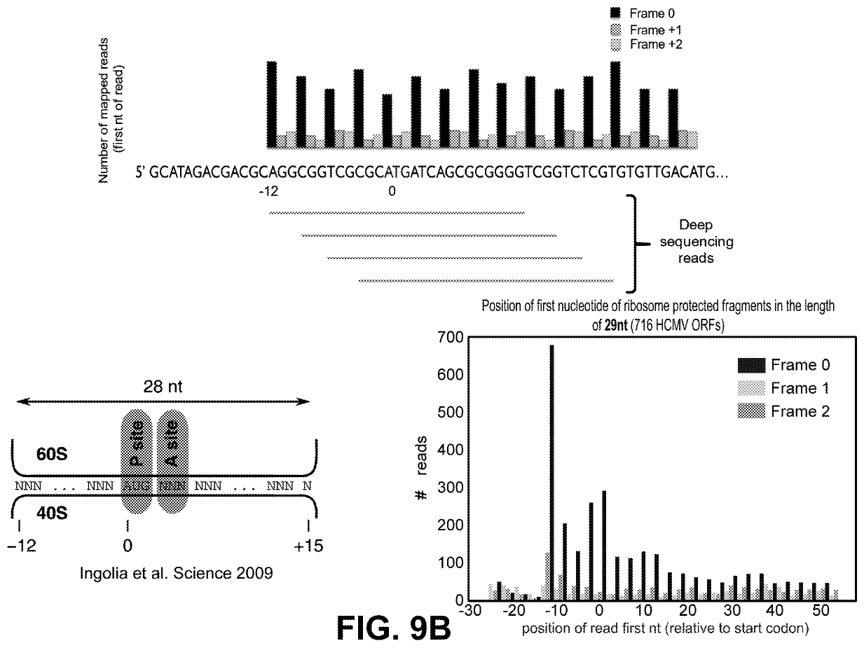

FIG. 7A-7B shows design and measurement of a ~5,000 oligo library towards previously reported viral ORFs to demonstrate accuracy of detection of ORFS using the massively parallel ribosome profiling approach of FIG. 5. Reported ORFs were used to validate the MPRP assay. To further validate the assay and to demonstrate its use for evaluating the effect of mutations, the reported start codon for the reported ORFs evaluated were mutated in the synthetic oligonucleotide and the MPRP assay was performed. As shown in FIG. 8, a reduction in ribosome occupancy is obtained when the reported start codon is mutated in the synthetic oligo. Periodicity in ORFs encoded from synthetic oligos was also determined (FIG. 9A-9B).

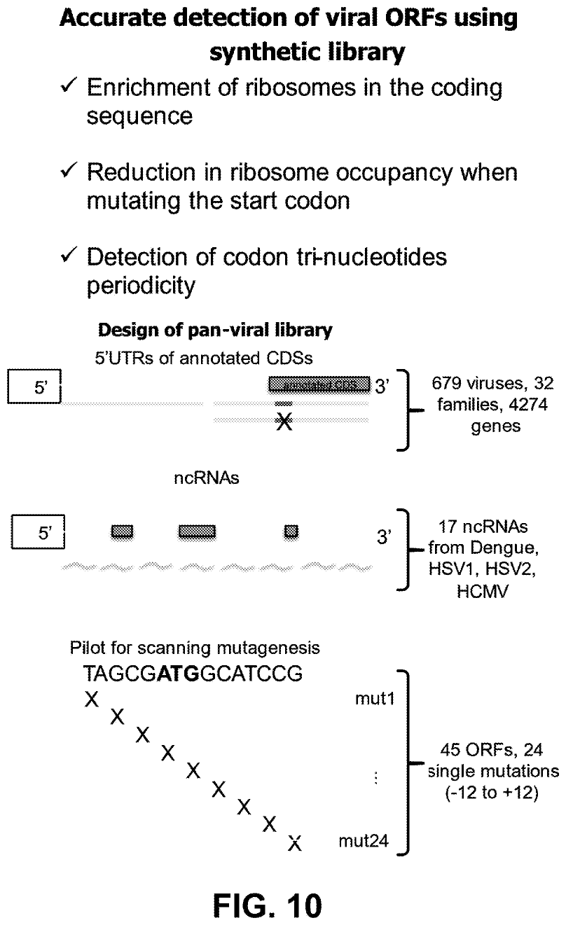

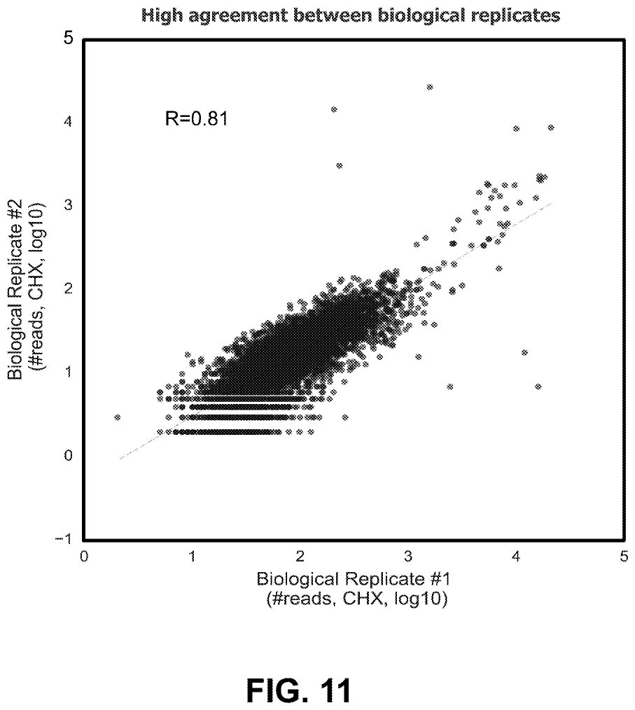

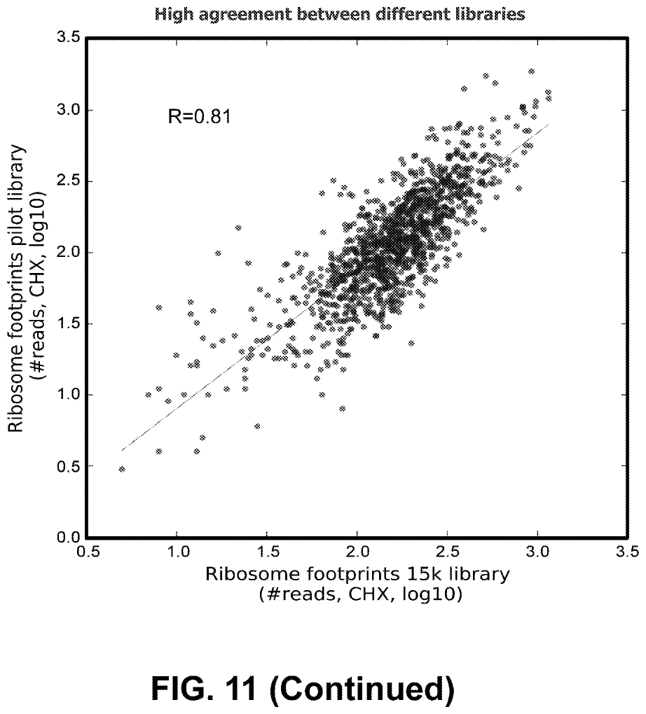

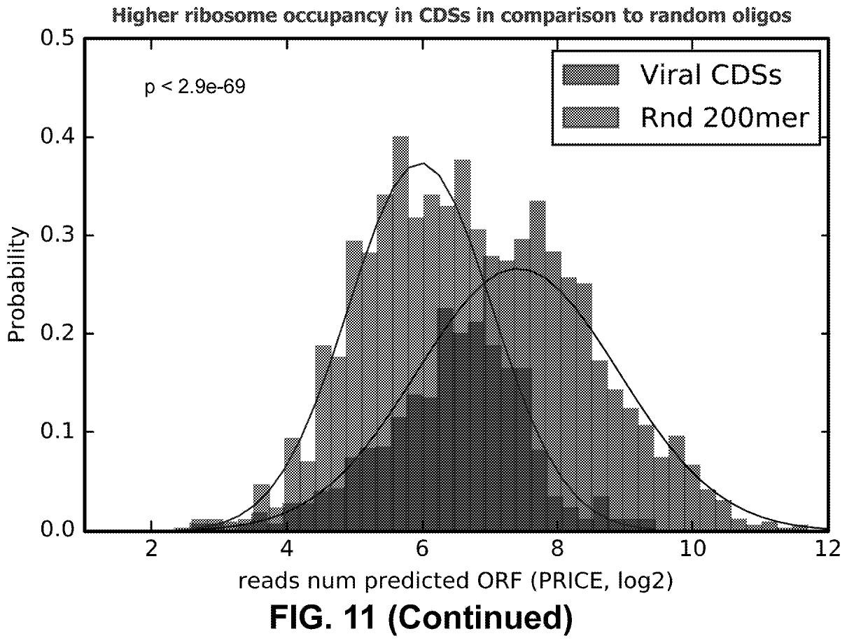

A pan viral library of 15,000 oligos for the discovery of upstream ORFs (uORFs) and short ORFs was designed. See e.g., FIG. 10. The MPRP assay allowed for reproducible and specific measurements of ribosome footprints (FIG. 11).

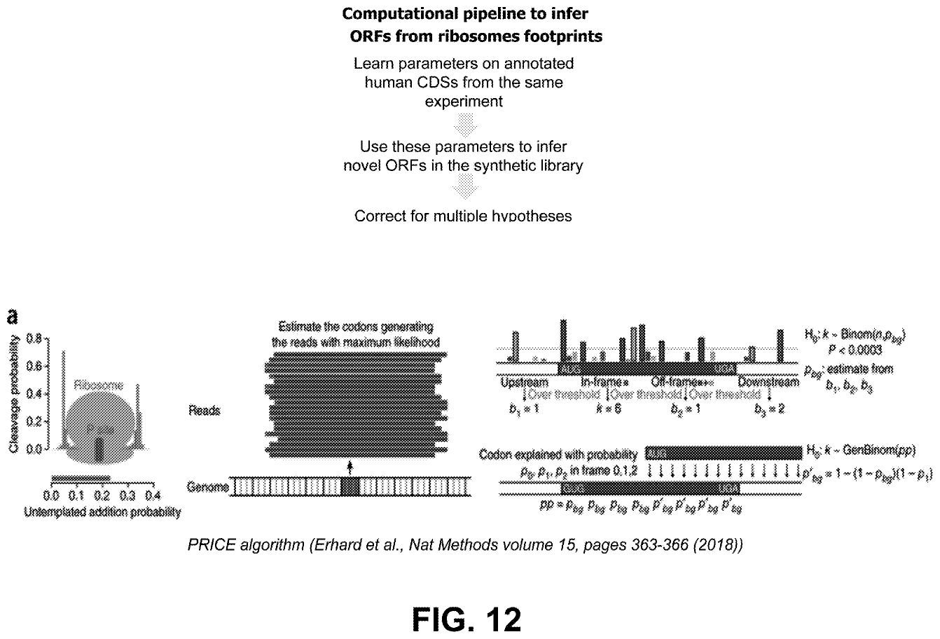

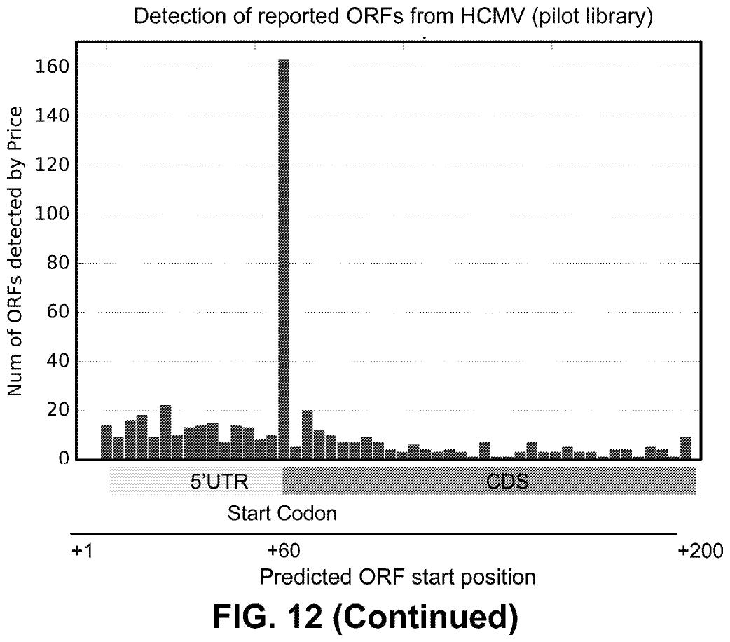

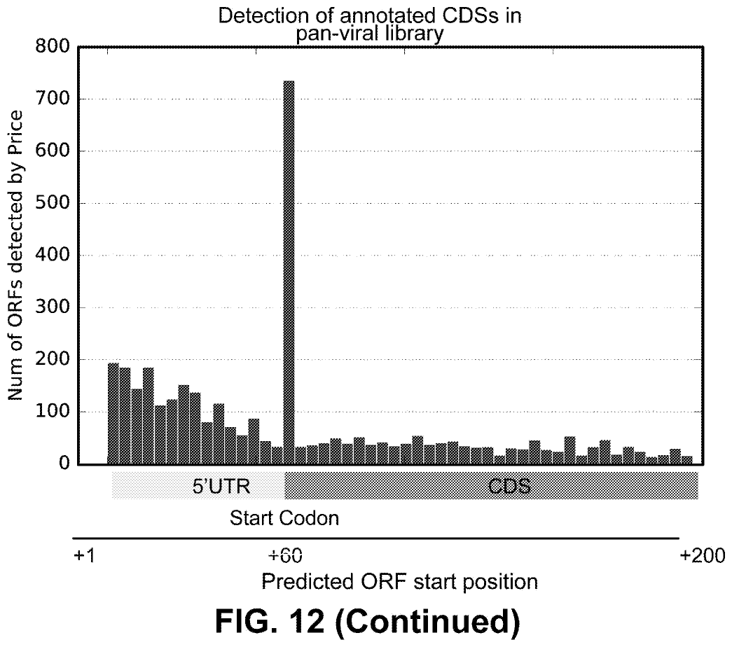

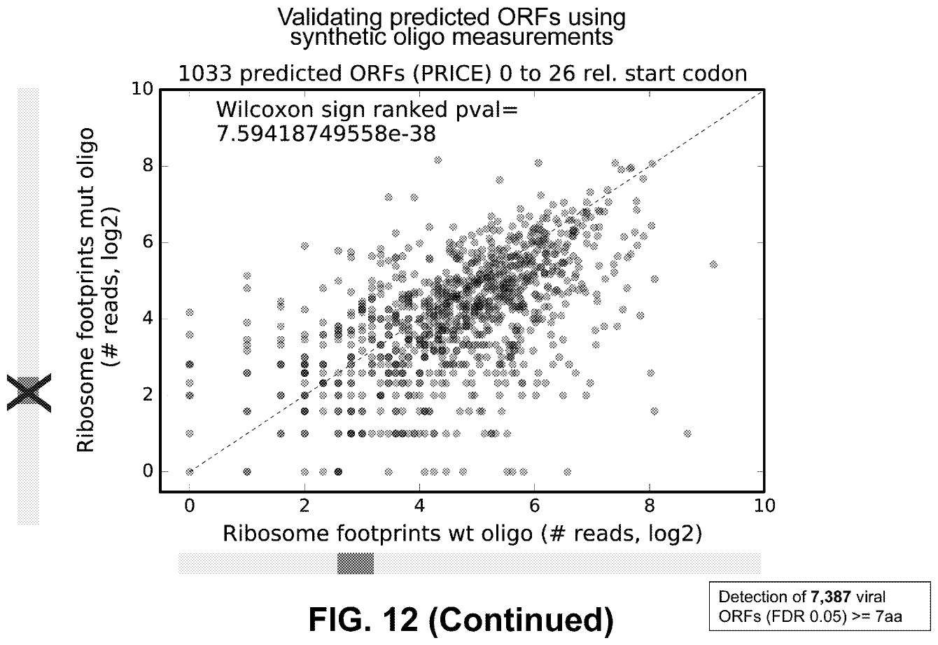

After generating the ribosome footprints, the MPRP assay utilizes a computational pipeline to infer ORFs from the ribosome footprints. See e.g., FIG. 12. This Example utilized the PRICE algorithm (Erhard et al., Nat Methods. 2018 May; 15(5):363-366. doi: 10.1038/nmeth.4631).

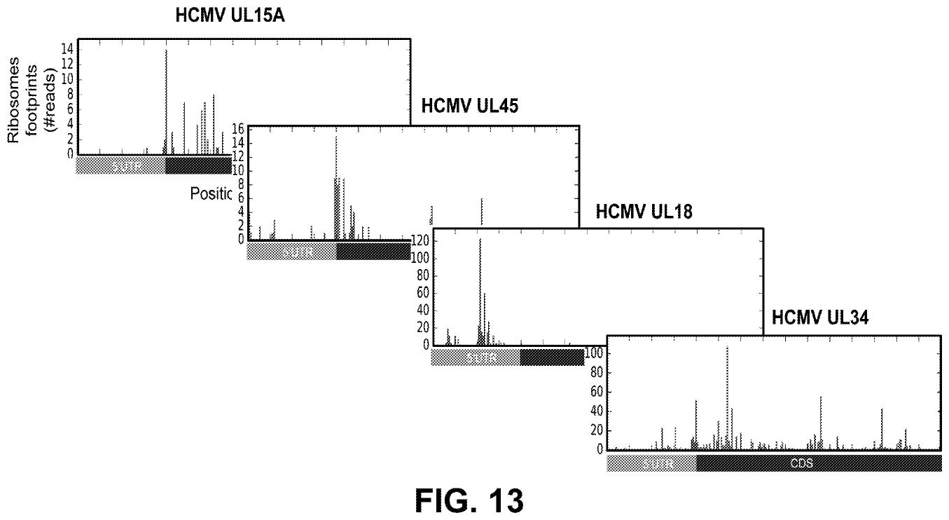

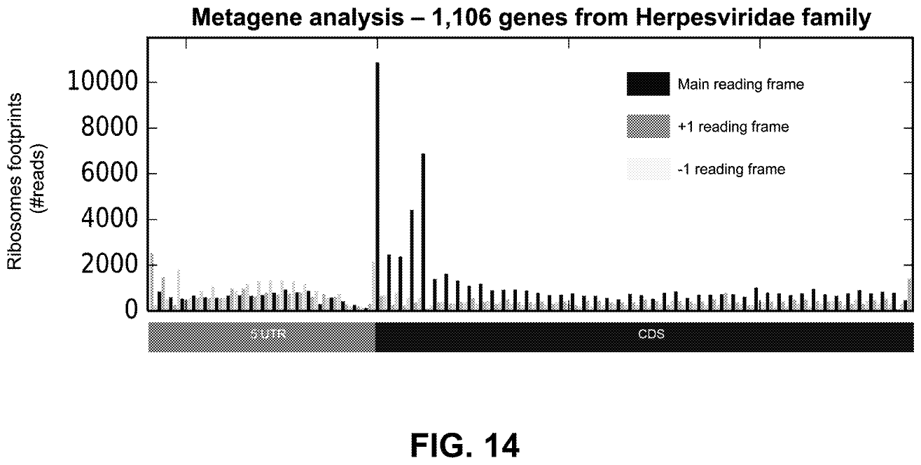

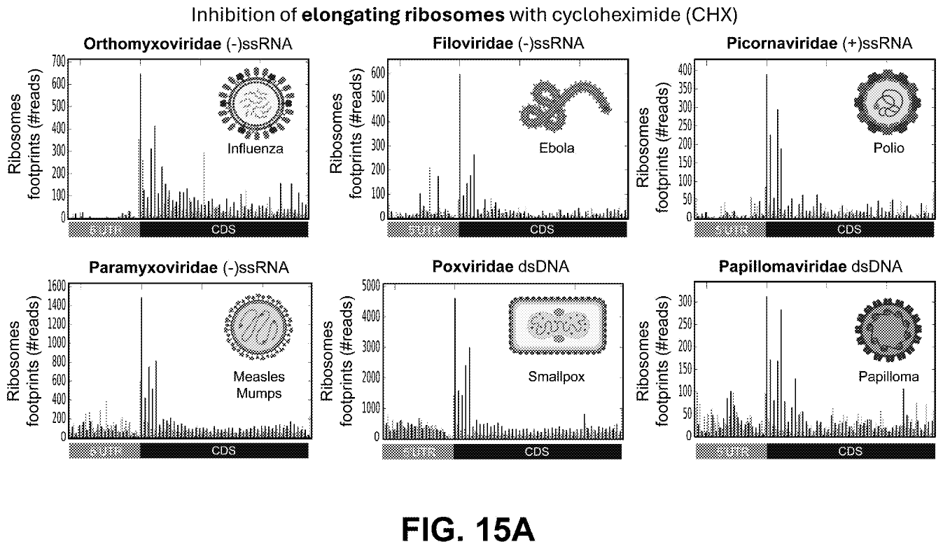

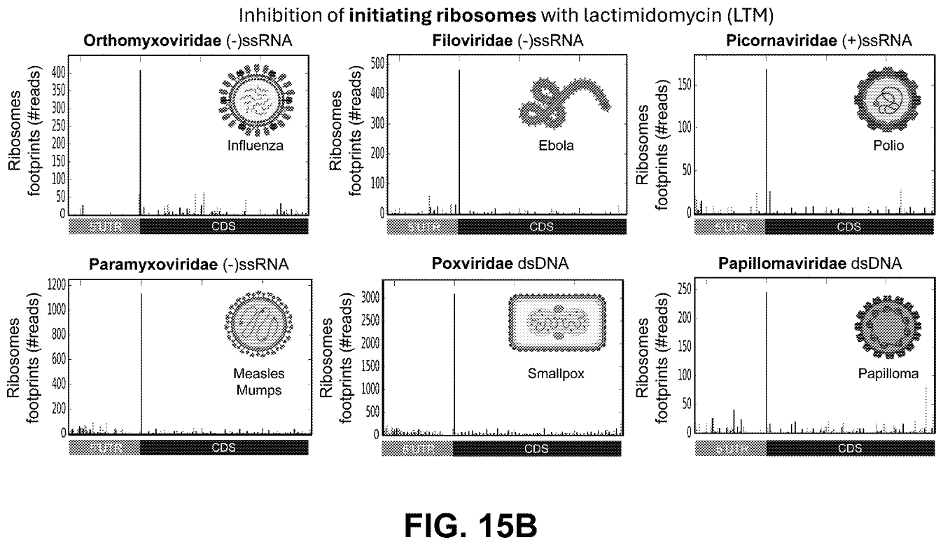

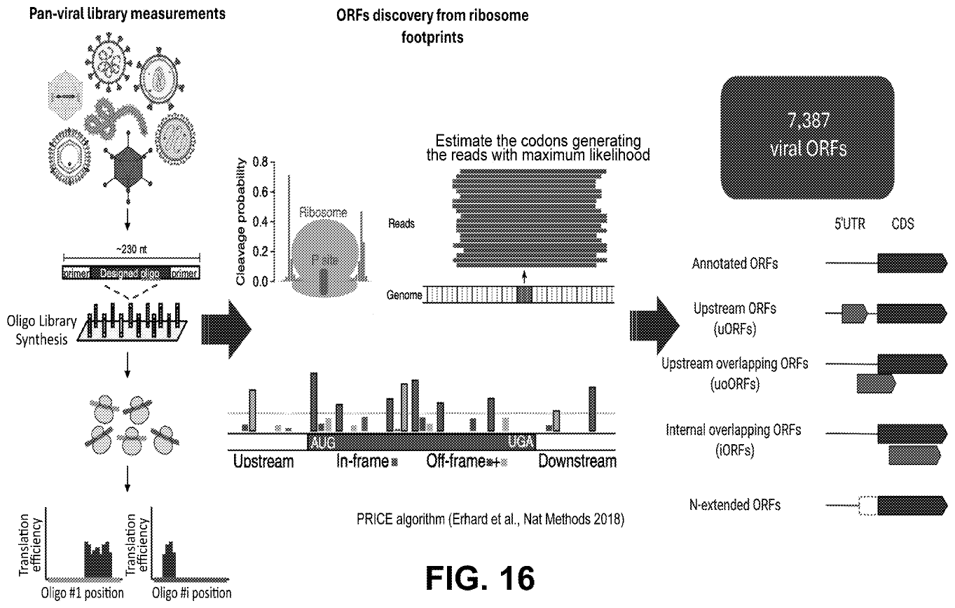

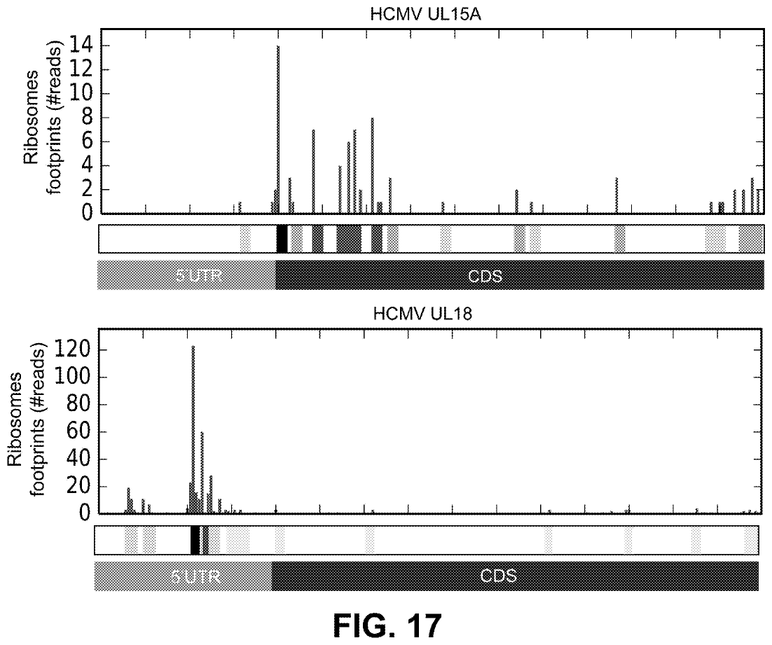

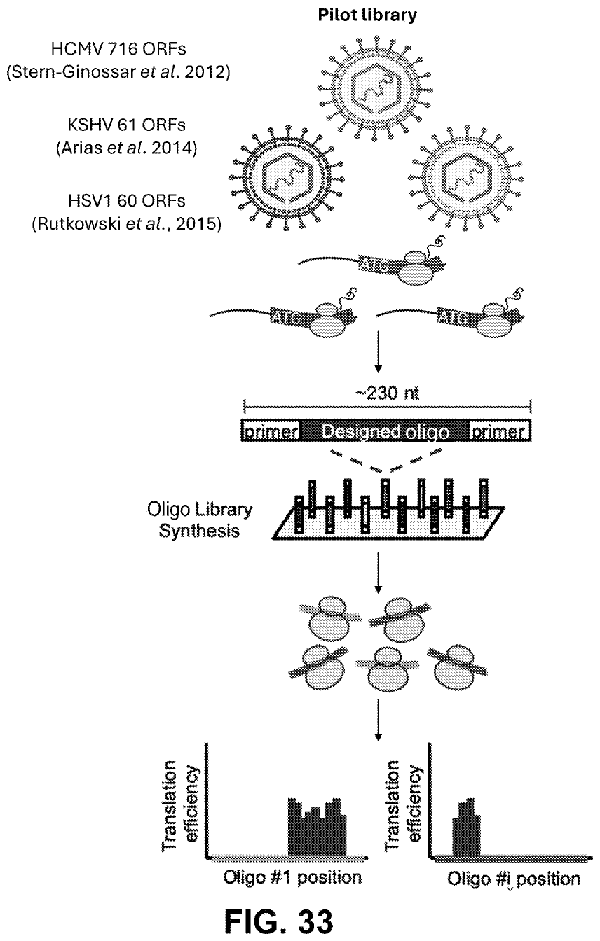

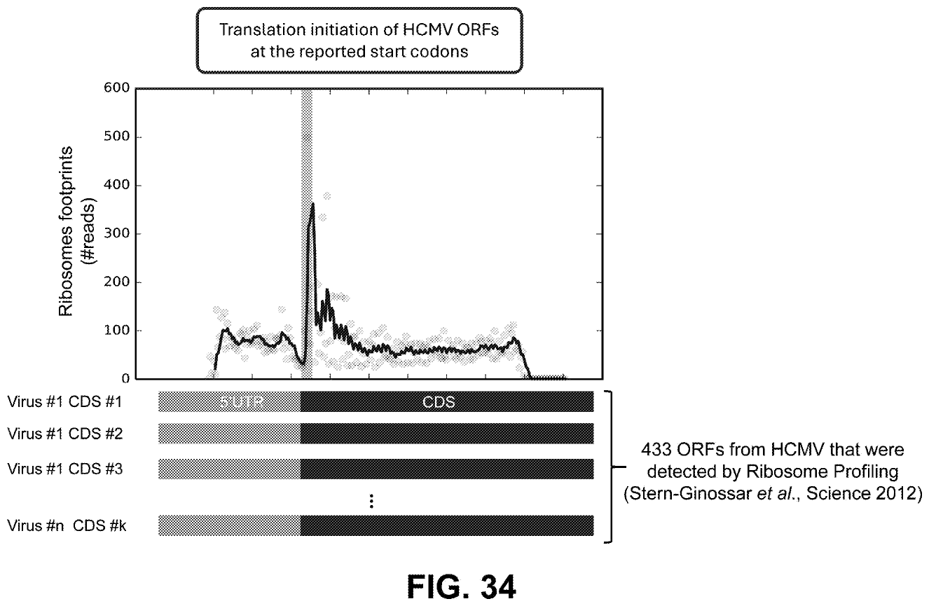

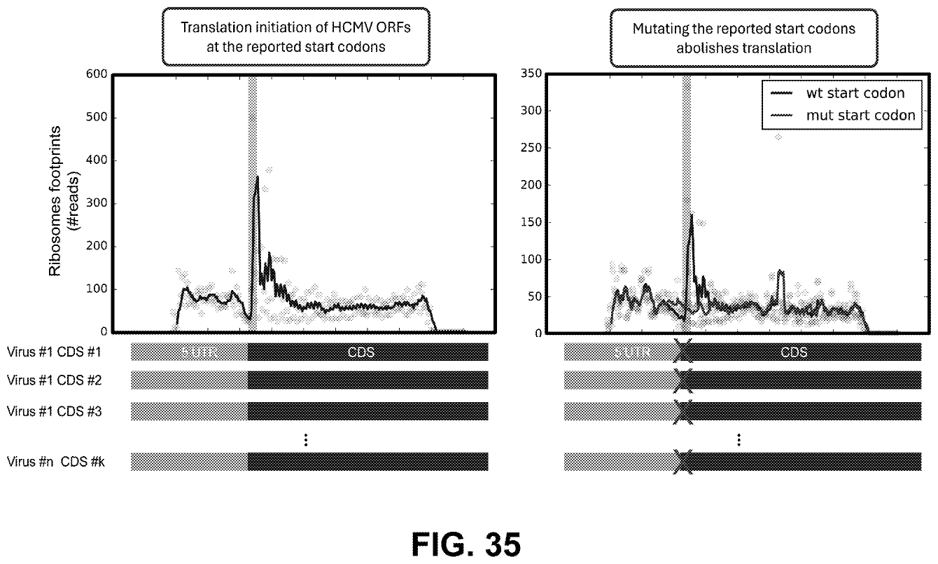

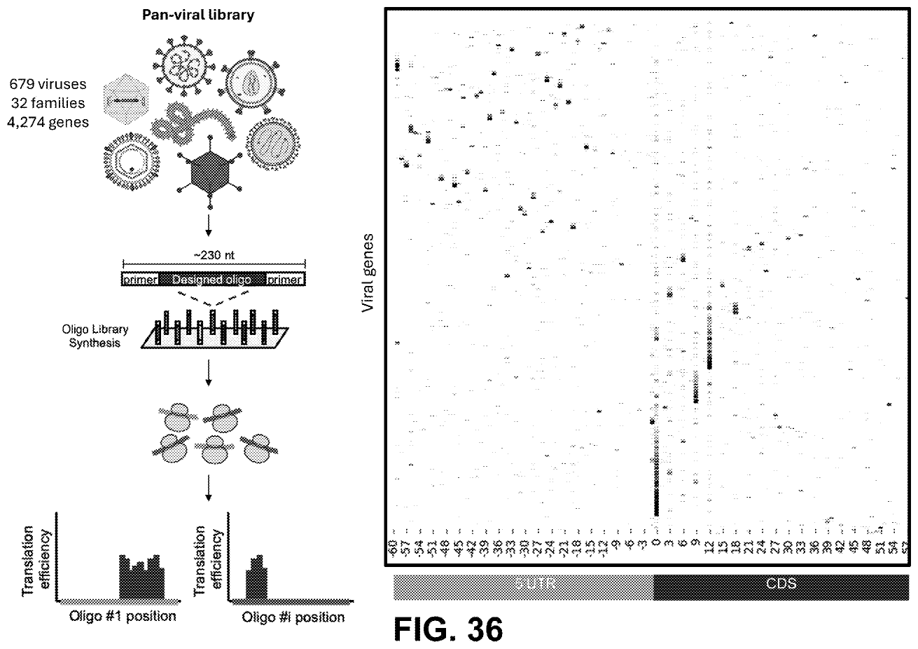

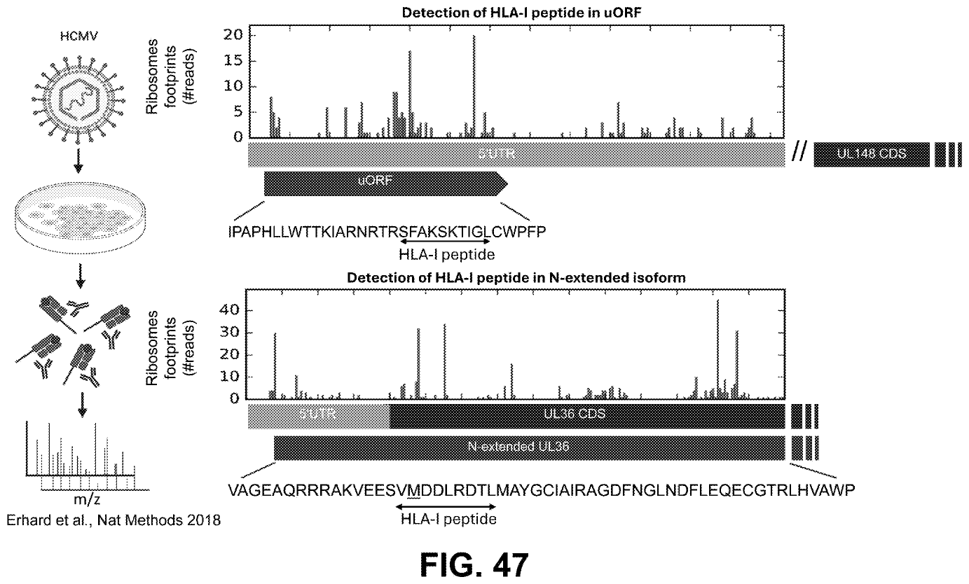

The performance of the synthetic library was evaluated using a predefined set of viral ORFs from ribosome profiling experiments (FIGS. 33-35). As a proof of concept, the MPRP assay accurately detected annotated coding sequences (FIGS. 13, 14, and 15A-15B). Measurements from a pan-viral library composed of 4,274 genes from 679 human viruses were used to uncover ~7,387 viral ORFs (FIGS. 16, 36). Ribosome density measurements were obtained across thousands of annotated viral coding sequences and their 5′ untranslated region (UTR) (FIG. 17). Researching HLA-I peptidome of HCMV uncovers peptides from non-canonical ORFs detected by the pan-viral library (FIG. 47).

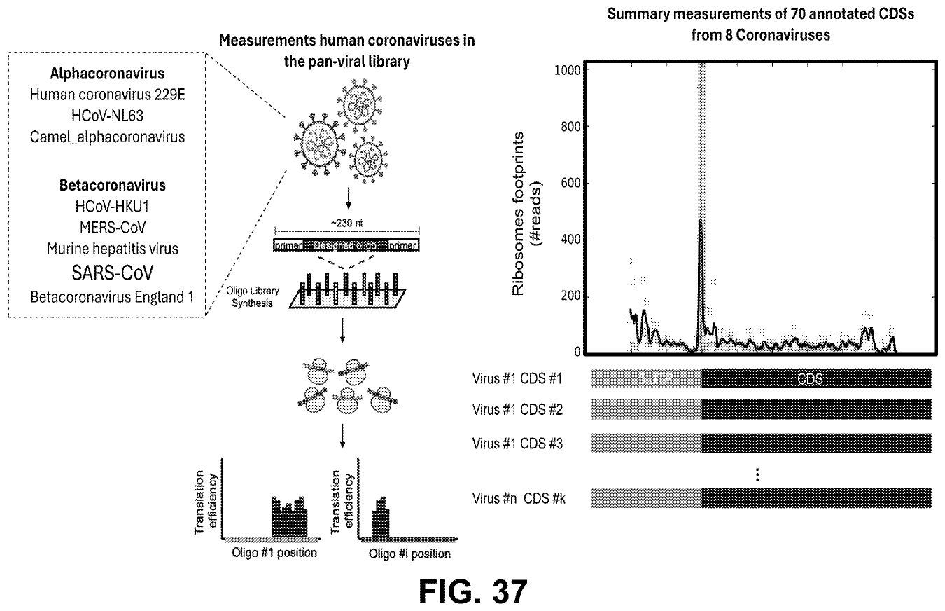

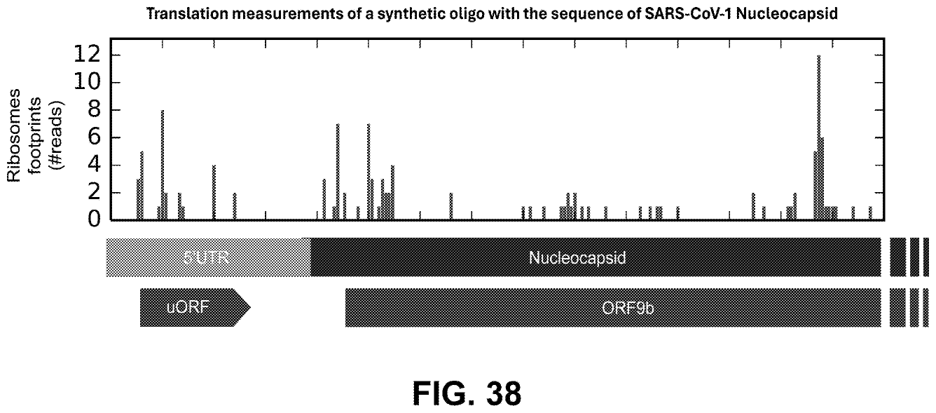

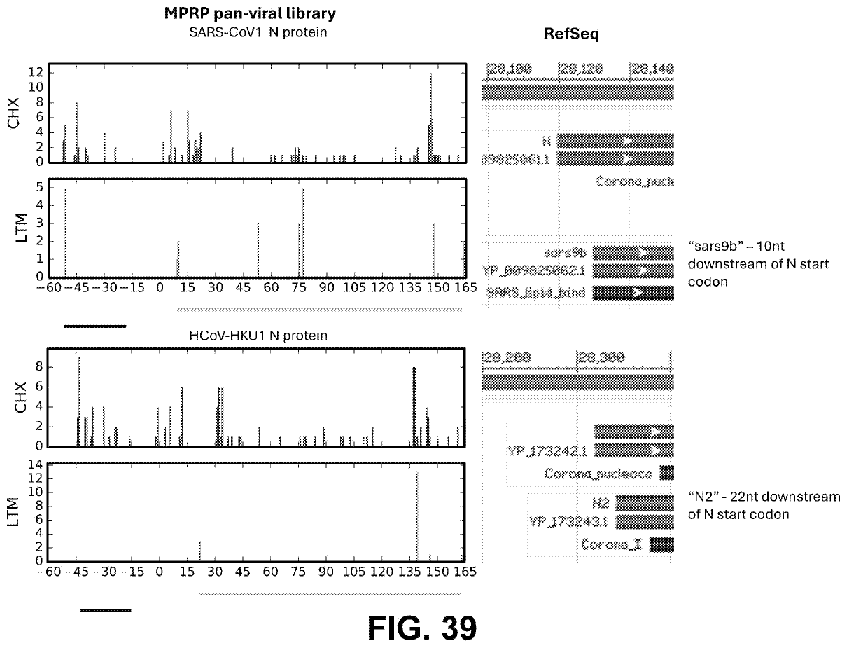

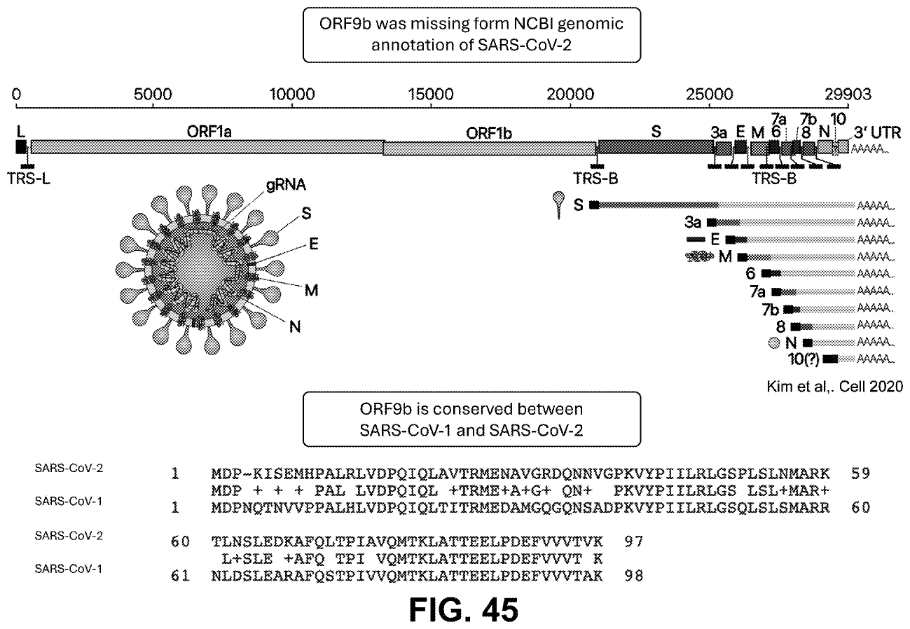

MPRP of the pan-viral library successfully detected annotated ORFs of human Coronaviruses (FIG. 37). MPRP on the pan-viral library detects internal out-of-frame ORFs in the nucleocapsid region of SARS-CoV-1 and HCoV-HKU1 (FIG. 39). MPRP detected the translation of ORF9b in the SARFS-Cov-1 genome (FIG. 38). Although ORF9b is conserved in SARS-CoV-1 it is missing from SARS-CoV2 genomic annotations (FIG. 45).

### Example 2—Gene Expression Regulation by Non-Canonical ORFs

The MPRP assay can be coupled with one or more other techniques to provide further insight into the function of translated regions of viral genomes at scale. Applicants coupled the MPRP assay with inducement of a stress (e.g., via eIF2alpha phosphorylation) to evaluate the uncovered ORFs, such as upstream ORFs and small/short ORFs, on gene expression regulation (see e.g., Khitun et al. Mol. Omics 2019, 15, 108). Upon eIF2alpha phosphorylation induced by arsenite treatment, a sensor of viral infection that attenuates cellular translation, Applicant observed a shift in ribosome occupancy toward the main CDS suggesting that these uORFs facilitate dynamic regulation of viral gene expression in response to environmental changes.

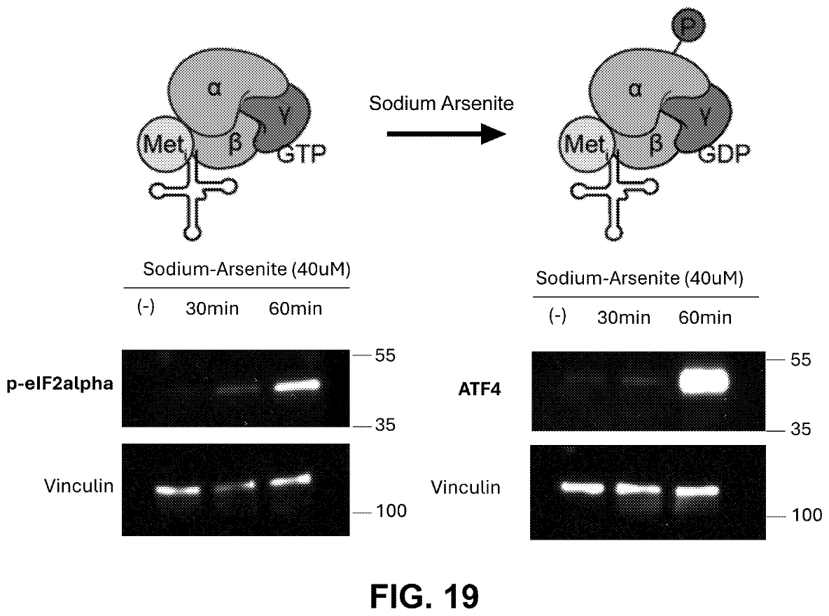

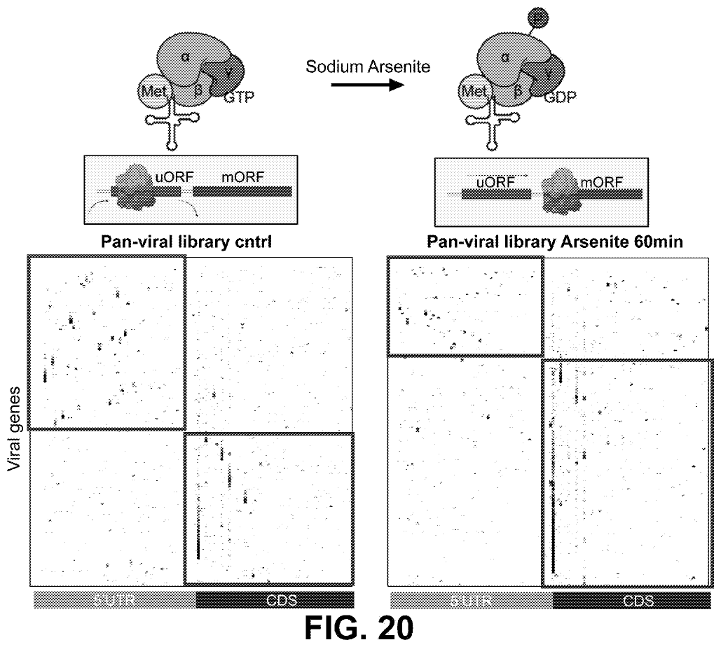

Upstream open reading frames (uORFs) are major gene expression regulatory elements in eukaryotes (see e.g., Barbosa et al., PLoS Genet. 2013 August; 9(8): e1003529) and have also been documented in viruses (see e.g., Shabman et al., PLoS Pathog. 2013 January; 9(1): e1003147). Small open reading frames encode small polypeptides (e.g., 100 amino acids or fewer) and mediate various physiological functions in humans and animals (see e.g., Couso and Patraquim. Nature Reviews Molecular Cell Biology volume 18, pages 575-589 (2017)). Similarly, viral short ORFs have been characterized. See e.g., Finkel et al., Proteomics. 2018 May; 18(10): 1700255). Ribosome footprints from the MPRP assay were compared with and without the stress to determine effect on gene regulation. FIG. 18A-18C shows discovery of hundreds of viral uORFs that negatively regulated the translation of the main coding sequence. FIGS. 19-20 shows that ribosomes are transitioned from the detected uORFs to the main coding sequences in response to eIF2alpha phosphorylation.

### Example 3—Massively Parallel Reporter Assay

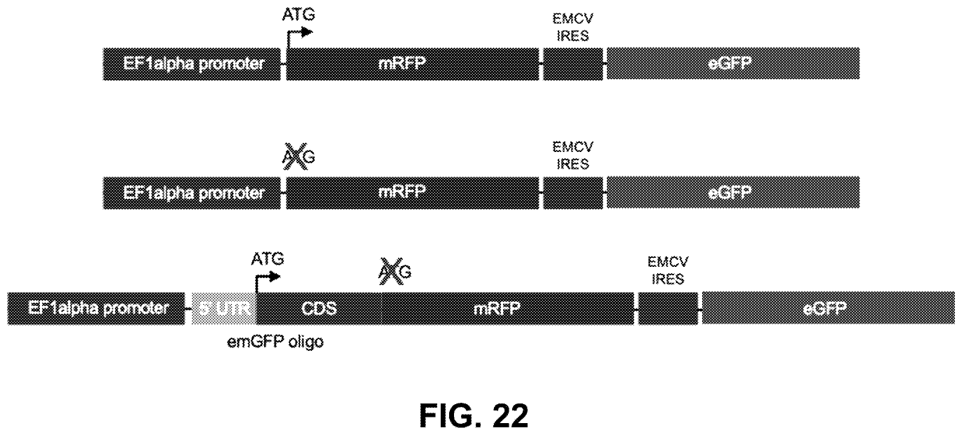

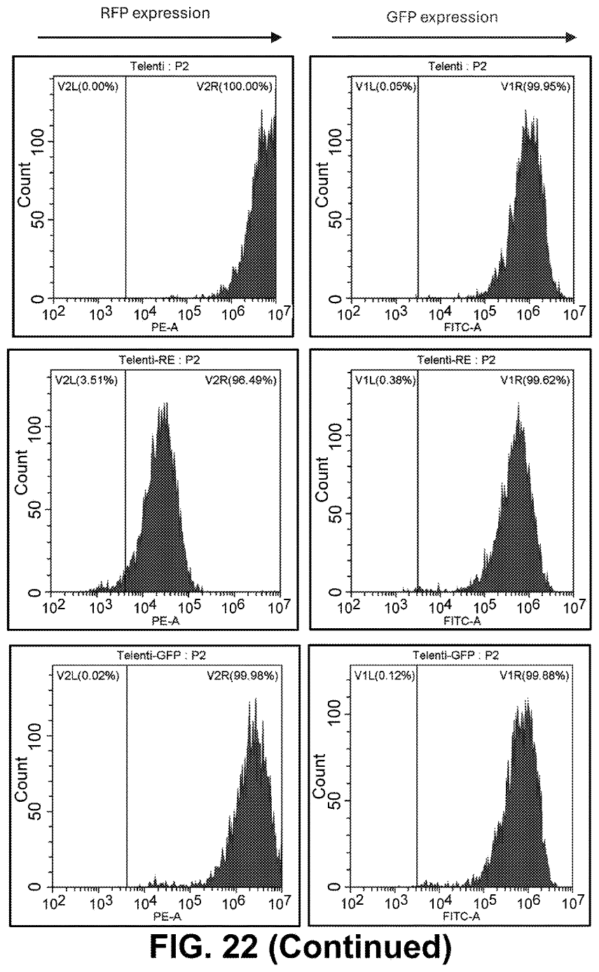

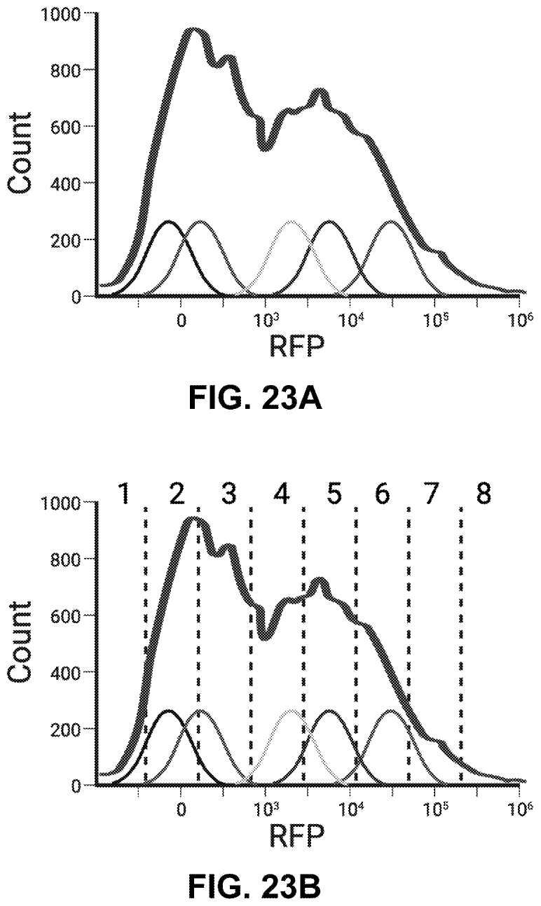

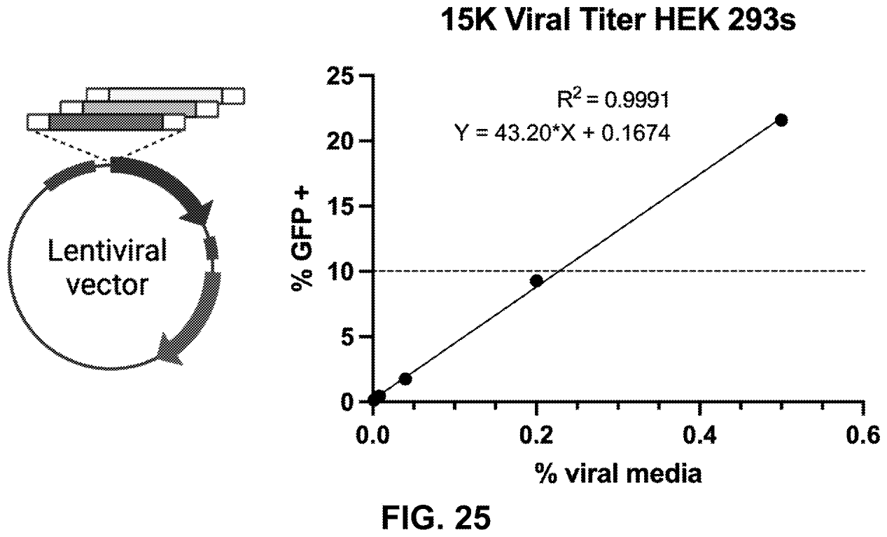

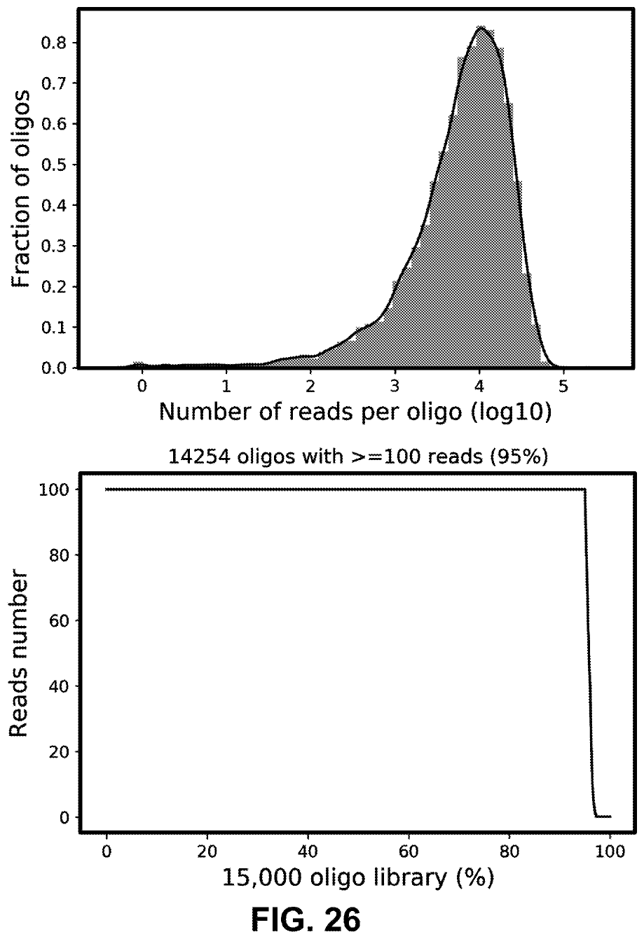

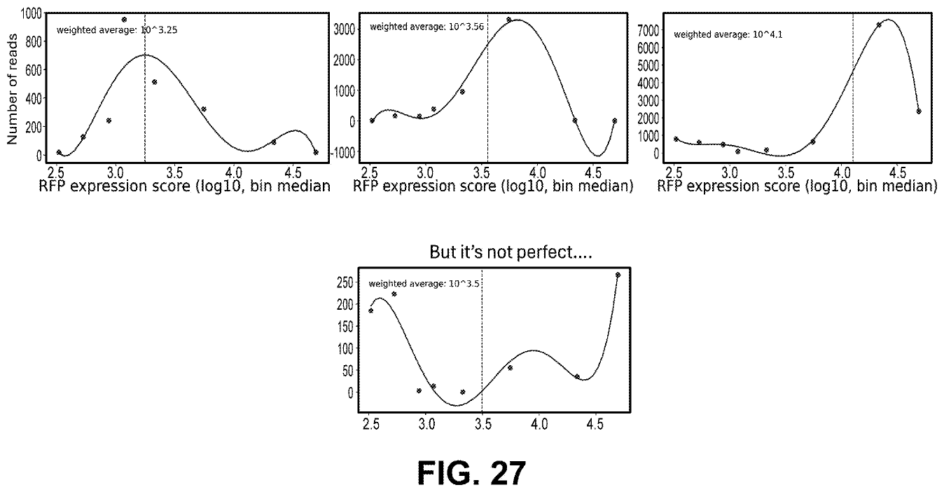

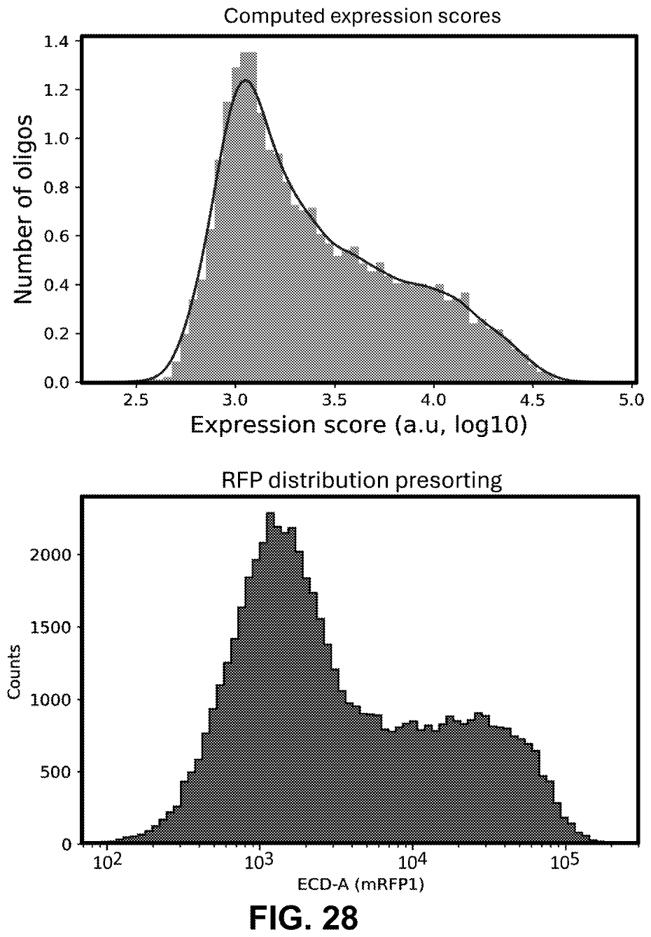

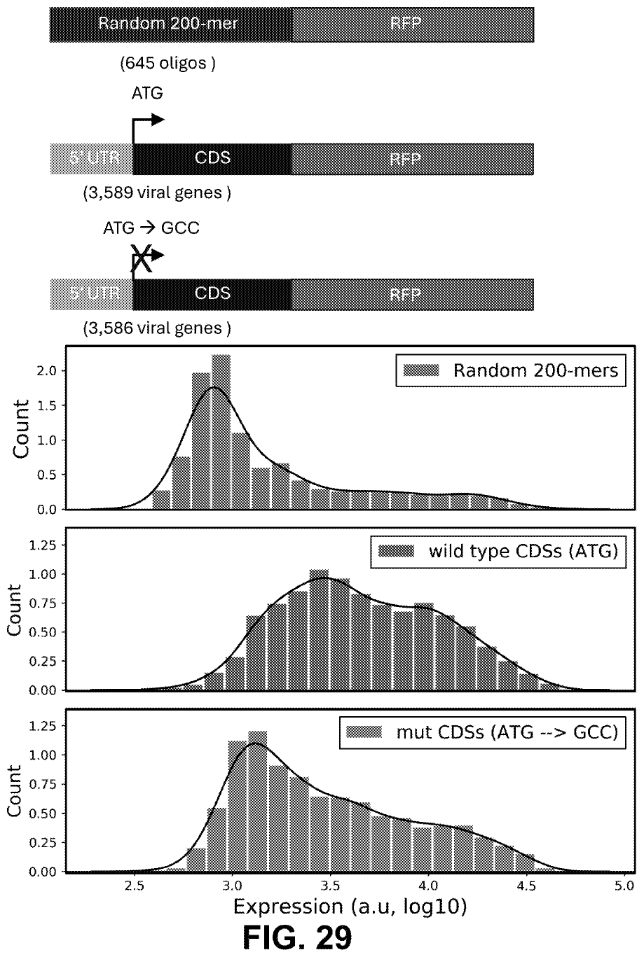

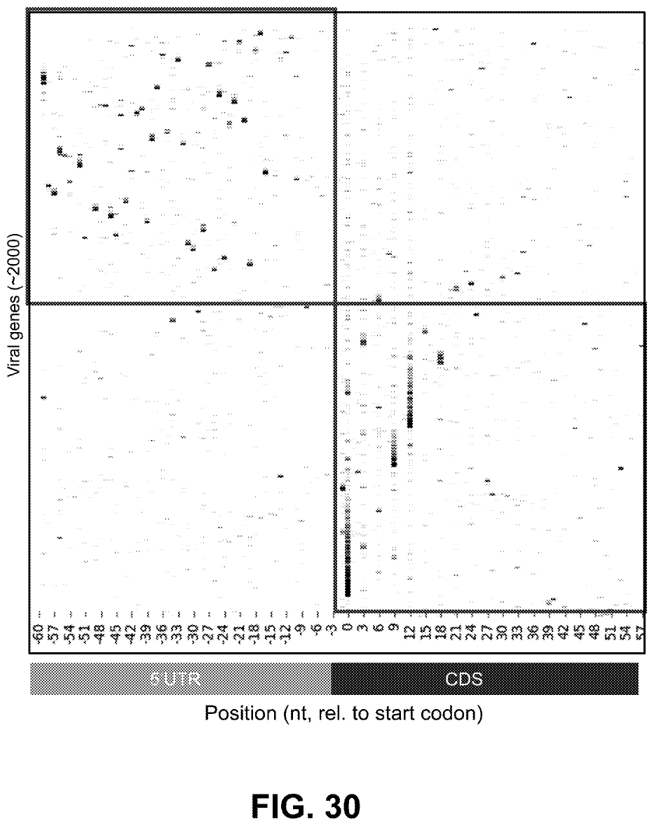

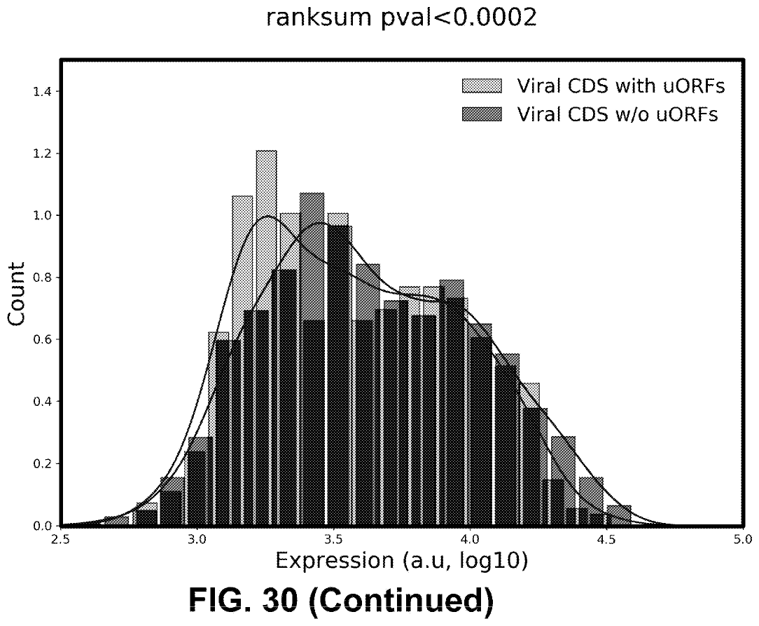

An optically active massively parallel reporter assay was developed for quantitative measurement of translation of ORFs. FIG. 21 shows exemplary optically active constructs for quantitative measurement of translation of viral ORFs. A an ORF, e.g., a viral ORF (CDS) was fused to a first optically active protein (e.g., a first fluorescent protein). This was followed by an IRES and subsequently a second optically active protein (e.g., a second fluorescent protein that fluoresces at a different wavelength than the first optically active protein). The second optically active protein serves as a control. FIG. 22 shows quantitative measurement of viral ORFs translation using an optically active reporter constructs. The reporter construct can have at least two different optically active polypeptides. FIG. 23A-23B demonstrate computationally inferring RFP (e.g., first optically active protein) expression of individual oligos from their representation in sorted expression bins. FIG. 24 shows development of a Massively Parallel Reporter Assay for quantitative translation measurements of 15,000 viral sequences. FIG. 25 demonstrates the use of constant expression of one of the at least two different optically active proteins of the construct of FIG. 22 to control for multiplicity of infection (MOI). FIG. 26 shows that uniform coverage of sequencing reads across library oligos was achieved. A weighted average for each oligo was computed from reads distribution across expression bins (FIG. 27). Applicant assessed the accuracy of the pooled expression measurements (FIG. 28). Applicant further demonstrated operation of the MPRA by using it to detect annotated viral ORFs (FIG. 29). This revealed that translation of viral ORFs is lower in genes with upstream ORFs in their 5′ UTRs (FIG. 30).

### Example 4—Pan-Viral ORFs Discovery Using Massively Parallel Ribosome Profiling

### Introduction

Despite remarkable advances in sequencing viral genomes, functional annotations of these genomes have lagged behind, hindering our understanding of the mechanisms by which viruses propagate and interact with the immune system. One important area of exploration is the translatome—the entire collection of viral mRNA sequences being translated into proteins. Beyond the annotated canonical ORFs, viral genomes encode non-canonical ORFs that do not fulfill the classical definition of ORFs, i.e. do not start with an ATG codon and/or are shorter than 100 amino acids (aa). These non-canonical ORFs and the resulting microproteins have been shown to play a critical role in modulating viral infection (Lulla et al. 2019; Ogden et al. 2019; Lulla and Firth 2020), regulating the expression of canonical viral proteins (A. Chen, Kao, and Brown 2005; Gould and Easton 2005; Shabman et al. 2013), and contributing to the immune response to viruses (Bansal et al. 2010; Ingolia et al. 2014; Yang et al. 2016; Weingarten-Gabbay et al. 2021). However, the deviation from classical ORF features challenges their detection computationally and relies mostly on experimental measurements. Thus, for decades of research this “hidden” source of proteins has remained mostly unknown.

The development of ribosome profiling over the past decade has transformed our ability to detect translated regions across genomes (Ingolia et al. 2009). Ribosome profiling (Ribo-seq) utilizes deep sequencing of ribosome-bound mRNA fragments to determine ribosome occupancy, indicating translated regions at single nucleotide resolution. Quickly adapted to different organisms, ribosome profiling uncovered a striking number of non-canonical ORFs in mammalian cells, yeast, bacteria and viruses including upstream ORFs (uORFs) in 5′UTRs, short ORFs in non-coding RNAs and overlapping internal ORFs in annotated coding sequence (iORFs) (Ingolia, Lareau, and Weissman 2011; Ingolia, Hussmann, and Weissman 2019).

Although the function of most of the non-canonical ORFs remain unknown, the translated polypeptides contribute to the collection of viral antigens that are presented on the surface of infected cells. When viruses infect cells, their proteins are processed and displayed on the cell surface by the class I human leukocyte antigen complex (HLA-I). These peptides in turn activate cytotoxic CD8+ T cells that eliminate the infected cell. While the majority of T cell studies focus on canonical proteins, there is growing appreciation of the contribution of non-canonical ORFs to HLA-I presentation (Starck and Shastri 2016; Holly and Yewdell 2023). Applicant recently showed that peptides derived from non-canonical ORFs in the SARS-CoV-2 genome are enriched on the HLA-I complex in infected cells (Weingarten-Gabbay et al. 2021) and contribute to HLA-II presentation (Weingarten-Gabbay et al. 2023). Notably, some of the non-canonical peptides elicited stronger CD8+ T cell responses in a humanized mouse model and COVID-19 patients than canonical peptides, including a few of the most immunodominant T cell epitopes reported to date. Our findings join others in the field, portraying non-canonical ORFs as an important source for antigen presentation in viral infection, uninfected cells, and cancer (J. Chen et al. 2020; Hickman et al. 2018; Ingolia et al. 2014; Maness et al. 2010; Ouspenskaia et al. 2022; Ruiz Cuevas et al. 2021; Yang et al. 2016). Thus, exposing non-canonical ORFs is poised to provide new targets for vaccine design and deepen our understanding of the interaction between viruses and the immune system.

In addition to being a rich source of microproteins, non-canonical ORFs also act as cis-regulatory sequences that determine the expression levels of canonical viral proteins. The translation of uORFs from a start codon in the 5′UTR attenuates the translation of the main ORF because reinitiation after stop codon is generally inefficient (Hinnebusch, Ivanov, and Sonenberg 2016). In addition, the presence of ribosomes on the uORF can create a “roadblock”, precluding scanning pre-initiation complexes (PICs) from initiating at the main ORF. The translation of uORFs is a dynamic process that changes in response to cellular stress, including viral infection, through the phosphorylation of eIF2alpha, a key translation initiation factor. Upon eIF2alpha phosphorylation, scanning PICs are more likely to “miss” the start codon of uORFs through leaky scanning and initiate translation at the main ORFs. uORFs were shown to regulate the expression of canonical viral proteins in Ebola virus, Hepatitis B virus (HBV), and Respiratory Syncytial Virus (RSV)(A. Chen, Kao, and Brown 2005; Gould and Easton 2005; Shabman et al. 2013). Exposing uORFs in viruses can enhance our understanding of the mechanisms by which viruses determine the expression of proteins in infected cells and along the viral life cycle.

Although undoubtedly important, the landscape of translated regions is still unknown for the majority of viruses. Ribosome profiling has been successfully employed to profile a handful of viruses; however, it is still limited in throughput with respect to the number of viruses that can be tested. Each virus requires a unique culturing system, exhibiting selective tropism to cells and growing conditions. Thus, each virus should be profiled in isolation, limiting the number of viruses that can be assayed in each experiment. In addition, researching highly pathogenic viruses necessitates high-containment facilities and strict safety protocols that challenge the execution of ribosome profiling within biosafety level 3 or 4 (BSL3/4) laboratories. Moreover, some viruses cannot be cultured in the laboratory, making it impossible to profile their translation in-vitro. Finally, since viruses are a moving target, their genome changes constantly, a method that will evaluate many variants in parallel can be beneficial.

Creating a platform for the discovery of ORFs across many viral genomes, independent of their culturing conditions and biosafety level, would transform our knowledge of viruses' translatomes and is poised to expose new biology. In this study, Applicant leveraged the power of a fully designed oligo synthesis library(Weingarten-Gabbay et al. 2016, 2019; Seo et al. 2023) and combined it with ribosome profiling to perform pan-viral ORF discovery. Applicant measured the translation of 20,170 synthetic sequences from 679 viral genomes in two human cell lines, resulting in the identification of thousands of new ORFs. Applicant estimated ORF discovery using the annotated CDSs in these genomes and non-canonical ORFs that were identified in traditional ribosome profiling of infected cells. Applicant then examined the function of the new ORFs that Applicant detected in two processes: HLA-I antigen presentation and uORF-mediated translation regulation of canonical viral proteins.

### Results

### Developing Massively Parallel Ribosome Profiling to Identity ORFs in 679 Viral Genomes

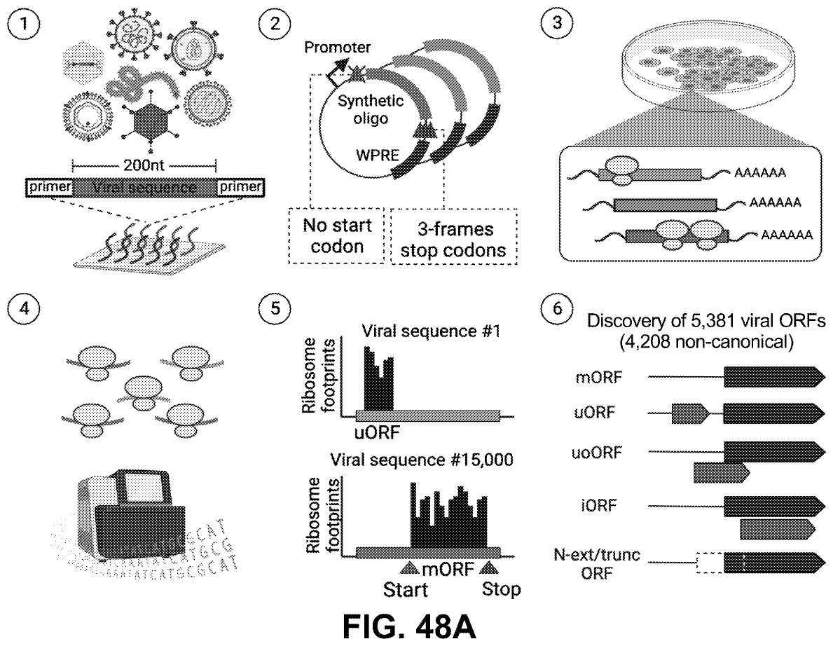

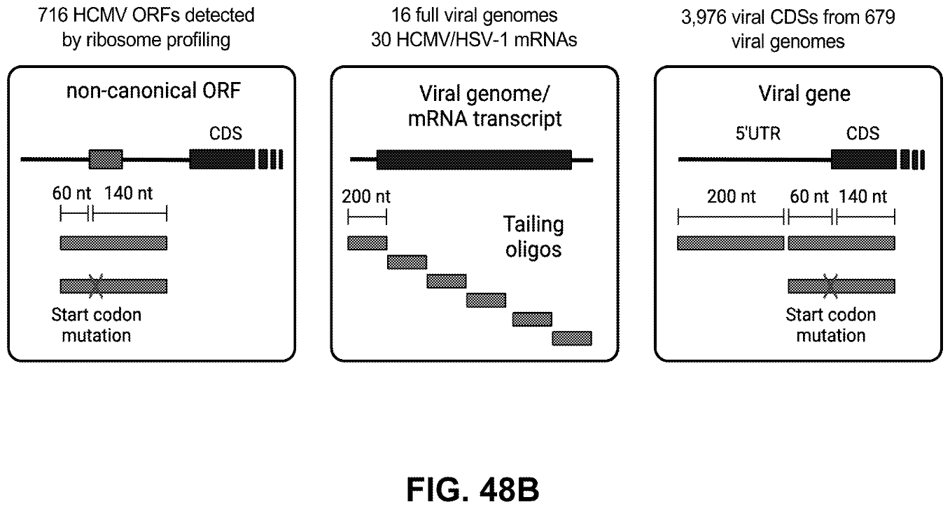

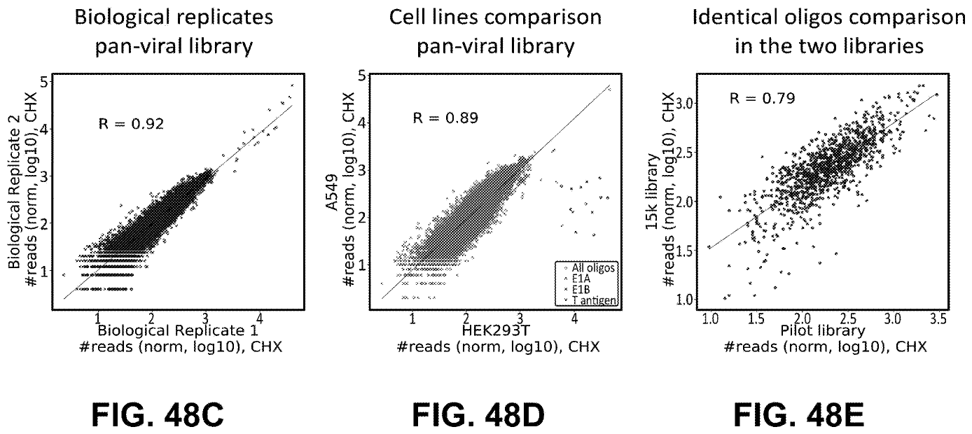

To measure ribosome occupancy and infer translated regions across hundreds of human viruses, Applicant developed Massively Parallel Ribosome Profiling (MPRP) (FIG. 48A). Instead of infecting cells with individual viruses, Applicant used oligonucleotides library synthesis technology to encapsulate thousands of viral sequences in a single pooled experiment. Each oligo contained 200 nt of viral sequence, flanked by constant primers. Applicant amplified the library using the constant primers and cloned it into an overexpression plasmid downstream of a CMV promoter and upstream of a Woodchuck Hepatitis Virus (WHV) Posttranscriptional Regulatory Element (WPRE) used to further enhance expression. To monitor the translation of ORFs in the designed oligos, Applicant excluded ATG start codons on the plasmid and the constant primer sequence upstream to the cloned oligo. Since the designed sequence is limited to 200 nt, some oligos contain the beginning of a viral ORF but not the end. Applicant ensured translation termination in the absence of the endogenous stop codon by inserting stop codons in all three potential reading frames on the plasmid, downstream of the cloned oligo. Applicant transfected the pooled library plasmid into HEK293T human embryonic kidney cells or A549 human lung cells. Applicant then performed a modified protocol of ribosome profiling on library-transfected cells (McGlincy and Ingolia 2017) (see methods) after treating cells with either cycloheximide (CHX) to inhibit elongating ribosomes across the entire ORF, or lactimidomycin (LTM) to specifically inhibit initiating ribosomes at the start codon. Applicant then mapped deep sequencing reads representing ribosome footprints to the synthetic library and identified ORFs using PRICE, a computational method for the detection of canonical and non-canonical ORFs in ribosome profiling experiments (Erhard et al. 2018) (see methods).

Applicant initially verified that our experimental system captures the translation of an ORF embedded within a synthetic oligo. Applicant performed ribosome profiling on cells that were transiently transfected with a full-length Green Fluorescent Protein (GFP) (FIG. 48A) and observed the expected ribosome footprints on the GFP CDS (FIG. 48B). Applicant then swapped the full-length GFP with the first 170 nt of the reporter that resembled the length of a synthetic oligo in the library. Although the cloned sequence contained only part of the original ORF and did not harbor the GFP stop codon, Applicant detected ribosome footprints on the truncated GFP regions, with successful termination in one of the three stop codons that Applicant added to the plasmid (FIG. 48C). Interestingly, Applicant observed unexpected ribosome footprints on the WPRE region. These footprints accounted for a large fraction of the sequencing reads in the MPRP experiment because they originated from a constant region of the overexpressed plasmid and. To remove WPRE-derived footprints prior to sample sequencing, Applicant implemented a home-made ribosomal RNA (rRNA) depletion step and appended WPRE-targeting probes in addition to the rRNAs probes (see methods).

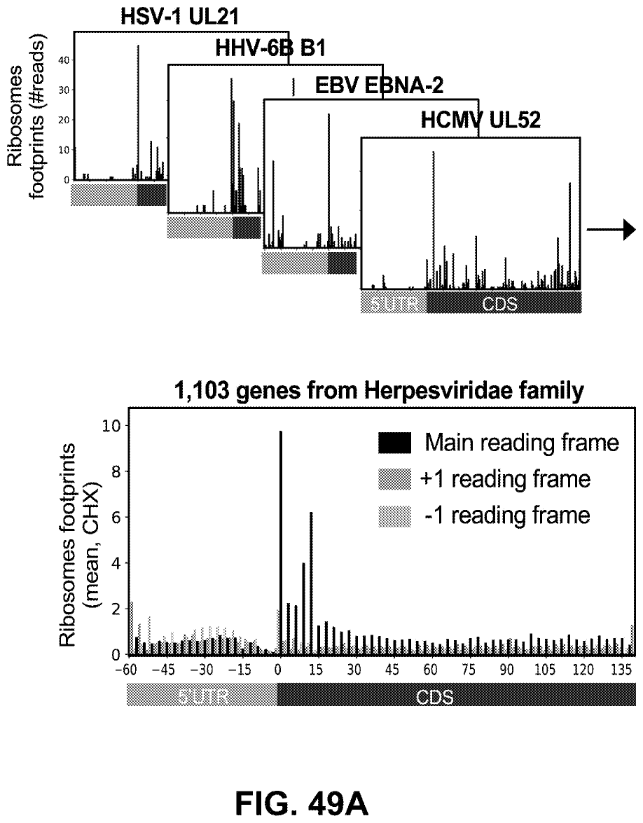

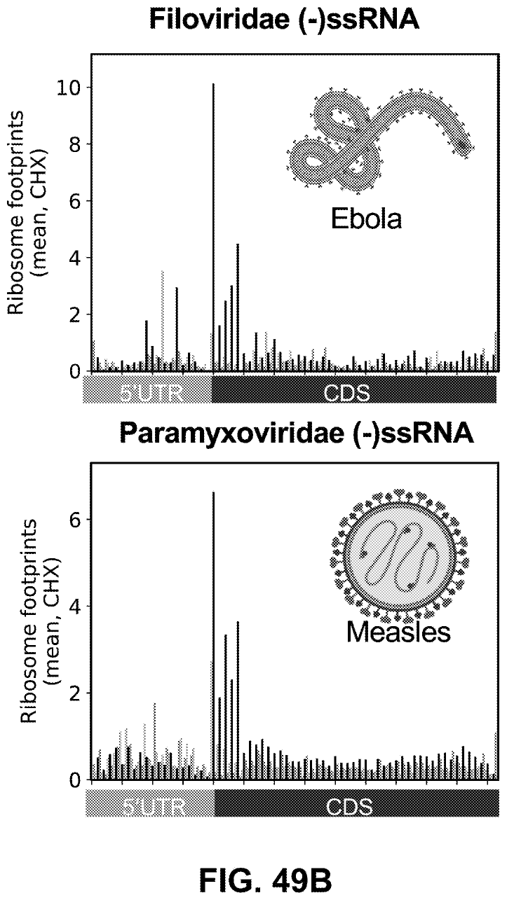

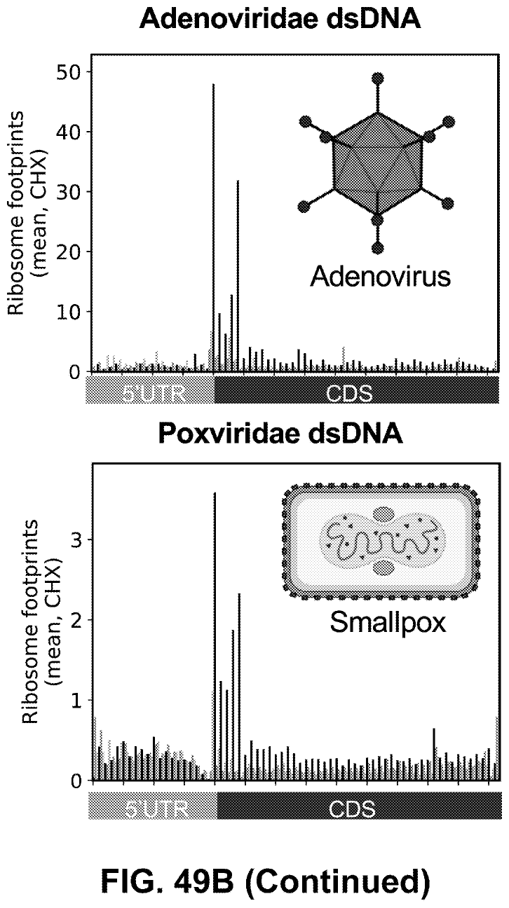

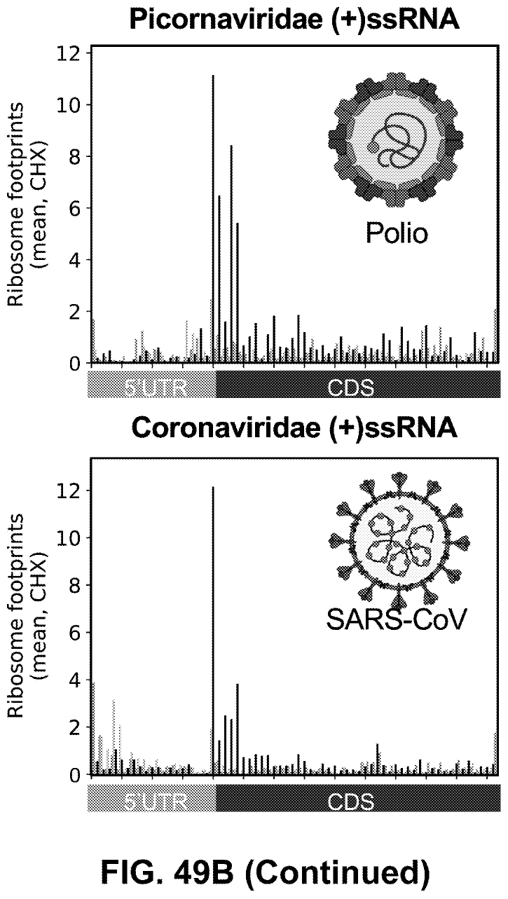

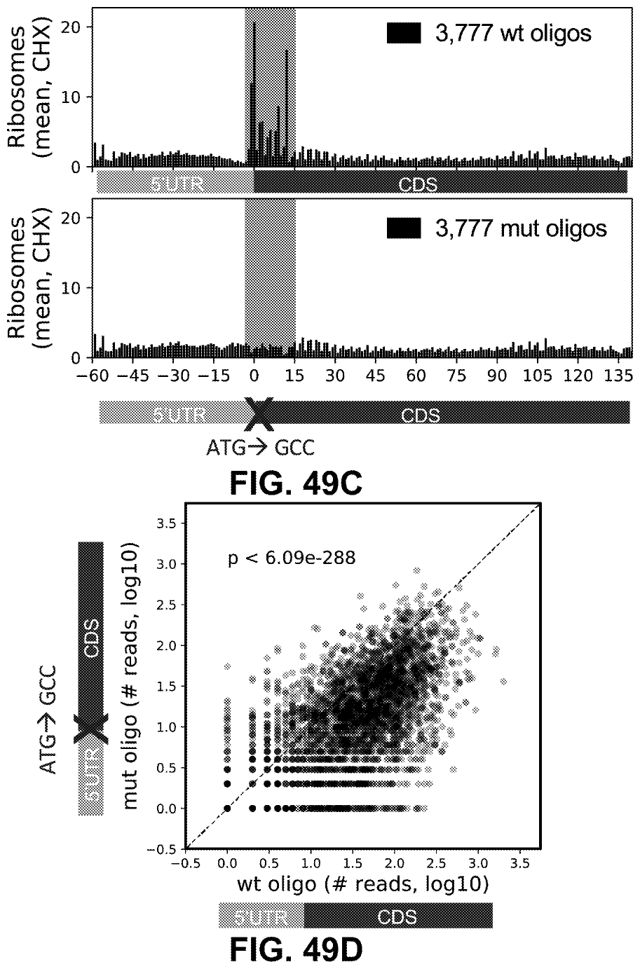

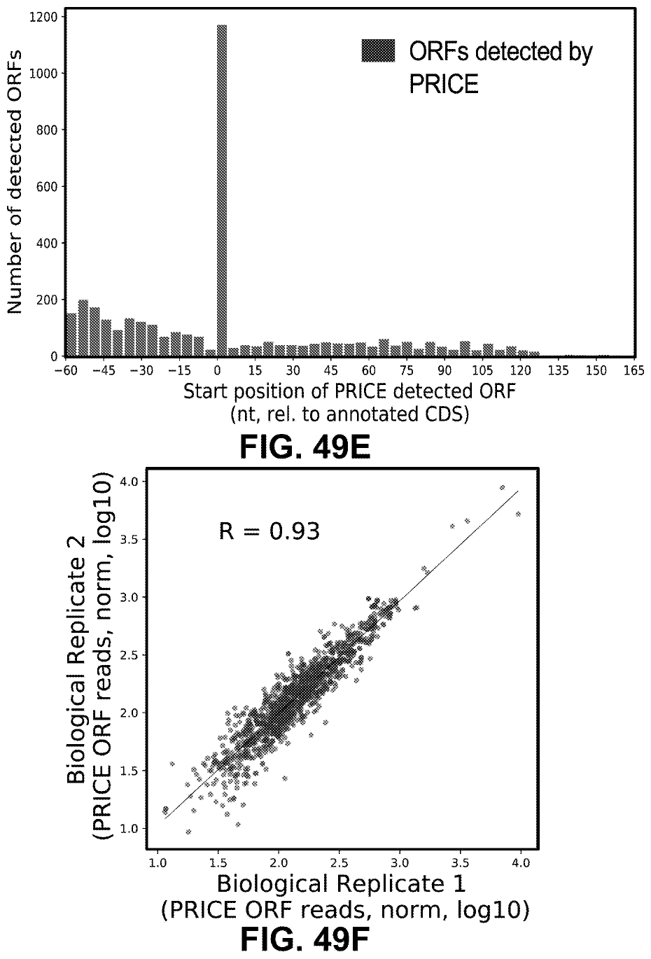

Applicant designed a pilot library of 5,170 oligos to estimate viral ORF discovery using MPRP. This library contained 716 ORFs that were identified in HCMV-infected cells by traditional ribosome profiling (Stern-Ginossar et al. 2012) (FIG. 48B). Each oligo was composed of 60 nt upstream of the start codon and 140 nt downstream of the start codon. In addition, for each ORF, Applicant also designed a mutated oligo in which Applicant replaced the reported start codon with GCC. To evaluate de novo discovery of ORFs, Applicant tiled the entire genome of 16 viruses, and 30 mRNAs annotated in the genomic datasets of HCMV and HSV-1 (NC_006273.2 and JN555585.1, respectively) (FIG. 49A). When examining the number of ribosome footprints across oligos from different regions of the mRNA transcripts, Applicant observed the expected low occupancy in the 5′UTR and enrichment of ribosomes at the start codon (FIG. 49B). However, Applicant also observed high occupancy along the CDS. Since 200 nt-long oligos in the CDS region do not contain the original mRNA start codon, ribosomes in these oligos represent independent translation initiation from an alternative start codon. However, in the context of the full transcript, these start codons are preceded by multiple start codons, making it unlikely that the ribosome will initiate at this position. Applicant reasoned that MPRP has higher accuracy at the beginning of annotated CDSs and 5′UTRs, which better mimic the native genomic context. Thus, Applicant decided to focus on these regions for the design of the pan-viral library.

Applicant designed a pan-viral library of 15,000 oligos to screen for novel ORFs in the 5′UTRs and the beginning of the CDS of 3,976 genes in 679 viral genomes. For each gene Applicant designs three oligos: a wild type oligo containing 60 nt of the 5′UTR and 140 nt of the CDS, a mutated oligo in which the annotated start codon was mutated to GCC, and a farther upstream oligo in the 5′UTR region, starting −260 nt relative to the CDS start codon (FIG. 48B). Applicant performed MPRP measurements on the pan-viral library and uncovered 5,381 viral ORFs including 4,208 non-canonical ORFs (uORFs, iORFs, N-extended isoforms, and N-truncated isoforms) (SEQ ID NO: 9891-16277). See also e.g., Weingarten-Gabbay et al., 2023. bioRxiv https://doi.org/10.1101/2023.09.26.55964, which is incorporated by reference herein, particularly at Table S1.

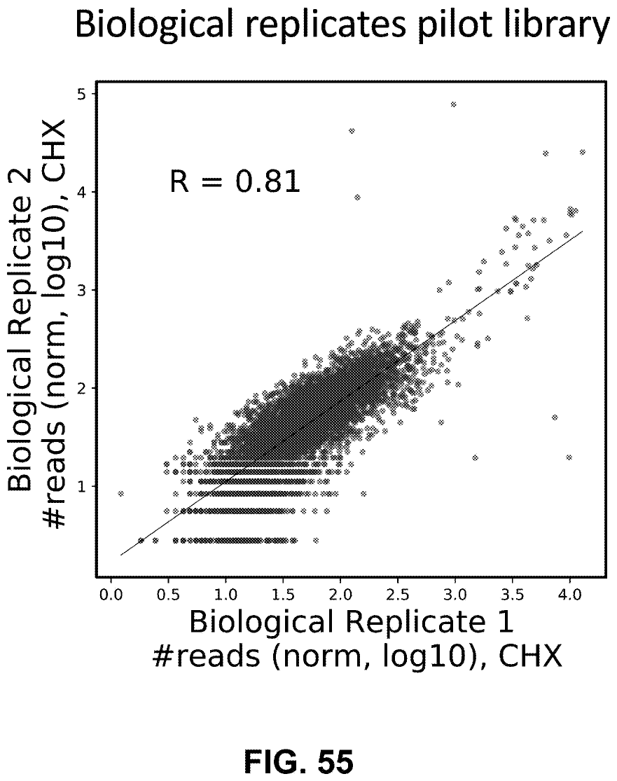

To gauge the reproducibility of the MPRP measurements, Applicant compared ribosomes occupancy on the synthetic oligos in different experiments. Measurements of the pilot and the pan-viral libraries were consistent between biological replicates in HEK293T cells (R=0.81 and R=0.92, respectively, FIG. 55 and FIG. 48C). Applicant also found high agreement between ribosome occupancies on the pan-viral library in HEK293T and A549 cells (R=0.89, FIG. 48D). A small number of oligos had higher occupancy in HEK293T than A549 cells; closer examination of these outliers reveal shared sequences with the adenoviral E1A/B genes and the SV40 large T-antigen that are endogenously expressed by HEK293T cells (Tan et al. 2021). Thus, the elevated occupancies likely stem from cross-mapping of endogenous ribosome footprints to the synthetic library. Finally, Applicant designed a set of 1,033 oligos with identical sequences in the two libraries and found good agreement between oligos that were independently synthesized, cloned and measured using MPRP (R=0.79, FIG. 48E). Altogether, these data indicate that MPRP produces reproducible ribosome occupancy measurements on thousands of viral sequences.

### Estimating ORF Discovery Using Annotated Viral Coding Sequences

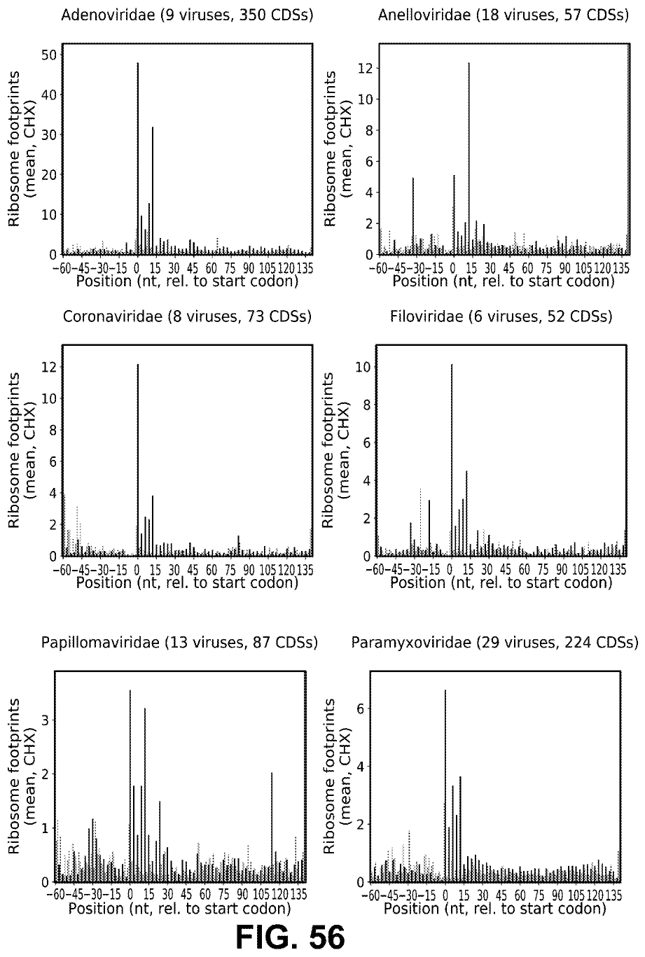

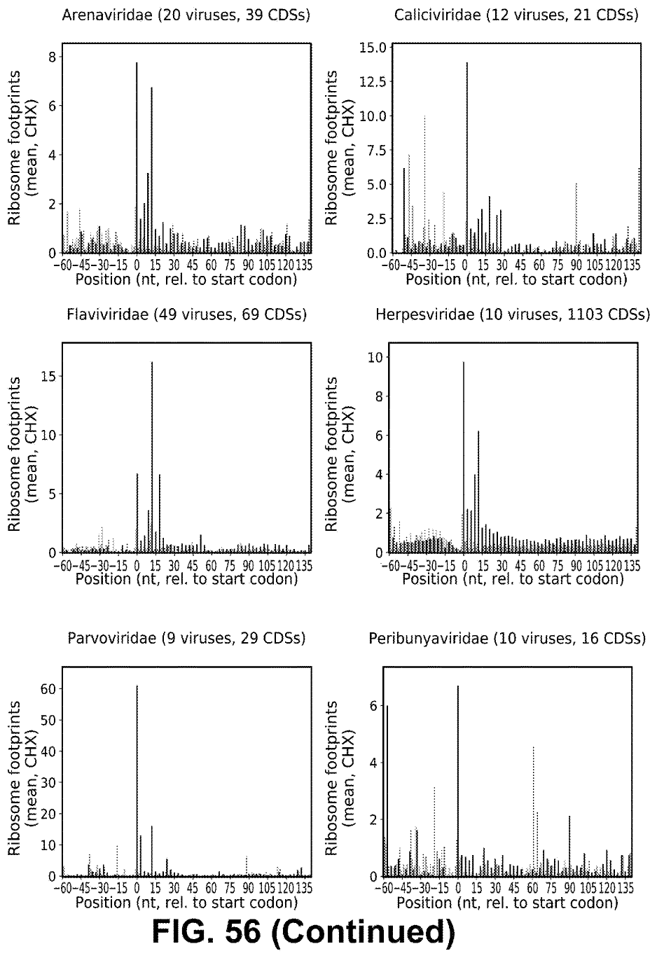

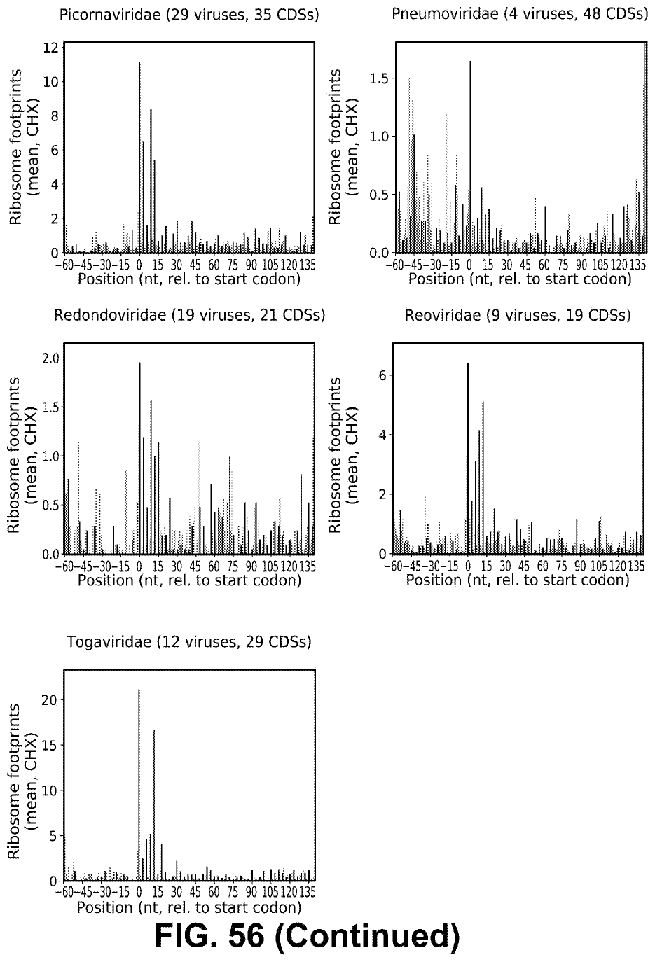

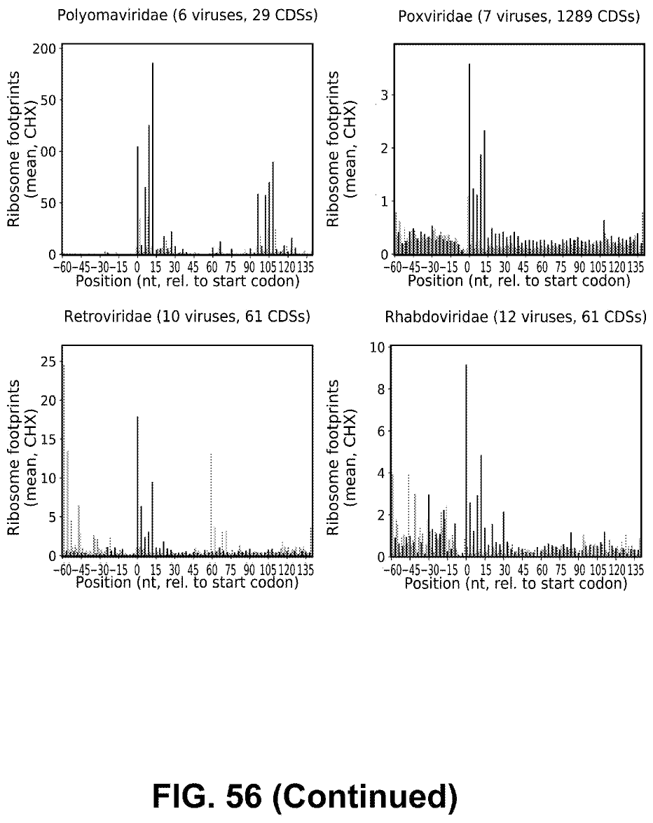

To estimate the detection of viral ORFs when expressed from a synthetic library, Applicant examined the distribution of ribosome footprints across oligos representing the annotated CDSs (FIG. 48B). Applicant performed metagene analysis for all the CDSs from each family by computing the average ribosome footprints in each position relative to the annotated start codon (FIG. 49A). As expected, Applicant found enrichment of ribosome footprints in the CDS region with higher density at the beginning of the sequence that is often observed in ribosome profiling measurements (FIG. 49A-49B). Since ribosome profiling provides single nucleotide resolution of ribosome footprints, it can be used to infer the reading frame in which the ribosome is translating an mRNA sequence. Applicant found the expected tri-nucleotides periodicity, indicating translation in the correct reading frame of the annotated CDSs. These two features were observed in the majority of the 21 families, indicating a robust identification of annotated CDSs in MPRP across different viruses (FIG. 56).

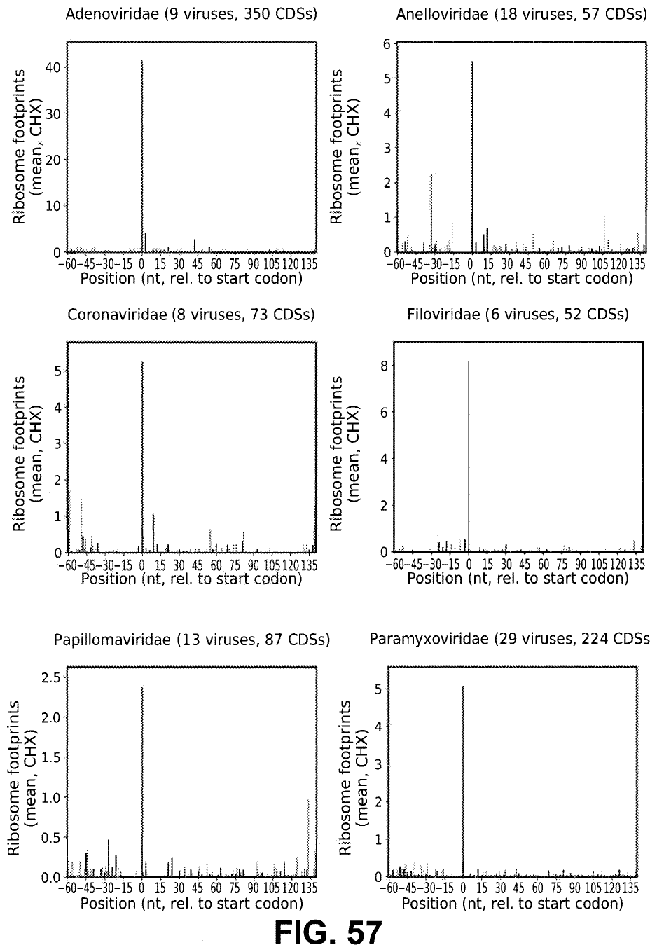

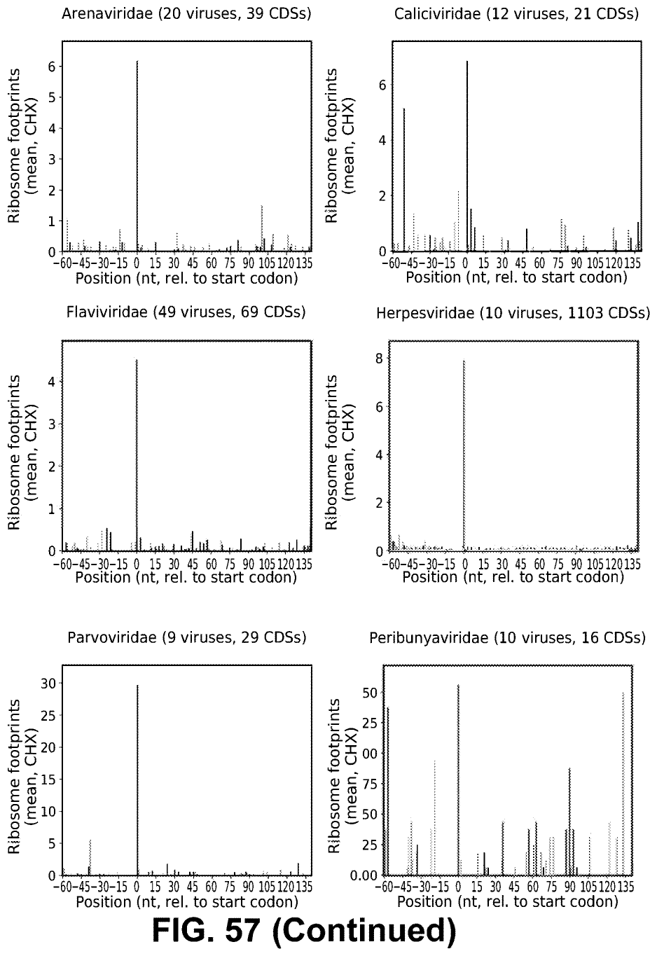

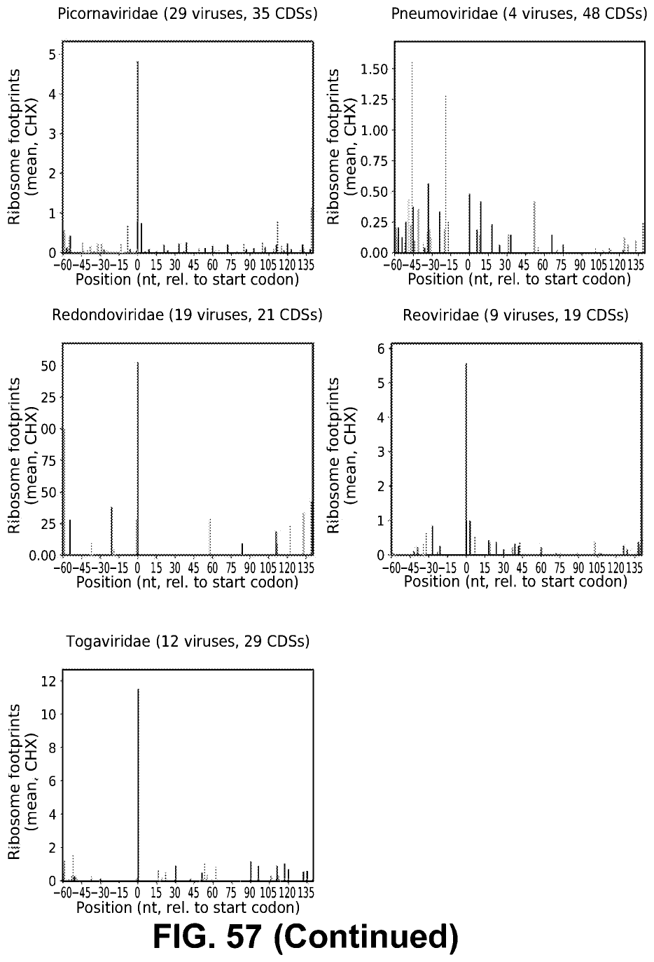

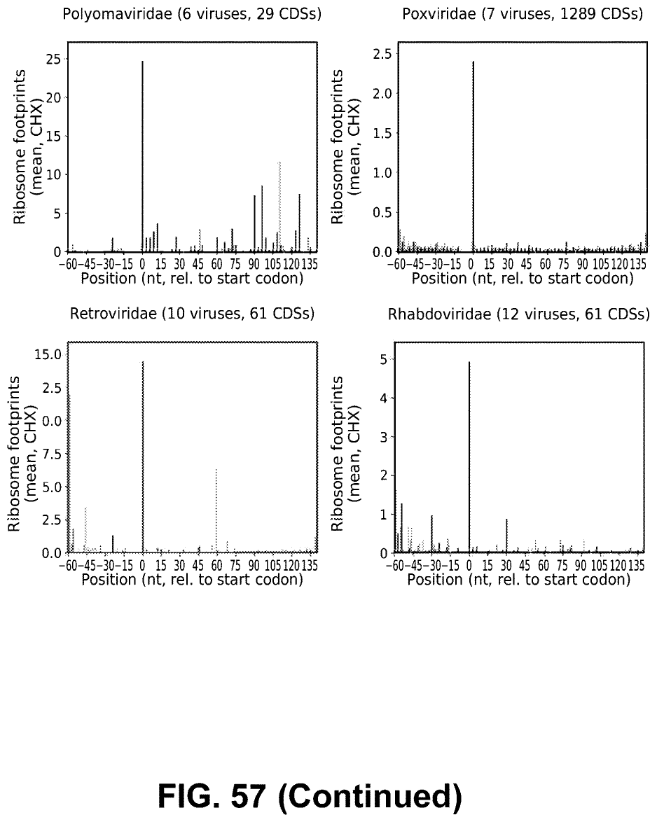

Applicant next confirmed that the detected enrichment of ribosomes in the CDS region resulted from translation initiation at the annotated start codon. Applicant examined ribosome footprints in the presence of an LTM inhibitor, which specifically inhibits initiating 80S complexes with an empty E-site. Applicant found a clear enrichment in the annotated start codon for 19 of the 21 viral families tested (FIG. 57). Applicant also used reverse genetics to test the contribution of the annotated start codon sequence to CDS translation in the MPRP. Applicant designed oligos with identical sequence to 3,777 annotated CDSs in which Applicant mutated the three nucleotides of the start codon to GCC. When comparing ribosome footprints in the region flanking the annotated start codon, Applicant found a substantial reduction in the number of footprints in the mutated oligos (FIG. 49C). It should be noted that since the size of the ribosome footprint is ~29 nt, footprints that do not span the region of three mutated nucleotides are mapped to both the mutated and wt oligo, resulting in similar ribosome occupancy profile downstream of the first few codons (outside the highlighted region in FIG. 49C). Applicant then performed pairwise comparison of the number of ribosome footprints for each viral CDS in the wt and mutated oligo. Here too, Applicant found significant reduction in the number of ribosome footprints on the mutated oligos (p<10⁻²⁸⁸, FIG. 49D).

Applicant used the annotated CDSs to evaluate the performances of the computational pipeline that Applicant used to infer ORFs in the MPRP experiment. When running PRICE, Applicant did not indicate which viral oligos contain annotated CDSs in order to evaluate their discovery in an unbiased fashion. Rather, Applicant used endogenous ribosome footprints from annotated human CDSs to train PRICE and learn essential parameters of the ribosome footprints in the experiment (see methods). PRICE has successfully captured 1,136 of 3,976 viral CDSs (28%) initiating at the annotated start codon (false discovery rate 0.05, FIG. 49E). Applicant also found high agreement between ribosome occupancy on PRICE-detected CDSs in the two biological replicates (R=0.93, FIG. 49F). Together, Applicant shows that MPRP successfully identifies translation initiation and elongation of ribosomes on annotated CDSs across multiple viral families.

### Comparing ORF Detection in MPRP and Ribosome Profiling of HCMV-Infected Cells

In addition to annotated CDSs, Applicant also estimated the discovery of ORFs by comparing MPRP to traditional ribosome profiling done in the context of native viral infection. As part of the design of the pilot library, Applicant included oligos representing the sequence of 716 ORFs that were identified in HCMV-infected cells by ribosome profiling (Stern-Ginossar et al. 2012), termed here “Ribo-seq ORF” (FIG. 48B).

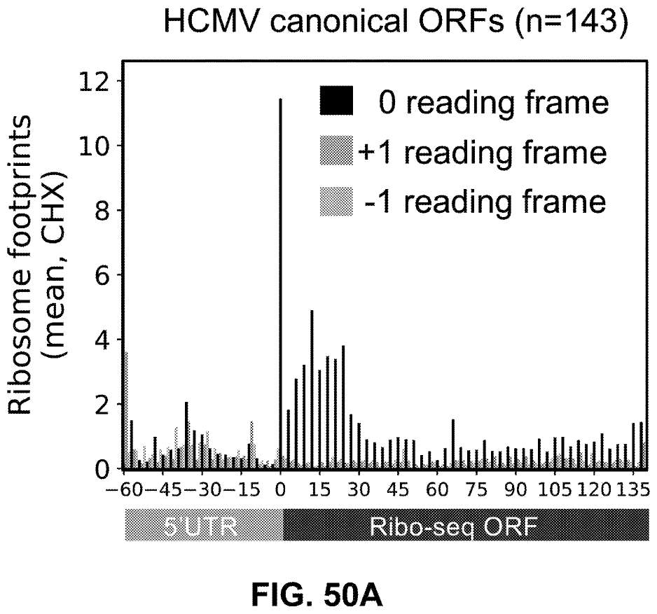

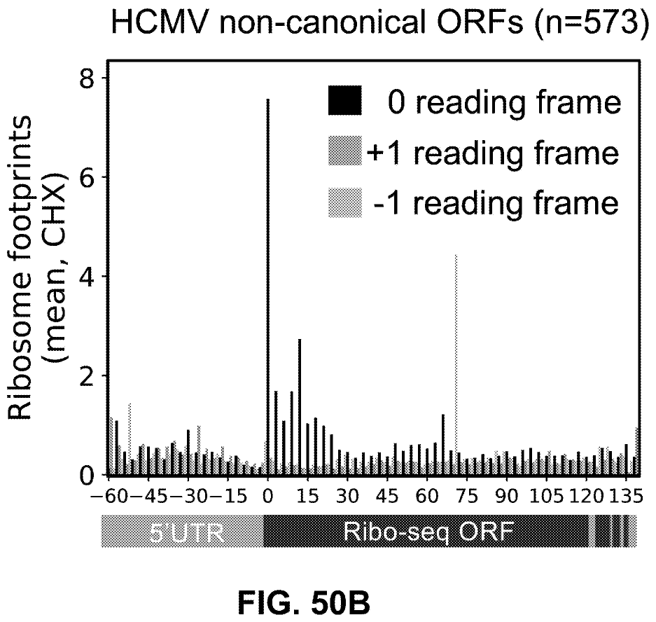

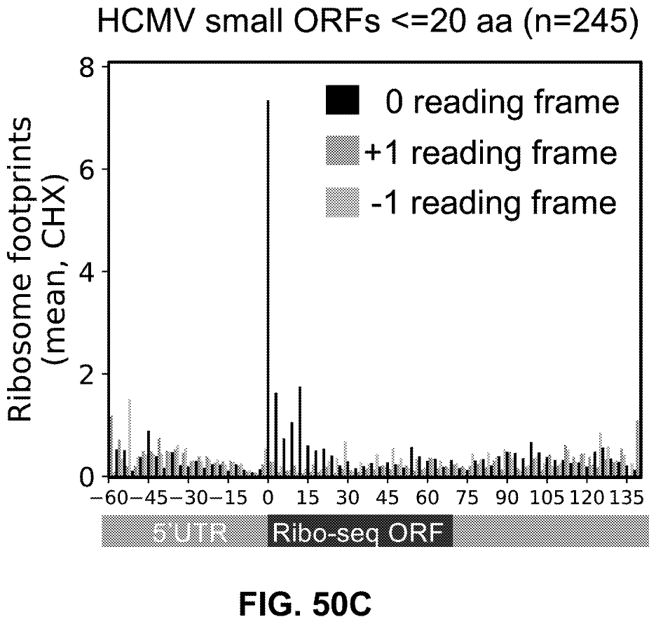

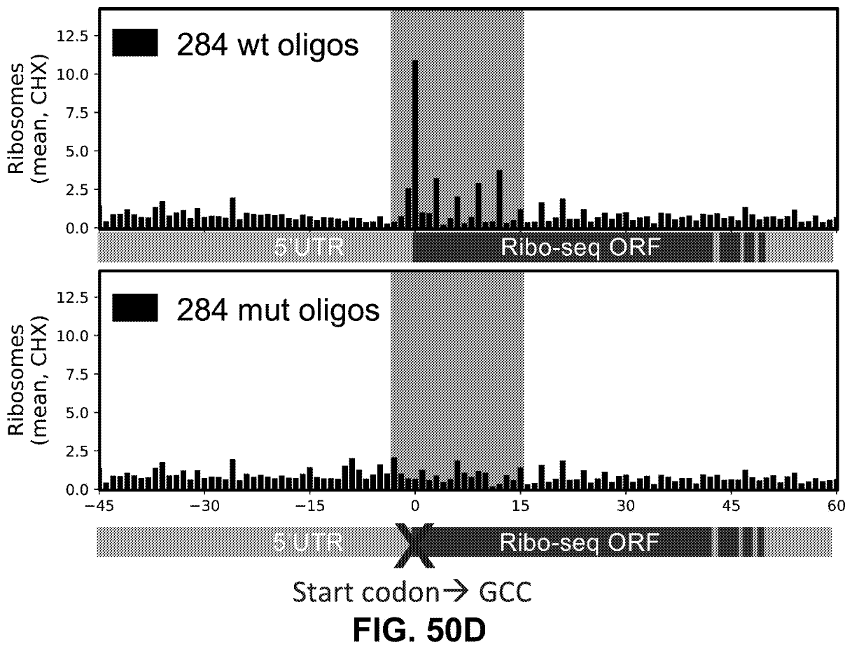

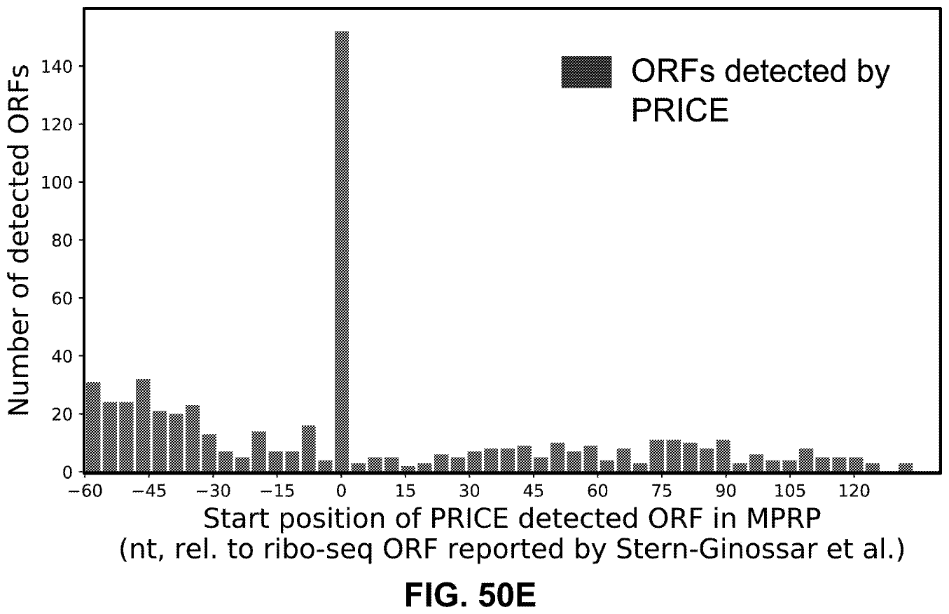

Similar to the annotated viral CDS, Applicant found clear enrichment of ribosome footprints along the reported Ribo-seq ORFs with tri-nucleotide periodicity indicating translation in the correct reading frame. Importantly, MPRP detected ribosome footprints on both canonical and non-canonical Ribo-seq ORFs, including ORFs with a non-AUG start codon (CUG, ACG, GUG, AUU, UUG, AUC, AUA, AGG, AAG) (FIG. 50A-50B). Moreover, MPRP successfully detected translating ribosomes on short ORFs, in the length of 20 aa or less (FIG. 50C).

Ribo-seq ORFs translation in the MPRP assay was dependent on the presence of the reported start codon. Mutating the start codon to GCC of 284 Ribo-seq ORFs (in the length of 7-45 aa) resulted in substantial reduction of ribosome footprints compared to wt oligos (FIG. 50D). Unlike annotated CDS that mostly initiate with a AUG start codon, the non-canonical Ribo-seq ORFs often initiate from a non-AUG codon. Our findings confirm that the non-AUG codons reported by Stern-Ginossar et al. are essential for translation initiation of the Ribo-seq ORFs and demonstrate how MPRP can be used to functionally characterize the start codon of non-canonical ORFs.

Finally, Applicant ran PRICE to assess the overlap between ORFs that were detected in the original ribosome profiling study and the MPRP experiment. Similar to the annotated CDS, the observed peak of the PRICE-detected ORF start codon matched the location of the Ribo-seq ORFs in the designed oligo, with 152 of the 716 Ribo-seq ORFs detected (21%) (false discovery rate 0.05, FIG. 50E). Altogether, Applicant shows that MPRP can capture both canonical and non-canonical ORFs that were identified by traditional ribosome profiling.

Expanding the Repertoire of Viral Antigens that are Presented on the HLA-I Complex

Microproteins that are encoded by non-canonical ORFs can contribute to the pool of viral antigens that are presented on the HLA-I complex. Our assay resulted in the discovery of 4,208 non-canonical ORFs, 3,686 of which encode proteins in the length of 8 aa or longer, making them candidates for HLA-I binding.

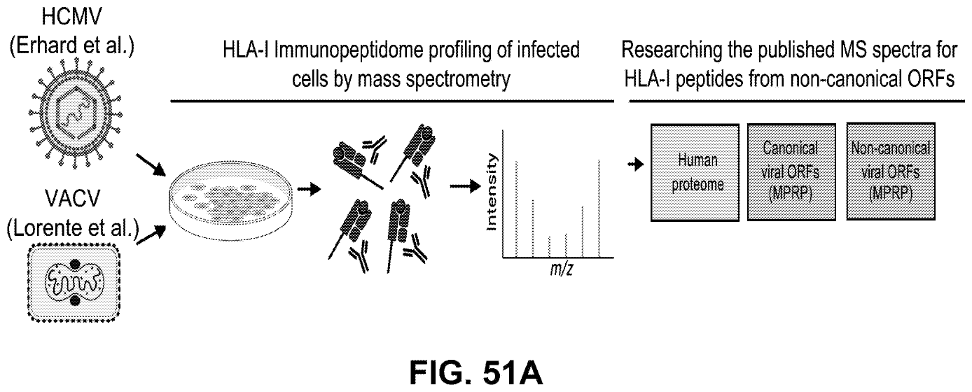

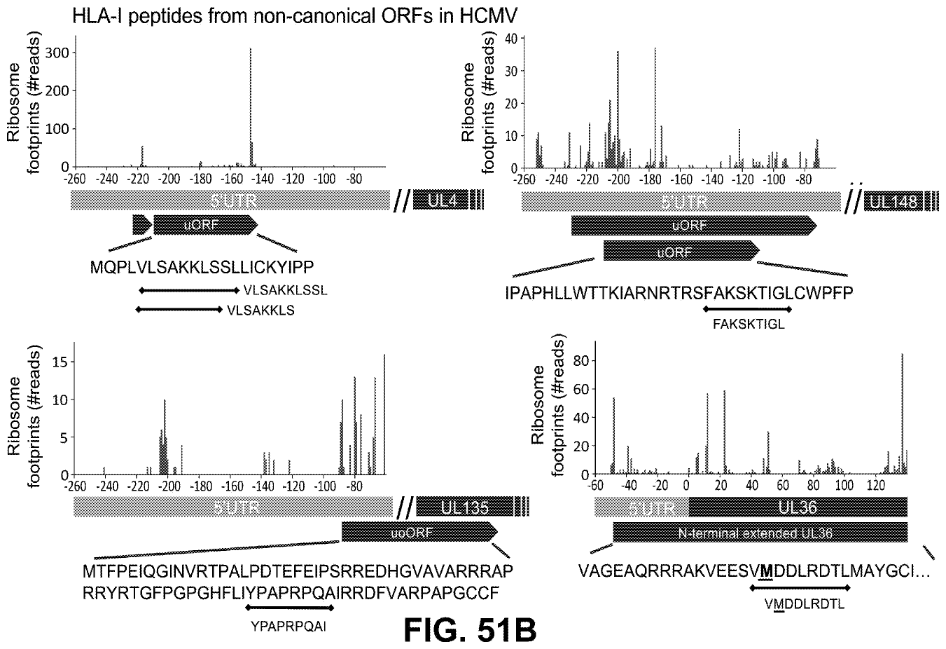

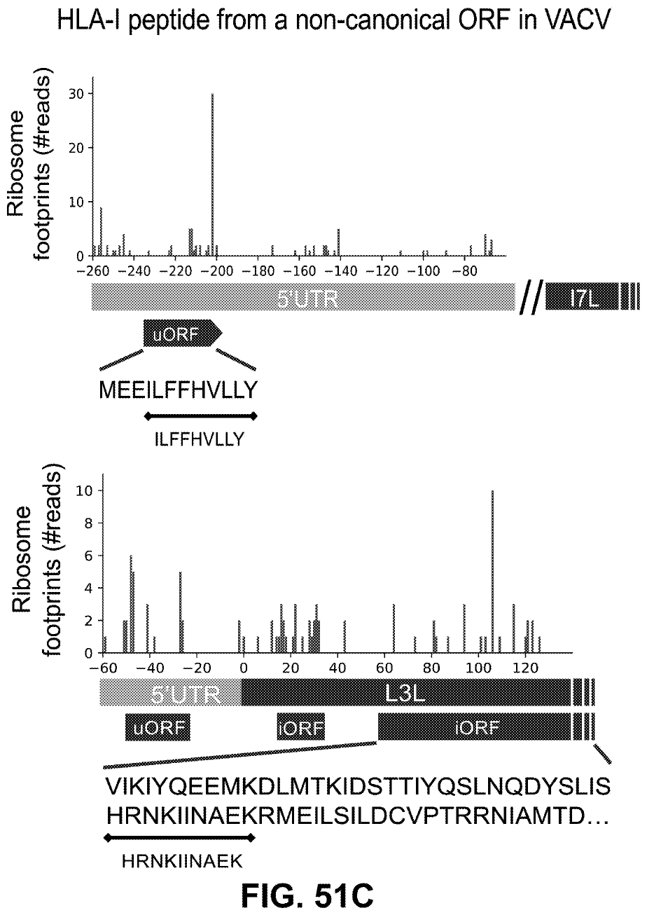

To assess if these non-canonical ORFs participate in HLA-I presentation, Applicant re-analyzed two HLA-I immunopeptidome datasets from cells that were infected with either HCMV (Erhard et al. 2018) or Vaccinia virus (VACV) (Lorente et al. 2017). In the two studies, the HLA-I complex was immunoprecipitated from infected cells using HLA specific antibodies and bound peptides were identified using mass spectrometry (MS) (FIG. 51A). For the reanalysis, Applicant appended the non-canonical proteins that were detected in HCMV or VACV in the MPRP assay to the canonical human proteome database and used this combined database to research the raw mass spectrometry files (Erhard et al. 2018; Lorente et al. 2017). Applicant detected HLA-I peptides that originated from non-canonical ORFs in the HCMV and VACV genomes (Table 3). See also e.g., Weingarten-Gabbay et al., 2023. bioRxiv https://doi.org/10.1101/2023.09.26.55964, which is incorporated by reference herein, particularly at Table S2. Overall, Applicant found five potential HLA-I peptides from four non-canonical ORFs in HCMV: an uORF in the 5′UTR of UL4 (VLSAKKLS (SEQ ID NO: 16288) (A0301;MSi rank 4.9)), and (VLSAKKLSSL (SEQ ID NO: 16287) (C0102; MSi rank 0.04)), an uORF in the 5′UTR of UL148 (FAKSKTIGL (SEQ ID NO: 16292) (B0801; MSi rank 0.02)), an Upstream Overlapping ORF (uoORF) in the 5′UTR and coding region of UL135 (YPAPRPQAI (SEQ ID NO: 16295) (B0801,B5101; MSi rank 0.5)), and an N-terminal extended isoform of the UL36 protein (VMDDLRDTL (SEQ ID NO: 16297) (C0102; MSi rank 2.0)) (FIG. 51B). Applicant noted detection of LSAKKLSSL (SEQ ID NO: 16289) and SAKKLSSL (SEQ ID NO: 16290) that appear to be in-source fragments of VLSAKKLSSL (SEQ ID NO: 16287), and not HLA-I binding peptides, as they elute within 0.1 minutes of each other and do not have strong HLAthena binding predictions. In VACV-infected cells, Applicant found two HLA-I peptides from an uORR in the 5′UTR of7L (ILFFHVLLY (SEQ ID NO: 16299) (A0301;MSi rank 0.01)) and from internal ORF overlapping the coding region of LUL (HIRNKIINAEK (SEQ ID NO: 16316) (1B2705; MSi rank 0.6)) (FIG. 51C). These observations confirm that non-canonical ORFs identified by MPRP can be translated in the context of native viral infection and that the resulting proteins were presented on the HLA-I complex.

TABLE 3
HLA-I peptides originating from non-canonical ORFs in HCMV and VACV

| directory | | | | | | |
|---|---|---|---|---|---|---|
| | parent_ | numPSMs | numLCruns | | rank | deltaForward |
| | charge | Observed | Observed | score | 2Score | ReverseScore |
| HF997_ | 2 | 3 | 3 | 12.98 | 4.96 | 9.95 |
| HCMVtreated | | | | | | |
| HF997_ | 2 | 11 | 3 | 16.25 | 12.32 | 3.24 |
| HCMVtreated | | | | | | |
| HF997_ | 2 | 2 | 2 | 14.76 | 8.97 | 4.15 |
| HCMVtreated | | | | | | |
| HF997_ | 2 | 1 | 1 | 8.99 | 7.49 | 0.45 |
| HCMVtreated | | | | | | |
| HF997_ | 2 | 32 | 3 | 18.4 | 9.48 | 8.29 |
| HCMVtreated | | | | | | |
| HF997_ | 2 | 17 | 3 | 14.89 | 8.11 | 9.07 |
| HCMVtreated | | | | | | |
| HF997_ | 2 | 5 | 3 | 15.69 | 7.37 | 6.8 |
| HCMVtreated | | | | | | |
| HOM2_w632_ | 2 | 1 | 1 | 14.31 | 8.99 | 4.76 |
| A03_C01_ | | | | | | |
| VACV | | | | | | |
| HOM2_B27_ | 3 | 1 | 1 | 8.48 | 3.59 | 8.48 |
| VACV | | | | | | |
| | | percent_ | backbone_ | | | |
| | deltaRank1 | scored_peak_ | cleavage_ | total | previous_ | |
| | Rank2Score | intensity | score | Intensity | aa | sequence |
| HF997_ | 8.01 | 72 | 7 | 1.33E+07 | (S) | FAKSKTIGL |
| HCMVtreated | | | | | | (SEQ ID NO: |
| | | | | | | 16292) |
| HF997_ | 3.92 | 76.8 | 8 | 8.94E+07 | (V) | [symbol] |
| HCMVtreated | | | | | | (SEQ ID NO: |
| | | | | | | 16289) |
| HF997_ | 5.79 | 86.9 | 6 | 4.42E+07 | (L) | [symbol] |
| HCMVtreated | | | | | | (SEQ ID NO: |
| | | | | | | 16290) |
| HF997_ | 1.49 | 39.2 | 5 | 1.68E+07 | (L) | VLSAKKLS |
| HCMVtreated | | | | | | (SEQ ID NO: |
| | | | | | | 16288) |
| HF997_ | 8.91 | 92.5 | 9 | 3.05E+09 | (L) | VLSAKKLSSL |
| HCMVtreated | | | | | | (SEQ ID NO: |
| | | | | | | 16287) |
| HF997_ | 6.77 | 79 | 6 | 2.64E+08 | (I) | YPAPRPQAI |
| HCMVtreated | | | | | | (SEQ ID NO: |
| | | | | | | 16295) |
| HF997_ | 8.32 | 88.3 | 8 | 4.51E+07 | (S) | VMDDLRDTL |
| HCMVtreated | | | | | | (SEQ ID NO: |
| | | | | | | 16297) |
| HOM2_w632_ | 5.33 | 80.7 | 6 | 9.92E+07 | (E) | ILFFHVLLY |
| A03_C01_ | | | | | | (SEQ ID NO: |
| VACV | | | | | | 16299) |
| HOM2_B27_ | 4.89 | 43.2 | 6 | 4.79E+07 | (S) | HRNKIINAEK |
| VACV | | | | | | (SEQ ID NO: |
| | | | | | | 16316) |
| | | sequence | retention | sequence | best. | best.MSi_ |
| | next_aa | Length | TimeMin | Map | MSi | allele |
| HF997_ | (C) | 9 | 25.68 | F/A\|K/S\| | 0.016324 | B0801 |
| HCMVtreated | | | | K\T I\|G- | | |
| | | | | L | | |
| HF997_ | (L) | 9 | 31.75 | L/S\|A/K/ | 1.238008 | B0801 |
| HCMVtreated | | | | K\L\S\|S\ | | |
| | | | | L | | |
| HF997_ | (L) | 8 | 31.75 | S/A\|K/K | 2.013671 | C0102 |
| HCMVtreated | | | | L\S\|S\L | | |
| HF997_ | (S) | 8 | 15.7 | V/L\|S/A/ | 4.91671 | A0301 |
| HCMVtreated | | | | K K L\S | | |
| HF997_ | (L) | 10 | 31.85 | V/L\|S\|A/ | 0.043597 | C0102 |
| HCMVtreated | | | | K/K\L\S\| | | |
| | | | | S\L | | |
| HF997_ | (R) | 9 | 36.37 | Y/P\|A\|P/ | 0.054413 | B0801, |
| HCMVtreated | | | | R P | | B5101 |
| | | | | Q\A\I | | |
| HF997_ | (M) | 9 | 51.32 | V/M\|D\|D\| | 2.000613 | C0102 |
| HCMVtreated | | | | L\|R\D\T\ | | |
| | | | | L | | |
| HOM2_w632_ | (—) | 9 | 133.32 | I/L\|F F | 0.010677 | A0301 |
| A03_C01_ | | | | H\V\L\|L\| | | |
| VACV | | | | Y | | |
| HOM2_B27_ | (R) | 10 | 14.95 | H/R N | 0.595316 | B2705 |
| VACV | | | | K\I\|I/N | | |
| | | | | A\E\|K | | |

| | in source | |
|---|---|---|
| | frag | Comment |
| HF997_ | | |
| HCMVtreated | | |
| HF997_ | in source | appear to be in-source fragments of VLSAKKLSSL |
| HCMVtreated | fragment | (SEQ ID NO: 16287), and not HLA-I binding peptides, |
| | | as they elute within 0.1 minutes of each other |
| | | and do not have strong HLAthena binding predictions |
| HF997_ | in source | appear to be in-source fragments of VLSAKKLSSL |
| HCMVtreated | fragment | (SEQ ID NO: 16287), and not HLA-I binding peptides, |
| | | as they elute within 0.1 minutes of each other |
| | | and do not have strong HLAthena binding predictions |
| HF997_ | | |
| HCMVtreated | | |
| HF997_ | in source | |
| HCMVtreated | parent | |
| HF997_ | | |
| HCMVtreated | | |
| HF997_ | | |
| HCMVtreated | | |
| HOM2_w632_ | | |
| A03_C01_ | | |
| VACV | | |
| HOM2_B27_ | | |
| VACV | | |

Exposing uORFs that Regulate the Translation of Canonical Viral Proteins

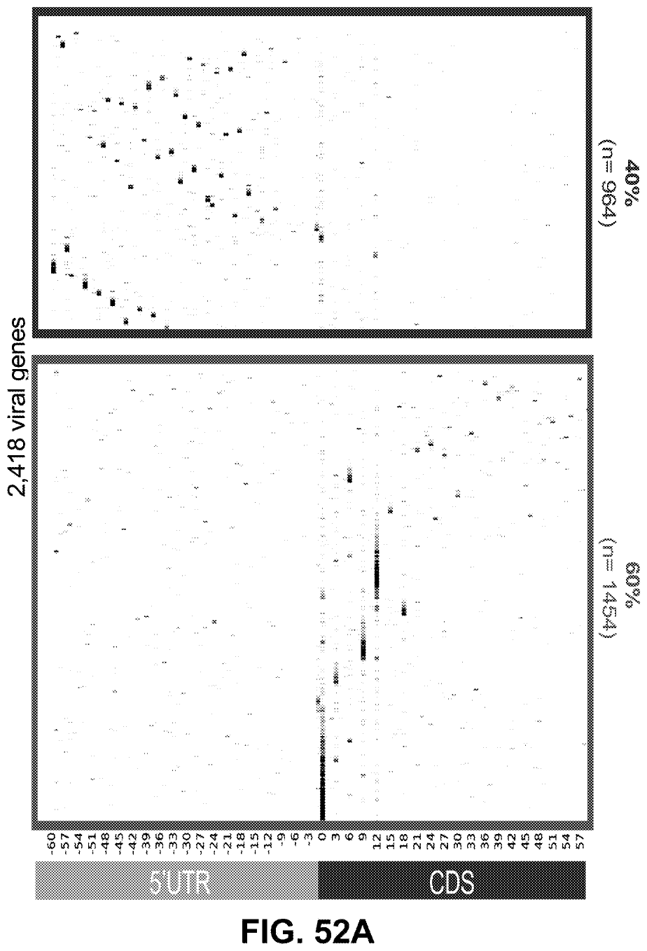

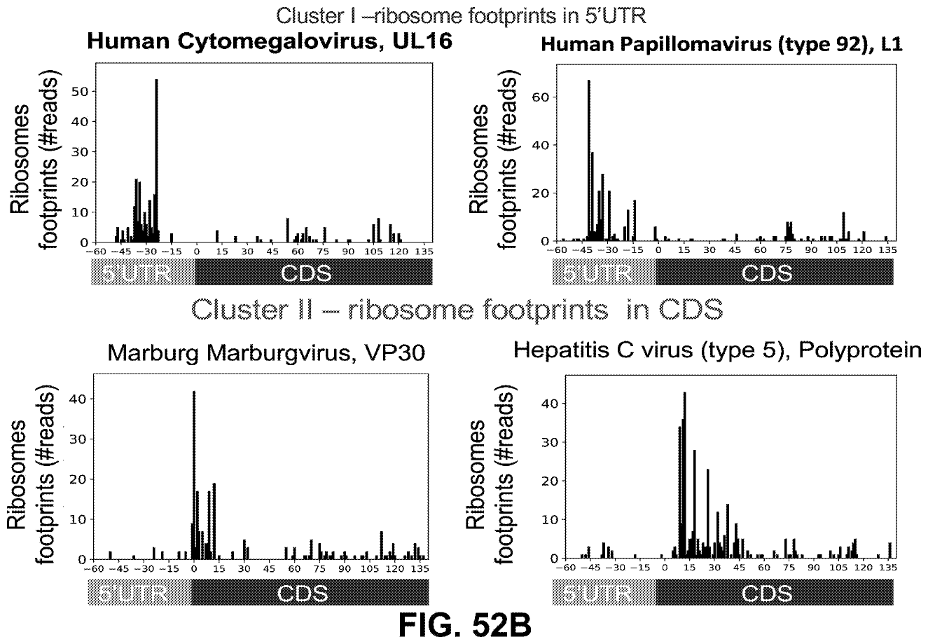

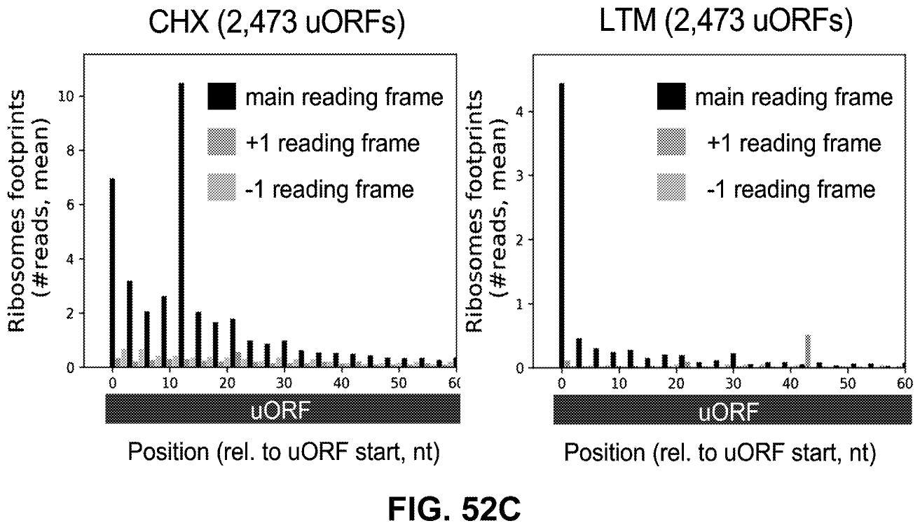

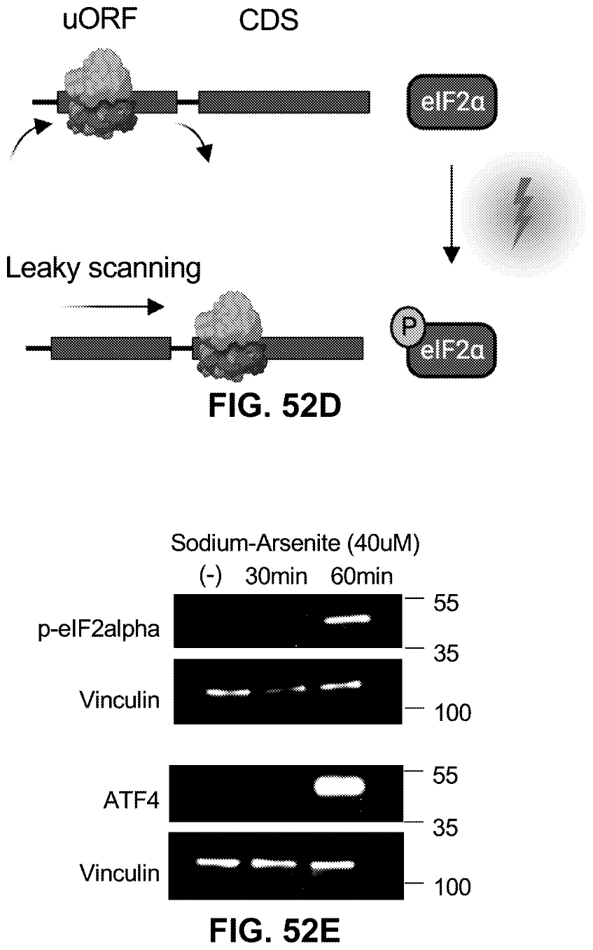

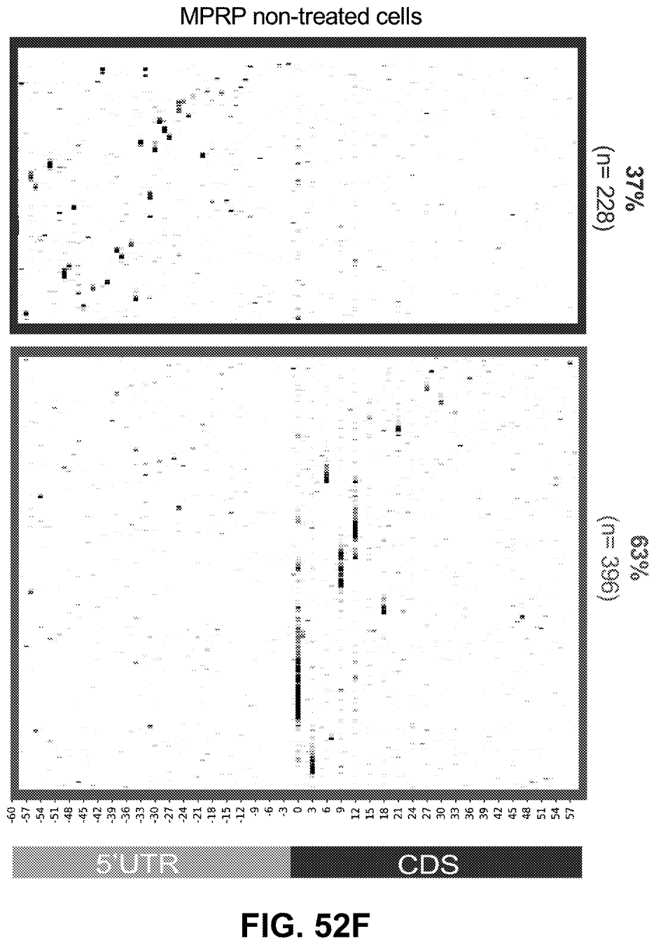

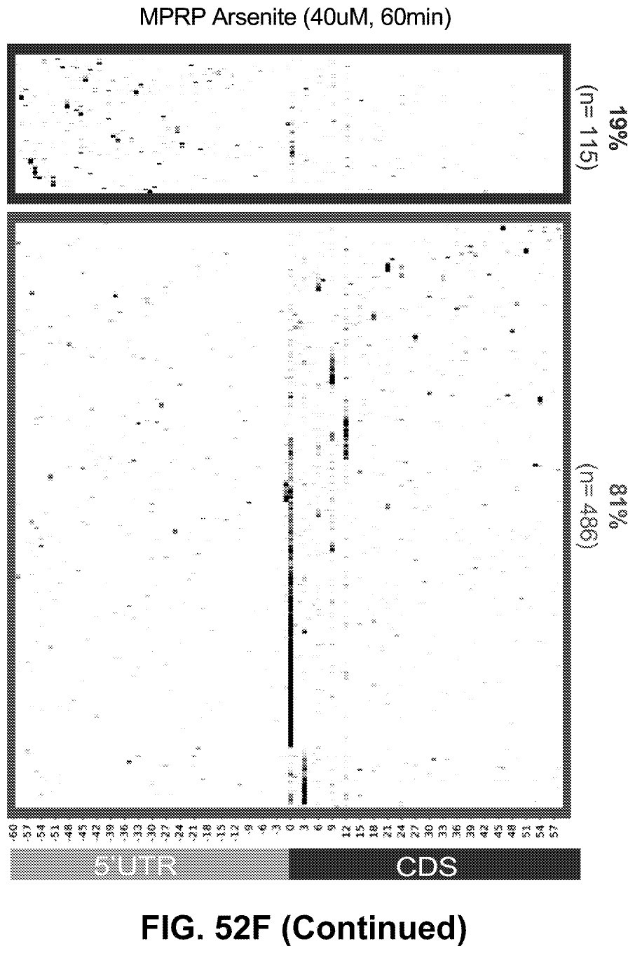

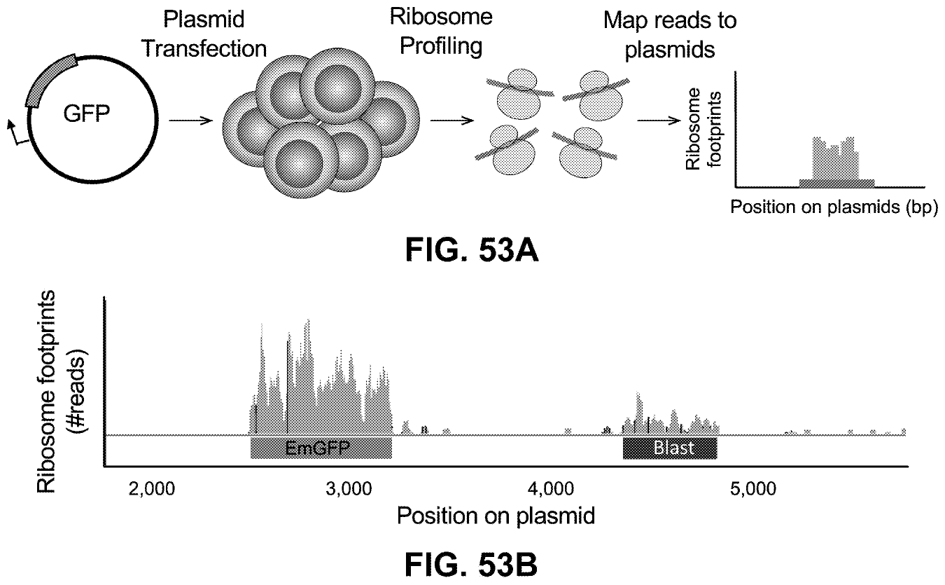

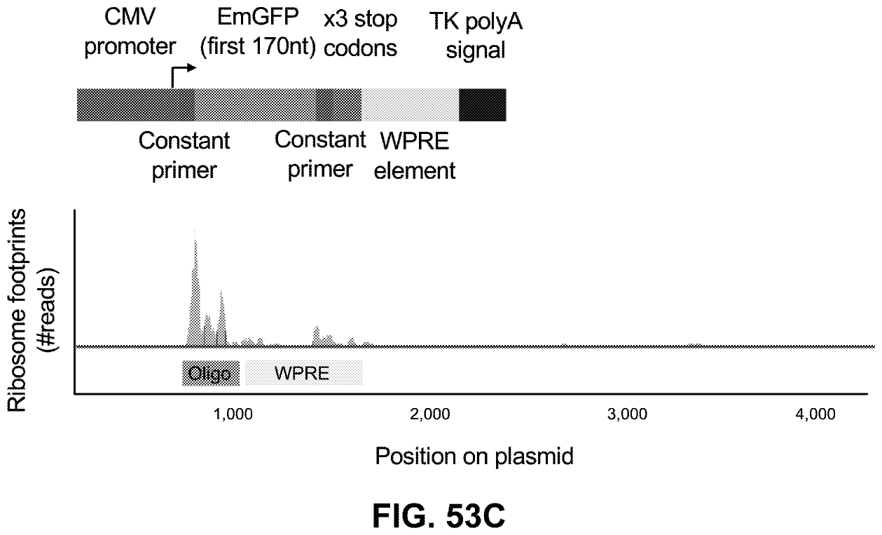

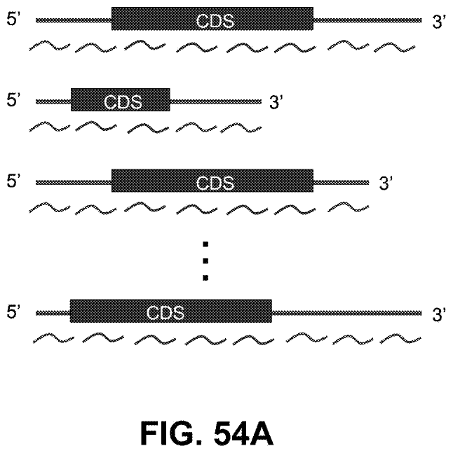

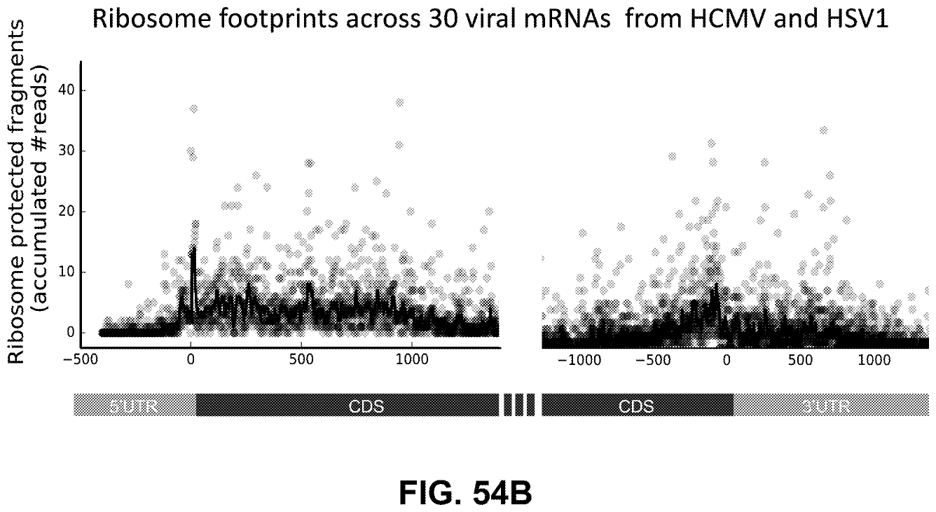

In addition to a list of canonical and non-canonical ORFs, our assay provides a detailed view of ribosome footprints across thousands of viral genes that can be harnessed to study translational regulation. Examining the distribution of ribosome footprints in 2,418 viral genes, Applicant identified two main clusters: a group of genes in which most of the footprints were observed in the 5′UTR with low occupancy in the CDS region (5′UTR cluster), and a group of genes in which most of the footprints were detected in the CDS with no evidence for ribosomes in the 5′UTR (CDS cluster) (FIG. 52A-52B). The tri-nucleotide periodicity observed in uORFs that were detected in the 5′UTR region indicates that they are actively translated by ribosomes (FIG. 52C). Moreover, Applicant found a strong signal of initiating ribosomes at the start codons of these uORFs in the presence of an LTM inhibitor.

Applicant hypothesized that viral genes in the 5′UTR cluster have uORFs that attenuate translation initiation from the main CDS (FIG. 52D). To test if the translation of the detected uORFs is regulated by cellular stress, Applicant treated cells with Sodium Arsenite (NaAsO2), a potent inducer of eIF2alpha phosphorylation (Andreev et al. 2015). In agreement with Andreev et al., treating HEK293T cells with 40 μM Sodium Arsenite resulted in rapid phosphorylation of eIF2alpha after 60 minutes (FIG. 52E). Applicant also observed strong induction of the cellular ATF4 protein levels, a well-studied gene that is regulated by uORFs, confirming enhancement of main CDS translation upon Arsenite treatment.

Applicant set out to investigate the distribution of ribosome footprints across the pan-viral library in response to Arsenite treatment. Applicant transfected HEK293T cells with the library plasmid pool and treated cells with 40 μM Sodium Arsenite for 60 minutes prior ribosome profiling. Applicant found a relative decrease in the fraction of viral genes in which ribosomes were “held” at the 5′UTR (37% to 19% in untreated and treated cells, respectively, FIG. 52F) and an increase in the fraction of genes containing ribosome footprints in the CDS (63% to 81% in untreated and treated cells, respectively). This result indicates that in response to eIF2alpha phosphorylation, ribosomes were more likely to “bypass” the uORFs in the 5′UTR and initiate translation at the main CDS, as expected in the case of inhibitory uORFs. Together, MPRP exposed hundreds of potential uORF that negatively regulate the translation of viral proteins.

### Discussion

Applicant presents a method to comprehensively screen the genomes of many viruses for translated regions in a single pooled experiment. Using MPRP, Applicant uncovered thousands of “hidden” ORFs in viral genomes, expanding the repertoire of viral targets for immune response and vaccine design. The high resolution of ribosome footprints across canonical and non-canonical ORFs facilitated systematic interrogation of the relationships between ribosome occupancies on the 5′UTR and CDS regions and the identification of hundreds of uORFs that negatively regulate the synthesis of canonical viral proteins.

MPRP has the capacity to identify translated ORFs in a broad spectrum of viruses. Various viral families exhibit unique life cycles, utilizing diverse nucleic acids as their genetic material and employing distinct strategies for replication, RNA transcription, and ribosome recruitment. Furthermore, different viruses replicate within specific subcellular compartments, such as the nucleus, cytoplasm, and viral factories. Despite these variations, Applicant have successfully detected annotated CDSs from many families. Given that all known viruses are unequivocally reliant on the host translation machinery (Stern-Ginossar et al. 2019), their genomes have evolved to be recognized by the 80S ribosome. It is possible that these inherent sequence features facilitate translation initiation when the CDS is expressed from a synthetic construct in uninfected cells.

MPRP's capabilities hold particular significance in the context of researching highly pathogenic viruses, as our comprehension of their biology is limited due to the scarcity of available high-containment facilities. MPRP, employing 200 nt-long viral fragments, can be conducted in a BSL-2 setting, rendering it accessible to laboratories worldwide. Our study effectively identified annotated CDSs in families of viruses designated as high-priority pathogens with the potential for outbreaks and global pandemics by the World Health Organization (WHO). Evident signatures of elongating and initiating ribosomes, including tri-nucleotide periodicity, were found in Filoviridae (Ebola virus disease and Marburg virus disease), Paramyxoviridae (Nipah and henipaviral diseases), Arenaviridae (Lassa fever), Coronaviridae (Middle East respiratory syndrome {MERS} and Severe Acute Respiratory Syndrome {SARS}), and Flaviviridae (Zika virus) (FIGS. 49B, 56, and 57). Subsequent MPRP experiments have the potential to deepen our comprehension of their translational regulation and dependencies on host factors, which will inform future therapeutics.

This study unveiled a previously unexplored realm of viral antigens that could contribute to HLA-I presentation and T cell recognition. The identification of peptides in MS experiments relies on a predefined dataset and requires a list of the proteins present in the sample. Consequently, the lack of comprehensive information regarding the complete translatome of viral genomes hinders the detection of HLA-I peptides. It has been suggested that incorporating data from ribosome profiling could enhance the optimization of false- and failed-discovery rates (Holly and Yewdell 2023). Utilizing the list of ORFs detected by our assay, Applicant revealed HLA-I peptides originating from non-canonical ORFs in HCMV and VACV (FIG. 51A-51C). As researchers generate immunopeptidome data for other viruses, our pan-viral non-canonical ORFs list can serve as a resource for the community, enabling rapid exploration of the new immunopeptidomes. Furthermore, these non-canonical ORFs can also be integrated into the design of peptide pools for T cell assays. While T cell assays almost exclusively assess responses against canonical proteins, Applicant recently demonstrated that some T cell epitopes from non-canonical ORFs induce more potent T cell responses compared to canonical epitopes (Weingarten-Gabbay et al. 2021). Thus, the incorporation of non-canonical ORFs into T cell assays has the potential to enhance their sensitivity and facilitate the identification of vaccine targets.

Applicant reveals here an unprecedented number of viral uORFs that likely play a role in gene expression regulation. Proper temporal expression of viral proteins is crucial for their life cycle. For instance, viruses must inhibit host antiviral genes before genome replication to avoid innate immune responses. Structural protein synthesis and virion assembly usually follow sufficient genome replication. Viruses employ ‘transcriptional programs’ for ‘early,’ ‘intermediate,’ and ‘late’ genes, often using distinct promoters or controlling transcription levels through the arrangements of genes along their genome. Recent research highlights translation control, particularly uORFs, in temporal viral gene expression regulation. Enrichment of uORFs in late genes of HHV-6 and HCMV suggests roles in temporal gene control (Finkel et al. 2020). However, unlike transcription-related mechanisms, uORF-mediated gene regulation in viruses remains less understood. Our study uncovers numerous potential uORFs in viral genomes, exhibiting signs of genuine translation, including tri-nucleotide periodicity and enriched initiating ribosomes at uORF start codons (FIG. 52C). Notably, many of these uORFs respond to eIF2alpha phosphorylation, resulting in enhanced translation of the primary CDS. Given that eIF2alpha phosphorylation levels rise during infection, the expression of viral genes controlled by these uORFs would be augmented in the later stages of infection. This suggests their function as cis-regulatory elements of ‘late’ genes.

It is important to acknowledge the limitations inherent in this study. The viral sequences examined here were assessed independently of the broader genome context, and were evaluated in non-infected cells. MPRP does not capture the distinctive biology occurring within cells infected by each of the many viruses studied here. Thus, it is plausible that our assay lacks host and/or viral proteins necessary for the translation of certain ORFs. While the results from this study should be interpreted with the caution typical of high-throughput screens, there is substantive evidence supporting the identification of genuine ORFs. This evidence arises from the anticipated signature of elongating and initiating ribosomes, the reproducibility of measurements across different cell types, the notable decrease in ribosome footprints upon mutating AUG and non-AUG start codons, the corroborative support from HLA-I peptides identified through mass spectrometry, and the responsiveness of uORFs to stress conditions. In total, our study yields numerous promising candidates for unexplored viral ORFs, which can enhance our understanding of viral biology and contribute to vaccine development.

### Methods

### ORF Discovery Using PRICE

Applicant used PRICE to identify ORFs from deep sequencing reads (Erhard et al. 2018). PRICE algorithm requires a predefined set of annotated CDSs in order to estimate the codons that have generated the observed ribosome footprints. When providing the reference genome of the synthetic library, Applicant did not indicate which oligo contains an annotated viral CDS, because Applicant used this information to estimate ORF discovery rate. Instead, Applicant used annotated human CDSs that were translated in library-transfected cells and thus, were exposed to the exact same experimental conditions (e.g., RNaseI concentration and incubation time that can impact footprints size). Applicant generated a chimeric reference genome composed of chromosome 1 in hg19 and an artificial chromosome composed of 15,000 oligos of the pan-viral library. In addition, Applicant generated a gtf file with the annotations of hg19 and library oligos required for PRICE predictions. For each experiment, Applicant mapped deep sequencing reads to the chimeric fasta file using Bowtie. Applicant ran PRICE with bam files from all the experiments done in HEK293T cells. Applicant filtered ORFs a p-value <=0.05 after correcting for false discovery rate (FDR). ORFs were then defined by extending each initiating codon to the next in-frame stop codon in the corresponding viral genome.

### Tri-Nucleotide Periodicity Analysis

To determine the reading frame, Applicant used ribosome footprints in the exact length of 29 nt and plotted the position of the first nt of the sequencing read. Positions were corrected for the P site offset (12 nt).

### Re-Analysis of HLA-I Immunopeptidome Datasets

Parameters for the Spectrum Mill MS/MS search module for HLA-I immunopeptidomes reported in (Lorente et al. 2017), (Lorente et al. 2017) included: no enzyme specificity; precursor and product mass tolerance of ±10 ppm; minimum matched peak intensity of 30%; ESI-ORBITRAP-CID-HLA-v3 scoring; fixed modification: cysteinylation of cysteine; variable modifications: oxidation of methionine, deamidation of asparagine, acetylation of protein N-termini, and pyroglutamic acid at peptide N-terminal glutamine; and precursor mass shift range of −18 to 136 Da.

Parameters for the Spectrum Mill MS/MS search module for HLA-I immunopeptidomes reported in Erhard et al., (Erhard et al. 2018) included: no enzyme specificity; precursor and product mass tolerance of ±10 ppm; minimum matched peak intensity of 30%; ESI-QEXACTIVE-HCD-HLA-v3 scoring; variable modifications: cysteinylation of cysteine; oxidation of methionine, deamidation of asparagine, acetylation of protein N-termini, and pyroglutamic acid at peptide N-terminal glutamine; and precursor mass shift range of −18 to 136 Da.

### HLAthena HLA-I Peptide Presentation Predictions

HLA peptide prediction was performed using HLAthena (Sarkizova et al. 2020). For HCMV infected HF99-7, HLA A*01:01, A*03:01, B*08:01, -B*51:01, C*07:01, C*01:02 were used for HLAthena predictions (Erhard et al. 2018). For VACV-infected HOM-2 cells, HLA A*03:01, B*27:05:02, and C*01:02 were used for HLAthena predictions (Lorente et al. 2017).

### REFERENCES FOR EXAMPLE 3

Andreev, Dmitry E., Patrick B. F. O'Connor, Ciara Fahey, Elaine M. Kenny, Ilya M. Terenin, Sergey E. Dmitriev, Paul Cormican, Derek W. Morris, Ivan N. Shatsky, and Pavel V. Baranov. 2015. “Translation of 5′ Leaders Is Pervasive in Genes Resistant to eIF2 Repression.” eLife 4 (January): e03971.
Bansal, Anju, Jonathan Carlson, Jiyu Yan, Olusimidele T. Akinsiku, Malinda Schaefer, Steffanie Sabbaj, Anne Bet, et al. 2010. “CD8 T Cell Response and Evolutionary Pressure to HIV-1 Cryptic Epitopes Derived from Antisense Transcription.” The Journal of Experimental Medicine 207 (1): 51-59.
Chen, Augustine, Y. F. Kao, and Chris M. Brown. 2005. “Translation of the First Upstream ORF in the Hepatitis B Virus Pregenomic RNA Modulates Translation at the Core and Polymerase Initiation Codons.” Nucleic Acids Research 33 (4): 1169-81.
Chen, Jin, Andreas-David Brunner, J. Zachery Cogan, James K. Nunez, Alexander P. Fields, Britt Adamson, Daniel N. Itzhak, et al. 2020. “Pervasive Functional Translation of Noncanonical Human Open Reading Frames.” Science 367 (6482): 1140-46.
Erhard, Florian, Anne Halenius, Cosima Zimmermann, Anne L'Hernault, Daniel J. Kowalewski, Michael P. Weekes, Stefan Stevanovic, Ralf Zimmer, and Lars Dolken. 2018. “Improved Ribo-Seq Enables Identification of Cryptic Translation Events.” Nature Methods 15 (5): 363-66.
Finkel, Yaara, Dominik Schmiedel, Julie Tai-Schmiedel, Aharon Nachshon, Roni Winkler, Martina Dobesova, Michal Schwartz, Ofer Mandelboim, and Noam Stern-Ginossar. 2020. “Comprehensive Annotations of Human Herpesvirus 6A and 6B Genomes Reveal Novel and Conserved Genomic Features.” eLife 9 (January). https://doi.org/10.7554/eLife.50960.
Gould, Phillip S., and Andrew J. Easton. 2005. “Coupled Translation of the Respiratory Syncytial Virus M2 Open Reading Frames Requires Upstream Sequences.” The Journal of Biological Chemistry 280 (23): 21972-80.
Hickman, Heather D., Jacqueline W. Mays, James Gibbs, Ivan Kosik, Javier G. Magadin, Kazuyo Takeda, Suman Das, et al. 2018. “Influenza A Virus Negative Strand RNA Is Translated for CD8+ T Cell Immunosurveillance.” Journal of Immunology 201 (4): 1222-28.
Hinnebusch, Alan G., Ivaylo P. Ivanov, and Nahum Sonenberg. 2016. “Translational Control by 5′-Untranslated Regions of Eukaryotic mRNAs.” Science 352 (6292): 1413-16.
Holly, Jaroslav, and Jonathan W. Yewdell. 2023. “Game of Omes: Ribosome Profiling Expands the MHC-I Immunopeptidome.” Current Opinion in Immunology 83 (May): 102342.
Ingolia, Nicholas T., Gloria A. Brar, Noam Stern-Ginossar, Michael S. Harris, Gaëlle J. S. Talhouarne, Sarah E. Jackson, Mark R. Wills, and Jonathan S. Weissman. 2014. “Ribosome Profiling Reveals Pervasive Translation outside of Annotated Protein-Coding Genes.” Cell Reports 8 (5): 1365-79.
Ingolia, Nicholas T., Sina Ghaemmaghami, John R. S. Newman, and Jonathan S. Weissman. 2009. “Genome-Wide Analysis in Vivo of Translation with Nucleotide Resolution Using Ribosome Profiling.” Science 324 (5924): 218-23.
Ingolia, Nicholas T., Jeffrey A. Hussmann, and Jonathan S. Weissman. 2019. “Ribosome Profiling: Global Views of Translation.” Cold Spring Harbor Perspectives in Biology 11 (5). https://doi.org/10.1101/cshperspect.a032698.
Ingolia, Nicholas T., Liana F. Lareau, and Jonathan S. Weissman. 2011. “Ribosome Profiling of Mouse Embryonic Stem Cells Reveals the Complexity and Dynamics of Mammalian Proteomes.” Cell 147 (4): 789-802.
Lorente, Elena, Alejandro Barriga, Juan Garcia-Arriaza, Frangois A. Lemonnier, Mariano Esteban, and Daniel López. 2017. “Complex Antigen Presentation Pathway for an HLA-A*0201-Restricted Epitope from Chikungunya 6K Protein.” PLoS Neglected Tropical Diseases 11 (10): e0006036.
Lulla, Valeria, Adam M. Dinan, Myra Hosmillo, Yasmin Chaudhry, Lee Sherry, Nerea Irigoyen, Komal M. Nayak, et al. 2019. “An Upstream Protein-Coding Region in Enteroviruses Modulates Virus Infection in Gut Epithelial Cells.” Nature Microbiology 4 (2): 280-92.
Lulla, Valeria, and Andrew E. Firth. 2020. “A Hidden Gene in Astroviruses Encodes a Viroporin.” Nature Communications 11 (1): 4070.
Maness, Nicholas J., Andrew D. Walsh, Shari M. Piaskowski, Jessica Furlott, Holly L. Kolar, Alexander T. Bean, Nancy A. Wilson, and David I. Watkins. 2010. “CD8+ T Cell Recognition of Cryptic Epitopes Is a Ubiquitous Feature of AIDS Virus Infection.” Journal of Virology 84 (21): 11569-74.
McGlincy, Nicholas J., and Nicholas T. Ingolia. 2017. “Transcriptome-Wide Measurement of Translation by Ribosome Profiling.” Methods 126 (August): 112-29.
Ogden, Pierce J., Eric D. Kelsic, Sam Sinai, and George M. Church. 2019. “Comprehensive AAV Capsid Fitness Landscape Reveals a Viral Gene and Enables Machine-Guided Design.” Science 366 (6469): 1139-43.
Ouspenskaia, Tamara, Travis Law, Karl R. Clauser, Susan Klaeger, Siranush Sarkizova, Frangois Aguet, Bo Li, et al. 2022. “Unannotated Proteins Expand the MHC—I-Restricted Immunopeptidome in Cancer.” Nature Biotechnology 40 (2): 209-17.
Ruiz Cuevas, Maria Virginia, Marie-Pierre Hardy, Jaroslav Holly, Eric Bonneil, Chantal Durette, Mathieu Courcelles, Joel Lanoix, et al. 2021. “Most Non-Canonical Proteins Uniquely Populate the Proteome or Immunopeptidome.” Cell Reports 34 (10): 108815.
Sarkizova, Siranush, Susan Klaeger, Phuong M. Le, Letitia W. Li, Giacomo Oliveira, Hasmik Keshishian, Christina R. Hartigan, et al. 2020. “A Large Peptidome Dataset Improves HLA Class I Epitope Prediction across Most of the Human Population.” Nature Biotechnology 38 (2): 199-209.
Seo, Jenny J., Soo-Jin Jung, Jihye Yang, Da-Eun Choi, and V. Narry Kim. 2023. “Functional Viromic Screens Uncover Regulatory RNA Elements.” Cell 186 (15): 3291-3306.e21.
Shabman, Reed S., Thomas Hoenen, Allison Groseth, Omar Jabado, Jennifer M. Binning, Gaya K. Amarasinghe, Heinz Feldmann, and Christopher F. Basler. 2013. “An Upstream Open Reading Frame Modulates Ebola Virus Polymerase Translation and Virus Replication.” PLoS Pathogens 9 (1): e1003147.
Starck, Shelley R., and Nilabh Shastri. 2016. “Nowhere to Hide: Unconventional Translation Yields Cryptic Peptides for Immune Surveillance.” Immunological Reviews 272 (1): 8-16.
Stern-Ginossar, Noam, Sunnie R. Thompson, Michael B. Mathews, and Ian Mohr. 2019. “Translational Control in Virus-Infected Cells.” Cold Spring Harbor Perspectives in Biology 11 (3). https://doi.org/10.1101/cshperspect.a033001.
Stern-Ginossar, Noam, Ben Weisburd, Annette Michalski, Vu Thuy Khanh Le, Marco Y. Hein, Sheng-Xiong Huang, Ming Ma, et al. 2012. “Decoding Human Cytomegalovirus.” Science 338 (6110): 1088-93.
Tan, Evan, Cara Sze Hui Chin, Zhi Feng Sherman Lim, and Say Kong Ng. 2021. “HEK293 Cell Line as a Platform to Produce Recombinant Proteins and Viral Vectors.” Frontiers in Bioengineering and Biotechnology 9 (December): 796991.
Weingarten-Gabbay, Shira, Da-Yuan Chen, Siranush Sarkizova, Hannah B. Taylor, Matteo Gentili, Leah R. Pearlman, Matthew R. Bauer, et al. 2023. “The HLA-II Immunopeptidome of SARS-CoV-2.” bioRxiv. https://doi.org/10.1101/2023.05.26.542482.
Weingarten-Gabbay, Shira, Shani Elias-Kirma, Ronit Nir, Alexey A. Gritsenko, Noam Stern-Ginossar, Zohar Yakhini, Adina Weinberger, and Eran Segal. 2016. “Comparative Genetics. Systematic Discovery of Cap-Independent Translation Sequences in Human and Viral Genomes.” Science 351 (6270). https://doi.org/10.1126/science.aad4939.
Weingarten-Gabbay, Shira, Susan Klaeger, Siranush Sarkizova, Leah R. Pearlman, Da-Yuan Chen, Kathleen M. E. Gallagher, Matthew R. Bauer, et al. 2021. “Profiling SARS-CoV-2 HLA-I Peptidome Reveals T Cell Epitopes from out-of-Frame ORFs.” Cell 184 (15): 3962-80.e17.
Weingarten-Gabbay, Shira, Ronit Nir, Shai Lubliner, Eilon Sharon, Yael Kalma, Adina Weinberger, and Eran Segal. 2019. “Systematic Interrogation of Human Promoters.” Genome Research 29 (2): 171-83.
Yang, Ning, James S. Gibbs, Heather D. Hickman, Glennys V. Reynoso, Arun K. Ghosh, Jack R. Bennink, and Jonathan W. Yewdell. 2016. “Defining Viral Defective Ribosomal Products: Standard and Alternative Translation Initiation Events Generate a Common Peptide from Influenza A Virus M2 and M1 mRNAs.” Journal of Immunology 196 (9): 3608-17.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. A polynucleotide comprising one or more viral polynucleotides selected from SEQ ID NOS: 1-9890. or SEQ ID NOS: 9891-16277.
2. A vector comprising:
a synthetic library expression construct comprising:
   a short synthetic polynucleotide;
   a pair of constant primers flanking the short synthetic polynucleotide, the pair of constant primers comprising a forward constant primer and a reverse constant primer, wherein the forward constant primer and the reverse constant primer are each independently coupled to a 5′ end or a 3′ end of the short synthetic polynucleotide;
   a stop codon polynucleotide comprising one or more stop codons, wherein the stop codon polynucleotide is coupled to a constant primer coupled to the 3′ end of the short synthetic polynucleotide such that the constant primer coupled to the 3′ end of the short synthetic polynucleotide is between the stop codon polynucleotide and the short synthetic polynucleotide;
   a poly A signal, wherein the poly A signal is operably coupled to the short synthetic polynucleotide, the pair of constant primers, and the stop codon polynucleotide; and
   a promoter, wherein the promoter is operably coupled to the short synthetic polynucleotide, the pair of constant primers, and the stop codon polynucleotide, and the poly A signal.
3. The vector of aspect 2, wherein the short synthetic polynucleotide is about 200 nucleotides.
4. The vector of any one of aspects 2-3, wherein the short synthetic polynucleotide has a sequence corresponding to a region of a genome.
5. The vector of any one of aspects 2-4, wherein the short synthetic polynucleotide has a sequence corresponding to a region of a human genome, a non-human animal genome, a plant genome, a microbial genome, or a viral genome.
6. The vector of any one of aspects 2-5, wherein the short synthetic polynucleotide has a sequence corresponding to
   a. an annotated region of a genome;
   b. an unannotated region of a genome;
   c. a mutation;
   d. a 5′UTR;
   e. a 3′UTR;
   f. an open reading frame; or
   g. any combination thereof.
7. The vector of any one of aspects 2-6, wherein the short synthetic polynucleotide
   a. is a sequence according to a sequence in SEQ ID NOS: 1-9890 or SEQ ID NOS: 9891-16277;
   b. is a synthetic in silico designed sequence;
   c. does not have 100 percent identity to a genome sequence, optionally a human genome, a non-human animal genome, a plant genome, a microbial genome, or a viral genome;
   d. is not native to a genome; optionally a human genome, a non-human animal genome, a plant genome, a microbial genome, or a viral genome; or
   e. any combination thereof.
8. The vector of any one of aspects 2-7, wherein the vector comprises two or more synthetic library expression constructs.
9. The vector of aspect 8, wherein at least two of the two or more synthetic library expression constructs comprises a different short synthetic polynucleotide.
10. The vector of aspect 8, wherein each of the two or more synthetic library expression constructs comprises a different short synthetic polynucleotide.
11. The vector of any one of aspects 8-10, wherein at least two of the two or more synthetic library constructs comprises the same short synthetic polynucleotide.
12. The vector of any one of aspects 2-11, further comprising an additional regulatory element, a reporter gene, an origin of replication, a cloning sites, an internal ribosome entry sites, a transcription termination sequence, an inverted terminal repeat, a long terminal repeats, a trans-activating response elements, a central polypurine tract, a Psi element, a Rev response element, a packaging protein gene, a polymerase gene, an envelope protein gene, a capsid protein gene, a Rep protein gene, a U3 element, a repeat element (R), a unique 5′ element (U5), an untranslated region stabilization element, or any combination thereof.
13. The vector of any one of aspects 2-12, further comprising a Woodchuck hepatitis virus post-transcriptional regulatory element (WPRE), wherein the WPRE is operably coupled to the short synthetic polynucleotide and the pair of constant primers.
14. The vector of any one of aspects 2-13, wherein the vector is a eukaryotic expression vector.
15. The vector of any one of aspects 2-14, wherein the vector is a viral expression vector.
16. The vector of any one of aspects 2-15, wherein the promoter is a constitutive promoter.
17. The vector of any one of aspects 2-16, wherein the promoter is an inducible promoter or a conditional promoter.
18. A vector library comprising a plurality of vectors, wherein each vector of the plurality of vectors is as in any one of aspects 2-17.
19. The vector library of aspect 18, wherein at least two or more of the vectors of the plurality of vectors comprise different short synthetic polynucleotides.
20. The vector library of aspect 18, wherein each vector of the plurality of vectors comprises a different short synthetic polynucleotide.
21. The vector library of any one of aspects 18-19, wherein at least two of the vectors of the plurality of vectors comprise the same short synthetic polynucleotides.
22. A cell comprising a vector or a vector library as in any one of aspects 2-21.
23. The cell of aspect 22, wherein the cell is a eukaryotic cell or a prokaryotic cell.
24. A high through put method of determining translated sequences, the method comprising:
   expressing a vector or a vector library as in any one of aspects 2-21 in one or more cells under conditions sufficient to produce translation products;
   obtaining a sample of ribosomes comprising ribosome-protected mRNA fragments (RPFs) from the cells;
   recovering ribosome footprints consisting essentially of the RPFs from the sample of ribosomes; and
   determining a sequence of the RPFs thereby identifying translated sequences.
25. The method of aspect 24, wherein determining the sequence of the RPFs comprises nucleotide sequencing.
26. The method of aspect 25, wherein nucleotide sequencing comprises RNA sequencing.
27. The method of any one of aspects 25, wherein nucleotide sequencing comprises generating cDNA from the RPFs for form RPF cDNA and DNA sequencing the RPF cDNA.
28. The method of any one of aspects 24-27, further comprising digesting unprotected mRNA prior to recovering ribosome footprints.
29. The method of any one of aspects 24-28, further comprising removing rRNA from the sample containing ribosomes.
30. A polynucleotide identified using the method as in any one of aspects 24-29.
31. A polynucleotide comprising one or more viral polynucleotides selected from those polynucleotides of aspect 30 and SEQ ID NOS: 1-9890. or SEQ ID NOS: 9891-16277.
32. A vector comprising the polynucleotide of any one of aspects 1 or 30-31.
33. The vector of aspect 32, wherein the vector is an expression vector.
34. The vector of any one of aspects 32-33, wherein the vector is a eukaryotic vector, prokaryotic vector, or viral vector.
35. A polypeptide encoded by the polynucleotide of any one of aspects 1 or 30-31 and/or the vector as in any one of aspects 32-34.
36. A delivery vehicle comprising: the polynucleotide of any one of aspects 1 or 30-31, a vector as any one of aspects 32-34, the polypeptide as in aspect 35, or any combination thereof.
37. The delivery vehicle of aspect 36, wherein the delivery vehicle comprises a particle, lipid particle, liposome, exosome, virus particle, virus-like particle, capsid, cell penetrating peptide, DNA nanoclew, supercharged protein, self-assembling nanoparticle, spherical nucleic acid, streptolysin, lipoplex, polyplex, sugar-based particle, stable nucleic-acid particles, mRNA vaccine, and any combination thereof.
38. A cell comprising:
   the polynucleotide of any one of aspects 1 or 30-31, a vector as any one of aspect 32-34, the polypeptide as in aspect 35, the delivery vehicle of any one of aspects 36-37, or any combination thereof.
39. An immunogenic composition comprising:
   the polynucleotide of any one of aspects 1 or 30-31, a vector as any one of aspects 32-34, the polypeptide as in aspect 35, the delivery vehicle of any one of aspects 36-37, the cell of aspect 38, or any combination thereof.
40. The immunogenic composition of aspect 39, wherein the polynucleotide of any one of aspects 1 or 30-31 and/or the polypeptide as in aspect 25 is stimulating a B-cell and/or T-cell response in a subject to which it is delivered.
41. The immunogenic composition of aspect 40, wherein the B-cell response comprises antibody production.
42. A therapeutic composition comprising:
   the immunogenic composition of any one of aspects 39-41; and
   an anti-viral therapeutic.
43. The immunogenic or therapeutic composition of any one of aspects 39-42, wherein the one or more polynucleotides is a synthetic mRNA vaccine.
44. A formulation comprising:
   the polynucleotide of any one of aspects 1 or 30-31, a vector as any one of aspects 32-34, the polypeptide as in aspect 35, the delivery vehicle of any one of aspects 36-37, the cell of aspect 38, the immunogenic composition of any one of aspects 39-41 or 43, a therapeutic composition as in any one of aspects 42-43; and
   a pharmaceutically acceptable carrier.
45. A method of inducing a B-cell response and/or T-cell response to a virus in a subject in need thereof, comprising:
   administering, to the subject, the immunogenic composition of any one of aspects 39-41 or 43, or the therapeutic composition of any one of aspects 42-43, or a pharmaceutical formulation thereof.
46. The method of aspect 45, wherein the B cell response comprises antibody production.
47. A method of treating a viral infection in a subject in need thereof comprising:
   administering, to the subject in need thereof, the immunogenic composition of any one of aspects 39-41 or 43, or the therapeutic composition of any one of aspects 42-43, or a pharmaceutical formulation thereof in combination with an antiviral therapeutic.
48. A method of determining an infection status of a subject comprising:
   contacting immune cells derived from a subject with the immunogenic composition of any one of aspects 39-41 or 43 or a pharmaceutical formulation thereof; and
   detecting cross-reactivity of the immune cells to the immunogenic composition.
49. A method of massively parallel antigen profiling comprising:
   delivering to and expressing in a plurality of cells a pan-genomic library or a polynucleotide as in any one of aspects 1 or 30-31, wherein the pan-genomic library comprises a plurality of synthetic library expression constructs, each comprising a short synthetic polynucleotide and a pair of constant primers flanking the short synthetic polynucleotide, wherein the short synthetic polynucleotide has a sequence corresponding to a region of a genome; and
   determining the antigens presented by the cells.
50. The method of aspect 49, wherein delivery comprises infecting the plurality of cells with the viral particles comprising the pan-genomic library or the polynucleotide.
51. The method of any one of aspects 49-50, wherein determining the antigens comprises protein sequencing, mass spectrometry, Raman spectroscopy, immunodetection, chromatography, centrifugation, isoelectric focusing, or any combination thereof.
52. The method of any one of aspects 49-51, wherein determining the antigens comprises isolating MHC complexes from the cells and detecting peptides loaded in the MHC, wherein the MHC is optionally an HLA.
53. The method of any one of aspects 49-52, wherein the method further comprises evaluating an immune response to the antigens presented.
54. A method of determining translational regulation comprising:
   determining a first set of translated sequences by a method comprising:
      expressing a pan-genomic library in a first plurality of cells, wherein the pan-genomic library comprises a plurality of synthetic library expression constructs, each comprising a short synthetic polynucleotide and a pair of constant primers flanking the short synthetic polynucleotide, wherein the short synthetic polynucleotide has a sequence corresponding to a region of a genome;
      obtaining a sample of ribosomes comprising ribosome-protected mRNA fragments (RPFs) from the first plurality of cells;
      recovering ribosome footprints consisting essentially of the RPFs from the sample of ribosomes; and
      determining the sequence of the RPFs thereby identifying translated sequences to form the first set of translated sequences;
   determining a second set of translated sequences by a method comprising:
      expressing the pan-genomic library in a second plurality of cells;
      applying a stress to the second plurality of cells;
      obtaining a sample of ribosomes comprising ribosome-protected mRNA fragments (RPFs) from the second plurality of cells;
      recovering ribosome footprints consisting essentially of the RPFs from the sample of ribosomes; and
      determining the sequence of the RPFs thereby identifying translated sequences to form the second set of translated sequences,
      whereby similarities and differences in the sequences of the first and the second set of translated sequences indicates sequences that are translationally regulated or regulate translation.
55. The method of aspect 54, wherein the short synthetic polynucleotide is about 200 nucleotides.
56. The method of any one of aspects 54-55, wherein the short synthetic polynucleotide has a sequence corresponding to a region of a genome.
57. The method of aspect 56, wherein the short synthetic polynucleotide has a sequence corresponding to a region of a human genome, a non-human animal genome, a plant genome, a microbial genome, or a viral genome.
58. The method of any one of aspects 54-57, wherein the short synthetic polynucleotide has a sequence corresponding to
   a. an annotated region of a genome;
   b. an unannotated region of a genome;
   c. a mutation;
   d. a 5′UTR;
   e. a 3′UTR;
   f. an open reading frame; or
   g. any combination thereof.
59. The method of any one of aspects 54-58, wherein the short synthetic polynucleotide
   a. is a sequence according to a sequence in SEQ ID NOS: 1-9890 or SEQ ID NOS: 9891-16277;
   b. is a synthetic in silico designed sequence;
   c. does not have 100 percent identity to a genome sequence, optionally a human genome, a non-human animal genome, a plant genome, a microbial genome, or a viral genome;
   d. is not native to a genome, optionally a human genome, a non-human animal genome, a plant genome, a microbial genome, or a viral genome; or
   e. any combination thereof.
60. The method of any one of aspects 54-59, wherein expressing a pan-genomic library in a first plurality of cells and expressing a pan-genomic library in a second plurality of cells comprises expressing one or more vectors as in any one of aspects 26-41 or a vector library as in any one of aspects 42-45 in the first plurality of cells and the second plurality of cells.
61. The method of any one of aspects 54-60, wherein the stress is a small molecule agent, a biologic agent, a physical stress, a chemical stress, or any combination thereof.
62. A vector comprising:
   an optically active reporter construct comprising:
      a test polynucleotide;
      a first optically active protein encoding polynucleotide, wherein the first optically active protein encoding polynucleotide is operatively coupled in-frame with the test polynucleotide;
      a second optically active protein encoding polynucleotide, wherein the second optically active protein is a different optically active protein that the first optically active protein;
      a promoter, wherein the test polynucleotide, the first optically active protein encoding polynucleotide, and the second optically active protein encoding polynucleotide are operatively coupled to the promoter; and
      an internal ribosomal entry site (IRES), wherein the IRES is between the first optically active protein encoding polynucleotide and the second optically active protein encoding polynucleotide.
63. The vector of aspect 62, further comprising one or more additional regulatory elements, one or more additional reporters, viral replication elements and/or encoding polynucleotides, viral packaging elements and/or encoding polynucleotides, viral envelope protein encoding polynucleotides, long-terminal repeats, viral poly or any combination thereof.
64. The vector of any one of aspects 62-63, wherein the promoter is a constitutive promoter or an inducible promoter.
65. The vector of any one of aspects 62-64, further comprising one or more untranslated regions (UTRs), wherein the one or more untranslated regions are operatively coupled at a 5′ and/or a 3′ end of the test polynucleotide and/or the first optically active protein encoding polynucleotide.
66. The vector of aspect 65, wherein the one or more untranslated regions comprise or consist of 5′ UTRs, 3′ UTRs, or both.
67. The vector of any one of aspects 62-66, wherein the test polynucleotide comprises or consists of a short synthetic polynucleotide.
68. The vector of aspect 67, wherein the short synthetic polynucleotide is about 200 nucleotides.
69. The vector of any one of aspects 67-68, wherein the short synthetic polynucleotide has a sequence corresponding to a region of a genome.
70. The vector of aspect 69, wherein the short synthetic polynucleotide has a sequence corresponding to a region of a human genome, a non-human animal genome, a plant genome, a microbial genome, or a viral genome.
71. The vector of any one of aspects 67-70, wherein the short synthetic polynucleotide has a sequence corresponding to
   a. an annotated region of a genome;
   b. an unannotated region of a genome;
   c. a mutation;
   d. a 5′UTR;
   e. a 3′UTR;
   f. an open reading frame; or
   g. any combination thereof.
72. The vector of any one of aspects 67-71, wherein the short synthetic polynucleotide
   a. is a sequence according to a sequence in SEQ ID NOS: 1-9890 or SEQ ID NOS: 9891-16277;
   b. is a synthetic in silico designed sequence;
   c. does not have 100 percent identity to a genome sequence, optionally a human genome, a non-human animal genome, a plant genome, a microbial genome, or a viral genome;
   d. is not native to a genome, optionally a human genome, a non-human animal genome, a plant genome, a microbial genome, or a viral genome; or
   e. any combination thereof.
73. The vector of any one of aspects 62-72, wherein the vector is a viral vector, optionally a retroviral vector, lentiviral vector, adenoviral vector, or adeno-associated viral vector.
74. A vector system comprising:
   one or more vectors as in any one of aspects 62-73, an optionally one or more a viral envelope protein vectors, one or more viral packaging vectors, or any combination thereof.
75. The vector or vector system of any one of aspects 62-74, wherein the vector or vector system are capable of producing viral particles comprising the optically active reporter construct.
76. A pan-genomic, optionally pan-viral genomic, optically active reporter construct library, comprising:
   a plurality of vectors as in any one of aspects 67-74, wherein at least two test polynucleotides are different or the same, or wherein each test polynucleotide is different, and the test polynucleotides in each of the vectors of the plurality of vectors comprises or consists of a short synthetic polynucleotide.
77. An engineered viral particle comprising:
   a cargo comprising an optically active reporter construct, wherein the optically active reporter construct cargo comprises:
      a test polynucleotide;
      a first optically active protein encoding polynucleotide, wherein the first optically active protein encoding polynucleotide is operatively coupled in-frame with the test polynucleotide;
      a second optically active protein encoding polynucleotide, wherein the second optically active protein is a different optically active protein that the first optically active protein;
      a promoter, wherein the test polynucleotide, the first optically active protein encoding polynucleotide, and the second optically active protein encoding polynucleotide are operatively coupled to the promoter; and
      an internal ribosomal entry site (IRES), wherein the IRES is between the first optically active protein encoding polynucleotide and the second optically active protein encoding polynucleotide.
78. The engineered viral particle of aspect 77, wherein the promoter is a constitutive promoter or an inducible promoter.
79. The engineered viral particle of any one of aspects 77-78, further comprising one or more untranslated regions (UTRs), wherein the one or more untranslated regions are operatively coupled at a 5′ and/or a 3′ end of the test polynucleotide and/or the first optically active protein encoding polynucleotide.
80. The engineered viral particle of aspect 79, wherein the one or more untranslated regions comprise or consist of 5′ UTRs, 3′ UTRs, or both.
81. The engineered viral particle of any one of aspects 77-80, wherein the test polynucleotide comprises or consists of a short synthetic polynucleotide.
82. The engineered viral particle of aspect 81, wherein the short synthetic polynucleotide is about 200 nucleotides.
83. The engineered viral particle of any one of aspects 81-82, wherein the short synthetic polynucleotide has a sequence corresponding to a region of a genome.
84. The engineered viral particle of any one of aspects 81-83, wherein the short synthetic polynucleotide has a sequence corresponding to a region of a human genome, a non-human animal genome, a plant genome, a microbial genome, or a viral genome.
85. The engineered viral particle of any one of aspects 81-84, wherein the short synthetic polynucleotide has a sequence corresponding to
   a. an annotated region of a genome;
   b. an unannotated region of a genome;
   c. a mutation;
   d. a 5′UTR;
   e. a 3′UTR;
   f. an open reading frame; or
   g. any combination thereof.
86. The engineered viral particle of any one of aspects 81-85, wherein the short synthetic polynucleotide
   a. is a sequence according to a sequence in SEQ ID NOS: 1-9890 or SEQ ID NOS: 9891-16277;
   b. is a synthetic in silico designed sequence;
   c. does not have 100 percent identity to a genome sequence, optionally a human genome, a non-human animal genome, a plant genome, a microbial genome, or a viral genome;
   d. is not native to a genome, optionally a human genome, a non-human animal genome, a plant genome, a microbial genome, or a viral genome; or
   e. any combination thereof.
87. The engineered viral particle of any one of aspects 77-86, wherein the engineered viral particle is a viral particle, optionally a retroviral viral particle, lentiviral viral particle, adenoviral viral particle, or adeno-associated viral particle.
88. A pan-genomic, optionally pan-viral genomic, engineered viral particle library comprising:
   a plurality of engineered viral particles of any one of aspects 81-87, wherein at least two test polynucleotides are different or the same or wherein each test polynucleotide of the at least two test polynucleotides is different, and
   wherein the test polynucleotides in each of the vectors of the plurality of vectors comprises or consists of a short synthetic polynucleotide.
89. A method comprising:
   a. transducing one or more cells with one or more viral particles or a pan-viral engineered viral particle library as in any one of aspects 77-88 and allowing for genomic integration and expression of the optically active reporter construct in the one or more cells;
   b. selecting cells for genomic integration of the optically active reporter construct via sorting by detecting an optical signal produced from the first optically active protein and selecting cells that produce the optical signal from the second optically active protein;
   c. sorting selected cells from (b) into expression bins, wherein sorting selected cells from (b) comprises detecting an optical signal produced by the first optically active protein;
   d. sequencing one or more nucleic acids of the sorted selected cells by expression bin; and
   e. computing an expression score for each test polynucleotide by expression bin.
90. The method of aspect 89, wherein sequencing comprises DNA sequencing, RNA sequencing, or both.
91. The method of any one of aspects 89-90, wherein sequencing comprises genomic DNA sequencing.
92. The method of any one of aspects 89-91, wherein sequencing comprises next generation sequencing or third generation sequencing.
93. The method of any one of aspects 89-92, wherein sequencing comprises deep sequencing.
94. The method of any one of aspects 89-93, wherein sequencing comprises single cell sequencing.
95. A method of screening test agents and/or conditions comprising:
   a. transducing a first set of one or more cells with one or more viral particles or a pan-viral engineered viral particle library as in any one of aspects 77-88 and allowing for genomic integration and expression of the optically active reporter construct in the one or more cells;
   b. transducing a second set of the one or more cells with the one or more viral particles or a pan-viral engineered viral particle library as in any one of aspects 77-88 and allowing for genomic integration and expression of the optically active reporter construct in the one or more cells, wherein the second set of one or more cells comprises the same one or more cells as the first set of one or more cells, and wherein the one or more viral particles or the pan-viral engineered viral particle library used to transduce the second set of one or more cells is the same as the one or more viral particles or the pan-viral engineered viral particle library used to transduce the first set of one or more cells;
   c. exposing the second set of one or more cells to one or more test agents and/or conditions;
   d. selecting cells in each of the first set of one or more cells and second set of one or more cells for genomic integration of the optically active reporter construct via sorting by detecting an optical signal produced from the first optically active protein and selecting cells that produce the optical signal from the second optically active protein;
   e. sorting selected cells from (d) into expression bins, wherein sorting selected cells from (d) comprises detecting an optical signal produced by the first optically active protein;
   f. sequencing one or more nucleic acids of the sorted selected cells by expression bin; and;
   g. computing an expression score for each test polynucleotide by expression bin;
   h. comparing expression scores for each test polynucleotide between the first and second set of cells to determine an effect of the test agent and/or condition.
96. The method of aspect 95, wherein the test agent or condition is a small molecule agent,
a biologic agent, a physical stress, a chemical stress, or any combination thereof.
97. The method of any one of aspects 95-96, wherein sequencing comprises DNA sequencing, RNA sequencing, or both.
98. The method of any one of aspects 95-97, wherein sequencing comprises genomic DNA sequencing.
99. The method of any one of aspects 95-98, wherein sequencing comprises next generation sequencing or third generation sequencing.
100. The method of any one of aspects 95-99, wherein sequencing comprises deep sequencing.
101. The method of any one of aspects 95-100, wherein sequencing comprises single cell sequencing.

1. A polynucleotide comprising one or more viral polynucleotides selected from SEQ ID NOS: 1-9890 or SEQ ID NOS: 9891-16277.

2. A vector comprising:
a synthetic library expression construct comprising:
a short synthetic polynucleotide;
a pair of constant primers flanking the short synthetic polynucleotide, the pair of constant primers comprising a forward constant primer and a reverse constant primer, wherein the forward constant primer and the reverse constant primer are each independently coupled to a 5′ end or a 3′ end of the short synthetic polynucleotide;
a stop codon polynucleotide comprising one or more stop codons, wherein the stop codon polynucleotide is coupled to a constant primer coupled to the 3′ end of the short synthetic polynucleotide such that the constant primer coupled to the 3′ end of the short synthetic polynucleotide is between the stop codon polynucleotide and the short synthetic polynucleotide;
a poly A signal, wherein the poly A signal is operably coupled to the short synthetic polynucleotide, the pair of constant primers, and the stop codon polynucleotide; and
a promoter, wherein the promoter is operably coupled to the short synthetic polynucleotide, the pair of constant primers, and the stop codon polynucleotide, and the poly A signal.

3. The vector of claim 2, wherein the short synthetic polynucleotide is about 200 nucleotides, optionally having a sequence corresponding to a region of a human genome, a non-human animal genome, a plant genome, a microbial genome, or a viral genome, optionally an annotated or unannotated region, a mutation, a 5′UTR, a 3′UTR, an open reading frame, or any combination thereof; and wherein the short synthetic polynucleotide optionally is a sequence in SEQ ID NOS: 1-9890 or SEQ ID NOS: 9891-16277, is a synthetic in silico designed sequence, does not have 100% identity to a genome sequence, is not native to any genome, or any combination thereof.

4. (canceled)

5. (canceled)

6. (canceled)

7. (canceled)

8. (canceled)

9. (canceled)

10. (canceled)

11. (canceled)

12. The vector of claim 2, further comprising an additional regulatory element, a reporter gene, an origin of replication, a cloning sites, an internal ribosome entry sites, a transcription termination sequence, an inverted terminal repeat, a long terminal repeats, a trans-activating response elements, a central polypurine tract, a Psi element, a Rev response element, a packaging protein gene, a polymerase gene, an envelope protein gene, a capsid protein gene, a Rep protein gene, a U3 element, a repeat element (R), a unique 5′ element (U5), an untranslated region stabilization element, or any combination thereof; and/or wherein the vector comprises two or more synthetic library expression constructs, optionally wherein at least two constructs comprise a different short synthetic polynucleotide, optionally wherein each construct comprises a different short synthetic polynucleotide.

13. The vector of claim 2, further comprising a Woodchuck hepatitis virus post-transcriptional regulatory element (WPRE), wherein the WPRE is operably coupled to the short synthetic polynucleotide and the pair of constant primers.

14. (canceled)

15. The vector of claim 2, wherein the vector is a eukaryotic expression vector or a viral expression vector.

16. The vector of claim 2, wherein the promoter is a constitutive promoter, an inducible promoter or a conditional promoter.

17. (canceled)

18. A vector library comprising a plurality of vectors, wherein each vector of the plurality of vectors is as in claim 2, and wherein at least two vectors comprise different short synthetic polynucleotides, optionally wherein each vector comprises a different short synthetic polynucleotide, optionally wherein at least two of the vectors of the plurality of vectors comprise the same short synthetic polynucleotides.

19. (canceled)

20. (canceled)

21. (canceled)

22. (canceled)

23. (canceled)

24. A high through put method of determining translated sequences, the method comprising:
expressing a vector as in claim 2 in one or more cells under conditions sufficient to produce translation products;
obtaining a sample of ribosomes comprising ribosome-protected mRNA fragments (RPFs) from the cells;
recovering ribosome footprints consisting essentially of the RPFs from the sample of ribosomes; and
determining a sequence of the RPFs thereby identifying translated sequences; and optionally further comprising expressing the one or more vectors or vector library in a second plurality of cells under a stress condition, obtaining a sample of ribosomes comprising ribosome-protected mRNA fragments (RPFs) from the second plurality of cells, recovering ribosome footprints from the sample, determining a sequence of the RPFs, and comparing the translated sequences of the first and second plurality of cells to identify translationally regulated sequences.

25. The method of claim 24, wherein determining the sequence of the RPFs comprises nucleotide sequencing, optionally RNA sequencing or DNA sequencing of cDNA generated from the RPFs, optionally by next generation sequencing or third generation sequencing.

26. (canceled)

27. (canceled)

28. The method of claim 24, further comprising digesting unprotected mRNA prior to recovering ribosome footprints and/or removing rRNA from the sample containing ribosomes.

29. (canceled)

30. (canceled)

31. (canceled)

32. (canceled)

33. (canceled)

34. (canceled)

35. (canceled)

36. A delivery vehicle comprising:
the polynucleotide of claim 1, a vector comprising the polynucleotide, a polypeptide encoded by the polynucleotide or vector, or any combination thereof.

37. (canceled)

38. (canceled)

39. An immunogenic composition comprising:
the polynucleotide of claim 1, a vector comprising the polynucleotide, a polypeptide encoded by the polynucleotide or vector, a delivery vehicle comprising the polynucleotide, polypeptide, and/or vector, a cell of claim comprising the polynucleotide, vector, polypeptide, and/or delivery vehicle, or any combination thereof.

40. (canceled)

41. (canceled)

42. A therapeutic composition comprising:
the immunogenic composition of claim 39; and
an anti-viral therapeutic.

43. The immunogenic composition of claim 39 or a therapeutic composition comprising the immunogenic composition and an antiviral therapeutic, wherein the one or more polynucleotides is a synthetic mRNA vaccine.

44. A formulation comprising:
the polynucleotide of claim 1, a vector comprising the polynucleotide, a polypeptide encoded by the polynucleotide or vector, a delivery vehicle comprising the polynucleotide, the vector, the polypeptide or any combination thereof, a cell comprising the polynucleotide, a polypeptide encoded by the polynucleotide or vector, the delivery vehicle or any combination thereof, an immunogenic composition comprising the polynucleotide, the vector, the polypeptide, the delivery vehicle, the cell or any combination thereof, and/or a therapeutic composition comprising the immunogenic composition and an anti-viral therapeutic, or any combination thereof and
a pharmaceutically acceptable carrier.

45. (canceled)

46. (canceled)

47. (canceled)

48. (canceled)

49. A method of massively parallel antigen profiling comprising:
delivering to and expressing in a plurality of cells a pan-genomic library or a polynucleotide as in claim 1, wherein the pan-genomic library comprises a plurality of synthetic library expression constructs, each comprising a short synthetic polynucleotide and a pair of constant primers flanking the short synthetic polynucleotide, wherein the short synthetic polynucleotide has a sequence corresponding to a region of a genome;-and-, optionally wherein delivering comprises infecting the plurality of cells with viral particles comprising the pan-genomic library or the polynucleotide; and
determining the antigens presented by the cells, optionally by protein sequencing, mass spectrometry, Raman spectroscopy, immunodetection, chromatography, centrifugation, isoelectric focusing, or isolation of MHC complexes, optionally wherein isolating MHC complexes comprises detecting peptides loaded in the MHC complex, wherein the MHC is optionally an HLA; and optionally further comprising evaluating an immune response to the antigens presented.

50. (canceled)

51. (canceled)

52. (canceled)

53. (canceled)

54. A method of determining translational regulation comprising:
determining a first set of translated sequences by a method comprising:
expressing a pan-genomic library in a first plurality of cells, wherein the pan-genomic library comprises a plurality of synthetic library expression constructs, each comprising a short synthetic polynucleotide and a pair of constant primers flanking the short synthetic polynucleotide, wherein the short synthetic polynucleotide has a sequence corresponding to a region of a genome;
obtaining a sample of ribosomes comprising ribosome-protected mRNA fragments (RPFs) from the first plurality of cells;
recovering ribosome footprints consisting essentially of the RPFs from the sample of ribosomes; and
determining the sequence of the RPFs thereby identifying translated sequences to form the first set of translated sequences;
determining a second set of translated sequences by a method comprising:
expressing the pan-genomic library in a second plurality of cells;
applying a stress to the second plurality of cells;
obtaining a sample of ribosomes comprising ribosome-protected mRNA fragments (RPFs) from the second plurality of cells;
recovering ribosome footprints consisting essentially of the RPFs from the sample of ribosomes; and
determining the sequence of the RPFs thereby identifying translated sequences to form the second set of translated sequences,
whereby similarities and differences in the sequences of the first and the second set of translated sequences indicates sequences that are translationally regulated or regulate translation.

55. The method of claim 54, wherein the short synthetic polynucleotide is about 200 nucleotides, optionally having a sequence corresponding to a region of a human genome, a non-human animal genome, a plant genome, a microbial genome, or a viral genome, optionally an annotated or unannotated region, a mutation, a 5′UTR, a 3′UTR, an open reading frame, or any combination thereof; and/or wherein the short synthetic polynucleotide optionally is a synthetic in silico designed sequence, does not have 100% identity to a genome sequence, or is not native to any genome; and/or wherein the stress is a small molecule agent, a biologic agent, a physical stress, a chemical stress, or any combination thereof.

56. (canceled)

57. (canceled)

58. (canceled)

59. (canceled)

60. (canceled)

61. (canceled)

62. A vector comprising:
an optically active reporter construct comprising:
a test polynucleotide;
a first optically active protein encoding polynucleotide, wherein the first optically active protein encoding polynucleotide is operatively coupled in-frame with the test polynucleotide;
a second optically active protein encoding polynucleotide, wherein the second optically active protein is a different optically active protein that the first optically active protein;
a promoter, wherein the test polynucleotide, the first optically active protein encoding polynucleotide, and the second optically active protein encoding polynucleotide are operatively coupled to the promoter; and
an internal ribosomal entry site (IRES), wherein the IRES is between the first optically active protein encoding polynucleotide and the second optically active protein encoding polynucleotide.

63. The vector of claim 62, further comprising one or more additional regulatory elements, one or more additional reporters, viral replication elements and/or encoding polynucleotides, viral packaging elements and/or encoding polynucleotides, viral envelope protein encoding polynucleotides, long-terminal repeats, viral poly or any combination thereof; optionally further comprising one or more untranslated regions (UTRs) operatively coupled at a 5′ and/or 3′ end of the test polynucleotide and/or the first optically active protein encoding polynucleotide, optionally comprising 5′ UTRs, 3′ UTRs, or both: wherein the promoter is optionally a constitutive promoter or an inducible promoter; and/or wherein the vector is a viral vector, optionally a retroviral vector, lentiviral vector, adenoviral vector, or adeno-associated viral vector.

64. (canceled)

65. (canceled)

66. (canceled)

67. The vector of claim 62, wherein the test polynucleotide comprises or consists of a short synthetic polynucleotide, optionally about 200 nucleotides, optionally having a sequence corresponding to a region of a human genome, a non-human animal genome, a plant genome, a microbial genome, or a viral genome, optionally an annotated or unannotated region, a mutation, a 5′UTR, a 3′UTR, an open reading frame, or any combination thereof; and/or wherein the short synthetic polynucleotide optionally is a sequence in SEQ ID NOS: 1-9890 or SEQ ID NOS: 9891-16277, is a synthetic in silico designed sequence, does not have 100% identity to a genome sequence, or is not native to any genome.

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. A method comprising:
a. transducing one or more cells with one or more viral particles or a pan-viral engineered viral particle library comprising the optically active reporter construct of claim 62 and allowing for genomic integration and expression of the optically active reporter construct in the one or more cells;
b. selecting cells for genomic integration of the optically active reporter construct via sorting by detecting an optical signal produced from the first optically active protein and selecting cells that produce the optical signal from the second optically active protein;
c. sorting selected cells from (b) into expression bins, wherein sorting selected cells from (b) comprises detecting an optical signal produced by the first optically active protein;
d. sequencing one or more nucleic acids of the sorted selected cells by expression bin; and
e. computing an expression score for each test polynucleotide by expression bin, optionally wherein sequencing comprises DNA sequencing, RNA sequencing, or both, optionally comprising next generation sequencing, third generation sequencing, deep sequencing, or single cell sequencing.

90. (canceled)

91. (canceled)

92. (canceled)

93. (canceled)

94. (canceled)

95. A method of screening test agents and/or conditions comprising:
a. transducing a first set of one or more cells with one or more viral particles or a pan-viral engineered viral particle library comprising the optically active reporter construct of claim 62 and allowing for genomic integration and expression of the optically active reporter construct in the one or more cells;
b. transducing a second set of the one or more cells with the one or more viral particles or a pan-viral engineered viral particle library comprising the optically active reporter construct of claim 62 and allowing for genomic integration and expression of the optically active reporter construct in the one or more cells, wherein the second set of one or more cells comprises the same one or more cells as the first set of one or more cells, and wherein the one or more viral particles or the pan-viral engineered viral particle library used to transduce the second set of one or more cells is the same as the one or more viral particles or the pan-viral engineered viral particle library used to transduce the first set of one or more cells;
c. exposing the second set of one or more cells to one or more test agents and/or conditions;
d. selecting cells in each of the first set of one or more cells and second set of one or more cells for genomic integration of the optically active reporter construct via sorting by detecting an optical signal produced from the first optically active protein and selecting cells that produce the optical signal from the second optically active protein;
e. sorting selected cells from (d) into expression bins, wherein sorting selected cells from (d) comprises detecting an optical signal produced by the first optically active protein;
f. sequencing one or more nucleic acids of the sorted selected cells by expression bin; and;
g. computing an expression score for each test polynucleotide by expression bin;
h. comparing expression scores for each test polynucleotide between the first and second set of cells to determine an effect of the test agent and/or condition: wherein the test agent or condition optionally comprises a small molecule agent, a biologic agent, a physical condition, or a chemical condition, and wherein sequencing optionally comprises DNA sequencing, RNA sequencing, or both, optionally comprising next generation sequencing, third generation sequencing, deep sequencing, or single cell sequencing.

96. (canceled)

97. (canceled)

98. (canceled)

99. (canceled)

100. (canceled)

101. (canceled)
